(19) United States
(12) Patent Application Publication
(10) Pub. No.: US 2026/0297092 A1
(43) Pub. Date: Oct. 1, 2026

(54) PYRAZOLOPYRIMIDINE DERIVATIVES AS INHIBITORS OF NLRP3

(71) Applicant: Takeda Pharmaceutical Company Limited, Osaka (JP)

(72) Inventors: Satoshi MIKAMI, Kanagawa (JP); Misaki HOMMA, Kanagawa (JP); Tomoaki HASUI, Kanagawa (JP); Yuta TANAKA, Kanagawa (JP); Chunxiang WANG, Kanagawa (JP); Masaki DAINI, Kanagawa (JP); Keisuke IMAMURA, Kanagawa (JP); Fumiaki KIKUCHI, Kanagawa (JP); Shinji MORIMOTO, Kanagawa (JP); Akinori TOITA, Kanagawa (JP); Takafumi YUKAWA, Kanagawa (JP); Dilhumar UYGHUR, Kanagawa (JP); Tomohiro OHASHI, Kanagawa (JP); Kosuke HIDAKA, Kanagawa (JP); Shinji NAKAMURA, Kanagawa (JP); Makoto FUSHIMI, Kanagawa (JP); Takeshi WAKABAYASHI, Kanagawa (JP); Takeshi YAMAMOTO, Kanagawa (JP); Norio OYABU, Kanagawa (JP); Ignacio IBANEZ SANCHEZ, Kanagawa (JP); Masashi TOYOFUKU, Kanagawa (JP)

(21) Appl. No.: 19/474,980

(22) PCT Filed: Apr. 11, 2024
(86) PCT No.: PCT/IB2024/053566

§ 371 Date: Oct. 13, 2025

Related U.S. Application Data
(60) Provisional application No. 63/496,053, filed on Apr. 14, 2023.
(60) Provisional application No. 63/515,968, filed on Jul. 27, 2023.

(51) Int. Cl. C07D 473/06; A61K 9/20; A61K 9/48; C07D 519/00

(52) U.S. Cl. CPC C07D 473/06; A61K 9/2018; A61K 9/2054; A61K 9/4858; A61K 9/4866; C07D 519/00

(57) ABSTRACT

The invention provides a compound of Formula (I), or a pharmaceutically acceptable salt thereof: wherein L, X, Y, R¹, R², R³, R⁴, R⁵, R⁶, R⁷ and R⁸ are as defined in the specification, for treatment of a disease, disorder or condition associated with NLRP3, including a disease, disorder or condition associated with a heterozygous gain of function mutation in the NLRP3 gene such as cryopyrin-associated periodic syndrome (CAPS).

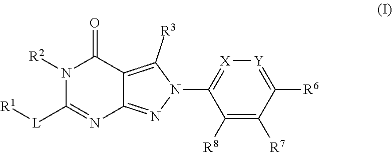

### FIELD OF THE INVENTION

This invention relates to heterocyclic compounds which are inhibitors of the NLRP3 inflammasome, to medicaments which contain them, and to their use to treat diseases, disorders and/or conditions associated with NLRP3, including neurodegenerative diseases, such as Parkinson's disease, Alzheimer's disease, Huntington's disease, amyotrophic lateral sclerosis, prion disease and obesity with certain additional risk factors for cardiovascular disease.

### BACKGROUND OF THE INVENTION

More than 1% of the world's population suffers from neurodegenerative diseases, including Parkinson's disease (PD), Alzheimer's disease (AD), Huntington's disease (HD), amyotrophic lateral sclerosis (ALS) and prion disease, all of which lack effective therapies. The incidence of neurodegenerative diseases is expected to double in the coming decades, especially affecting countries with an aging population. See I. Fernández-Cruz and E. Reynaud, “Proteasome Subunits Involved in Neurodegenerative Diseases,” Arch Med Res. 52(1):1-14 (2021).

One of the pathological hallmarks of neurodegenerative diseases is the aggregation of certain proteins into oligomers or fibrils. These conformational changes result in neurotoxicity, leading to inflammation and neurodegeneration. Although the clinical presentations of these diseases are heterogeneous, they often share common underlying mechanisms and pathophysiologies. See B. N. Dugger and D. W. Dickson, “Pathology of Neurodegenerative Diseases,” Cold Spring Harbor Perspect Biol 9(7): a028035 (2017). Indeed, systemic activation of the innate immune system, which is the first line of host defense against pathogens and tissue injury, and subsequent neuroinflammation play a key role in the onset and the progression of these diseases. See S. Amor, F. Puentes, D. Baker, et al., “Inflammation in neurodegenerative diseases,” Immunology 129(2):154-69 (2010). Neuroinflammation is a physiological response to exogenous and endogenous insults that target the central nervous system (CNS) and represents a protective response in the brain. However, excessive inflammatory responses are detrimental to the CNS. See L. I. Labzin, M. T. Heneka and E. Latz, “Innate Immunity and Neurodegeneration,” Annu Rev Med 69:437-449 (2018).

Microglia, which are myeloid cells of the CNS, play a major role during innate immune responses in the CNS. They express pattern recognition receptors (PRRs) which enable the host to recognize pathogen-associated molecular patterns (PAMPS) and host- or environment-derived danger-associated molecular patterns (DAMPS). See R. M. Ransohoff, M. A. Brown, “Innate immunity in the central nervous system,” J Clin Invest 122(4):1164-71 (2012). PRRs include Toll-like receptors, C-type lectin receptors, RIG-1 like receptors, and nucleotide-binding oligomerization domain-like receptors (NLRs). See P. Broz and V. M. Dixit, “Inflammasomes: mechanism of assembly, regulation and signaling,” Nat Rev Immunol 16(7):407-20 (2016). Engagement of PRRs activates a variety of inflammatory signaling pathways to eliminate infection and repair damaged tissue. The ongoing inflammation found in a variety of neurodegenerative diseases can be maintained by the key innate immune sensor for danger signals, the inflammasomes. There are several different inflammasomes, all defined by the PRRs they contain. Among the PRRs from the NLR family, the NLRs—NLRP1, NLRP3, NLRC4—and two other PRRs—Pyrin and AIM2—are known to form inflammasomes. See D. Zheng, T. Liwinski and E. Elinav, “Inflammasome activation and regulation: toward a better understanding of complex mechanisms,” Cell Discov 6:36 (2020).

The NLRP3 (nucleotide-binding domain (NOD), leucine-rich repeats-containing domain (LRR), and pyrin domain-containing 3) inflammasome has been the subject of intense interest in the past decade. See N. Kelley, D. Jeltema, Y. Duan, et al., “The NLRP3 Inflammasome: An Overview of Mechanisms of Activation and Regulation,” Int J Mol Sci 20(13):3328 (2019). The NLRP3 inflammasome consists of three main components: a pattern recognition receptor (PRR) protein, NLRP3; an apoptosis-associated speck-like protein (ASC) containing a caspase activation and recruitment domain (CARD), which functions as a central adaptor protein; and an inflammatory caspase, caspase-1. See Kelley et al. (2019). NLRP3 is comprised of three domains: an amino-terminal pyrin domain (PYD); a central NACHT domain, having ATPase activity that is vital for NLRP3 self-association and oligomerization; and a carboxy-terminal LRR domain. See Broz and Dixit (2016).

The activation of NLRP3 inflammasome involves a two-step process. A first “priming” signal is generated by the detection of PAMPs or DAMPs via TLRs. This priming signal results in NF-κB-dependent transcriptional upregulation of NLRP3 and pro-IL-1, but also controls post-translational modifications of NLRP3. See J. Yang, Z. Liu and T. S. Xiao, “Post-translational regulation of inflammasomes,” Cell Mol Immunol 14(1):65-79 (2017). The initial trigger is followed by a second “activation” signal (β-amyloid, α-synuclein and other proteinaceous insults, ATP, crystals, nucleic acids, toxins) that induces conformational change of the various inflammasome components to subsequently assemble and nucleate the oligomerization of monomeric NLRP3, leading to the formation and activation of the NLRP3 inflammasome. See A. Lu, V. G Magupalli, J. Ruan, et al., “Unified polymerization mechanism for the assembly of ASC-dependent inflammasomes,” Cell 156(6):1193-1206 (2014). This large multimeric protein acts via caspase-1 dependent proteolytic cleavage of several proteins, including pro-interleukin (pro-IL)-18 and pro-IL-1β to their mature inflammatory cytokines, IL-18 and IL-1β. See Kelley et al. (2019). Caspase-1 can also cleave gasdermin D (GSDMD), which facilitates GSDMD's insertion into cellular membranes to form pores, thus initiating a specific kind of cell death called pyroptosis that releases the soluble intracellular fraction which fuels the inflammatory response. See S. L. Fink and B. T Cookson, “Caspase-1-dependent pore formation during pyroptosis leads to osmotic lysis of infected host macrophages,” Cell Microbiol 8(11):1812-25 (2006).

Besides this “canonical” NLRP3 inflammasome activation pathway, a “noncanonical” NLRP3 activation pathway has been described in the literature. The noncanonical pathway involves the activation of caspase-4/5 (or its mouse ortholog caspase-11) by cytosolic LPS, the induction of pyroptosis through the cleavage of GSDMD, and the release of high mobility group box 1 protein (HMGB1), resulting in the production of IL-1β. See M. Lamkanfi and V. M. Dixit, “Mechanisms and functions of inflammasomes,” Cell 157(5):1013-22 (2014); F. Shi, Y. Yang, M. Kouadir M, et al., “Inhibition of phagocytosis and lysosomal acidification suppresses neurotoxic prion peptide-induced NALP3 inflammasome activation in BV2 microglia,” J Neuroimmunol 260(1-2):121-5 (2013). In both pathways, the activation of NLRP3 inflammasome results in the generation of the biologically active form of pro-inflammatory cytokines IL-1β and IL-18 that initiate inflammatory signaling cascades, contributing to neuroinflammation, neuronal injury and cell death. See S. M Allan, P. J. Tyrrell and N. J. Rothwell, “Interleukin-1 and neuronal injury,” Nat Rev Immunol, 5(8):629-40 (2005); A. Alboni, D. Cervia, S. Sugama, et al., “Interleukin 18 in the CNS,” J Neuroinflammation, 7:9 (2010).

Heterozygous gain of function mutations in the NLRP3 gene have been associated with the development of an autoinflammatory condition called cryopyrin-associated periodic syndromes (CAPS). See L. M. Booshehri and H. M. Hoffman, “CAPS and NLRP3,” J Clin Immunol 39(3):277-286 (2019). This is a rare inherited autoinflammatory disorder characterized by systemic, cutaneous, musculoskeletal and central nervous system inflammation, and is estimated to affect about 1 to 3 individuals per million people worldwide. See L. Cuisset, I. Jeru, B. Dumont, et al., “Mutations in the autoinflammatory cryopyrin-associated periodic syndrome gene: epidemiological study and lessons from eight years of genetic analysis in France,” Ann Rheum Dis 70(3):495-9 (2011); Erratum in: Ann Rheum Dis 71(7):1264 (2012). Clinicians classify CAPS disorders based on the severity of symptoms. The most severe form of CAPS is known as neonatal-onset multisystem inflammatory disease (NOMID/CINCA). An intermediate form of CAPS is called Muckle-Wells syndrome (MWS). The familial cold autoinflammatory syndrome (FCAS) is a milder form of CAPS, which is triggered by low temperatures. See Booshehri and Hoffman (2019). Current anti-IL-1 therapies (anakinra, rilonacept, canakinumab) have proven successful in treating CAPS, but clinical experience over the last decade has shown that some CAPS patients are less responsive over time and require higher or more frequent dosing or switching of therapies. See R. Caorsi, L. Lepore, F. Zulian, et al., “The schedule of administration of canakinumab in cryopyrin associated periodic syndrome is driven by the phenotype severity rather than the age,” Arthritis Res Ther 15(1): R33 (2013); S. Urien, C. Bardin, B. Bader-Meunier, et al., “Anakinra pharmacokinetics in children and adolescents with systemic-onset juvenile idiopathic arthritis and autoinflammatory syndromes,” BMC Pharmacol Toxicol 14:40 (2013).

Beyond neurodegenerative diseases, the consequences of NLRP3 inflammasome hyper-activation is systemic chronic low-grade inflammation, a cardinal feature of obesity and insulin resistance. Obesity, an excessive accumulation of body fat, is recognized as the cause of a plethora of health complications. It is now recognized that obesity is associated with the onset of low-grade metabolic inflammation in both peripheral tissues and the brain in particular the hypothalamus, the brain area responsible for appetite and satiety regulation (Sonnefeld et al., “Is human obesity an inflammatory disease of the hypothalamus?” Eur J Endocrinol. 188(3):R37-R45 (2023)).

The prolonged inflammation that characterizes obesity is induced throughout the proliferating tissue (Hotamisligil and Erbay “Nutrient sensing and inflammation in metabolic diseases” Nat Rev Immunol. (12):923-34 (2008); Odegaard and Chawla “Mechanisms of macrophage activation in obesity-induced insulin resistance” Nat Clin Pract Endocrinol Metab. (11):619-26 (2008)). The accumulation of excessive fat mass, accompanied by the development of adipocytes promotes macrophage infiltration inside tissues (Weisberg et al., “Obesity is associated with macrophage accumulation in adipose tissue” J Clin Invest. 112(12):1796-808 (2003)). The increased inflammation of tissue is cytokine in nature and contributes to the progression of diabetes mellitus (Olefsky and Glass “Macrophages, inflammation, and insulin resistance” Annu Rev Physiol. 72:219-46 (2010); Shoelson et al. “Obesity, inflammation, and insulin resistance” Gastroenterology 132(6):2169-80 (2007)). In addition, IL-1β is thought to be connected with the progression of obesity-associated insulin resistance (Jager et al., “Interleukin-1beta-induced insulin resistance in adipocytes through down-regulation of insulin receptor substrate-1 expression” Endocrinology 148(1):241-51 (2007); Netea et al., “Deficiency of interleukin-18 in mice leads to hyperphagia, obesity and insulin resistance” Nat Med. 12(6):650-6 (2006); Zorrilla et al., “Interleukin-18 controls energy homeostasis by suppressing appetite and feed efficiency” Proc Natl Acad Sci USA. 104(26):11097-102 (2007)). Furthermore, overfeeding leads to stimulation of caspase-1 in adipose tissue in experimental animals (Stienstra et al., “The inflammasome-mediated caspase-1 activation controls adipocyte differentiation and insulin sensitivity” Cell Metab. 12(6):593-605 (2010)).

Microglia and astrocytes, which are brain resident glia cells, can predispose individuals to excessive weight gain by impairing the hypothalamic energy homeostasis system (Yoo et al., “Tanycyte ablation in the arcuate nucleus and median eminence increases obesity susceptibility by increasing body fat content in male mice” Glia 68(10):1987-2000 (2020); Douglass et al., “Obesity-associated microglial inflammatory activation paradoxically improves glucose tolerance” Cell Metab. 35(9):1613-1629.e8 (2023); Sonnefeld et al., “Is human obesity an inflammatory disease of the hypothalamus?” Eur J Endocrinol. 188(3):R37-R45 (2023)). It is a predisposition because the gliosis occurs rapidly before the actual weight gain. This inflammation impairs the local signaling of insulin and leptin leading to dysfunction of the regulation of energy balance and thus, weight gain. Although not fully elucidated yet, the mechanism by which this gliosis response occurs involves the passage of dietary saturated fatty acids into the cerebrospinal fluid (Melo et al., “Palmitate Is Increased in the Cerebrospinal Fluid of Humans with Obesity and Induces Memory Impairment in Mice via Pro-inflammatory TNF-α” Cell Rep. 30(7):2180-2194.e8 (2020)), thus promoting inflammatory activation of hypothalamic microglia, potentially via Toll-like receptor 4-dependent mechanisms (Milanski et al., “Saturated fatty acids produce an inflammatory response predominantly through the activation of TLR4 signaling in hypothalamus: implications for the pathogenesis of obesity” 29(2):359-70 (2009); Valdearcos et al., “Microglia dictate the impact of saturated fat consumption on hypothalamic inflammation and neuronal function” Cell Rep. 9(6):2124-38 (2014); Folick et al., “Metabolic factors in the regulation of hypothalamic innate immune responses in obesity” 54(4):393-402 (2022)). Dysregulated hypothalamic circuits change the interaction between neuronal and non-neuronal cells, contributing to the establishment of inflammatory processes. Interventions that block this gliosis response have demonstrated reductions of the excess weight gain (Valdearcos et al., “Microglia dictate the impact of saturated fat consumption on hypothalamic inflammation and neuronal function” Cell Rep. 9(6):2124-38 (2014); Douglass et al., “Obesity-associated microglial inflammatory activation paradoxically improves glucose tolerance” Cell Metab. 35(9):1613-1629.e8 (2023)). NLRP3 deficiency has been reported to inhibit the progression of obesity-linked insulin resistance (Stienstra et al., “The inflammasome-mediated caspase-1 activation controls adipocyte differentiation and insulin sensitivity” Cell Metab. 12(6):593-605 (2010)).

Several small molecule inhibitors have recently been reported that block the NLRP3 inflammasome pathways. These include the prototype NLRP3 inhibitor MCC-950. See R. C. Coll, J. R. Hill, C. J. Day, et al., “MCC950 directly targets the NLRP3 ATP-hydrolysis motif for inflammasome inhibition,” Nat Chem Biol 15(6):556-559 (2019); R. C. Coll, A. A. Robertson, J. J. Chae, et al., “A small-molecule inhibitor of the NLRP3 inflammasome for the treatment of inflammatory diseases,” Nat Med 21(3):248-55 (2015). Other NLRP3 inhibitors include Bay 11-7082, CY-09, oridonin, tranilast, INF-39, glyburide and JC-124. See W. Jiang, M. Li, F. He, et al., “Inhibition of NLRP3 inflammasome attenuates spinal cord injury-induced lung injury in mice,” J Cell Physiol 234(5):6012-6022 (2019). MCC-950 has been used in many studies as a pharmacological tool to demonstrate NLRP3 inflammasome as a viable drug target to development therapeutics for human diseases. See S. E. Corcoran, R. Halai and M. A. Cooper, “Pharmacological Inhibition of the Nod-Like Receptor Family Pyrin Domain Containing 3 Inflammasome with MCC950,” Pharmacol Rev 73(3):968-1000 (2021).

Inhibitors of the NLRP3 inflammasome pathways are expected to be useful for treating neurodegenerative diseases, including Parkinson's disease, Alzheimer's disease, Huntington's disease, amyotrophic lateral sclerosis and prion disease for treating CAPS associated with heterozygous gain of function mutations in the NLRP3 gene and for treating obesity with certain additional risk factors for cardiovascular disease.

WO 2023/032987 discloses the following 6-aminopyrazolopyrimidine derivatives as compounds having an inhibitory activity on NLRP3.

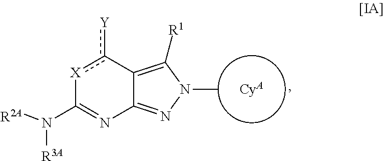

WO 2024/048519 discloses the following 6-aminopyrazolopyrimidine derivatives as compounds having an inhibitory activity on NLRP3.

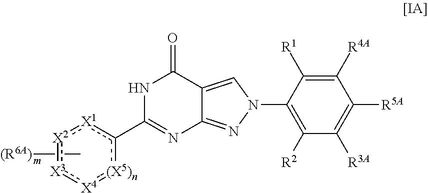

U.S. Pat. No. 9,688,681 discloses heterocyclic compounds for controlling animal pests including following three compounds.

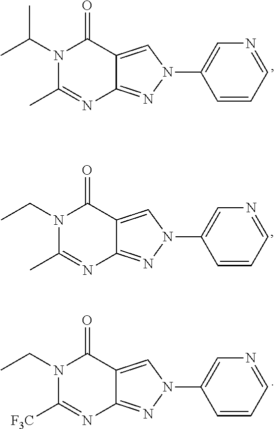 [SMILES: Cc1nc2nn(-c3cccnc3)cc2c(=O)n1C(C)C ; CCn1c(C)nc2nn(-c3cccnc3)cc2c1=O ; CCn1c(C(F)(F)F)nc2nn(-c3cccnc3)cc2c1=O]

### SUMMARY OF THE INVENTION

This invention provides heterocyclic compounds, or pharmaceutically acceptable salts thereof, or solvates thereof, or hydrates thereof. This invention also provides medicaments that contain pyrazolopyrimidone derivatives and provides for their use to treat diseases, disorders and/or conditions associated with NLRP3, including Parkinson's disease, Alzheimer's disease, Huntington's disease, amyotrophic lateral sclerosis and prion disease, and other neurodegenerative disorders.

One aspect of the invention provides [1] a compound of Formula (I), or a pharmaceutically acceptable salt thereof:

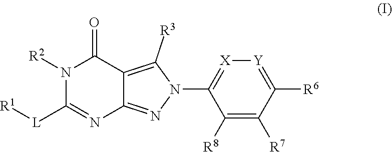

wherein
L is O or a bond;
X is N or CR⁴;
Y is N or CR⁵;
R¹ is an optionally substituted C₁₋₆ alkyl group, an optionally substituted C₃₋₈ cycloalkyl group, an optionally substituted C₆₋₁₄ aryl group, or an optionally substituted 4- to 6-membered heterocyclic group with the proviso that when L is a bond, then the 4- to 6-membered heterocyclic group is a 4- to 6-membered non-aromatic heterocyclic group which is linked to the pyrazolopyrimidone ring by a carbon-carbon bond;
R² is a hydrogen atom, an optionally substituted C₁₋₆ alkyl group, or an optionally substituted C₃₋₈ cycloalkyl group;
R³ is a hydrogen atom, an optionally substituted C₁₋₆ alkyl group, an optionally substituted C₃₋₈ cycloalkyl group, or a halogen atom; and
R⁴, R⁵, R⁶, R⁷ and R⁸ are each independently a hydrogen atom, an optionally substituted C₁₋₆ alkyl group, an optionally substituted C₃₋₈ cycloalkyl group, an optionally substituted C₂₋₆ alkenyl group, a halogen atom, a hydroxy group, an optionally substituted C₁₋₆ alkoxy group, an optionally substituted 5- or 6-membered heterocyclic group, a cyano group, an amino group or a nitro group;
with the proviso that (1) 2,5-dihydro-6-methyl-5-(1-methylethyl)-2-(3-pyridinyl)-4H-pyrazolo[3,4-d]pyrimidin-4-one, (2) 5-ethyl-2,5-dihydro-6-methyl-2-(3-pyridinyl)-4H-pyrazolo[3,4-d]pyrimidin-4-one and (3) 5-ethyl-2,5-dihydro-2-(3-pyridinyl)-6-(trifluoromethyl)-4H-pyrazolo[3,4-d]pyrimidin-4-one are excluded. (hereinafter, it is sometimes also referred as “compound (I)”).

Another aspect of the invention provides the following [2] to [33].

[2] The compound or pharmaceutically acceptable salt thereof as defined in the above [1], wherein
L is O or a bond;
X is N or CR⁴;
Y is N or CR⁵;
R¹ is
(1) a C₁₋₆ alkyl group optionally substituted by 1 to 3 substituents selected from
   (a) a halogen atom,
   (b) a hydroxy group,
   (c) an optionally halogenated C₁₋₆ alkoxy group,
   (d) a di-C₁₋₆ alkylamino group,
   (e) a C₇₋₁₆ aralkyloxy group,
   (f) a 5- or 6-membered aromatic heterocyclic group,
   (g) a 5- or 6-membered non-aromatic heterocyclic group,
   (h) a carboxy group,
   (i) a 4- to 6-membered non-aromatic heterocyclyloxy group, and
   (j) a cyano group,
(2) a C₃₋₈ cycloalkyl group optionally substituted by 1 to 3 substituents selected from
   (a) a C₁₋₆ alkyl group,
   (b) a C₁₋₆ alkoxy group,
   (c) a halogen atom,
   (d) a cyano group, and
   (e) a group represented by the formula: —(CH₂)ₐ—O—(CH₂)_{b}—, wherein each of a and b is the integer of 0 to 3 and the sum of a and b is 2 to 4,
(3) a C₆₋₁₄ aryl group optionally substituted by 1 to 3 substituents selected from
   (a) a halogen atom, and
   (b) a C₁₋₆ alkoxy group,
(4) a 5- or 6-membered aromatic heterocyclic group optionally substituted by 1 to 3 substituents selected from
   (a) a halogen atom,
   (b) a C₁₋₆ alkyl group optionally substituted by 1 to 3 substituents selected (i) halogen atom and (ii) C₁₋₆ alkoxy group,
   (c) a C₁₋₆ alkoxy group, and
   (d) a group represented by the formula: —(CH₂)ₐ—O—(CH₂)_{b}—, together with the 5- or 6-membered aromatic heterocyclic group to which it is attached, forming a fused 8- to 10-membered heterocyclic group, wherein each of a and b is the integer of 0 to 3 and the sum of a and b is 2 to 4, or
(5) a 4- to 6-membered non-aromatic heterocyclic group optionally substituted by 1 to 3 substituents selected from
   (a) an oxo group,
   (b) a C₁₋₆ alkyl group,
   (c) a halogen atom, and
   (d) a C₁₋₆ alkoxy group;
R² is
(1) a hydrogen atom, or
(2) a C₁₋₆ alkyl group;
R³ is
(1) a hydrogen atom,
(2) a C₁₋₆ alkyl group, or
(3) a halogen atom;
R⁴ and R⁸ are each independently
(1) a hydrogen atom,
(2) a C₁₋₆ alkyl group optionally substituted by 1 to 4 substituents selected from
   (i) halogen atoms,
   (ii) hydroxy group, and
   (iii) C₁₋₆ alkoxy group,
(3) a C₃₋₈ cycloalkyl group optionally substituted by 1 to 3 halogen atoms,
(4) a halogen atom,
(5) a hydroxy group,
(6) a C₁₋₆ alkoxy group optionally substituted by 1 to 3 halogen atoms,
(7) a 5- or 6-membered aromatic heterocyclic group, or
(8) a cyano group;
R⁵ and R⁷ are each independently
(1) a hydrogen atom,
(2) a C₁₋₆ alkyl group optionally substituted by 1 to 3 halogen atoms,
(3) a halogen atom, or
(4) a C₃₋₈ cycloalkyl group; and
R⁶ is
(1) a hydrogen atom,
(2) a C₁₋₆ alkyl group optionally substituted by 1 to 3 halogen atoms,
(3) a C₃₋₈ cycloalkyl group optionally substituted by 1 to 3 halogen atoms,
(4) a C₂₋₆ alkenyl group,
(5) a halogen atom,
(6) a C₁₋₆ alkoxy group optionally substituted by 1 to 3 halogen atoms,
(7) a 5- or 6-membered aromatic heterocyclic group,
(8) an amino group, or
(9) a nitro group.

[3] The compound or pharmaceutically acceptable salt thereof as defined in the above [1], wherein
L is O or bond;
X is CR⁴;
Y is CR⁵;
R¹ is a C₁₋₆ alkyl group optionally substituted by an optionally halogenated C₁₋₆ alkoxy group;
R² is a hydrogen atom;
R³ is a hydrogen atom;
R⁴ and R⁸ are each independently
(1) a C₁₋₆ alkyl group optionally substituted by 1 to 3 halogen atoms, or
(2) a halogen atom;
R⁵ and R⁷ are both hydrogen atoms; and
R⁶ is
(1) a C₃₋₈ cycloalkyl group optionally substituted by 1 to 3 halogen atoms,
(2) a halogen atom, or
(3) a C₁₋₆ alkoxy group optionally substituted by 1 to 3 halogen atoms.

[4] The compound or pharmaceutically acceptable salt thereof as defined in the above [1], wherein
L is O;
X is N or CR⁴;
Y is N or CR⁵;
R¹ is an optionally substituted C₁₋₆ alkyl group, an optionally substituted C₃₋₈ cycloalkyl group, an optionally substituted C₆₋₁₄ aryl group, or an optionally substituted 4- to 6-membered heterocyclic group;
R² is a hydrogen atom, or an optionally substituted C₁₋₆ alkyl group;
R³ is a hydrogen atom, an optionally substituted C₁₋₆ alkyl group, or a halogen atom;
R⁴ and R⁸ are each independently a hydrogen atom, an optionally substituted C₁₋₆ alkyl group, an optionally substituted C₃₋₈ cycloalkyl group, a halogen atom, a hydroxy group, an optionally substituted C₁₋₆ alkoxy group, an optionally substituted 5- or 6-membered heterocyclic group or a cyano group;
R⁵ and R⁷ are each independently a hydrogen atom, an optionally substituted C₁₋₆ alkyl group, or a halogen atom; and
R⁶ is a hydrogen atom, an optionally substituted C₁₋₆ alkyl group, an optionally substituted C₃₋₈ cycloalkyl group, an optionally substituted C₂₋₆ alkenyl group, a halogen atom, an optionally substituted C₁₋₆ alkoxy group, or an optionally substituted 5- or 6-membered heterocyclic group.

[5] The compound or pharmaceutically acceptable salt thereof as defined in the above [1], wherein
L is O;
X is N or CR⁴;
Y is N or CR⁵;
R¹ is
(1) a C₁₋₆ alkyl group optionally substituted by 1 to 3 substituents selected from
   (a) a halogen atom,
   (b) a hydroxy group,
   (c) an optionally halogenated C₁₋₆ alkoxy group,
   (d) a di-C₁₋₆ alkylamino group, and
   (e) a C₇₋₁₆ aralkyloxy group,
(2) a C₃₋₈ cycloalkyl group optionally substituted by 1 to 3 substituents selected from
   (a) a C₁₋₆ alkoxy group,
   (b) a cyano group, and
   (c) a group represented by the formula: —(CH₂)ₐ—O—(CH₂)_{b}—, wherein each of a and b is the integer of 0 to 3 and the sum of a and b is 2 to 4,
(3) a C₆₋₁₄ aryl group, or
(4) a 4- to 6-membered non-aromatic heterocyclic group;
R² is
(1) a hydrogen atom, or
(2) a C₁₋₆ alkyl group;
R³ is
(1) a hydrogen atom,
(2) a C₁₋₆ alkyl group, or
(3) a halogen atom;
R⁴ and R⁸ are each independently
(1) a hydrogen atom,
(2) a C₁₋₆ alkyl group optionally substituted by 1 to 4 substituents selected from
   (i) halogen atoms,
   (ii) hydroxy group, and
   (iii) C₁₋₆ alkoxy group,
(3) a C₃₋₈ cycloalkyl group optionally substituted by 1 to 3 halogen atoms,
(4) a halogen atom,
(5) a hydroxy group,
(6) a C₁₋₆ alkoxy group,
(7) a 5- or 6-membered aromatic heterocyclic group, or
(8) a cyano group;
R⁵ and R⁷ are each independently
(1) a hydrogen atom,
(2) a C₁₋₆ alkyl group, or
(3) a halogen atom; and
R⁶ is
(1) a hydrogen atom,
(2) a C₁₋₆ alkyl group optionally substituted by 1 to 3 halogen atoms,
(3) a C₃₋₈ cycloalkyl group optionally substituted by 1 to 3 halogen atoms,
(4) a C₂₋₆ alkenyl group,
(5) a halogen atom,
(6) a C₁₋₆ alkoxy group optionally substituted by 1 to 3 halogen atoms, or
(7) a 5- or 6-membered aromatic heterocyclic group.

[6] The compound or pharmaceutically acceptable salt thereof as defined in the above [1], wherein
L is O;
X is CR⁴;
Y is CR⁵;
R¹ is a C₁₋₆ alkyl group optionally substituted by 1 to 3 C₁₋₆ alkoxy group;
R² is a hydrogen atom;
R³ is a hydrogen atom;
R⁴ and R⁸ are each independently
(1) a C₁₋₆ alkyl group optionally substituted by 1 to 3 halogen atoms, or
(2) a halogen atom;
R⁵ and R⁷ are both hydrogen atoms; and
R⁶ is
(1) a C₃₋₈ cycloalkyl group optionally substituted by 1 to 3 halogen atoms,
(2) a halogen atom, or
(3) a C₁₋₆ alkoxy group optionally substituted by 1 to 3 halogen atoms.

[7] The compound or pharmaceutically acceptable salt thereof as defined in the above [1], wherein
L is O;
X is CR⁴;
Y is CR⁵;
R¹ is a C₁₋₆ alkyl group;
R² is a hydrogen atom;
R³ is a hydrogen atom;
R⁴ and R⁸ are both C₁₋₆ alkyl groups;
R⁵ and R⁷ are both hydrogen atoms; and
R⁶ is a C₃₋₈ cycloalkyl group.

[8] The compound or pharmaceutically acceptable salt thereof as defined in the above [1], wherein
L is a bond;
X is N or CR⁴;
Y is N or CR⁵;
R¹ is an optionally substituted C₁₋₆ alkyl group, an optionally substituted C₃₋₈ cycloalkyl group, an optionally substituted C₆₋₁₄ aryl group, or an optionally substituted 4 to 6-membered non-aromatic heterocyclic group;
R² is a hydrogen atom, or an optionally substituted C₁₋₆ alkyl group;
R³ is a hydrogen atom, an optionally substituted C₁₋₆ alkyl group, or a halogen atom;
R⁴ and R⁸ are each independently a hydrogen atom, an optionally substituted C₁₋₆ alkyl group, an optionally substituted C₃₋₈ cycloalkyl group, a halogen atom, a hydroxy group, an optionally substituted C₁₋₆ alkoxy group, an optionally substituted 5- or 6-membered heterocyclic group or a cyano group;
R⁵ and R⁷ are each independently a hydrogen atom, an optionally substituted C₁₋₆ alkyl group, a halogen atom, or an optionally substituted C₃₋₈ cycloalkyl group; and
R⁶ is a hydrogen atom, an optionally substituted C₁₋₆ alkyl group, an optionally substituted C₃₋₈ cycloalkyl group, an optionally substituted C₂₋₆ alkenyl group, a halogen atom, an optionally substituted C₁₋₆ alkoxy group, an optionally substituted 5- or 6-membered heterocyclic group, an amino group, or a nitro group.

[9] The compound or pharmaceutically acceptable salt thereof as defined in the above [1], wherein
L is a bond;
X is CR⁴;
Y is CR⁵;
R¹ is
(1) a C₁₋₆ alkyl group optionally substituted by 1 to 3 substituents selected from
   (a) a halogen atom,
   (b) a hydroxy group,
   (c) an optionally halogenated C₁₋₆ alkoxy group,
   (d) a C₇₋₁₆ aralkyloxy group,
   (e) a 5- or 6-membered aromatic heterocyclic group,
   (f) a 5- or 6-membered non-aromatic heterocyclic group,
   (g) a carboxy group,
   (h) a 4- to 6-membered non-aromatic heterocyclyloxy group, and
   (i) a cyano group,
(2) a C₃₋₈ cycloalkyl group optionally substituted by 1 to 3 substituents selected from
   (a) a C₁₋₆ alkyl group,
   (b) a C₁₋₆ alkoxy group,
   (c) a halogen atom, and
   (d) a cyano group,
(3) a C₆₋₁₄ aryl group optionally substituted by 1 to 3 substituents selected from
   (a) a halogen atom, and
   (b) a C₁₋₆ alkoxy group, or
(4) a 4- to 6-membered non-aromatic heterocyclic group optionally substituted by 1 to 3 substituents selected from
   (a) an oxo group,
   (b) a C₁₋₆ alkyl group,
   (c) a halogen atom, and
   (d) a C₁₋₆ alkoxy group;
R² is
(1) a hydrogen atom, or
(2) a C₁₋₆ alkyl group;
R³ is
(1) a hydrogen atom, or
(2) a C₁₋₆ alkyl group;
R⁴ and R⁸ are each independently
(1) a hydrogen atom,
(2) a C₁₋₆ alkyl group optionally substituted by 1 to 4 halogen atoms,
(3) a halogen atom,
(4) a hydroxy group, or
(5) a C₁₋₆ alkoxy group;
R⁵ and R⁷ are each independently
(1) a hydrogen atom,
(2) a C₁₋₆ alkyl group optionally substituted by 1 to 3 halogen atoms,
(3) a halogen atom, or
(4) a C₃₋₈ cycloalkyl group; and
R⁶ is
(1) a hydrogen atom,
(2) a C₁₋₆ alkyl group
(3) a C₃₋₈ cycloalkyl group optionally substituted by 1 to 3 halogen atoms,
(4) a halogen atom,
(5) an amino group, or
(6) a nitro group.

[10] The compound or pharmaceutically acceptable salt thereof as defined in the above [1], wherein
L is bond;
X is CR⁴;
Y is CR⁵;
R¹ is a C₁₋₆ alkyl group optionally substituted by an optionally halogenated C₁₋₆ alkoxy group;
R² is a hydrogen atom;
R³ is a hydrogen atom;
R⁴ and R⁸ are both C₁₋₆ alkyl groups optionally substituted by 1 to 3 halogen atoms;
R⁵ and R⁷ are both hydrogen atoms; and
R⁶ is a C₃₋₈ cycloalkyl group.

[11] The compound or pharmaceutically acceptable salt thereof as defined in the above [1], wherein the compound is selected from:
2-(4-bromo-2-fluoro-6-methylphenyl)-6-ethoxy-2,5-dihydro-4H-pyrazolo[3,4-d]pyrimidin-4-one;
2-(4-cyclopropyl-2-fluoro-6-methylphenyl)-6-ethoxy-2,5-dihydro-4H-pyrazolo[3,4-d]pyrimidin-4-one;
2-[2-bromo-4-(difluoromethoxy)-6-methylphenyl]-6-ethoxy-2,5-dihydro-4H-pyrazolo[3,4-d]pyrimidin-4-one;
2-(4-cyclopropyl-2,6-dimethylphenyl)-6-methoxy-2,5-dihydro-4H-pyrazolo[3,4-d]pyrimidin-4-one;
2-[4-cyclopropyl-2-(difluoromethyl)-6-fluorophenyl]-6-(2-methoxyethoxy)-2,5-dihydro-4H-pyrazolo[3,4-d]pyrimidin-4-one;
2-(4-cyclopropyl-2,6-dimethylphenyl)-6-[(difluoromethoxy)methyl]-2,5-dihydro-4H-pyrazolo[3,4-d]pyrimidin-4-one;
2-[4-(1-fluorocyclopropyl)-2,6-dimethylphenyl]-6-methoxy-2,5-dihydro-4H-pyrazolo[3,4-d]pyrimidin-4-one; and
2-[4-cyclopropyl-2-(difluoromethyl)-6-methylphenyl]-6-(methoxymethyl)-2,5-dihydro-4H-pyrazolo[3,4-d]pyrimidin-4-one.

[12]A method of treating a disease, disorder or condition in a subject, which comprises administering to the subject an effective amount of a compound or pharmaceutically acceptable salt as defined in the above [1], wherein the disease, disorder or condition is associated with NLRP3.

[13]A method of treating a disease, disorder or condition in a subject, which comprises administering to the subject an effective amount of a compound or pharmaceutically acceptable salt as defined in the above [1], wherein the disease, disorder or condition is associated with a heterozygous gain of function mutation in the NLRP3 gene.

[14]A method of treating a cryopyrin-associated periodic syndrome (CAPS) in a subject, which comprises administering to the subject an effective amount of a compound or pharmaceutically acceptable salt as defined in the above [1].

[15] The method according to the above [14], wherein the cryopyrin-associated periodic syndrome is selected from the group consisting of neonatal-onset multisystem inflammatory disease (NOMID/CINCA), Muckle-Wells syndrome (MWS), and familial cold autoinflammatory syndrome (FCAS).

[16]A method of treating a neurodegenerative disease, disorder or condition in a subject, which comprises administering to the subject an effective amount of a compound or pharmaceutically acceptable salt as defined in the above [1].

[17]A method of treating Parkinson's disease, Alzheimer's disease, Huntington's disease, amyotrophic lateral sclerosis or prion disease in a subject, which comprises administering to the subject an effective amount of a compound or pharmaceutically acceptable salt as defined in the above [1].

[18]A medicament comprising a compound or pharmaceutically acceptable salt as defined in the above [1].

[19] The medicament according to the above [18], which is an agent for the treatment of disease, disorder or condition associated with NLRP3.

[20] The medicament according to the above [18], which is an agent for the treatment of disease, disorder or condition associated with a heterozygous gain of function mutation in the NLRP3 gene.

[21] The medicament according to the above [18], which is an agent for the treatment of a cryopyrin-associated periodic syndrome (CAPS).

[22] The medicament according to the above [21], wherein the cryopyrin-associated periodic syndrome is selected from the group consisting of neonatal-onset multisystem inflammatory disease (NOMID/CINCA), Muckle-Wells syndrome (MWS), and familial cold autoinflammatory syndrome (FCAS).

[23]A compound or pharmaceutically acceptable salt as defined in the above [1] for use as a medicament.

[24]A compound or pharmaceutically acceptable salt as defined in the above [1] for use in treating a disease, disorder or condition associated with NLRP3.

[25]A compound or pharmaceutically acceptable salt as defined in the above [1] for use in treating a disease, disorder or condition associated with a heterozygous gain of function mutation in the NLRP3 gene.

[26]A compound or pharmaceutically acceptable salt as defined in the above [1] for use in treating a cryopyrin-associated periodic syndrome (CAPS).

[27]A Compound or pharmaceutically acceptable salt as defined in the above [26] wherein the cryopyrin-associated periodic syndrome is selected from the group consisting of neonatal-onset multisystem inflammatory disease (NOMID/CINCA), Muckle-Wells syndrome (MWS), and familial cold autoinflammatory syndrome (FCAS)

[28] Use of a compound or pharmaceutically acceptable salt as defined the above [1] for the manufacture of a medicament for the treatment of disease, disorder or condition associated with NLRP3.

[29] Use of a compound or pharmaceutically acceptable salt as defined in the above [1] for the manufacture of a medicament for the treatment of disease, disorder or condition associated with a heterozygous gain of function mutation in the NLRP3 gene.

[30] Use of a compound or pharmaceutically acceptable salt as defined in the above [1] for the manufacture of a medicament for the treatment of a cryopyrin-associated periodic syndrome (CAPS).

[31] The use according to the above [30], wherein the cryopyrin-associated periodic syndrome is selected from the group consisting of neonatal-onset multisystem inflammatory disease (NOMID/CINCA), Muckle-Wells syndrome (MWS), and familial cold autoinflammatory syndrome (FCAS).

[32]A combination comprising a compound or pharmaceutically acceptable salt as defined in the above [1], and at least one additional pharmacologically active agent (hereinafter, it is sometimes also referred as “pharmacologically active compound”).

[33] The combination according to the above [32], wherein the additional pharmacologically active agent is selected from the group consisting of beta-secretase inhibitors, gamma-secretase inhibitors, HMG-CoA reductase inhibitors, nonsteroidal anti-inflammatory drugs, vitamin E, anti-amyloid antibodies, antidepressants, antipsychotics, anxiolytics, and anticonvulsants.

### DETAILED DESCRIPTION OF THE INVENTION

Unless otherwise indicated, this disclosure uses definitions provided below.

“Substituted,” when used in connection with a chemical substituent or moiety (e.g., a C₁₋₆ alkyl group), means that one or more hydrogen atoms of the substituent or moiety have been replaced with one or more non-hydrogen atoms or groups, provided valence requirements are met and a chemically stable compound results from the substitution.

“About” or “approximately,” when used in connection with a measurable numerical variable, refers to the indicated value of the variable and to all values of the variable that are within the experimental error of the indicated value or within ±10 percent of the indicated value, whichever is greater.

“Alkyl” refers to straight chain and branched saturated hydrocarbon groups, generally having a specified number of carbon atoms (e.g., C₁₋₃ alkyl refers to an alkyl group having 1 to 3 (i.e., 1, 2, or 3) carbon atoms, C₁₋₄ alkyl refers to an alkyl group having 1 to 4 (i.e., 1, 2, 3 or 4) carbon atoms, C₁₋₆ alkyl refers to an alkyl group having 1 to 6 (i.e., 1, 2, 3, 4, 5 or 6) carbon atoms, and so on). Examples of alkyl groups include methyl, ethyl, n-propyl, i-propyl (isopropyl), n-butyl, s-butyl (sec-butyl), i-butyl (isobutyl), t-butyl (tert-butyl), pent-1-yl, pent-2-yl, pent-3-yl, 3-methylbut-1-yl, 3-methylbut-2-yl, 2-methylbut-2-yl, 2,2,2-trimethyleth-1-yl, n-hexyl, and the like.

“Alkanediyl” refers to divalent alkyl groups, where alkyl is defined above, and generally having a specified number of carbon atoms (e.g., C₁₋₄ alkanediyl refers to an alkanediyl group having 1 to 4 (i.e., 1, 2, 3 or 4) carbon atoms, C₁₋₆ alkanediyl refers to an alkanediyl group having 1 to 6 (i.e., 1, 2, 3, 4, 5 or 6) carbon atoms, and so on). Examples of alkanediyl groups include methylene, ethane-1,1-diyl, ethane-1,2-diyl, propane-1,3-diyl, propane-1,2-diyl, propane-1,1-diyl, propane-2,2-diyl, butane-1,4-diyl, butane-1,3-diyl, butane-1,2-diyl, butane-1,1-diyl, isobutane-1,3-diyl, isobutane-1,1-diyl, isobutane-1,2-diyl, and the like.

“Alkenyl” refers to straight chain and branched hydrocarbon groups having one or more carbon-carbon double bonds, and generally having a specified number of carbon atoms (e.g., C₂₋₆ alkenyl refers to an alkenyl group having 2 to 6 (i.e., 2, 3, 4, 5 or 6) carbon atoms, and so on). Examples of alkenyl groups include ethenyl, 1-propen-1-yl, 1-propen-2-yl, 2-propen-1-yl, 1-buten-1-yl, 1-buten-2-yl, 3-buten-1-yl, 3-buten-2-yl, 2-buten-1-yl, 2-buten-2-yl, 2-methyl-1-propen-1-yl, 2-methyl-2-propen-1-yl, 1,3-butadien-1-yl, 1,3-butadien-2-yl, and the like.

“Alkynyl” refers to straight chain or branched hydrocarbon groups having one or more triple carbon-carbon bonds, and generally having a specified number of carbon atoms (e.g., C₂₋₆ alkynyl refers to an alkynyl group having 2 to 6 (i.e., 2, 3, 4, 5 or 6) carbon atoms, and so on). Examples of alkynyl groups include ethynyl, 1-propyn-1-yl, 2-propyn-1-yl, 1-butyn-1-yl, 3-butyn-1-yl, 3-butyn-2-yl, 2-butyn-1-yl, and the like.

“Alkoxy” refers to straight chain and branched saturated hydrocarbon groups attached through an oxygen atom, generally having a specified number of carbon atoms (e.g., C₁₋₄ alkoxy refers to an alkoxy group having 1 to 4 (i.e., 1, 2, 3 or 4) carbon atoms, C₁₋₆ alkoxy refers to an alkoxy group having 1 to 6 (i.e., 1, 2, 3, 4, 5 or 6) carbon atoms, and so on). Examples of alkoxy groups include methoxy, ethoxy, n-propoxy, i-propoxy, n-butoxy, s-butoxy, i-butoxy, t-butoxy, pent-1-yloxy, pent-2-yloxy, pent-3-yloxy, 3-methylbut-1-yloxy, 3-methylbut-2-yloxy, 2-methylbut-2-yloxy, 2,2,2-trimethyleth-1-yloxy, n-hexoxy, and the like.

“Alkyl-carbonyl” and “alkylsulfonyl” refer to an alkyl group as defined above, which is attached, respectively, through a carbonyl (C(O)) group or a sulfonyl (SO₂) group, and generally having a specified number of carbon atoms (e.g., C₁₋₆ alkyl-carbonyl refers to an alkyl-carbonyl group having 1 to 6 (i.e., 1, 2, 3, 4, 5 or 6) carbon atoms, excluding the carbonyl moiety, C₁₋₆ alkylsulfonyl refers to an alkylsulfonyl group having 1 to 6 (i.e., 1, 2, 3, 4, 5 or 6) carbon atoms, and so on). Examples of alkyl-carbonyl groups include methylcarbonyl (acetyl), ethylcarbonyl, i-propylcarbonyl (propanoyl), n-propylcarbonyl, 2-methylpropanoyl, and the like. Examples of alkylsulfonyl groups include methylsulfonyl, ethylsulfonyl, i-propylsulfonyl, n-propylsulfonyl, and the like.

“Alkylamino” including mono- or di-alkylamino group refers to an alkyl group as defined above, which is attached through at least one amino group, and generally having a specified number of carbon atoms (e.g., C₁₋₆ alkylamino refers to a mono- or di-alkylamino group having 1 to 6 (i.e., 1, 2, 3, 4, 5 or 6) carbon atoms, and so on). Examples of mono- or di-alkylamino groups include methylamino, ethylamino, propylamino, isopropylamino, butylamino, dimethylamino, diethylamino, dipropylamino, dibutylamino, N-ethyl-N-methylamino, and the like.

“Alkyl-carbamoyl” including mono- or di-alkyl-carbamoyl group refers to an alkyl group as defined above, which is attached through a carbamoyl (CONH₂) group, and generally having a specified number of carbon atoms (e.g., C₁₋₆ alkyl-carbamoyl refers to a mono- or di-alkyl-carbamoyl group having 1 to 6 (i.e., 1, 2, 3, 4, 5 or 6) carbon atoms, excluding the carbamoyl moiety, and so on). Examples of mono- or di-alkyl-carbamoyl groups include methylcarbamoyl, ethylcarbamoyl, dimethylcarbamoyl, diethylcarbamoyl and N-ethyl-N-methylcarbamoyl, and the like.

“Alkyl-carbonylamino” refers to an alkyl-carbonyl as defined above, which is attached through an amino moiety, and generally having a specified number of carbon atoms (e.g., C₁₋₆ alkyl-carbonylamino refers to an alkyl-carbonylamino group having 1 to 6 (i.e., 1, 2, 3, 4, 5 or 6) carbon atoms, excluding the carbonyl moiety, and so on). Examples of C₁₋₆ alkyl-carbonylamino groups include methylcarbonylamino (acetylamino), ethylcarbonylamino, and the like.

“Alkoxy-carbonyl” refers to an alkoxy group as defined above, which is attached through a carbonyl (C(O)) group, and generally having a specified number of carbon atoms (e.g., C₁₋₆ alkoxy-carbonyl refers to an alkoxy-carbonyl group having 1 to 6 (i.e., 1, 2, 3, 4, 5 or 6) carbon atoms, excluding the carbonyl moiety, and so on). Examples of C₁₋₆ alkoxy-carbonyl groups include methoxycarbonyl, ethoxycarbonyl, and the like.

“Halo,” “halogen” and “halogeno” may be used interchangeably and refer to fluoro, chloro, bromo, and iodo.

“Haloalkyl,” “haloalkenyl,” and “haloalkynyl,” refer, respectively, to alkyl, alkenyl, and alkynyl groups substituted with one or more halogen atoms, where alkyl, alkenyl, and alkynyl are defined above, and generally having a specified number of carbon atoms. Examples of haloalkyl groups include fluoromethyl, difluoromethyl, trifluoromethyl, chloromethyl, dichloromethyl, trichloromethyl, 1-fluoroethyl, 1,1-difluoroethyl, 1-chloroethyl, 1,1-dichloroethyl, 1-fluoro-1-methylethyl, 1-chloro-1-methylethyl, and the like.

“Cycloalkyl” refers to saturated monocyclic and bicyclic hydrocarbon groups, generally having a specified number of carbon atoms that comprise the ring or rings (e.g., C₃₋₈ cycloalkyl refers to a cycloalkyl group having 3 to 8 (i.e., 3, 4, 5, 6, 7 or 8) carbon atoms as ring members). Bicyclic hydrocarbon groups may include isolated rings (two rings sharing no carbon atoms), spiro rings (two rings sharing one carbon atom), fused rings (two rings sharing two carbon atoms and the bond between the two common carbon atoms), and bridged rings (two rings sharing two carbon atoms, but not a common bond). The cycloalkyl group may be attached through any ring atom unless such attachment would violate valence requirements, and where indicated, may optionally include one or more non-hydrogen substituents unless such substitution would violate valence requirements.

Examples of monocyclic cycloalkyl groups include cyclopropyl, cyclobutyl, cyclopentyl, cyclohexyl, and the like. Examples of fused bicyclic cycloalkyl groups include bicyclo[2.1.0]pentanyl (i.e., bicyclo[2.1.0]pentan-1-yl, bicyclo[2.1.0]pentan-2-yl, and bicyclo[2.1.0]pentan-5-yl), bicyclo[3.1.0]hexanyl, bicyclo[3.2.0]heptanyl, bicyclo[4.1.0]heptanyl, bicyclo[3.3.0]octanyl, bicyclo[4.2.0]octanyl, bicyclo[4.3.0]nonanyl, bicyclo[4.4.0]decanyl, and the like. Examples of bridged cycloalkyl groups include bicyclo[2.1.1]hexanyl, bicyclo[2.2.1]heptanyl, bicyclo[3.1.1]heptanyl, bicyclo[2.2.2]octanyl, bicyclo[3.2.1]octanyl, bicyclo[4.1.1]octanyl, bicyclo[3.3.1]nonanyl, bicyclo[4.2.1]nonanyl, bicyclo[3.3.2]decanyl, bicyclo[4.2.2]decanyl, bicyclo[4.3.1]decanyl, bicyclo[3.3.3]undecanyl, bicyclo[4.3.2]undecanyl, bicyclo[4.3.3]dodecanyl, and the like. Examples of spiro cycloalkyl groups include spiro[3.3]heptanyl, spiro[2.4]heptanyl, spiro[3.4]octanyl, spiro[2.5]octanyl, spiro[3.5]nonanyl, and the like. Examples of isolated bicyclic cycloalkyl groups include those derived from bi(cyclobutane), cyclobutanecyclopentane, bi(cyclopentane), cyclobutanecyclohexane, cyclopentanecyclohexane, bi(cyclohexane), etc.

“Cycloalkanediyl” refers to divalent cycloalkyl groups, where cycloalkyl is defined above, and generally having a specified number of carbon atoms (e.g., C₃₋₈ cycloalkanediyl refers to a cycloalkanediyl group having 3 to 8 (i.e., 3, 4, 5, 6, 7 or 8) carbon atoms, and so on). Examples of cycloalkanediyl groups include cyclopropane-1,1-diyl, cyclopropane-1,2-diyl, cyclobutane-1,1-diyl, cyclobutane-1,2-diyl, and the like.

“Cycloalkylidene” refers to divalent monocyclic cycloalkyl groups, where cycloalkyl is defined above, which are attached through a single carbon atom of the group, and generally having a specified number of carbon atoms that comprise the ring (e.g., C₃₋₈ cycloalkylidene refers to a cycloalkylidene group having 3 to 8 (i.e., 3, 4, 5, 6, 7 or 8) carbon atoms as ring members). Examples of cycloalkylidene groups include cyclopropylidene, cyclobutylidene, cyclopentylidene, and cyclohexylidene.

“Cycloalkenyl” refers to partially unsaturated monocyclic and bicyclic hydrocarbon groups, generally having a specified number of carbon atoms that comprise the ring or rings (e.g., C₃₋₈ cycloalkenyl refers to a cycloalkenyl group having 3 to 8 (i.e., 3, 4, 5, 6, 7 or 8) carbon atoms, and so on). As with cycloalkyl groups, the bicyclic cycloalkenyl groups may include isolated, spiro, fused, or bridged rings. Similarly, the cycloalkenyl group may be attached through any ring atom, and where indicated, may optionally include one or more non-hydrogen substituents unless such attachment or substitution would violate valence requirements. Examples of cycloalkenyl groups include the partially unsaturated analogs of the cycloalkyl groups described above, such as cyclobutenyl (i.e., cyclobuten-1-yl and cyclobuten-3-yl), cyclopentenyl, cyclohexenyl, bicyclo[2.2.1]hept-2-enyl, and the like.

“Cycloalkyl-carbonyl” or “cycloalkylsulfonyl” refers to a cycloalkyl group as defined above, which is attached, respectively, through a carbonyl (C(O)) group or a sulfonyl (SO₂) group, and generally having a specified number of carbon atoms (e.g., C₃₋₈ cycloalkyl-carbonyl refers to a cycloalkyl-carbonyl group having 3 to 8 (i.e., 3, 4, 5, 6, 7 or 8) carbon atoms, excluding the carbonyl moiety, as ring members of cycloalkyl group, C₃₋₈ cycloalkylsulfonyl refers to a cycloalkylsulfonyl group having 3 to 8 (i.e., 3, 4, 5, 6, 7 or 8) carbon atoms as ring members of cycloalkyl group, and so on). Examples of cycloalkyl-carbonyl groups include cyclopropylcarbonyl cyclobutylcarbonyl, cyclopentylcarbonyl, and the like. Examples of cycloalkylsulfonyl groups include cyclopropylsulfonyl cyclobutylsulfonyl, cyclopentylsulfonyl, and the like.

“Aryl” refers to fully unsaturated monocyclic aromatic hydrocarbons and to polycyclic hydrocarbons having at least one aromatic ring, both monocyclic and polycyclic aryl groups generally having a specified number of carbon atoms that comprise their ring members (e.g., C₆₋₁₄ aryl refers to an aryl group having 6 to 14 carbon atoms as ring members, and so on). The group may be attached through any ring atom, and where indicated, may optionally include one or more non-hydrogen substituents unless such attachment or substitution would violate valence requirements. Examples of aryl groups include phenyl, biphenyl, cyclobutabenzenyl, indenyl, naphthyl, benzocycloheptanyl, biphenylenyl, fluorenyl, groups derived from cycloheptatriene cation, and the like.

“Acyl group” include a formyl group, a carboxy group, a carbamoyl group, a thiocarbamoyl group, a sulfino group, a sulfo group, a sulfamoyl group and a phosphono group, each optionally having “1 or 2 substituents selected from a C₁₋₆ alkyl group, a C₂₋₆ alkenyl group, a C₃₋₁₀ cycloalkyl group, a C₃₋₁₀ cycloalkenyl group, a C₆₋₁₄ aryl group, a C₇₋₁₆ aralkyl group, a 5- to 14-membered aromatic heterocyclic group and a 3- to 14-membered non-aromatic heterocyclic group, each of which optionally has 1 to 3 substituents selected from a halogen atom, an optionally halogenated C₁₋₆ alkoxy group, a hydroxy group, a nitro group, a cyano group, an amino group and a carbamoyl group”. Examples of the “acyl group” also include a hydrocarbon-sulfonyl group, a heterocyclylsulfonyl group, a hydrocarbon-sulfinyl group and a heterocyclylsulfinyl group. Here, the hydrocarbon-sulfonyl group means a hydrocarbon group-bonded sulfonyl group, the heterocyclylsulfonyl group means a heterocyclic group-bonded sulfonyl group, the hydrocarbon-sulfinyl group means a hydrocarbon group-bonded sulfinyl group and the heterocyclylsulfinyl group means a heterocyclic group-bonded sulfinyl group.

Preferable examples of the “acyl group” include a formyl group, a carboxy group, a C₁₋₆ alkyl-carbonyl group, a C₂₋₆ alkenyl-carbonyl group (e.g., crotonoyl), a C₃₋₁₀ cycloalkyl-carbonyl group (e.g., cyclobutanecarbonyl, cyclopentanecarbonyl, cyclohexanecarbonyl, cycloheptanecarbonyl), a C₃₋₁₀ cycloalkenyl-carbonyl group (e.g., 2-cyclohexenecarbonyl), a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a C₆₋₁₄ aryloxy-carbonyl group (e.g., phenyloxycarbonyl, naphthyloxycarbonyl), a C₇₋₁₆ aralkyloxy-carbonyl group (e.g., benzyloxycarbonyl, phenethyloxycarbonyl), a carbamoyl group, a mono- or di-C₁₋₆ alkyl-carbamoyl group, a mono- or di-C₂₋₆ alkenyl-carbamoyl group (e.g., diallylcarbamoyl), a mono- or di-C₃₋₁₀ cycloalkyl-carbamoyl group (e.g., cyclopropylcarbamoyl), a mono- or di-C₆₋₁₄ aryl-carbamoyl group (e.g., phenylcarbamoyl), a mono- or di-C₇₋₁₆ aralkyl-carbamoyl group, a 5- to 14-membered aromatic heterocyclylcarbamoyl group (e.g., pyridylcarbamoyl), a thiocarbamoyl group, a mono- or di-C₁₋₆ alkyl-thiocarbamoyl group (e.g., methylthiocarbamoyl, N-ethyl-N-methylthiocarbamoyl), a mono- or di-C₂₋₆ alkenyl-thiocarbamoyl group (e.g., diallylthiocarbamoyl), a mono- or di-C₃₋₁₀ cycloalkyl-thiocarbamoyl group (e.g., cyclopropylthiocarbamoyl, cyclohexylthiocarbamoyl), a mono- or di-C₆₋₁₄ aryl-thiocarbamoyl group (e.g., phenylthiocarbamoyl), a mono- or di-C₇₋₁₆ aralkyl-thiocarbamoyl group (e.g., benzylthiocarbamoyl, phenethylthiocarbamoyl), a 5- to 14-membered aromatic heterocyclylthiocarbamoyl group (e.g., pyridylthiocarbamoyl), a sulfino group, a C₁₋₆ alkylsulfinyl group (e.g., methylsulfinyl, ethylsulfinyl), a sulfo group, a C₁₋₆ alkylsulfonyl group, a C₆₋₁₄ arylsulfonyl group, a phosphono group and a mono- or di-C₁₋₆ alkylphosphono group (e.g., dimethylphosphono, diethylphosphono, diisopropylphosphono, dibutylphosphono).

“Aralkyl” refers to an alkyl group as defined above, wherein one of its hydrogens is substituted by an aryl group as defined above, and generally having a specified number of carbon atoms (e.g., C₇₋₁₆ aralkyl refers to an aralkyl group having 7 to 16 carbon atoms, and so on). Examples of aralkyl groups include benzyl, phenethyl, naphthylmethyl, phenylpropyl, and the like.

“Aralkyloxy” refers to a hydroxy group whose hydrogen is substituted by an aralkyl group as defined above, and generally having a specified number of carbon atoms (e.g., C₇₋₁₆ aralkyloxy refers to an aralkyloxy group having 7 to 16 carbon atoms, and so on). Examples of aralkyloxy groups include benzyloxy, phenethyloxy, naphthylmethoxy, phenylpropyloxy, and the like.

“Aralkyloxy-carbonyl” refers to an aralkyloxy group as defined above, which is attached through a carbonyl (C(O)) group, and generally having a specified number of carbon atoms (e.g., C₇₋₁₆ aralkyloxy-carbonyl refers to an aralkyloxy-carbonyl group having 7 to 16 carbon atoms, excluding the carbonyl moiety, and so on). Examples of aralkyloxy-carbonyl groups include benzyloxycarbonyl, phenethyloxycarbonyl, naphthylmethoxycarbonyl, phenylpropyloxycarbonyl, and the like.

“Arylene” refers to divalent aryl groups, where aryl is defined above, and generally having a specified number of carbon atoms that comprise their ring members (e.g., C₆₋₁₄ arylene refers to an arylene group having 6 to 14 carbon atoms as ring members, and so on). Examples of arylene groups include o-phenylene (i.e., benzene-1,2-diyl).

“Heterocycle”, “heterocyclic” and “heterocyclyl” may be used interchangeably and refer to saturated or partially unsaturated monocyclic or bicyclic groups having ring atoms composed of carbon atoms and one or more heteroatoms independently selected from nitrogen, oxygen, and sulfur. Both the monocyclic and bicyclic groups generally have a specified number of carbon atoms in their ring or rings (e.g., C₂₋₆ heterocyclyl refers to a heterocyclyl group having 2 to 6 (i.e., 2, 3, 4, 5 or 6) carbon atoms and, e.g., 1 to 4 (i.e., 1, 2, 3 or 4) heteroatoms, as ring members, while 5- or 6-membered heterocyclic group refers to a heterocyclyl group having 5 or 6 atoms as ring members in total of carbon atoms and heteroatoms). As with bicyclic cycloalkyl groups, bicyclic heterocyclyl groups may include isolated rings, spiro rings, fused rings, and bridged rings. The heterocyclyl group may be attached through any ring atom, and where indicated, may optionally include one or more non-hydrogen substituents unless such attachment or substitution would violate valence requirements or result in a chemically unstable compound. Examples of heterocyclyl groups include oxiranyl, thiiranyl, aziridinyl (e.g., aziridin-1-yl and aziridin-2-yl), oxetanyl, thietanyl, azetidinyl, tetrahydrofuranyl, tetrahydrothienyl, pyrrolidinyl, tetrahydropyranyl, tetrahydrothiopyranyl, piperidinyl, 1,4-dioxanyl, 1,4-oxathianyl, morpholinyl, 1,4-dithianyl, piperazinyl, 1,4-azathianyl, oxepanyl, thiepanyl, azepanyl, 1,4-dioxepanyl, 1,4-oxathiepanyl, 1,4-oxaazepanyl, 1,4-dithiepanyl, 1,4-thiazepanyl, 1,4-diazepanyl, 3,4-dihydro-2H-pyranyl, 3,6-dihydro-2H-pyranyl, 2H-pyranyl, 1,2-dihydropyridinyl, 1,2,3,4-tetrahydropyridinyl, 1,2,5,6-tetrahydropyridinyl, 1,6-dihydropyrimidinyl, 1,2,3,4-tetrahydropyrimidinyl, and 1,2-dihydropyrazolo[1,5-d][1,2,4]triazinyl.

“Heterocycle-diyl” refers to heterocyclyl groups which are attached through two ring atoms of the group, where heterocyclyl is defined above. They generally have a specified number of carbon atoms in their ring or rings (e.g., C₂₋₆ heterocycle-diyl refers to a heterocycle-diyl group having 2 to 6 (i.e., 2, 3, 4, 5 or 6) carbon atoms and, e.g., 1 to 4 (i.e., 1, 2, 3 or 4) heteroatoms, as ring members). Examples of heterocycle-diyl groups include the multivalent analogs of the heterocycle groups described above, such as morpholine-3,4-diyl, pyrrolidine-1,2-diyl, 1-pyrrolidinyl-2-ylidene, 1-pyridinyl-2-ylidene, 1-(4H)-pyrazolyl-5-ylidene, 1-(3H)-imidazolyl-2-ylidene, 3-oxazolyl-2-ylidene, 1-piperidinyl-2-ylidene, 1-piperazinyl-6-ylidene, and the like.

“Heteroaromatic”, “aromatic heterocyclyl/heterocyclic” and “heteroaryl” may be used interchangeably and refer to unsaturated monocyclic aromatic groups and to polycyclic groups having at least one aromatic ring, each of the groups having ring atoms composed of carbon atoms and one or more heteroatoms independently selected from nitrogen, oxygen, and sulfur. Both the monocyclic and polycyclic groups generally have a specified number of carbon atoms as ring members (e.g., C₁₋₉ heteroaryl refers to a heteroaryl group having 1 to 9 (i.e., 1, 2, 3, 4, 5, 6, 7, 8 or 9) carbon atoms and, e.g., 1 to 4 (i.e., 1, 2, 3 or 4) heteroatoms, as ring members) and may include any bicyclic group in which any of the above-listed monocyclic heterocycles are fused to a benzene ring. The heteroaryl group may be attached through any ring atom (or ring atoms for fused rings), and where indicated, may optionally include one or more non-hydrogen substituents unless such attachment or substitution would violate valence requirements or result in a chemically unstable compound. Examples of heteroaryl groups include monocyclic groups such as pyrrolyl (e.g., pyrrol-1-yl, pyrrol-2-yl, and pyrrol-3-yl), furyl, thienyl, pyrazolyl, imidazolyl, isoxazolyl, oxazolyl, isothiazolyl, thiazolyl, 1,2,3-triazolyl, 1,3,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, tetrazolyl, pyridinyl, pyridazinyl, pyrimidinyl, and pyrazinyl.

Examples of heteroaryl groups also include bicyclic groups such as benzofuranyl, isobenzofuranyl, benzothienyl, benzo[c]thienyl, 1H-indolyl, 3H-indolyl, isoindolyl, 1H-isoindolyl, indolinyl, isoindolinyl, benzimidazolyl, 1H-indazolyl, 2H-indazolyl, benzotriazolyl, 1H-pyrrolo[2,3-b]pyridinyl, 1H-pyrrolo[2,3-c]pyridinyl, 1H-pyrrolo[3,2-c]pyridinyl, 1H-pyrrolo[3,2-b]pyridinyl, 3H-imidazo[4,5-b]pyridinyl, 3H-imidazo[4,5-c]pyridinyl, 1H-pyrazolo[4,3-b]pyridinyl, 1H-pyrazolo[4,3-c]pyridinyl, 1H-pyrazolo[3,4-c]pyridinyl, 1H-pyrazolo[3,4-b]pyridinyl, 7H-purinyl, indolizinyl, imidazo[1,2-a]pyridinyl, imidazo[1,5-a]pyridinyl, pyrazolo[1,5-a]pyridinyl, pyrrolo[1,2-b]pyridazinyl, imidazo[1,2-c]pyrimidinyl, quinolyl, isoquinolyl, cinnolinyl, quinazolinyl, quinoxalinyl, phthalazinyl, 1,6-naphthyridinyl, 1,7-naphthyridinyl, 1,8-naphthyridinyl, 1,5-naphthyridinyl, 2,6-naphthyridinyl, 2,7-naphthyridinyl, pyrido[3,2-d]pyrimidinyl, pyrido[4,3-d]pyrimidinyl, pyrido[3,4-d]pyrimidinyl, pyrido[2,3-d]pyrimidinyl, pyrido[2,3-b]pyrazinyl, pyrido[3,4-b]pyrazinyl, pyrimido[5,4-d]pyrimidinyl, pyrazino[2,3-b]pyrazinyl, pyrimido[4,5-d]pyrimidinyl, 1,2,3,4-tetrahydropyrido[2,3-b]pyrazinyl, 2,3-dihydrobenzo[b][1,4]dioxinyl, 3,4-dihydro-2H-pyrido[3,2-b][1,4]oxazinyl, 2,3-dihydro-1H-benzo[d]imidazolyl, benzo[d]thiazolyl, 2,3-dihydro-1H-pyrrolo[2,3-b]pyridinyl, [1,2,4]triazolo[1,5-a]pyridinyl, 2,3-dihydro-1H-imidazo[4,5-b]pyridinyl, tetrazolo[1,5-a]pyridinyl, 7H-pyrrolo[2,3-d]pyrimidinyl, pyrazolo[1,5-a]pyrimidinyl, imidazo[1,2-a]pyrimidinyl, 4,5-dihydro-1H-pyrazolo[3,4-d]pyrimidinyl, 2,3,6,7-tetrahydro-1H-purinyl, 5H-pyrrolo[2,3-b]pyrazinyl, imidazo[1,2-a]pyrazinyl, imidazo[1,2-b]pyridazinyl, and 4,5,6,7-tetrahydropyrazolo[1,5-a]pyrazinyl.

“Heteroarylene” refers to heteroaryl groups which are attached through two ring atoms of the group, where heteroaryl is defined above. They generally have a specified number of carbon atoms in their ring or rings (e.g., C₃₅ heteroarylene refers to a heteroarylene group having 3 to 5 carbon atoms and, e.g., 1 to 4 (i.e., 1, 2, 3 or 4) heteroatoms, as ring members). Examples of heteroarylene groups include the multivalent analogs of the heteroaryl groups described above, such as pyridine-2,3-diyl, pyridine-3,4-diyl, pyrazole-4,5-diyl, pyrazole-3,4-diyl, and the like.

“Non-aromatic heterocyclic/heterocyclyl” (including “3- to 8-membered non-aromatic heterocyclic group)” refers to heterocyclic group other than heteroaryl groups as mentioned above. Preferable examples of the “non-aromatic heterocyclic group” include 3- to 8-membered monocyclic non-aromatic heterocyclic groups such as aziridinyl, oxiranyl, thiiranyl, azetidinyl, oxetanyl, thietanyl, tetrahydrothienyl, tetrahydrofuranyl, pyrrolinyl, pyrrolidinyl, imidazolinyl, imidazolidinyl, oxazolinyl, oxazolidinyl, pyrazolinyl, pyrazolidinyl, thiazolinyl, thiazolidinyl, tetrahydroisothiazolyl, tetrahydrooxazolyl, tetrahydroisooxazolyl, piperidinyl, piperazinyl, tetrahydropyridinyl, dihydropyridinyl, dihydrothiopyranyl, tetrahydropyrimidinyl, tetrahydropyridazinyl, dihydropyranyl, tetrahydropyranyl, tetrahydrothiopyranyl, morpholinyl, thiomorpholinyl, azepanyl, diazepanyl, azepinyl, oxepanyl, azocanyl, diazocanyl and the like; and

9- to 14-membered fused polycyclic (preferably bi or tricyclic) non-aromatic heterocyclic groups such as dihydrobenzofuranyl, dihydrobenzimidazolyl, dihydrobenzoxazolyl, dihydrobenzothiazolyl, dihydrobenzisothiazolyl, dihydronaphtho[2,3-b]thienyl, tetrahydroisoquinolyl, tetrahydroquinolyl, 4H-quinolizinyl, indolinyl, isoindolinyl, tetrahydrothieno[2,3-c]pyridinyl, tetrahydrobenzazepinyl, tetrahydroquinoxalinyl, tetrahydrophenanthridinyl, hexahydrophenothiazinyl, hexahydrophenoxazinyl, tetrahydrophthalazinyl, tetrahydronaphthyridinyl, tetrahydroquinazolinyl, tetrahydrocinnolinyl, tetrahydrocarbazolyl, tetrahydro-p-carbolinyl, tetrahydroacrydinyl, tetrahydrophenazinyl, tetrahydrothioxanthenyl, octahydroisoquinolyl and the like.

In the present specification, examples of the “nitrogen-containing heterocyclic group” include a “heterocyclic group” containing at least one nitrogen atom as a ring-constituting atom.

In the present specification, examples of the “optionally substituted heterocyclic group” include a heterocyclic group optionally having substituent(s) selected from the substituent group A as described later.

“Oxo” refers to a double bonded oxygen (═O).

Examples of the “substituent” (including “hetero-containing substituents”) include a halogen atom, a cyano group, a nitro group, an optionally substituted hydrocarbon group, an optionally substituted heterocyclic group, an acyl group, an optionally substituted amino group, an optionally substituted carbamoyl group, an optionally substituted thiocarbamoyl group, an optionally substituted sulfamoyl group, an optionally substituted hydroxy group, an optionally substituted sulfanyl (SH) group and an optionally substituted silyl group.

Examples of the “hydrocarbon group” (including “hydrocarbon group” of “optionally substituted hydrocarbon group”) include a C₁₋₆ alkyl group, a C₂₋₆ alkenyl group, a C₂₋₆ alkynyl group, a C₃₋₁₀ cycloalkyl group, a C₃₋₁₀ cycloalkenyl group, a C₆₋₁₄ aryl group and a C₇₋₁₆ aralkyl group.

“Hetero-containing substituents” refers to substituents containing at least one heteroatom. Examples of the “hetero-containing substituents” include a halogen atom, a cyano group, a nitro group, a heterocyclyl group, a heteroaryl group, an alkyl group substituted by hetero-containing substituents (such as halo-alkyl, amino-alkyl, cyano-alkyl, alkoxy-alkyl, and the like), a cycloalkyl substituted by hetero-containing substituents (such as halo-cycloalkyl, cyano-cycloalkyl, hydroxy-cycloalkyl, and the like), an optionally substituted alkoxy group, and the like.

Examples of the “optionally substituted hydrocarbon group” include a hydrocarbon group optionally having substituent(s) selected from the following substituent group A.

### [Substituent Group A]

(1) a halogen atom,
(2) a nitro group,
(3) a cyano group,
(4) an oxo group,
(5) a hydroxy group,
(6) an optionally halogenated C₁₋₆ alkoxy group,
(7) a C₆₋₁₄ aryloxy group (e.g., phenoxy, naphthoxy),
(8) a C₇₋₁₆ aralkyloxy group (e.g., benzyloxy),
(9) a 5- to 14-membered aromatic heterocyclyloxy group (e.g., pyridyloxy),
(10) a 3- to 14-membered non-aromatic heterocyclyloxy group (e.g., morpholinyloxy, piperidinyloxy),
(11) a C₁₋₆ alkyl-carbonyloxy group (e.g., acetoxy, propanoyloxy),
(12) a C₆₋₁₄ aryl-carbonyloxy group (e.g., benzoyloxy, 1-naphthoyloxy, 2-naphthoyloxy),
(13) a C₁₋₆ alkoxy-carbonyloxy group (e.g., methoxycarbonyloxy, ethoxycarbonyloxy, propoxycarbonyloxy, butoxycarbonyloxy),
(14) a mono- or di-C₁₋₆ alkyl-carbamoyloxy group (e.g., methylcarbamoyloxy, ethylcarbamoyloxy, dimethylcarbamoyloxy, diethylcarbamoyloxy),
(15) a C₆₋₁₄ aryl-carbamoyloxy group (e.g., phenylcarbamoyloxy, naphthylcarbamoyloxy),
(16) a 5- to 14-membered aromatic heterocyclylcarbonyloxy group (e.g., nicotinoyloxy),
(17) a 3- to 14-membered non-aromatic heterocyclylcarbonyloxy group (e.g., morpholinylcarbonyloxy, piperidinylcarbonyloxy),
(18) an optionally halogenated C₁₋₆ alkylsulfonyloxy group (e.g., methylsulfonyloxy, trifluoromethylsulfonyloxy),
(19) a C₆₋₁₄ arylsulfonyloxy group optionally substituted by a C₁₋₆ alkyl group (e.g., phenylsulfonyloxy, toluenesulfonyloxy),
(20) an optionally halogenated C₁₋₆ alkylthio group,
(21) a 5- to 14-membered aromatic heterocyclic group,
(22) a 3- to 14-membered non-aromatic heterocyclic group,
(23) a formyl group,
(24) a carboxy group,
(25) an optionally halogenated C₁₋₆ alkyl-carbonyl group,
(26) a C₆₋₁₄ aryl-carbonyl group,
(27) a 5- to 14-membered aromatic heterocyclylcarbonyl group,
(28) a 3- to 14-membered non-aromatic heterocyclylcarbonyl group,
(29) a C₁₋₆ alkoxy-carbonyl group,
(30) a C₆₋₁₄ aryloxy-carbonyl group (e.g., phenyloxycarbonyl, 1-naphthyloxycarbonyl, 2-naphthyloxycarbonyl),
(31) a C₇₋₁₆ aralkyloxy-carbonyl group (e.g., benzyloxycarbonyl, phenethyloxycarbonyl),
(32) a carbamoyl group,
(33) a thiocarbamoyl group,
(34) a mono- or di-C₁₋₆ alkyl-carbamoyl group,
(35) a C₆₋₁₄ aryl-carbamoyl group (e.g., phenylcarbamoyl),
(36) a 5- to 14-membered aromatic heterocyclylcarbamoyl group (e.g., pyridylcarbamoyl, thienylcarbamoyl),
(37) a 3- to 14-membered non-aromatic heterocyclylcarbamoyl group (e.g., morpholinylcarbamoyl, piperidinylcarbamoyl),
(38) an optionally halogenated C₁₋₆ alkylsulfonyl group,
(39) a C₆₋₁₄ arylsulfonyl group,
(40) a 5- to 14-membered aromatic heterocyclylsulfonyl group (e.g., pyridylsulfonyl, thienylsulfonyl),
(41) an optionally halogenated C₁₋₆ alkylsulfinyl group,
(42) a C₆₋₁₄ arylsulfinyl group (e.g., phenylsulfinyl, 1-naphthylsulfinyl, 2-naphthylsulfinyl),
(43) a 5- to 14-membered aromatic heterocyclylsulfinyl group (e.g., pyridylsulfinyl, thienylsulfinyl),
(44) an amino group,
(45) a mono- or di-C₁₋₆ alkylamino group (e.g., methylamino, ethylamino, propylamino, isopropylamino, butylamino, dimethylamino, diethylamino, dipropylamino, dibutylamino, N-ethyl-N-methylamino),
(46) a mono- or di-C₆₋₁₄ arylamino group (e.g., phenylamino),
(47) a 5- to 14-membered aromatic heterocyclylamino group (e.g., pyridylamino),
(48) a C₇₋₁₆ aralkylamino group (e.g., benzylamino),
(49) a formylamino group,
(50) a C₁₋₆ alkyl-carbonylamino group (e.g., acetylamino, propanoylamino, butanoylamino),
(51) a (C₁₋₆ alkyl)(C₁₋₆ alkyl-carbonyl)amino group (e.g., N-acetyl-N-methylamino),
(52) a C₆₋₁₄ aryl-carbonylamino group (e.g., phenylcarbonylamino, naphthylcarbonylamino),
(53) a C₁₋₆ alkoxy-carbonylamino group (e.g., methoxycarbonylamino, ethoxycarbonylamino, propoxycarbonylamino, butoxycarbonylamino, tert-butoxycarbonylamino),
(54) a C₇₋₁₆ aralkyloxy-carbonylamino group (e.g., benzyloxycarbonylamino),
(55) a C₁₋₆ alkylsulfonylamino group (e.g., methylsulfonylamino, ethylsulfonylamino),
(56) a C₆₋₁₄ arylsulfonylamino group optionally substituted by a C₁₋₆ alkyl group (e.g., phenylsulfonylamino, toluenesulfonylamino),
(57) an optionally halogenated C₁₋₆ alkyl group,
(58) a C₂₋₆ alkenyl group,
(59) a C₂₋₆ alkynyl group,
(60) a C₃₋₁₀ cycloalkyl group,
(61) a C₃₋₁₀ cycloalkenyl group,
(62) a C₆₋₁₄ aryl group and
(63) a group represented by the formula: —(CH₂)ₐ—O—(CH₂)_{b}—, wherein each of a and b is the integer of 0 to 3 and the sum of a and b is 2 to 4.

The number of the above-mentioned substituents in the “optionally substituted hydrocarbon group” is, for example, 1 to 5, preferably 1 to 3. When the number of the substituents is two or more, the respective substituents may be the same or different.

“Leaving group” refers to any group that leaves a molecule during a fragmentation process, including substitution reactions, elimination reactions, and addition-elimination reactions. Leaving groups may be nucleofugal, in which the group leaves with a pair of electrons that formerly served as the bond between the leaving group and the molecule, or may be electrofugal, in which the group leaves without the pair of electrons. The ability of a nucleofugal leaving group to leave depends on its base strength, with the strongest bases being the poorest leaving groups. Common nucleofugal leaving groups include nitrogen (e.g., from diazonium salts); sulfonates, including alkylsulfonates (e.g., mesylate), fluoroalkylsulfonates (e.g., triflate, hexaflate, nonaflate, and tresylate), and arylsulfonates (e.g., tosylate, brosylate, closylate, and nosylate). Others include carbonates, halide ions, carboxylate anions, phenolate ions, and alkoxides. Some stronger bases, such as NH₂⁻ and OH can be made better leaving groups by treatment with an acid. Common electrofugal leaving groups include the proton, CO₂, and metals.

“Opposite enantiomer” refers to a molecule that is a non-superimposable mirror image of a reference molecule, which may be obtained by inverting all the stereogenic centers of the reference molecule. For example, if the reference molecule has S absolute stereochemical configuration, then the opposite enantiomer has R absolute stereochemical configuration. Likewise, if the reference molecule has S,S absolute stereochemical configuration, then the opposite enantiomer has R,R stereochemical configuration, and so on.

“Stereoisomer” and “stereoisomers” of a compound with given stereochemical configuration refer to the opposite enantiomer of the compound and to any diastereoisomers, including geometrical isomers (Z/E) of the compound. For example, if a compound has S,R,Z stereochemical configuration, its stereoisomers would include its opposite enantiomer having R,S,Z configuration, and its diastereomers having S,S,Z configuration, R,R,Z configuration, S,R,E configuration, R,S,E configuration, S,S,E configuration, and R,R,E configuration. If the stereochemical configuration of a compound is not specified, then “stereoisomer” refers to any one of the possible stereochemical configurations of the compound.

“Substantially pure stereoisomer” and variants thereof refer to a sample containing a compound having a specific stereochemical configuration and which comprises at least about 95% of the sample.

“Pure stereoisomer” and variants thereof refer to a sample containing a compound having a specific stereochemical configuration and which comprises at least about 99.5% of the sample.

“Subject” refers to a mammal, including a human.

“Pharmaceutically acceptable” substances refer to those substances which are suitable for administration to subjects.

“Treating” refers to reversing, alleviating, inhibiting the progress of, or preventing a disease, disorder or condition to which such term applies, or to reversing, alleviating, inhibiting the progress of, or preventing one or more symptoms of such disease, disorder or condition.

“Treatment” refers to the act of “treating,” as defined immediately above.

“Drug,” “drug substance,” “active pharmaceutical ingredient,” and the like, refer to a compound (e.g., compounds of Formula (I), including subgeneric compounds and compounds specifically named in the specification, or pharmaceutically acceptable salts thereof, or solvates thereof, or hydrates thereof) that may be used for treating a subject in need of treatment.

“Effective amount” of a drug, “therapeutically effective amount” of a drug, and the like, refer to the quantity of the drug that may be used for treating a subject and may depend on the weight and age of the subject and the route of administration, among other things.

“Excipient” refers to any diluent or vehicle for a drug.

“Medicament” refers to the combination of one or more drug substances and one or more excipients. Sometimes such combination is also described as “formulation” or “pharmaceutical composition”.

“Drug product,” “pharmaceutical dosage form,” “dosage form,” “final dosage form” and the like, refer to a pharmaceutical composition or a medicament suitable for treating a subject in need of treatment and generally may be in the form of tablets, capsules, sachets containing powder or granules, liquid solutions or suspensions, patches, films, and the like.

“Disease, disorder or condition associated with NLRP3” and similar phrases relate to a disease, disorder or condition in a subject for which inhibition of the NLRP3 inflammasome pathway may provide a therapeutic or prophylactic benefit.

The following abbreviations may be used in the specification: Ac (acetyl); Ac₂O (acetic anhydride); ACN (acetonitrile); AIBN (azo-bis-isobutyronitrile); AmPhos (bis(di-tert-butyl(4-dimethylaminophenyl)phosphine)dichloropalladium(II)); API (active pharmaceutical ingredient); aq (aqueous); BINAP (2,2′-bis(diphenylphosphino)-1,1′-binaphthyl); Bn (benzyl); Boc (tert-butoxycarbonyl); BrettPhos (2-(dicyclohexylphosphino)3,6-dimethoxy-2′,4′,6′-triisopropyl-1,1′-biphenyl); BrettPhos-Pd-G3 ([(2-di-cyclohexylphosphino-3,6-dimethoxy-2′,4′,6′-triisopropyl-1,1′-biphenyl)-2-(2′-amino-1,1′-biphenyl)]palladium(II)methanesulfonate); Cbz (carbobenzyloxy); Troc (2,2,2-trichloroethoxycarbonyl); dba (dibenzylideneacetone); DBU (1,8-diazabicyclo[5.4.0]undec-7-ene); DCC (1,3-dicyclohexylcarbodiimide); DCE (1,1-dichloroethane); DCM (dichloromethane); DEA (diethylamine); DIAD (diisopropyl azodicarboxylate); DIPEA (N,N-diisopropylethylamine, Hunig's Base); DMA (N,N-dimethylacetamide); DMAP (4-dimethylaminopyridine); DME (1,2-dimethoxyethane); DMF (N,N-dimethylformamide); DMP (Dess-Martin periodinane); DMSO (dimethylsulfoxide); dppf (1,1′-bis(diphenylphosphino)ferrocene); DTT (dithiothreitol); EC₅₀ (effective concentration at half maximal response); EDA (ethoxylated dodecyl alcohol, Brj®35); EDC (N-(3-dimethylaminopropyl)-N′-ethylcarbodiimide); EDTA (ethylenediaminetetraacetic acid); ee (enantiomeric excess); ELS (evaporative light scattering); eq (equivalents); Et (ethyl); Et₃N (triethylamine); EtOAc (ethyl acetate); EtOH (ethanol); FA (formic acid); HATU (2-(3H-[1,2,3]triazolo[4,5-b]pyridin-3-yl)-1,1,3,3-tetramethyluronium hexafluorophosphate(V)); HEPES (4-(2-hydroxyethyl)piperazine-1-ethanesulfonic acid); HOAc (acetic acid); HOBt (1H-benzo[d][1,2,3]triazol-1-ol); IC₅₀ (concentration at 50% inhibition); IPA (isopropanol); IPAc (isopropyl acetate); IPE (isopropyl ether); LDA (lithium diisopropylamide); LiHMDS (lithium bis(trimethylsilyl)amide); mCPBA (m-chloroperoxybenzoic acid); Me (methyl); MeOH (methanol); MTBE (methyl tert-butyl ether); mp (melting point); NaOt-Bu (sodium tertiary butoxide); NMM (N-methylmorpholine); NMP 1-methylpyrrolidin-2-one); OTf (triflate); PE (petroleum ether); Ph (phenyl); pEC₅₀ (−log₁₀(EC₅₀), where EC₅₀ is given in molar (M) units); pIC₅₀ (−log₁₀(IC₅₀), where IC₅₀ is given in molar (M) units); PMB (p-methoxylbenzyl); Pr (propyl); c-Pr (cyclopropyl), i-Pr (isopropyl); PTFE (polytetrafluoroethylene); PyBOP ((benzotriazol-1-yloxy)tripyrrolidinophosphonium hexafluorophosphate); PyBroP® (bromotripyrrolidinophosphonium hexafluorophosphate); PCy₃ (tricyclohexylphosphine); RT (room temperature, approximately 20° C. to 25° C.); SFC (supercritical fluid chromatography); T3P (2,4,6-tripropyl-1,3,5,2,4,6-trioxatriphosphinane 2,4,6-trioxide); TCEP (tris(2-carboxyethyl)phosphine); TFA (trifluoroacetic acid); TFAA (2,2,2-trifluoroacetic anhydride); THE (tetrahydrofuran); TMS (trimethylsilyl); Tris buffer (2-amino-2-hydroxymethyl-propane-1,3-diol buffer); XPhos (2-dicyclohexylphosphino-2′,4′,6′-triisopropylbiphenyl); and XPhos-Pd-G2 (chloro(2-dicyclohexylphosphino-2′,4′,6′-triisopropyl-1,1′-biphenyl)[2-(2′-amino-1,1′-biphenyl)]palladium(II)).

As described below, this disclosure concerns compounds of Formula (I), or pharmaceutically acceptable salts thereof, or solvates thereof, or hydrates thereof (collectively, sometimes to be referred to as compound (I) in the present specification). This disclosure also concerns materials and methods for preparing compounds of Formula (I), or pharmaceutically acceptable salts thereof, or solvates thereof, or hydrates thereof, medicaments which contain them, and the use of compounds of Formula (I), or pharmaceutically acceptable salts thereof, or solvates thereof, or hydrates thereof, (optionally in combination with other pharmacologically active agent(s)) for treating neurodegenerative diseases such as Parkinson's disease, Alzheimer's disease, Huntington's disease, amyotrophic lateral sclerosis, prion disease and other diseases, disorders and/or conditions associated with NLRP3.

The definition of each variable in Formula (I) is explained in detail in the following.

L is O or a bond.

As one embodiment, L is preferably O.

As another embodiment, L is preferably bond.

X is N or CR⁴ wherein R⁴ is as defined below.

X is preferably CR⁴ wherein R⁴ is as defined below.

Y is N or CR⁵ wherein R⁵ is as defined below.

Y is preferably CR⁵ wherein R⁵ is as defined below.

R¹ is an optionally substituted C₁₋₆ alkyl group, an optionally substituted C₃₋₈ cycloalkyl group, an optionally substituted C₆₋₁₄ aryl group, or an optionally substituted 4- to 6-membered heterocyclic group with the proviso that when L is a bond, then the 4- to 6-membered heterocyclic group is linked to the pyrazolopyrimidone ring by a carbon-carbon bond.

As one embodiment, R¹ is preferably an optionally substituted C₁₋₆ alkyl group.

As another embodiment, R¹ is preferably
(1) a C₁₋₆ alkyl group (e.g., methyl, ethyl, n-propyl, isopropyl, n-butyl, isobutyl, tert-butyl, neopentyl) optionally substituted by 1 to 3 substituents selected from
   (a) a halogen atom (e.g., a fluorine atom),
   (b) a hydroxy group,
   (c) an optionally halogenated C₁₋₆ alkoxy group (e.g., methoxy, difluoromethoxy, trifluoromethoxy, ethoxy, difluoroethoxy),
   (d) a di-C₁₋₆ alkylamino group (e.g., dimethylamino),
   (e) a C₇₋₁₆ aralkyloxy group (e.g., benzyloxy),
   (f) a 5- or 6-membered aromatic heterocyclic group (e.g., pyrazolyl),
   (g) a 5- or 6-membered non-aromatic heterocyclic group (e.g., tetrahydrofuryl, tetrahydropyranyl, morpholinyl, oxazolidinyl),
   (h) a carboxy group, and
   (i) a 4- to 6-membered non-aromatic heterocyclyloxy group (e.g., oxetanyloxy), and
   (j) a cyano group,
(2) a C₃₋₈ cycloalkyl group (e.g., cyclopropyl, cyclobutyl, cyclohexyl) optionally substituted by 1 to 3 substituents selected from
   (a) a C₁₋₆ alkyl group (e.g., methyl),
   (b) a C₁₋₆ alkoxy group (e.g., methoxy),
   (c) a halogen atom (e.g., a fluorine atom),
   (d) a cyano group, and
   (e) a group represented by the formula: —(CH₂)ₐ—O—(CH₂)_{b}—, wherein each of a and b is the integer of 0 to 3 and the sum of a and b is 2 to 4 (e.g., —CH₂OCH₂—),
(3) a C₆₋₁₄ aryl group (e.g., phenyl) optionally substituted by 1 to 3 substituents selected from
   (a) a halogen atom (e.g., a fluorine atom, a chlorine atom), and
   (b) a C₁₋₆ alkoxy group (e.g., methoxy),
(4) a 5- or 6-membered aromatic heterocyclic group (e.g., pyrazolyl, imidazolyl, triazolyl, oxazolyl, isoxazolyl, pyridyl, pyrimidinyl, pyridazinyl, pyrazinyl) optionally substituted by 1 to 3 substituents selected from
   (a) a halogen atom (e.g., a fluorine atom),
   (b) a C₁₋₆ alkyl group (e.g., methyl, ethyl) optionally substituted by 1 to 3 substituents selected from (i) halogen atom (e.g., a fluorine atom and (ii) C₁₋₆ alkoxy group (e.g., methoxy),
   (c) a C₁₋₆ alkoxy group (e.g., methoxy), and
   (d) a group represented by the formula: —(CH₂)ₐ—O—(CH₂)_{b}—, together with the 5- or 6-membered aromatic heterocyclic group to which it is attached, forming a fused 8- to 10-membered heterocyclic group, wherein each of a and b is the integer of 0 to 3 and the sum of a and b is 2 to 4 (e.g., —(CH₂)₂OCH₂—, —(CH₂)₂O—, —(CH₂)₃O—)
with the proviso that when L is a bond, then the 5- or 6-membered aromatic heterocyclic group is linked to the pyrazolopyrimidone ring by a carbon-carbon bond, or
(5) a 4- to 6-membered non-aromatic heterocyclic group (e.g., oxetanyl, tetrahydrofuryl, dihydropyranyl, tetrahydropyranyl, morpholinyl, dihydropyridyl, piperidyl, dioxanyl) optionally substituted by 1 to 3 substituents selected from
   (a) an oxo group,
   (b) a C₁₋₆ alkyl group (e.g., methyl), and
   (c) a halogen atom (e.g., fluorine atom), and
   (d) a C₁₋₆ alkoxy group (e.g., methoxy) with the proviso that when L is a bond, then the 5- or 6-membered non-aromatic heterocyclic group is linked to the pyrazolopyrimidone ring by a carbon-carbon bond.

R¹ is more preferably a C₁₋₆ alkyl group (e.g., methyl, ethyl) optionally substituted by an optionally halogenated C₁₋₆ alkoxy group (e.g., methoxy, difluoromethoxy). R¹ is more preferably a C₁₋₆ alkyl group (e.g., methyl).

R² is a hydrogen atom, an optionally substituted C₁₋₆ alkyl group, or an optionally substituted C₃₋₈ cycloalkyl group.

R² is preferably a hydrogen atom, or an optionally substituted C₁₋₆ alkyl group.

R² is more preferably
(1) a hydrogen atom, or
(2) a C₁₋₆ alkyl group (e.g., methyl).

R² is particularly preferably a hydrogen atom.

R³ is a hydrogen atom, an optionally substituted C₁₋₆ alkyl group, an optionally substituted C₃₋₈ cycloalkyl group, or a halogen atom.

R³ is preferably a hydrogen atom, an optionally substituted C₁₋₆ alkyl group, or a halogen atom.

R³ is more preferably
(1) a hydrogen atom,
(2) a C₁₋₆ alkyl group (e.g., methyl, ethyl), or
(3) a halogen atom (e.g., a chlorine atom).

R³ is particularly preferably a hydrogen atom.

R⁴ and R⁸ are each independently a hydrogen atom, an optionally substituted C₁₋₆ alkyl group, an optionally substituted C₃₋₈ cycloalkyl group, an optionally substituted C₂₋₆ alkenyl group, a halogen atom, a hydroxy group, an optionally substituted C₁₋₆ alkoxy group, an optionally substituted 5- or 6-membered heterocyclic group, a cyano group, an amino group or a nitro group.

R⁴ and R⁸ are preferably each independently a hydrogen atom, an optionally substituted C₁₋₆ alkyl group, an optionally substituted C₃₋₈ cycloalkyl group, a halogen atom, a hydroxy group, an optionally substituted C₁₋₆ alkoxy group, or an optionally substituted 5- or 6-membered heterocyclic group (preferably an optionally substituted 5- or 6-membered aromatic heterocyclic group).

As one embodiment, R⁴ and R⁸ are more preferably each independently an optionally substituted C₁₋₆ alkyl group, or a halogen atom.

As another embodiment, R⁴ and R⁸ are more preferably each independently
(1) a hydrogen atom,
(2) a C₁₋₆ alkyl group (e.g., methyl, ethyl) optionally substituted by 1 to 4, preferably 1 to 3 substituents selected from
   (i) halogen atoms (e.g., a fluorine atom),
   (ii) hydroxy group, and
   (iii) C₁₋₆ alkoxy group (e.g., methoxy),
(3) a C₃₋₈ cycloalkyl group (e.g., cyclopropyl) optionally substituted by 1 to 3 halogen atoms (e.g., a fluorine atom),
(4) a halogen atom (e.g., a fluorine atom, a chlorine atom, a bromine atom),
(5) a hydroxy group,
(6) a C₁₋₆ alkoxy group (e.g., methoxy, ethoxy) optionally substituted by 1 to 3 halogen atoms (e.g., a fluorine atom), or
(7) a 5- or 6-membered aromatic heterocyclic group (e.g., pyrazolyl)
(8) a cyano group.

R⁴ and R⁸ are further more preferably each independently
(1) a C₁₋₆ alkyl group (e.g., methyl) optionally substituted by 1 to 3 halogen atoms (e.g., a fluorine atom), or
(2) a halogen atom (e.g., a fluorine atom, bromine atom).

Still more preferably, one of R⁴ and R⁸ is a C₁₋₆ alkyl group (e.g., methyl), and the other is (1) a C₁₋₆ alkyl group (e.g., methyl) or (2) a halogen atom (e.g., a fluorine atom, bromine atom). R⁴ and R⁸ are most preferably both C₁₋₆ alkyl groups (e.g., methyl).

R⁵ and R⁷ are each independently a hydrogen atom, an optionally substituted C₁₋₆ alkyl group, an optionally substituted C₃₋₈ cycloalkyl group, an optionally substituted C₂₋₆ alkenyl group, a halogen atom, a hydroxy group, an optionally substituted C₁₋₆ alkoxy group, an optionally substituted 5- or 6-membered heterocyclic group, a cyano group, an amino group or a nitro group.

R⁵ and R⁷ are preferably each independently a hydrogen atom, an optionally substituted C₁₋₆ alkyl group, or a halogen atom.

R⁵ and R⁷ are more preferably each independently
(1) a hydrogen atom,
(2) a C₁₋₆ alkyl group (e.g., methyl, ethyl) optionally substituted by 1 to 3 halogen atoms (e.g., fluorine atoms),
(3) a halogen atom (e.g., a bromine atom, a fluorine atom), or
(4) a C₃₋₈ cycloalkyl group.

R⁵ and R⁷ are particularly preferably both hydrogen atoms.

R⁶ is a hydrogen atom, an optionally substituted C₁₋₆ alkyl group, an optionally substituted C₃₋₈ cycloalkyl group, an optionally substituted C₂₋₆ alkenyl group, a halogen atom, a hydroxy group, an optionally substituted C₁₋₆ alkoxy group, an optionally substituted 5- or 6-membered heterocyclic group, a cyano group, an amino group or a nitro group.

R⁶ is preferably a hydrogen atom, an optionally substituted C₁₋₆ alkyl group, an optionally substituted C₃₋₈ cycloalkyl group, an optionally substituted C₂₋₆ alkenyl group, a halogen atom, an optionally substituted C₁₋₆ alkoxy group, or an optionally substituted 5- or 6-membered heterocyclic group (preferably an optionally substituted 5- or 6-membered aromatic heterocyclic group).

As one embodiment, R⁶ is more preferably an optionally substituted C₃₋₈ cycloalkyl group, or a halogen atom.

As another embodiment, R⁶ is more preferably
(1) a hydrogen atom,
(2) a C₁₋₆ alkyl group (e.g., methyl, ethyl, propyl, isopropyl) optionally substituted by 1 to 3 halogen atoms (e.g., a fluorine atom),
(3) a C₃₋₈ cycloalkyl group (e.g., cyclopropyl) optionally substituted by 1 to 3 halogen atoms (e.g., a fluorine atom),
(4) a C₂₋₆ alkenyl group (e.g., vinyl),
(5) a halogen atom (e.g., a fluorine atom, a chlorine atom, a bromine atom),
(6) a C₁₋₆ alkoxy group (e.g., methoxy, ethoxy) optionally substituted by 1 to 3 halogen atoms (e.g., a fluorine atom), or
(7) a 5- or 6-membered aromatic heterocyclic group (e.g., pyrazolyl)
(8) an amino group, or
(9) a nitro group.

R⁶ is further more preferably
(1) a C₃₋₈ cycloalkyl group (e.g., cyclopropyl) optionally substituted by 1 to 3 halogen atoms (a fluorine atom),
(2) a halogen atom (e.g., a bromine atom), or
(3) a C₁₋₆ alkoxy group (e.g., methoxy) optionally substituted by 1 to 3 halogen atoms (e.g., a fluorine atom).

R⁶ is most preferably a C₃₋₈ cycloalkyl group (e.g., cyclopropyl).

Preferable embodiment of a compound of Formula (I) includes the following compounds.

### [Compound A]

L is O or a bond;
X is N or CR⁴;
Y is N or CR⁵;
R¹ is an optionally substituted C₁₋₆ alkyl group, an optionally substituted C₃₋₈ cycloalkyl group, an optionally substituted C₆₋₁₄ aryl group, or an optionally substituted 4- to 6-membered heterocyclic group with the proviso that when L is a bond, then the 4 to 6-membered heterocyclic group is a 4- to 6-membered non-aromatic heterocyclic group which is linked to the pyrazolopyrimidone ring by a carbon-carbon bond;
R² is a hydrogen atom, or an optionally substituted C₁₋₆ alkyl group;
R³ is a hydrogen atom, an optionally substituted C₁₋₆ alkyl group, or a halogen atom;
R⁴ and R⁸ are each independently a hydrogen atom, an optionally substituted C₁₋₆ alkyl group, an optionally substituted C₃₋₈ cycloalkyl group, a halogen atom, a hydroxy group, an optionally substituted C₁₋₆ alkoxy group, an optionally substituted 5- or 6-membered heterocyclic group (preferably an optionally substituted 5- or 6-membered aromatic heterocyclic group), or a cyano group;
R⁵ and R⁷ are each independently a hydrogen atom, an optionally substituted C₁₋₆ alkyl group, a halogen atom or a C₃₋₈ cycloalkyl group; and
R⁶ is a hydrogen atom, an optionally substituted C₁₋₆ alkyl group, an optionally substituted C₃₋₈ cycloalkyl group, an optionally substituted C₂₋₆ alkenyl group, a halogen atom, an optionally substituted C₁₋₆ alkoxy group, an optionally substituted 5- or 6-membered heterocyclic group (preferably an optionally substituted 5- or 6-membered aromatic heterocyclic group), an amino group, or a nitro group; with the proviso that (1) 2,5-dihydro-6-methyl-5-(1-methylethyl)-2-(3-pyridinyl)-4H-pyrazolo[3,4-d]pyrimidin-4-one, (2) 5-ethyl-2,5-dihydro-6-methyl-2-(3-pyridinyl)-4H-pyrazolo[3,4-d]pyrimidin-4-one and (3) 5-ethyl-2,5-dihydro-2-(3-pyridinyl)-6-(trifluoromethyl)-4H-pyrazolo[3,4-d]pyrimidin-4-one are excluded.

### [Compound B]

L is O or a bond;
X is N or CR⁴;
Y is N or CR⁵;
R¹ is
(1) a C₁₋₆ alkyl group (e.g., methyl, ethyl, n-propyl, isopropyl, n-butyl, isobutyl, tert-butyl, neopentyl) optionally substituted by 1 to 3 substituents selected from
   (a) a halogen atom (e.g., a fluorine atom),
   (b) a hydroxy group,
   (c) an optionally halogenated C₁₋₆ alkoxy group (e.g., methoxy, difluoromethoxy, trifluoromethoxy, ethoxy, difluoroethoxy),
   (d) a di-C₁₋₆ alkylamino group (e.g., dimethylamino),
   (e) a C₇₋₁₆ aralkyloxy group (e.g., benzyloxy),
   (f) a 5- or 6-membered aromatic heterocyclic group (e.g., pyrazolyl),
   (g) a 5- or 6-membered non-aromatic heterocyclic group (e.g., tetrahydrofuryl, tetrahydropyranyl, morpholinyl, oxazolidinyl),
   (h) a carboxy group,
   (i) a 4- to 6-membered non-aromatic heterocyclyloxy group (e.g., oxetanyloxy), and
   (j) a cyano group,
(2) a C₃₋₈ cycloalkyl group (e.g., cyclopropyl, cyclobutyl, cyclohexyl) optionally substituted by 1 to 3 substituents selected from
   (a) a C₁₋₆ alkyl group (e.g., methyl),
   (b) a C₁₋₆ alkoxy group (e.g., methoxy),
   (c) a halogen atom (e.g., a fluorine atom),
   (d) a cyano group, and
   (e) a group represented by the formula: —(CH₂)ₐ—O—(CH₂)_{b}—, wherein each of a and b is the integer of 0 to 3 and the sum of a and b is 2 to 4 (e.g., —CH₂OCH₂—),
(3) a C₆₋₁₄ aryl group (e.g., phenyl) optionally substituted by 1 to 3 substituents selected from
   (a) a halogen atom (e.g., a fluorine atom, a chlorine atom), and
   (b) a C₁₋₆ alkoxy group (e.g., methoxy),
(4) a 5- or 6-membered aromatic heterocyclic group (e.g., pyrazolyl, imidazolyl, triazolyl, oxazolyl, isoxazolyl, pyridyl, pyrimidinyl, pyridazinyl, pyrazinyl) optionally substituted by 1 to 3 substituents selected from
   (a) a halogen atom (e.g., a fluorine atom),
   (b) a C₁₋₆ alkyl group (e.g., methyl, ethyl) optionally substituted by 1 to 3 substituents selected (i) halogen atom (e.g., a fluorine atom) and (ii) C₁₋₆ alkoxy group (e.g., methoxy),
   (c) a C₁₋₆ alkoxy group (e.g., methoxy), and
   (d) a group represented by the formula: —(CH₂)ₐ—O—(CH₂)_{b}—, together with the 5- or 6-membered aromatic heterocyclic group to which it is attached, forming a fused 8- to 10-membered heterocyclic group, wherein each of a and b is the integer of 0 to 3 and the sum of a and b is 2 to 4 (e.g., —(CH₂)₂OCH₂—, —(CH₂)₂O—, —(CH₂)₃O—), or
(5) a 4- to 6-membered non-aromatic heterocyclic group (e.g., oxetanyl, tetrahydrofuryl, dihydropyranyl, tetrahydropyranyl, morpholinyl, dihydropyridyl, piperidyl, dioxanyl) optionally substituted by 1 to 3 substituents selected from
   (a) an oxo group,
   (b) a C₁₋₆ alkyl group (e.g., methyl),
   (c) a halogen atom (e.g., fluorine atom), and
   (d) a C₁₋₆ alkoxy group (e.g., methoxy);
R² is
(1) a hydrogen atom, or
(2) a C₁₋₆ alkyl group (e.g., methyl);
R³ is
(1) a hydrogen atom,
(2) a C₁₋₆ alkyl group (e.g., methyl, ethyl), or
(3) a halogen atom (e.g., a chlorine atom);
R⁴ and R⁸ are each independently
(1) a hydrogen atom,
(2) a C₁₋₆ alkyl group (e.g., methyl, ethyl) optionally substituted by 1 to 4, preferably 1 to 3 substituents selected from
   (i) halogen atoms (e.g., a fluorine atom),
   (ii) hydroxy group, and
   (iii) C₁₋₆ alkoxy group (e.g., methoxy),
(3) a C₃₋₈ cycloalkyl group (e.g., cyclopropyl) optionally substituted by 1 to 3 halogen atoms (e.g., a fluorine atom),
(4) a halogen atom (e.g., a fluorine atom, a chlorine atom, a bromine atom),
(5) a hydroxy group,
(6) a C₁₋₆ alkoxy group (e.g., methoxy, ethoxy) optionally substituted by 1 to 3 halogen atoms (e.g., a fluorine atom),
(7) a 5- or 6-membered aromatic heterocyclic group (e.g., pyrazolyl), or
(8) a cyano group;
R⁵ and R⁷ are each independently
(1) a hydrogen atom,
(2) a C₁₋₆ alkyl group (e.g., methyl, ethyl) optionally substituted by 1 to 3 halogen atoms (e.g., fluorine atoms),
(3) a halogen atom (e.g., a bromine atom, a fluorine atom), or
(4) a C₃₋₈ cycloalkyl group; and
R⁶ is
(1) a hydrogen atom,
(2) a C₁₋₆ alkyl group (e.g., methyl, ethyl, propyl, isopropyl) optionally substituted by 1 to 3 halogen atoms (e.g., a fluorine atom),
(3) a C₃₋₈ cycloalkyl group (e.g., cyclopropyl) optionally substituted by 1 to 3 halogen atoms (e.g., a fluorine atom),
(4) a C₂₋₆ alkenyl group (e.g., vinyl),
(5) a halogen atom (e.g., a fluorine atom, a chlorine atom, a bromine atom),
(6) a C₁₋₆ alkoxy group (e.g., methoxy, ethoxy) optionally substituted by 1 to 3 halogen atoms (e.g., a fluorine atom),
(7) a 5- or 6-membered aromatic heterocyclic group (e.g., pyrazolyl),
(8) an amino group, or
(9) a nitro group;
with the proviso that (1) 2,5-dihydro-6-methyl-5-(1-methylethyl)-2-(3-pyridinyl)-4H-pyrazolo[3,4-d]pyrimidin-4-one, (2) 5-ethyl-2,5-dihydro-6-methyl-2-(3-pyridinyl)-4H-pyrazolo[3,4-d]pyrimidin-4-one and (3) 5-ethyl-2,5-dihydro-2-(3-pyridinyl)-6-(trifluoromethyl)-4H-pyrazolo[3,4-d]pyrimidin-4-one are excluded.

### [Compound C]

L is O or bond;
X is CR⁴;
Y is CR⁵;
R¹ is a C₁₋₆ alkyl group (e.g., methyl, ethyl) optionally substituted by an optionally halogenated C₁₋₆ alkoxy group (e.g., methoxy, difluoromethoxy);
R² is a hydrogen atom;
R³ is a hydrogen atom;
R⁴ and R⁸ are each independently
(1) a C₁₋₆ alkyl group (e.g., methyl) optionally substituted by 1 to 3 halogen atoms (e.g., a fluorine atom), or
(2) a halogen atom (e.g., a fluorine atom, bromine atom) (preferably, one of R⁴ and R⁸ is a C₁₋₆ alkyl group (e.g., methyl) optionally substituted by 1 to 3 halogen atoms (e.g., a fluorine atom), and the other is (1) a C₁₋₆ alkyl group (e.g., methyl) or (2) a halogen atom (e.g., a fluorine atom, bromine atom));
R⁵ and R⁷ are both hydrogen atoms; and
R⁶ is
(1) a C₃₋₈ cycloalkyl group (e.g., cyclopropyl) optionally substituted by 1 to 3 halogen atoms (a fluorine atom),
(2) a halogen atom (e.g., a bromine atom), or
(3) a C₁₋₆ alkoxy group (e.g., methoxy) optionally substituted by 1 to 3 halogen atoms (e.g., a fluorine atom).

### [Compound D]

L is O;
X is CR⁴;
Y is CR⁵;
R¹ is a C₁₋₆ alkyl group (e.g., methyl, ethyl) optionally substituted by 1 to 3 C₁₋₆ alkoxy group (e.g., methoxy);
R² is a hydrogen atom;
R³ is a hydrogen atom;
R⁴ and R⁸ are each independently
(1) a C₁₋₆ alkyl group (e.g., methyl) optionally substituted by 1 to 3 halogen atoms (e.g., a fluorine atom), or
(2) a halogen atom (e.g., a fluorine atom, bromine atom) (preferably, one of R⁴ and R⁸ is a C₁₋₆ alkyl group (e.g., methyl), and the other is (1) a C₁₋₆ alkyl group (e.g., methyl) optionally substituted by 1 to 3 halogen atoms (e.g., a fluorine atom) or (2) a halogen atom (e.g., a fluorine atom, bromine atom));
R⁵ and R⁷ are both hydrogen atoms; and
R⁶ is
(1) a C₃₋₈ cycloalkyl group (e.g., cyclopropyl) optionally substituted by 1 to 3 halogen atoms (e.g., a fluorine atom),
(2) a halogen atom (e.g., a bromine atom), or
(3) a C₁₋₆ alkoxy group (e.g., methoxy) optionally substituted by 1 to 3 halogen atoms (e.g., a fluorine atom).

### [Compound G]

L is O;
X is CR⁴;
Y is CR⁵;
R¹ is a C₁₋₆ alkyl group (e.g., methyl);
R² is a hydrogen atom;
R³ is a hydrogen atom;
R⁴ and R⁸ are both C₁₋₆ alkyl groups (e.g., methyl);
R⁵ and R⁷ are both hydrogen atoms; and
R⁶ is a C₃₋₈ cycloalkyl group (e.g., cyclopropyl).

### [Compound H]

L is a bond;
X is N or CR⁴;
Y is N or CR⁵;
R¹ is an optionally substituted C₁₋₆ alkyl group, an optionally substituted C₃₋₈ cycloalkyl group, an optionally substituted C₆₋₁₄ aryl group, or an optionally substituted 4- to 6-membered non-aromatic heterocyclic group;
R² is a hydrogen atom, or an optionally substituted C₁₋₆ alkyl group;
R³ is a hydrogen atom, an optionally substituted C₁₋₆ alkyl group, or a halogen atom;
R⁴ and R⁸ are each independently a hydrogen atom, an optionally substituted C₁₋₆ alkyl group, an optionally substituted C₃₋₈ cycloalkyl group, a halogen atom, a hydroxy group, an optionally substituted C₁₋₆ alkoxy group, an optionally substituted 5- or 6-membered heterocyclic group (preferably an optionally substituted 5- or 6-membered aromatic heterocyclic group) or a cyano group;
R⁵ and R⁷ are each independently a hydrogen atom, an optionally substituted C₁₋₆ alkyl group, a halogen atom, or optionally substituted C₃₋₈ cycloalkyl group; and
R⁶ is a hydrogen atom, an optionally substituted C₁₋₆ alkyl group, an optionally substituted C₃₋₈ cycloalkyl group, an optionally substituted C₂₋₆ alkenyl group, a halogen atom, an optionally substituted C₁₋₆ alkoxy group, an optionally substituted 5- or 6-membered heterocyclic group (preferably an optionally substituted 5- or 6-membered aromatic heterocyclic group), an amino group, or a nitro group;
with the proviso that (1) 2,5-dihydro-6-methyl-5-(1-methylethyl)-2-(3-pyridinyl)-4H-pyrazolo[3,4-d]pyrimidin-4-one, (2) 5-ethyl-2,5-dihydro-6-methyl-2-(3-pyridinyl)-4H-pyrazolo[3,4-d]pyrimidin-4-one and (3) 5-ethyl-2,5-dihydro-2-(3-pyridinyl)-6-(trifluoromethyl)-4H-pyrazolo[3,4-d]pyrimidin-4-one are excluded.

### [Compound I]

L is a bond;
X is CR⁴;
Y is CR⁵;
R¹ is
(1) a C₁₋₆ alkyl group (e.g., methyl, ethyl, n-propyl, isopropyl, n-butyl, isobutyl, tert-butyl, neopentyl) optionally substituted by 1 to 3 substituents selected from
   (a) a halogen atom (e.g., a fluorine atom),
   (b) a hydroxy group,
   (c) an optionally halogenated C₁₋₆ alkoxy group (e.g., methoxy, difluoromethoxy, trifluoromethoxy, ethoxy, difluoroethoxy),
   (d) a C₇₋₁₆ aralkyloxy group (e.g., benzyloxy),
   (e) a 5- or 6-membered aromatic heterocyclic group (e.g., pyrazolyl),
   (f) a 5- or 6-membered non-aromatic heterocyclic group (e.g., tetrahydrofuryl, tetrahydropyranyl, morpholinyl, oxazolidinyl),
   (g) a carboxy group,
   (h) a 4- to 6-membered non-aromatic heterocyclyloxy group (e.g., oxetanyloxy), and
   (i) a cyano group,
(2) a C₃₋₈ cycloalkyl group (e.g., cyclopropyl, cyclobutyl, cyclohexyl) optionally substituted by 1 to 3 substituents selected from
   (a) a C₁₋₆ alkyl group (e.g., methyl),
   (b) a C₁₋₆ alkoxy group (e.g., methoxy),
   (c) a halogen atom (e.g., a fluorine atom), and
   (d) a cyano group,
(3) a C₆₋₁₄ aryl group (e.g., phenyl) optionally substituted by 1 to 3 substituents selected from
   (a) a halogen atom (e.g., a fluorine atom, a chlorine atom), and
   (b) a C₁₋₆ alkoxy group (e.g., methoxy), or
(4) a 4- to 6-membered non-aromatic heterocyclic group (e.g., oxetanyl, tetrahydrofuryl, dihydropyranyl, tetrahydropyranyl, morpholinyl, dihydropyridyl, piperidyl, dioxanyl) optionally substituted by 1 to 3 substituents selected from
   (a) an oxo group,
   (b) a C₁₋₆ alkyl group (e.g., methyl),
   (c) a halogen atom (e.g., fluorine atom), and
   (d) a C₁₋₆ alkoxy group (e.g., methoxy);
R² is
(1) a hydrogen atom, or
(2) a C₁₋₆ alkyl group (e.g., methyl);
R³ is
(1) a hydrogen atom, or
(2) a C₁₋₆ alkyl group (e.g., methyl);
R⁴ and R⁸ are each independently
(1) a hydrogen atom,
(2) a C₁₋₆ alkyl group (e.g., methyl, ethyl) optionally substituted by 1 to 4, preferably 1 to 3 halogen atoms (e.g., a fluorine atom),
(3) a halogen atom (e.g., a fluorine atom, a chlorine atom),
(4) a hydroxy group, or
(5) a C₁₋₆ alkoxy group (e.g., ethoxy);
R⁵ and R⁷ are each independently
(1) a hydrogen atom,
(2) a C₁₋₆ alkyl group (e.g., methyl) optionally substituted by 1 to 3 halogen atoms (e.g., fluorine atoms),
(3) a halogen atom (e.g., a bromine atom), or
(4) a C₃₋₈ cycloalkyl group; and
R⁶ is
(1) a hydrogen atom,
(2) a C₁₋₆ alkyl group (e.g., methyl,
(3) a C₃₋₈ cycloalkyl group (e.g., cyclopropyl) optionally substituted by 1 to 3 halogen atoms (e.g., a fluorine atom),
(4) a halogen atom (e.g., a chlorine atom, a bromine atom),
(5) an amino group, or
(6) a nitro group.

### [Compound J]

L is bond;
X is CR⁴;
Y is CR⁵;
R¹ is a C₁₋₆ alkyl group (e.g., methyl) optionally substituted by an optionally halogenated C₁₋₆ alkoxy group (e.g., methoxy, difluoromethoxy);
R² is a hydrogen atom;
R³ is a hydrogen atom;
R⁴ and R⁸ are both C₁₋₆ alkyl groups (e.g., methyl) optionally substituted by 1 to 3 halogen atoms (e.g, a fluorine atom);
R⁵ and R⁷ are both hydrogen atoms; and
R⁶ is a C₃₋₈ cycloalkyl group (e.g., cyclopropyl).

Specific examples of a compound of Formula (I) include the compounds of Examples 1 to 326.

The favorable compounds of Formula (I) are
2-(4-bromo-2-fluoro-6-methylphenyl)-6-ethoxy-2,5-dihydro-4H-pyrazolo[3,4-d]pyrimidin-4-one (Example 24);
2-(4-cyclopropyl-2-fluoro-6-methylphenyl)-6-ethoxy-2,5-dihydro-4H-pyrazolo[3,4-d]pyrimidin-4-one (Example 39);
2-[2-bromo-4-(difluoromethoxy)-6-methylphenyl]-6-ethoxy-2,5-dihydro-4H-pyrazolo[3,4-d]pyrimidin-4-one (Example 142);
2-(4-cyclopropyl-2,6-dimethylphenyl)-6-methoxy-2,5-dihydro-4H-pyrazolo[3,4-d]pyrimidin-4-one (Example 146);
2-[4-cyclopropyl-2-(difluoromethyl)-6-fluorophenyl]-6-(2-methoxyethoxy)-2,5-dihydro-4H-pyrazolo[3,4-d]pyrimidin-4-one (Example 186);
2-(4-cyclopropyl-2,6-dimethylphenyl)-6-[(difluoromethoxy)methyl]-2,5-dihydro-4H-pyrazolo[3,4-d]pyrimidin-4-one (Example 224);
2-[4-(1-fluorocyclopropyl)-2,6-dimethylphenyl]-6-methoxy-2,5-dihydro-4H-pyrazolo[3,4-d]pyrimidin-4-one (Example 235); and
2-[4-cyclopropyl-2-(difluoromethyl)-6-methylphenyl]-6-(methoxymethyl)-2,5-dihydro-4H-pyrazolo[3,4-d]pyrimidin-4-one (Example 321);

Compounds of Formula (I) include embodiments described in the preceding paragraphs and compounds specifically named in the examples, may exist as salts, complexes, solvates, hydrates, and liquid crystals. Likewise, compounds of Formula (I) that are salts may exist as complexes, solvates, hydrates, and liquid crystals.

Compounds of Formula (I) may form pharmaceutically acceptable complexes, salts, solvates and hydrates. These salts include acid addition salts (including di-acids) and base salts. Pharmaceutically acceptable acid addition salts include salts derived from inorganic acids such as hydrochloric acid, nitric acid, phosphoric acid, sulfuric acid, hydrobromic acid, hydroiodic acid, hydrofluoric acid, and phosphorous acids, as well nontoxic salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts include acetate, adipate, aspartate, benzoate, besylate, bicarbonate, carbonate, bisulfate, sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate, hydrogen phosphate, dihydrogen phosphate, pyroglutamate, saccharate, stearate, succinate, tannate, tartrate, tosylate, trifluoroacetate and xinofoate salts.

Pharmaceutically acceptable base salts include salts derived from bases, including metal cations, such as an alkali or alkaline earth metal cation, as well as amines. Examples of suitable metal cations include sodium, potassium, magnesium, calcium, zinc, and aluminum. Examples of suitable amines include arginine, N,N′-dibenzylethylenediamine, chloroprocaine, choline, diethylamine, diethanolamine, dicyclohexylamine, ethylenediamine, glycine, lysine, N-methylglucamine, olamine, 2-amino-2-hydroxymethyl-propane-1,3-diol, and procaine. For a discussion of useful acid addition and base salts, see S. M. Berge et al., J. Pharm. Sci. (1977) 66:1-19; see also Stahl and Wermuth, Handbook of Pharmaceutical Salts: Properties, Selection, and Use (2002).

Pharmaceutically acceptable salts may be prepared using various methods. For example, a compound of Formula (I) may be reacted with an appropriate acid or base to give the desired salt. Alternatively, a precursor of the compound of Formula (I) may be reacted with an acid or base to remove an acid- or base-labile protecting group or to open a lactone or lactam group of the precursor. Additionally, a salt of the compound of Formula (I) may be converted to another salt (or free form) through treatment with an appropriate acid or base or through contact with an ion exchange resin. Following reaction, the salt may be isolated by filtration if it precipitates from solution, or by evaporation to recover the salt. The degree of ionization of the salt may vary from completely ionized to almost non-ionized.

Compounds of Formula (I) may exist in a continuum of solid states ranging from fully amorphous to fully crystalline. The term “amorphous” refers to a state in which the material lacks long range order at the molecular level and, depending upon temperature, may exhibit the physical properties of a solid or a liquid. Typically, such materials do not give distinctive X-ray diffraction patterns and, while exhibiting the properties of a solid, are more formally described as a liquid. Upon heating, a change from solid to liquid properties occurs which is characterized by a change of state, typically second order (“glass transition”). The term “crystalline” refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (“melting point”).

Compounds of Formula (I) may also exist in unsolvated and solvated forms. The term “solvate” describes a molecular complex comprising the compound and one or more pharmaceutically acceptable solvent molecules (e.g., ethanol) which is other than water. The term “hydrate” means a solvate in which the solvent is water. Pharmaceutically acceptable solvates include those in which the solvent may be isotopically substituted (e.g., D₂O, acetone-d₆, DMSO-d₆).

A currently accepted classification system for solvates and hydrates of organic compounds is one that distinguishes between isolated site, channel, and metal-ion coordinated solvates and hydrates. See, e.g., K. R. Morris (H. G. Brittain ed.) Polymorphism in Pharmaceutical Solids (1995). Isolated site solvates and hydrates are ones in which the solvent (e.g., water) molecules are isolated from direct contact with each other by intervening molecules of the organic compound. In channel solvates, the solvent molecules lie in lattice channels where they are next to other solvent molecules. In metal-ion coordinated solvates, the solvent molecules are bonded to the metal ion.

When the solvent or water is tightly bound, the complex will have a well-defined stoichiometry independent of humidity. When, however, the solvent or water is weakly bound, as in channel solvates and in hygroscopic compounds, the water or solvent content will depend on humidity and drying conditions. In such cases, non-stoichiometry will typically be observed.

Compounds of Formula (I) may also exist as multi-component complexes (other than salts and solvates) in which the compound (drug) and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions but could also be a complex of a neutral molecule with a salt. Co-crystals may be prepared by melt crystallization, by recrystallization from solvents, or by physically grinding the components together. See, e.g., O. Almarsson and M. J. Zaworotko, Chem. Commun. (2004) 17:1889-1896. For a general review of multi-component complexes, see J. K. Haleblian, J. Pharm. Sci. (1975) 64(8):1269-88.

When subjected to suitable conditions, compounds of Formula (I) may exist in a mesomorphic state (mesophase or liquid crystal). The mesomorphic state lies between the true crystalline state and the true liquid state (either melt or solution). Mesomorphism arising as the result of a change in temperature is described as “thermotropic” and mesomorphism resulting from the addition of a second component, such as water or another solvent, is described as “lyotropic.” Compounds that have the potential to form lyotropic mesophases are described as “amphiphilic” and include molecules which possess a polar ionic moiety (e.g., —COO⁻Na⁺, —COO⁻K⁺, —SO₃⁻Na⁺) or polar non-ionic moiety (such as —N⁻N⁺(CH₃)₃). See, e.g., N. H. Hartshorne and A. Stuart, Crystals and the Polarizing Microscope (4th ed, 1970).

Each compound of Formula (I) may exist as polymorphs, stereoisomers, tautomers, or some combination thereof, may be isotopically-labeled, may result from the administration of a prodrug, or form a metabolite following administration.

“Prodrugs” refer to compounds having little or no pharmacological activity that can, when metabolized in vivo, undergo conversion to compounds having desired pharmacological activity. Prodrugs may be prepared by replacing appropriate functionalities present in pharmacologically active compounds with “pro-moieties” as described, for example, in H. Bundgaar, Design of Prodrugs (1985). Examples of prodrugs include ester, ether or amide derivatives of compounds of Formula (I) having carboxylic acid, hydroxy, or amino functional groups, respectively. For further discussions of prodrugs, see e.g., T. Higuchi and V. Stella “Pro-drugs as Novel Delivery Systems,” ACS Symposium Series 14 (1975) and E. B. Roche ed., Bioreversible Carriers in Drug Design (1987).

“Metabolites” refer to compounds formed in vivo upon administration of pharmacologically active compounds. Examples include hydroxymethyl, hydroxy, secondary amino, primary amino, phenol, and carboxylic acid derivatives of compounds of Formula (I) having methyl, alkoxy, tertiary amino, secondary amino, phenyl, and amide groups, respectively.

Compounds of Formula (I) may exist as stereoisomers that result from the presence of one or more stereogenic centers, one or more double bonds, or both. The stereoisomers may be pure, substantially pure, or mixtures. Such stereoisomers may also result from acid addition or base salts in which the counter-ion is optically active, for example, when the counter-ion is D-lactate or L-lysine.

Compounds of Formula (I) may exist as tautomers, which are isomers resulting from tautomerization. Tautomeric isomerism includes, for example, imine-enamine, keto-enol, oxime-nitroso, and amide-imidic acid tautomerism.

Compounds of Formula (I) may exhibit more than one type of isomerism.

Geometrical (cis/trans) isomers may be separated by conventional techniques such as chromatography and fractional crystallization.

Conventional techniques for preparing or isolating a compound having a specific stereochemical configuration include chiral synthesis from a suitable optically pure precursor or resolution of the racemate (or the racemate of a salt or derivative) using, for example, chiral high-pressure liquid chromatography (HPLC). Alternatively, the racemate (or a racemic precursor) may be reacted with a suitable optically active compound, for example, an alcohol, or, in the case where the compound of Formula (I) contains an acidic or basic moiety, an acid or base such as tartaric acid or 1-phenylethylamine. The resulting diastereomeric mixture may be separated by chromatography, fractional crystallization, etc., and the appropriate diastereoisomer converted to the compound having the requisite stereochemical configuration. For a further discussion of techniques for separating stereoisomers, see E. L. Eliel and S. H. Wilen, Stereochemistry of Organic Compounds (1994).

Compounds of Formula (I) may possess isotopic variations, in which at least one atom is replaced by an atom having the same atomic number, but an atomic mass different from the atomic mass usually found in nature. Isotopes suitable for inclusion in compounds of Formula (I) include, for example, isotopes of hydrogen, such as ²H and ³H; isotopes of carbon, such as ¹¹C, ¹³C and ¹⁴C; isotopes of nitrogen, such as ¹³N and ¹⁵N; isotopes of oxygen, such as ¹⁵O, ¹⁷O and ¹⁸O; isotopes of sulfur, such as ³⁵S; isotopes of fluorine, such as ¹⁸F; isotopes of chlorine, such as ³⁶Cl, and isotopes of iodine, such as ¹²³I and ¹²⁵I. Use of isotopic variations (e.g., deuterium, ²H) may afford certain therapeutic advantages resulting from greater metabolic stability, for example, increased in vivo half-life or reduced dosage requirements. Additionally, certain isotopic variations of the disclosed compounds may incorporate a radioactive isotope (e.g., tritium, ³H, or ¹⁴C), which may be useful in drug and/or substrate tissue distribution studies. Substitution with positron emitting isotopes, such as ¹¹C, ¹⁸F, ¹⁵O and ¹³N, may be useful in Positron Emission Topography (PET) studies for examining substrate receptor occupancy. Isotopically-labeled compounds may be prepared by processes analogous to those described elsewhere in the disclosure using an appropriate isotopically-labeled reagent in place of a non-labeled reagent.

Compound (I) may be prepared using the techniques described below. Some of the methods and examples may omit details of common reactions, including oxidations, reductions, and so on, separation techniques (extraction, evaporation, precipitation, chromatography, filtration, trituration, crystallization, and the like), and analytical procedures, which are known to persons of ordinary skill in the art of organic chemistry. The details of such reactions and techniques can be found in several treatises, including Richard Larock, Comprehensive Organic Transformations (1999), and the multi-volume series edited by Michael B. Smith and others, Compendium of Organic Synthetic Methods (1974 et seq.). Starting materials and reagents may be obtained from commercial sources or may be prepared using literature methods. Some of the reaction schemes may omit minor products resulting from chemical transformations (e.g., an alcohol from the hydrolysis of an ester, CO₂ from the decarboxylation of a di-acid, etc.). In addition, in some instances, reaction intermediates may be used in subsequent steps without isolation or purification (i.e., in situ).

In the methods and examples below, certain compounds may be prepared using protecting groups, which prevent undesirable chemical reaction at otherwise reactive sites. Protecting groups may also be used to enhance solubility or otherwise modify physical properties of a compound. For a discussion of protecting group strategies, a description of materials and methods for installing and removing protecting groups, and a compilation of useful protecting groups for common functional groups, including amines, carboxylic acids, alcohols, ketones, aldehydes, and so on, see T. W. Greene and P. G. Wuts, Protecting Groups in Organic Chemistry (1999) and P. Kocienski, Protective Groups (2000).

Generally, the chemical transformations described throughout the specification may be carried out using substantially stoichiometric amounts of reactants, though certain reactions may benefit from using an excess of one or more of the reactants. Additionally, many of the reactions disclosed throughout the specification may be carried out at about room temperature (RT) and ambient pressure, but depending on reaction kinetics, yields, and so on, some reactions may be run at elevated pressures or employ higher temperatures (e.g., reflux conditions) or lower temperatures (e.g., −78° C. to 0° C.). Any reference in the disclosure and claims to a stoichiometric range, a temperature range, a pH range, etc., whether expressly using the word “range,” also includes the indicated endpoints.

Many of the chemical transformations may also employ one or more compatible solvents, which may influence the reaction rate and yield. Depending on the nature of the reactants, the one or more solvents may be polar protic solvents (including water), polar aprotic solvents, non-polar solvents, or some combination. Representative solvents include saturated aliphatic hydrocarbons (e.g., n-pentane, n-hexane, n-heptane, n-octane, cyclohexane, methylcyclohexane); aromatic hydrocarbons (e.g., benzene, toluene, xylenes); halogenated hydrocarbons (e.g., methylene chloride, chloroform, carbon tetrachloride); aliphatic alcohols (e.g., methanol, ethanol, propan-1-ol, propan-2-ol, butan-1-ol, 2-methyl-propan-1-ol, butan-2-ol, 2-methyl-propan-2-ol, pentan-1-ol, 3-methyl-butan-1-ol, hexan-1-ol, 2-methoxy-ethanol, 2-ethoxy-ethanol, 2-butoxy-ethanol, 2-(2-methoxy-ethoxy)-ethanol, 2-(2-ethoxy-ethoxy)-ethanol, 2-(2-butoxy-ethoxy)-ethanol); ethers (e.g., diethyl ether, di-isopropyl ether, dibutyl ether, 1,2-dimethoxy-ethane, 1,2-diethoxy-ethane, 1-methoxy-2-(2-methoxy-ethoxy)-ethane, 1-ethoxy-2-(2-ethoxy-ethoxy)-ethane, tetrahydrofuran, 1,4-dioxane); ketones (e.g., acetone, methyl ethyl ketone); esters (methyl acetate, ethyl acetate); nitrogen-containing solvents (e.g., formamide, N,N-dimethylformamide, acetonitrile, N-methyl-pyrrolidone, pyridine, quinoline, nitrobenzene); sulfur-containing solvents (e.g., carbon disulfide, dimethyl sulfoxide, tetrahydro-thiophene-1,1,-dioxide); and phosphorus-containing solvents (e.g., hexamethylphosphoric triamide).

In the schemes, below, substituent identifiers (e.g., X, L, R¹, R², R³, R⁴, R⁵, R⁶, R⁷, and R⁸) are as defined above for Formula (I). As mentioned earlier, some of the starting materials and intermediates may include protecting groups, which are removed prior to the final product. In such cases, the substituent identifier refers to moieties defined in Formula (I) and to those moieties with appropriate protecting groups. For example, a starting material or intermediate in the synthetic methods may include a potentially reactive (secondary) amine. In such cases, the amine would include the moiety with or without, say, a Boc or Cbz group attached to the amine.

The production method of the compound of the present invention is explained below.

The raw material compound and reagent used and the compound obtained in each step in the following production method may be each in a form of a salt, and examples of such salt include those similar to the salts of the compound of the present invention and the like.

When the compound obtained in each step is a free form, it can be converted to the objective salt according to a method known per se. When the compound obtained in each step is a salt, it can be converted to the objective free form or the other salt according to a method known per se.

The compound obtained in each step can be used directly as the reaction mixture or as a crude product for the next reaction. Alternatively, the compound obtained in each step can be isolated and purified from a reaction mixture according to a method known per se, for example, a separation means such as concentration, crystallization, recrystallization, distillation, solvent extraction, fractional distillation, column chromatography and the like.

When the raw material compound and reagent used in each step are commercially available, the commercially available product can also be used directly.

In the reaction in each step, while the reaction time varies depending on the kind of the reagent and solvent to be used, it is generally 1 minute to 48 hours, preferably 10 minutes to 8 hours, unless otherwise specified.

In the reaction in each step, while the reaction temperature varies depending on the kind of the reagent and solvent to be used, it is generally −78° C. to 300° C., preferably −78° C. to 150° C., unless otherwise specified.

In the reaction in each step, while the pressure varies depending on the kind of the reagent and solvent to be used, it is generally 1 atm to 20 atm, preferably 1 atm to 3 atm, unless otherwise specified.

Microwave synthesizer such as Initiator manufactured by Biotage and the like may be used for the reaction in each step. While the reaction temperature varies depending on the kind of the reagent and solvent to be used, it is generally room temperature to 300° C., preferably 50° C. to 250° C., unless otherwise specified. While the reaction time varies depending on the kind of the reagent and solvent to be used, it is generally 1 minute to 48 hours, preferably 1 minute to 8 hours, unless otherwise specified.

In the reaction in each step, the reagent is used in an amount of 0.5 equivalents to 20 equivalents, preferably 0.8 equivalents to 5 equivalents, relative to the substrate, unless otherwise specified. When the reagent is used as a catalyst, the reagent is used in an amount of 0.001 equivalent to 1 equivalent, preferably 0.01 equivalent to 0.2 equivalent, relative to the substrate. When the reagent is used as a reaction solvent, the reagent is used in a solvent amount.

Unless otherwise specified, the reaction in each step is carried out without solvent, or by dissolving or suspending the raw material compound in a suitable solvent. Examples of the solvent include those described in Examples and the following solvents.
alcohols: methanol, ethanol, tert-butyl alcohol, 2-methoxyethanol and the like;
ethers: diethyl ether, diphenyl ether, tetrahydrofuran, 1,2-dimethoxyethane and the like;
aromatic hydrocarbons: chlorobenzene, toluene, xylene and the like;
saturated hydrocarbons: cyclohexane, hexane and the like;
amides: N,N-dimethylformamide, N-methylpyrrolidone and the like;
halogenated hydrocarbons: dichloromethane, carbon tetrachloride and the like;
nitriles: acetonitrile and the like;
sulfoxides: dimethyl sulfoxide and the like;
aromatic organic bases: pyridine and the like;
anhydrides: acetic anhydride and the like;
organic acids: formic acid, acetic acid, trifluoroacetic acid and the like;
inorganic acids: hydrochloric acid, sulfuric acid and the like;
esters: ethyl acetate and the like;
ketones: acetone, methyl ethyl ketone and the like;
water.

The above-mentioned solvent can be used in a mixture of two or more kinds thereof in an appropriate ratio.

When a base is used for the reaction in each step, examples thereof include those described in Examples and the following bases.
inorganic bases: sodium hydroxide, magnesium hydroxide, sodium carbonate, calcium carbonate, sodium hydrogen carbonate and the like;
organic bases: triethylamine, diethylamine, pyridine, 4-dimethylaminopyridine, N,N-dimethylaniline, 1,4-diazabicyclo[2.2.2]octane, 1,8-diazabicyclo[5.4.0]-7-undecene, imidazole, piperidine and the like;
metal alkoxides: sodium ethoxide, potassium tert-butoxide and the like;
alkali metal hydrides: sodium hydride and the like;
metal amides: sodium amide, lithium diisopropylamide, lithium hexamethyldisilazide and the like;
organic lithiums: n-butyllithium and the like.

When an acid or an acid catalyst is used for the reaction in each step, examples thereof include those described in Examples and the following acids and acid catalysts.
inorganic acids: hydrochloric acid, sulfuric acid, nitric acid, hydrobromic acid, phosphoric acid and the like;
organic acids: acetic acid, trifluoroacetic acid, citric acid, p-toluenesulfonic acid, 10-camphorsulfonic acid and the like;
Lewis acid: boron trifluoride diethyl ether complex, zinc iodide, anhydrous aluminum chloride, anhydrous zinc chloride, anhydrous iron chloride and the like.

Unless otherwise specified, the reaction in each step is carried out according to a method known per se, for example, the method described in Jikken Kagaku Kouza, 5th Edition, vol. 13-19 (the Chemical Society of Japan ed.); Shin Jikken Kagaku Kouza, vol. 14-15 (the Chemical Society of Japan ed.); Fine Organic Chemistry, Revised 2nd Edition (L. F. Tietze, Th. Eicher, Nankodo); Organic Name Reactions, the Reaction Mechanism and Essence, Revised Edition (Hideo Togo, Kodansha); ORGANIC SYNTHESES Collective Volume I-VII (John Wiley & Sons Inc.); Modern Organic Synthesis in the Laboratory A Collection of Standard Experimental Procedures (Jie Jack Li, OXFORD UNIVERSITY); Comprehensive Heterocyclic Chemistry III, Vol. 1-Vol. 14 (Elsevier Japan); Strategic Applications of Named Reactions in Organic Synthesis (translated by Kiyoshi Tomioka, Kagakudojin); Comprehensive Organic Transformations (VCH Publishers Inc.), 1989, or the like, or the method described in Examples.

In each step, the protection or deprotection reaction of a functional group is carried out according to a method known per se, for example, the method described in “Protective Groups in Organic Synthesis, 4th Ed”, Wiley-Interscience, Inc., 2007 (Theodora W. Greene, Peter G. M. Wuts); “Protecting Groups 3rd Ed.” Thieme, 2004 (P. J. Kocienski), or the like, or the method described in Examples.

Examples of the protecting group for a hydroxy group of an alcohol and the like and a phenolic hydroxy group include ether-type protecting groups such as methoxymethyl ether, benzyl ether, methyl ether, tert-butyldimethylsilyl ether, tetrahydropyranyl ether and the like; carboxylate ester-type protecting groups such as acetate ester and the like; sulfonate ester-type protecting groups such as methanesulfonate ester and the like; carbonate ester-type protecting groups such as tert-butylcarbonate and the like, and the like.

Examples of the protecting group for a carbonyl group of an aldehyde include acetal-type protecting groups such as dimethylacetal and the like; cyclic acetal-type protecting groups such as 1,3-dioxane and the like, and the like.

Examples of the protecting group for a carbonyl group of a ketone include ketal-type protecting groups such as dimethylketal and the like; cyclic ketal-type protecting groups such as 1,3-dioxolane, 1,3-dioxane and the like; oxime-type protecting groups such as O-methyloxime and the like; hydrazone-type protecting groups such as N,N-dimethylhydrazone and the like, and the like.

Examples of the protecting group for a carboxyl group include ester-type protecting groups such as methyl ester and the like; amide-type protecting groups such as N,N-dimethylamide and the like, and the like.

Examples of the protecting group for a thiol include ether-type protecting groups such as benzyl thioether and the like; ester-type protecting groups such as thioacetate ester, thiocarbonate, thiocarbamate and the like, and the like.

Examples of the protecting group for an amino group and an aromatic heterocycle such as imidazole, pyrrole, indole and the like include carbamate-type protecting groups such as benzyl carbamate and the like; amide-type protecting groups such as acetamide and the like; alkyl amine-type protecting groups such as N-triphenylmethylamine and the like; sulfonamide-type protecting groups such as methanesulfonamide and the like, and the like.

The protecting groups can be removed according to a method known per se, for example, by employing a method using acid, base, ultraviolet rays, hydrazine, phenylhydrazine, sodium N-methyldithiocarbamate, tetrabutylammonium fluoride, palladium acetate, trialkylsilyl halide (e.g., trimethylsilyl iodide, trimethylsilyl bromide) and the like, a reduction method, and the like.

When reduction reaction is carried out in each step, examples of the reducing agent to be used include metal hydrides such as lithium aluminum hydride, sodium triacetoxyborohydride, sodium cyanoborohydride, diisobutylaluminum hydride (DIBAL-H), sodium borohydride, tetramethylammonium triacetoxyborohydride and the like; boranes such as borane tetrahydrofuran complex and the like; Raney nickel; Raney cobalt; hydrogen; formic acid; triethylsilane; iron; zinc and the like. When carbon-carbon double bond or triple bond is reduced, a method using a catalyst such as palladium-carbon, Lindlar's catalyst and the like may be employed.

When oxidation reaction is carried out in each step, examples of the oxidizing agent to be used include peroxides such as m-chloroperbenzoic acid (mCPBA), hydrogen peroxide, tert-butylhydroperoxide and the like; perchlorates such as tetrabutylammonium perchlorate and the like; chlorates such as sodium chlorate and the like; chlorites such as sodium chlorite and the like; periodates such as sodium periodate and the like; hypervalent iodine reagents such as iodosylbenzene and the like; reagents containing manganese such as manganese dioxide, potassium permanganate and the like; leads such as lead tetraacetate and the like; reagents containing chromium such as pyridinium chlorochromate (PCC), pyridinium dichromate (PDC), Jones reagent and the like; halogen compounds such as N-bromosuccinimide (NBS) and the like; oxygen; ozone; sulfur trioxide-pyridine complex; osmium tetroxide; selenium dioxide; 2,3-dichloro-5,6-dicyano-1,4-benzoquinone (DDQ) and the like.

When radical reaction is carried out in each step, examples of the radical initiator to be used include azo compounds such as azobisisobutyronitrile (AIBN) and the like; water-soluble radical initiators such as 4,4′-azobis-4-cyanopentanoic acid (ACPA) and the like; triethylboron in the presence of air or oxygen; benzoyl peroxide and the like. Examples of the radical reagent to be used include tributylstannane, tristrimethylsilylsilane, 1,1,2,2-tetraphenyldisilane, diphenylsilane, samarium iodide and the like.

When Wittig reaction is carried out in each step, examples of the Wittig reagent to be used include alkylidene phosphoranes and the like. The alkylidene phosphoranes can be prepared according to a method known per se, for example, by reacting a phosphonium salt with a strong base.

When Horner-Emmons reaction is carried out in each step, examples of the reagent to be used include phosphonoacetates such as methyl dimethylphosphonoacetate, ethyl diethylphosphonoacetate and the like; and bases such as alkali metal hydrides, organic lithiums and the like.

When Friedel-Crafts reaction is carried out in each step, a combination of a Lewis acid and an acid chloride or a combination of a Lewis acid and an alkylating agent (e.g., an alkyl halide, an alcohol, an olefin etc.) is used as a reagent. Alternatively, an organic acid or an inorganic acid can also be used instead of a Lewis acid, and an anhydride such as acetic anhydride and the like can also be used instead of an acid chloride.

When aromatic nucleophilic substitution reaction is carried out in each step, a nucleophile (e.g., an amine, imidazole, alcohol etc.) and a base (e.g., an inorganic base, an organic base etc.) are used as a reagent.

When nucleophilic addition reaction by a carbo anion, nucleophilic 1,4-addition reaction (Michael addition reaction) by a carbo anion or nucleophilic substitution reaction by a carbo anion is carried out in each step, and examples of the base to be used for generation of the carbo anion include organic lithiums, metal alkoxides, inorganic bases, organic bases and the like.

When Grignard reaction is carried out in each step, examples of the Grignard reagent to be used include arylmagnesium halides such as phenylmagnesium bromide and the like; and alkylmagnesium halides such as methylmagnesium bromide and the like. The Grignard reagent can be prepared according to a method known per se, for example, by reacting an alkyl halide or an aryl halide with a metal magnesium in an ether or tetrahydrofuran as a solvent.

When Knoevenagel condensation reaction is carried out in each step, a compound having an activated methylene group with two electron withdrawing groups (e.g., malonic acid, diethyl malonate, malononitrile etc.) and a base (e.g., an organic base, a metal alkoxide, an inorganic base) are used as a reagent.

When Vilsmeier-Haack reaction is carried out in each step, phosphoryl chloride and an amide derivative (e.g., N,N-dimethylformamide etc.) are used as a reagent.

When azidation reaction of an alcohol, an alkyl halide or a sulfonate is carried out in each step, examples of the azidating agent to be used include diphenylphosphorylazide (DPPA), trimethylsilylazide, sodium azide and the like. For example, for the azidation reaction of an alcohol, a method using diphenylphosphorylazide and 1,8-diazabicyclo[5.4.0]undec-7-ene (DBU), a method using trimethylsilylazide and a Lewis acid, and the like are employed.

When reductive amination reaction or reductive alkylation reaction is carried out in each step, examples of the reducing agent to be used include sodium triacetoxyborohydride, sodium cyanoborohydride, hydrogen, formic acid and the like. When the substrate is an amine compound, examples of the carbonyl compound to be used include paraformaldehyde, aldehydes such as acetaldehyde and the like, and ketones such as cyclohexanone and the like. When the substrate is a carbonyl compound, examples of the amine to be used include ammonia, primary amines such as methylamine and the like; secondary amines such as dimethylamine and the like, and the like.

When Mitsunobu reaction is carried out in each step, a cyanomethylenetrialkyl phosphorane (e.g., cyanomethylenetrimethylphosphorane, cyanomethylenetributylphosphorane), or a combination of an azodicarboxylate (e.g., diethyl azodicarboxylate (DEAD), diisopropyl azodicarboxylate (DIAD) etc.) and a phosphine (e.g., triphenylphosphine, tri-n-butylphosphine) is used as a reagent.

When esterification reaction, amidation reaction or urea formation reaction is carried out in each step, examples of the reagent to be used include acyl halides such as acid chlorides, acid bromides and the like; activated carboxylic acids such as anhydrides, activated esters, sulfates and the like; esters, especially, for amidation reaction. Examples of the activating agent of the carboxylic acid include carbodiimide condensing agents such as 1-ethyl-3-(3-dimethylaminopropyl)carbodiimide hydrochloride (WSCD) and the like; triazine condensing agents such as 4-(4,6-dimethoxy-1,3,5-triazin-2-yl)-4-methylmorpholinium chloride n-hydrate (DMT-MM) and the like; carbonate condensing agents such as 1,1-carbonyldiimidazole (CDI) and the like; diphenylphosphorylazide (DPPA); benzotriazol-1-yloxy-trisdimethylaminophosphonium salt (BOP reagent); 2-chloro-1-methyl-pyridinium iodide (Mukaiyama reagent); thionyl chloride; lower alkyl haloformates such as ethyl chloroformate and the like; O-(7-azabenzotriazol-1-yl)-N,N,N′,N′-tetramethyluronium hexafluorophosphate (HATU); sulfuric acid; combinations thereof and the like. When carbodiimide condensing agent is used, an additive such as 1-hydroxybenzotriazole (HOBt), N-hydroxysuccinimide (HOSu), dimethylaminopyridine (DMAP) and the like may be added to the reaction system. When the esters are converted to the corresponding carboxamides, examples of the reagent include ammonia and the like.

When coupling reaction is carried out in each step, examples of the metal catalyst to be used include palladium compounds such as palladium(II) acetate, tetrakis(triphenylphosphine)palladium(0), dichlorobis(triphenylphosphine)palladium(II), dichlorobis(triethylphosphine)palladium(II), tris(dibenzylideneacetone)dipalladium(0), 1,1′-bis(diphenylphosphino)ferrocenepalladium(II) chloride and the like; nickel compounds such as tetrakis(triphenylphosphine)nickel(0) and the like; rhodium compounds such as tris(triphenylphosphine)rhodium(III) chloride and the like; cobalt compounds; copper compounds such as copper oxide, copper(I) iodide and the like; platinum compounds and the like. In addition, a base can be added to the reaction system, and examples thereof include inorganic bases, metal alkoxides and the like.

When thiocarbonylation reaction is carried out in each step, phosphorus pentasulfide is typically used as the thiocarbonylating agent. Alternatively, a reagent having a 1,3,2,4-dithiadiphosphetane-2,4-disulfide structure (e.g., 2,4-bis(4-methoxyphenyl)-1,3,2,4-dithiadiphosphetane-2,4-disulfide (Lawesson's reagent) etc.) can also be used instead of phosphorus pentasulfide.

When Wohl-Ziegler reaction is carried out in each step, examples of the halogenating agent to be used include N-iodosuccinimide, N-bromosuccinimide (NBS), N-chlorosuccinimide (NCS), bromine, sulfuryl chloride and the like. In addition, the reaction can be accelerated by subjecting a radical initiator such as heat, light, benzoyl peroxide, azobisisobutyronitrile and the like to the reaction system.

When halogenation reaction of a hydroxy group is carried out in each step, examples of the halogenating agent to be used include hydrohalic acids and acid halides of inorganic acids, specifically, hydrochloric acid, thionyl chloride, phosphorus oxychloride and the like for chlorination, 48% hydrobromic acid and the like for bromination. In addition, a method of producing an alkyl halide by reacting an alcohol with triphenylphosphine and carbon tetrachloride or carbon tetrabromide or the like can be employed. Alternatively, a method of producing an alkyl halide via two steps comprising converting an alcohol to the corresponding sulfonate, and then reacting the sulfonate with lithium bromide, lithium chloride or sodium iodide can also be employed.

When Arbuzov reaction is carried out in each step, examples of the reagent to be used include alkyl halides such as ethyl bromoacetate and the like; and phosphites such as triethyl phosphite, tri(isopropyl) phosphite and the like.

When sulfonate esterification reaction is carried out in each step, examples of the sulfonating agent to be used include methanesulfonyl chloride, p-toluenesulfonyl chloride, methanesulfonic anhydride, p-toluenesulfonic anhydride and the like.

When hydrolysis reaction is carried out in each step, an acid or a base is used as a reagent. For acid hydrolysis reaction of the tert-butyl ester, formic acid, triethylsilane and the like may be added to reductively-trap tert-butyl cation which is by-produced. For hydrolysis reaction of the cyano group, examples of the base include potassium carbonate, sodium hydroxide and the like. In addition, an oxidant may be added to the reaction system, and examples thereof include hydrogen peroxide and the like.

When dehydration reaction is carried out in each step, examples of the dehydrating agent to be used include sulfuric acid, diphosphorus pentaoxide, phosphorus oxychloride, N,N′-dicyclohexylcarbodiimide, alumina, polyphosphoric acid and the like.

When Chan-Lam reaction is carried out in each step, examples of the metal catalyst to be used include copper compounds such as copper(I) bromide, copper(I) iodide, copper(II) acetate and the like. In addition, a base may be added to the reaction system, and examples thereof include organic bases and the like.

When Ullmann reaction is carried out in each step, examples of the metal catalyst to be used include copper compounds such as copper(I) bromide, copper(I) iodide, copper(II) acetate and the like, and examples of the ligand include N,N,N′,N′-tetramethylethylene diamine and the like. In addition, a base may be added to the reaction system, and examples thereof include organic bases, inorganic bases and the like.

When alkylation reaction is carried out in each step, examples of the base to be used include potassium carbonate, tripotassium phosphate, triethylamine, N,N-diisopropylethylamine, pyridine, sodium ethoxide, potassium tert-butoxide, sodium hydride, lithiumhexamethyldisilazide, sodium hexamethyldisilazide, n-butyllithium and the like. In addition, an inorganic salt may be added to the reaction system, and examples thereof include lithium bromide and the like.

When deoxofluorination reaction is carried out in each step, examples of the fluorinating agent to be used include bis(2-methoxyethyl)aminosulfur trifluoride, diethylaminosulfur trifluoride, 4-tert-butyl-2,6-dimethylphenylsulfur trifluoride, N,N-diethyl-S,S-difluorosulfiliminium tetrafluoroborate, difluoro-4-morpholinylsulfonium tetrafluoroborate and the like.

When Hofmann rearrangement reaction is carried out in each step, examples of the reagent to be used include lead tetraacetate, iodobenzene diacetate and the like.

When thiourea formation reaction or thiocarbamation reaction is carried out in each step, examples of the reagent to be used include thiophosgene and the like, and examples of the base include organic bases, sodium hydride and the like.

When cyclopropanation reaction is carried out in each step, examples of the reagent to be used include diiodomethane, dibromomethane, dibromodifluoromethane, dibromofluoromethane, (trifluoromethyl)trimethylsilane, (bromodifluoromethyl)trimethylsilane, (dibromofluoromethyl)trimethylsilane, carbon tetrafluoride, sodium trifluoroacetate and the like.

When compound (I) and intermediate for the production of compound (I) have a convertible functional group (e.g., a carboxyl group, an amino group, a hydroxy group, a carbonyl group, a mercapto group, a C₁₋₆ alkoxy-carbonyl group, a C₆₋₁₄ aryloxy-carbonyl group, a C₇₋₁₆ aralkyloxy-carbonyl group, a sulfo group, a sulfide group, a halogen atom, an optionally halogenated C₁₋₆ alkylsulfonyloxy group, a cyano group, an aminocarbonyl group, a boryl group etc.), various compounds can be produced by converting such functional group according to a method known per se or a method analogous thereto.

Carboxyl group can be converted, for example, by reactions such as esterification, reduction, amidation, conversion reaction to optionally protected amino group and the like.

Amino group can be converted, for example, by reactions such as amidation, sulfonylation, nitrosation, alkylation, arylation, imidation and the like.

Hydroxy group can be converted, for example, by reactions such as esterification, carbamoylation, sulfonylation, alkylation, fluorination, arylation, oxidation, halogenation and the like.

Carbonyl group can be converted, for example, by reactions such as reduction, oxidation, fluorination, imination (including oximation, hydrazonation), (thio)ketalization, alkylidenation, thiocarbonylation and the like.

Mercapto group can be converted, for example, by reactions such as alkylation, oxidation and the like.

C₁₋₆ alkoxy-carbonyl group, C₆₋₁₄ aryloxy-carbonyl group and C₇₋₁₆ aralkyloxy-carbonyl group can be converted, for example, by reactions such as reduction, hydrolysis and the like.

Sulfo group can be converted, for example, by reactions such as sulfonamidation, reduction and the like.

Sulfide group can be converted, for example, by reactions such as oxidation and the like.

Halogen atom can be converted, for example, by various nucleophilic substitution reactions, various coupling reactions and the like.

Optionally halogenated C₁₋₆ alkylsulfonyloxy group can be converted, for example, by various nucleophilic substitution reactions, various coupling reactions and the like.

Cyano group can be converted, for example, by reactions such as reduction, hydrolysis and the like.

Aminocarbonyl group can be converted, for example, by reactions such as dehydration, reduction and the like.

Boryl group can be converted, for example, by oxidation, various coupling reactions and the like.

In each of the above-mentioned reactions, when the compound is obtained in a free form, it may be converted to a salt according to a conventional method. When it is obtained as a salt, it may be converted to a free form or other salt according to a conventional method.

The conversion of these functional group can be carried out according to a method known per se, for example, the method described in Comprehensive Organic Transformations, Second Edition, Wiley-VCH, Richard C. Larock, or the like.

Compound (I) obtained in each reaction scheme can be isolated and purified by known separation and purification means such as concentration, concentration under reduced pressure, solvent extraction, crystallization, recrystallization, phase transfer, chromatography and the like. In addition, each material compound used in each reaction scheme can be isolated and purified by those similar to the above-mentioned known separation and purification means. The material compound may be used directly in the next step as the reaction mixture without isolation.

When compound (I) has isomers such as an optical isomer, a stereoisomer, a regioisomer and a rotamer and the like, such isomers and a mixture thereof are also encompassed in compound (I). For example, when compound (I) has an optical isomer, the optical isomer resolved from racemate is also encompassed in compound (I). These isomers can be obtained as single products according to synthetic methods known per se, separation methods known per se (e.g., concentration, solvent extraction, column chromatography, recrystallization etc.), optical resolutions (e.g., fractional recrystallization method, chiral column method, diastereomer method and the like).

Compound (I) may be a crystal, and the crystal form may be single or a mixture of crystal forms, both of which are encompassed in compound (I). The crystal can be produced according to a crystallization method known per se.

The compound (I) may be a solvate (e.g., hydrate) or a non-solvate (e.g., non-hydrate etc.) and both are encompassed in compound (I).

The compounds labeled with isotopes (e.g., ³H, ¹⁴C, ³⁵S ¹²⁵I etc.) and the like are also encompassed in compound (I).

A deuterium conversion form wherein ¹H is converted to ²H(D) is also encompassed in compound (I).

Compound (I) labeled or substituted with an isotope can be used as, for example, a tracer (PET tracer) used for Positron Emission Tomography (PET), and therefore, it is useful in the fields of medical diagnosis and the like.

Compound (I) of the present invention can be synthesized according to the production method described below.

Each variable in the formulas of the reaction schemes is as defined above, unless otherwise specified.

Compounds (I-a) and (I-b), wherein L is O, can be produced from compounds (1), (2) and (3) according to the following scheme 1. In the scheme, R is an optionally substituted C₁₋₆ alkyl group, or an optionally substituted C₃₋₈ cycloalkyl group, PG is a protecting group for hydroxyl group, LG is a leaving group. Examples of the protecting group for hydroxyl group include a methoxy methylene group, a benzyl group, a 4-methoxybenzyl group, methyl group and the like. Examples of the leaving group include halogen atoms, optionally halogenated C₁₋₆ alkylsulfonyl (e.g., methanesulfonyl, ethanesulfonyl), optionally halogenated C₁₋₆ alkylsulfonyloxy (e.g., methanesulfonyloxy, ethanesulfonyloxy, trifluoromethanesulfonyloxy), C₆₋₁₄ arylsulfonyloxy optionally substituted by C₁₋₆ alkyl (e.g., benzenesulfonyloxy, toluenesulfonyloxy) and the like.

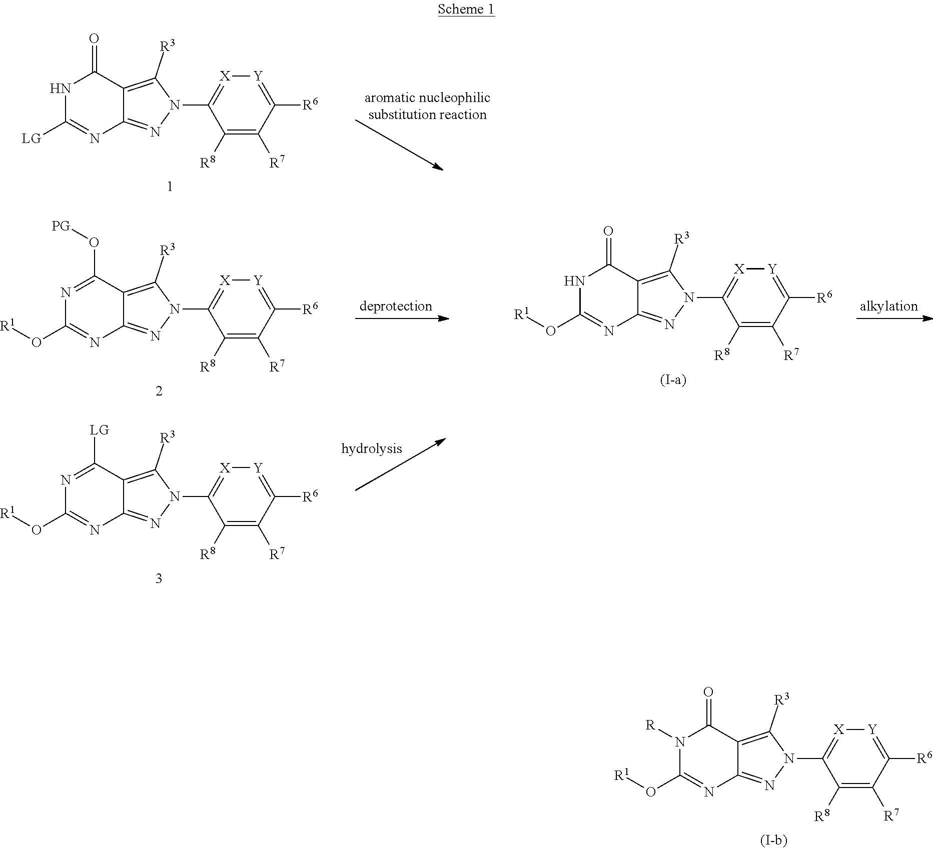

Compound (I-a) can be produced by subjecting compound (1) to an aromatic nucleophilic substitution reaction with the corresponding alcohol (R¹—OH), compound (2) to a deprotection reaction, or compound (3) to a hydrolysis reaction.

Compound (I-b) can be produced by subjecting compound (I-a) to an alkylation reaction.

Compounds (1), (2) and (3) used in the above-mentioned scheme 1 can be produced from compound (4) according to the following scheme 2. In the scheme, PG is a protecting group for hydroxyl group and LG¹, LG² and LG³ are leaving groups. Examples of the protecting group for hydroxyl group include a methoxy methylene group, a benzyl group, a 4-methoxybenzyl group, methyl group and the like. Examples of the leaving group include halogen atoms, optionally halogenated C₁₋₆ alkylsulfonyl (e.g., methanesulfonyl, ethanesulfonyl), optionally halogenated C₁₋₆ alkylsulfonyloxy (e.g., methanesulfonyloxy, ethanesulfonyloxy, trifluoromethanesulfonyloxy), C₆₋₁₄ arylsulfonyloxy optionally substituted by C₁₋₆ alkyl (e.g., benzenesulfonyloxy, toluenesulfonyloxy) and the like.

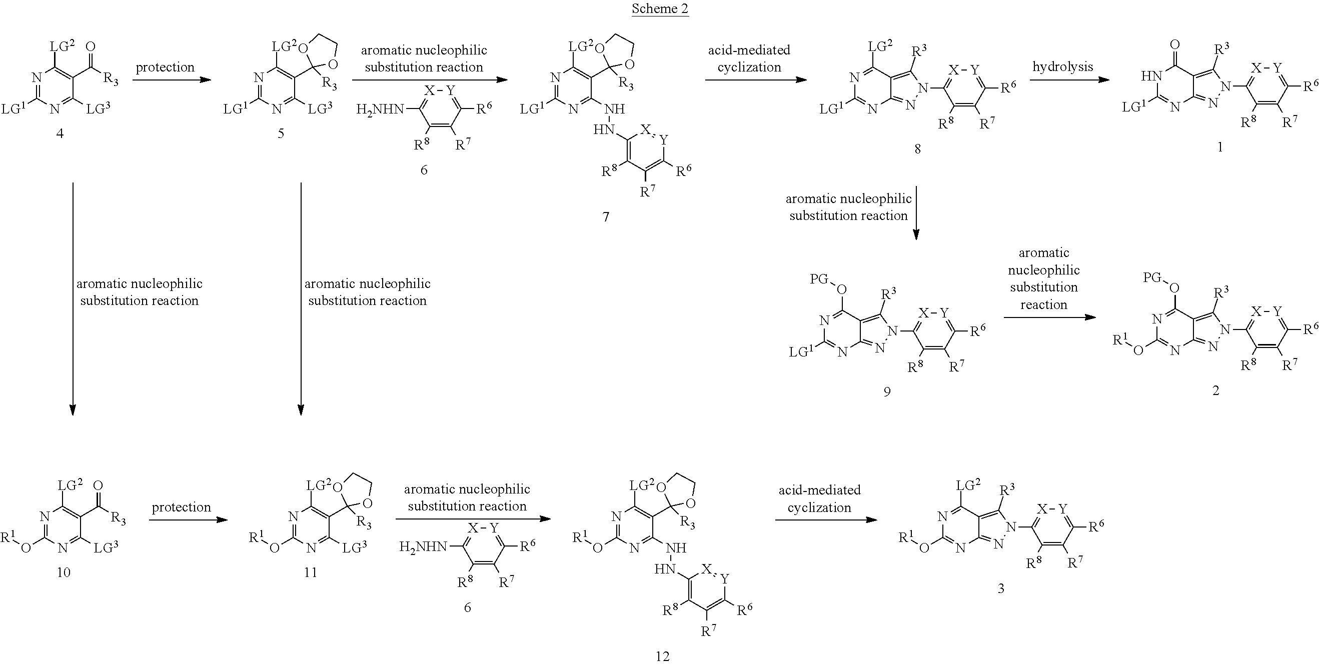

Compounds (4) and (6) may be commercially available or can be produced according to a method known per se.

Compound (5) can be produced by subjecting compound (4) to a protection reaction.

Compound (7) can be produced by subjecting compound (5) to an aromatic nucleophilic substitution reaction with compound (6).

Compound (8) can be produced by subjecting compound (7) to an acid-mediated cyclization reaction. Examples of the acid to be used include p-toluenesulfonic acid, acetic acid, trifluoroacetic acid and the like.

Compound (1) can be produced by subjecting compound (8) to a hydrolysis reaction.

Compound (9) can be produced by subjecting compound (8) to an aromatic nucleophilic substitution reaction with the corresponding alcohol (PG-OH).

Compound (2) can be produced by subjecting compound (9) to an aromatic nucleophilic substitution reaction with the corresponding alcohol (R¹—OH).

Compound (10) can be produced by subjecting compound (4) to an aromatic nucleophilic substitution reaction with the corresponding alcohol (R¹—OH).

Compound (11) can be produced by subjecting compound (10) to a protection reaction or by subjecting compound (5) to an aromatic nucleophilic substitution reaction with the corresponding alcohol (R¹—OH).

Compound (12) can be produced by subjecting compound (11) to an aromatic nucleophilic substitution reaction with compound (6).

Compound (3) can be produced by subjecting compound (12) to an acid-mediated cyclization reaction. Examples of the acid to be used include p-toluenesulfonic acid, acetic acid, trifluoroacetic acid and the like.

Compounds (I-c) and (I-d), which are compound (I) wherein L is a bond, can be produced from compound (1), compound (13) or compound (14) according to the following Scheme 3.

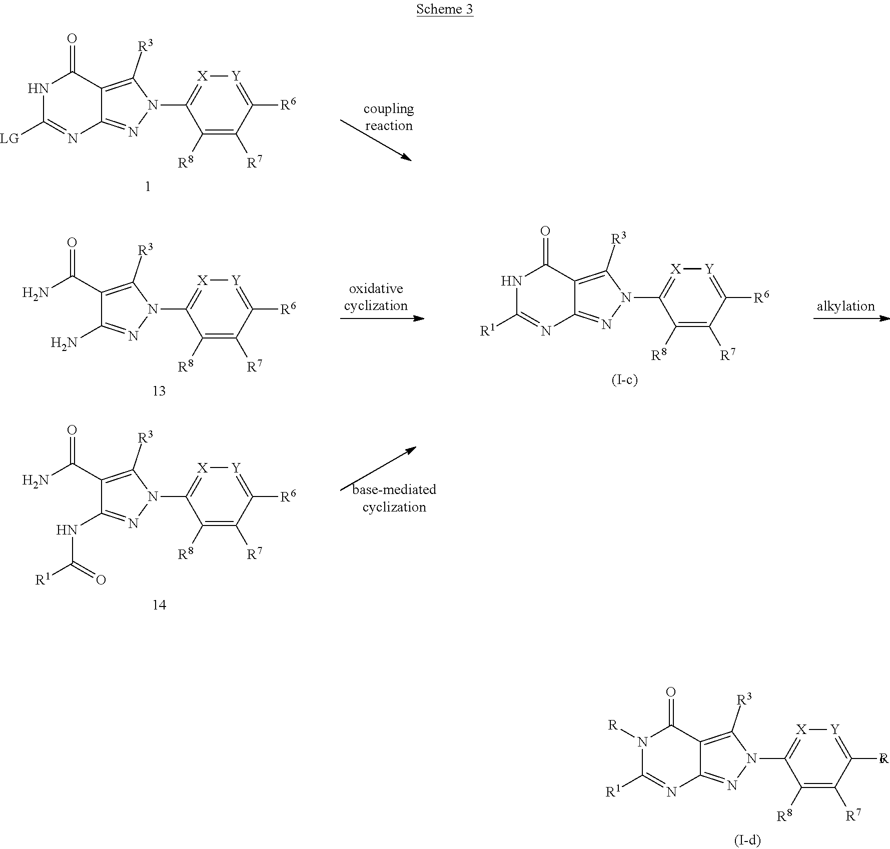

Compound (I-c) can be produced by subjecting compound (1) to a coupling reaction, compound (13) to a cyclization reaction with oxidant and the corresponding aldehyde (R¹—CHO), or compound (14) to a cyclization reaction with base. Examples of the oxidant to be used include iodine and the like. Examples of the base to be used include sodium hydroxide, potassium hydroxide, potassium tert-butoxide and the like.

Compound (I-d) can be produced by subjecting compound (I-c) to an alkylation reaction.

Compound (13) and (14) can be produced from compound (15) according to the following scheme 4. In the scheme, Z is dihydroxyboryl group, a pinacolboryl group (4,4,5,5-tetramethyl-1,3,2-dioxaborolan-2-yl) or a halogen atom. In the scheme, LG¹ and LG² are leaving groups. Examples of the leaving group include halogen atoms, optionally halogenated C₁₋₆ alkylsulfonyl (e.g., methanesulfonyl, ethanesulfonyl), optionally halogenated C₁₋₆ alkylsulfonyloxy (e.g., methanesulfonyloxy, ethanesulfonyloxy, trifluoromethanesulfonyloxy), C₆₋₁₄ arylsulfonyloxy optionally substituted by C₁₋₆ alkyl (e.g., benzenesulfonyloxy, toluenesulfonyloxy) and the like.

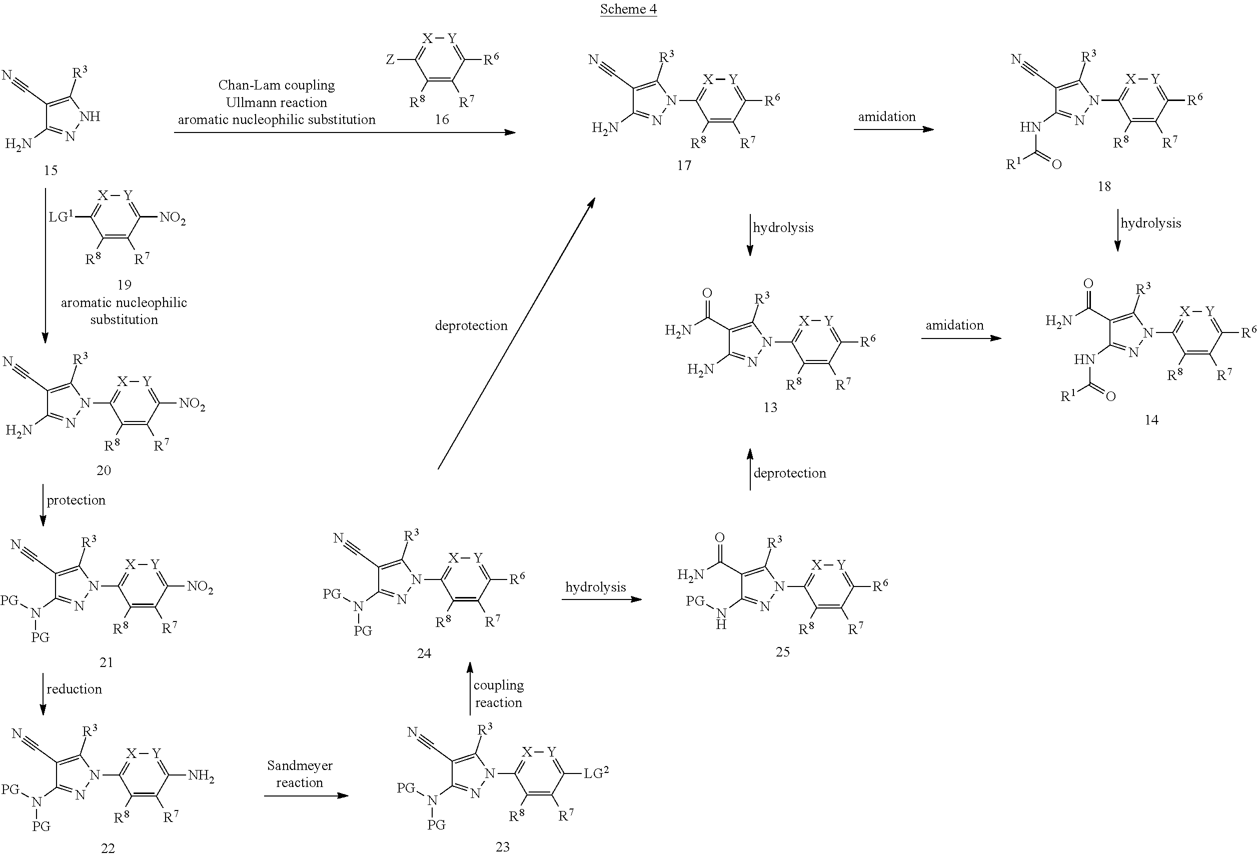

Compounds (15), (16) and (19) may be commercially available or can be produced according to a method known per se.

Compound (17) can be produced by subjecting compound (15) to Chan-Lam reaction with the corresponding aryl boronic acid ester or boronic acid (16), Ullmann reaction with the corresponding aryl halide (16), or an aromatic nucleophilic substitution reaction with the corresponding aryl halide (16). Alternatively, compound (17) can also be produced by subjecting compound (24) to a deprotection reaction.

Compound (18) can be produced by subjecting compound (17) to an amidation reaction.

Compound (14) can be produced by subjecting compound (18) to a hydrolysis reaction with hydrogen peroxide and base. Alternatively, compound (14) can also be produced by subjecting compound (13) to an amidation reaction.

Compound (13) can be produced by subjecting compound (17) to a hydrolysis reaction with hydrogen peroxide and base. Alternatively, compound (13) can also be produced by subjecting compound (25) to a deprotection reaction.

Compound (20) can be produced by subjecting compound (15) to an aromatic nucleophilic substitution reaction with compound (19).

Compound (21) can be produced by subjecting compound (20) to a protection reaction.

Compound (22) can be produced by subjecting compound (21) to a reduction reaction.

Compound (23) can be produced by subjecting compound (22) to Sandmeyer reaction.

Compound (24) can be produced by subjecting compound (23) to a coupling reaction with the corresponding boronic acid ester or boronic acid.

Compound (25) can be produced by subjecting compound (24) to a hydrolysis reaction with hydrogen peroxide and base.

Compound (13) can also be produced from compounds (26) and (6) according to the following scheme 5. In the scheme, R′ and R″ are alkyl groups, Z is dihydroxyboryl group, a pinacolboryl group (4,4,5,5-tetramethyl-1,3,2-dioxaborolan-2-yl) or a halogen atom.

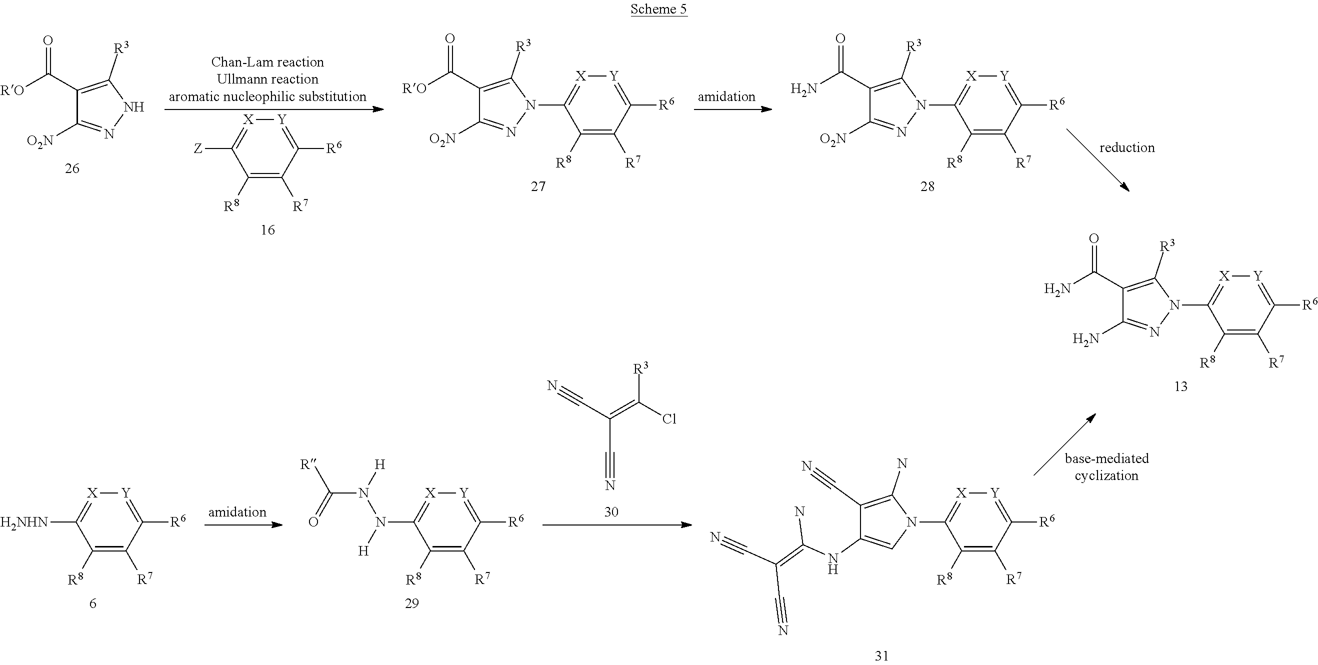

Compounds (26) and (30) may be commercially available or can be produced according to a method known per se.

Compound (27) can be produced by subjecting compound (26) to Chan-Lam reaction with the corresponding aryl boronic acid ester or boronic acid (16), Ullmann reaction with the corresponding aryl halide (16), or an aromatic nucleophilic substitution reaction with the corresponding aryl halide (16).

Compound (28) can be produced by subjecting compound (27) to an amidation reaction.

Compound (13) can be produced by subjecting compound (28) to a reduction reaction. Alternatively, compound (13) can be produced by subjecting compound (31) to a cyclization reaction with base. Examples of the base to be used include sodium hydroxide and the like.

Compound (29) can be produced by subjecting compound (6) to an amidation reaction.

Compound (31) can be produced by subjecting compound (29) to a reaction with compound (30).

Compounds of Formula (I), which include compounds named above, and their pharmaceutically acceptable complexes, salts, solvates and hydrates, should be assessed for their biopharmaceutical properties, such as solubility and solution stability across pH, permeability, and the like, to select an appropriate dosage form and route of administration. Compounds that are intended for pharmaceutical use may be administered as crystalline or amorphous products, and may be obtained, for example, as solid plugs, powders, or films by methods such as precipitation, crystallization, freeze drying, spray drying, evaporative drying, microwave drying, or radio frequency drying.

Compound (I) may be administered alone or in combination with one another or with one or more pharmacologically active agents which are different than Compound (I). When administering Compound (I) with a pharmacologically active agent (“concomitant drug”), the administration time of Compound (I) and the concomitant drug is not restricted, and Compound (I) or a pharmaceutical composition thereof, or the concomitant drug or a pharmaceutical composition thereof can be administered to a subject simultaneously, or may be administered at different times. The dosage of the concomitant drug may be determined according to the dose clinically used, and can be appropriately selected depending on a subject, administration route, disease, combination and the like.

The administration mode of the combination of Compound (I) and the concomitant drug is not particularly limited, and Compound (I) and the concomitant drug only need to be combined on administration. Examples of such administration mode include the following: (1) administration of a single preparation obtained by simultaneously processing Compound (I) and the concomitant drug, (2) simultaneous administration of two kinds of preparations of Compound (I) and the concomitant drug, which have been separately produced, by the same administration route, (3) administration of two kinds of preparations of Compound (I) and the concomitant drug, which have been separately produced, by the same administration route in a staggered manner, (4) simultaneous administration of two kinds of preparations of Compound (I) and the concomitant drug, which have been separately produced, by different administration routes, (5) administration of two kinds of preparations of Compound (I) and the concomitant drug, which have been separately produced, by different administration routes in a staggered manner (e.g., administration in the order of Compound (I) and the concomitant drug, or in the reverse order) and the like.

The dose of the concomitant drug can be appropriately determined based on the dose employed in clinical situations. The mixing ratio of Compound (I) and a concomitant drug can be appropriately determined depending on the administration subject, administration route, target disease, symptom, combination and the like.

For example, the content of Compound (I) in the combination with a concomitant drug differs depending on the form of a preparation, and usually from about 0.01 to about 100 wt %, preferably from about 0.1 to about 50 wt %, further preferably from about 0.5 to about 20 wt %, based on the whole preparation.

The content of the concomitant drug used in the combination with Compound (I) differs depending on the form of a preparation, and usually from about 0.01 to about 100 wt %, preferably from about 0.1 to about 50 wt %, further preferably from about 0.5 to about 20 wt %, based on the whole preparation.

The content of additives such as a carrier and the like used in the combination of Compound (I) and a concomitant drug differs depending on the form of a preparation, and usually from about 1 to about 99.99 wt %, preferably from about 10 to about 90 wt %, based on the preparation.

Similar contents may be employed even when Compound (I) and a concomitant drug are separately formulated into preparations.

Generally, one or more of these compounds are administered as a pharmaceutical composition (a formulation) in association with one or more pharmaceutically acceptable excipients. The choice of excipients depends on the mode of administration, the effect of the excipient on solubility and stability, and the nature of the dosage form, among other things. Useful pharmaceutical compositions and methods for their preparation may be found, for example, in A. R. Gennaro (ed.), Remington: The Science and Practice of Pharmacy (20th ed., 2000).

Compound (I) may be administered orally. Oral administration may involve swallowing in which case the compound enters the bloodstream via the gastrointestinal tract. Alternatively, or additionally, oral administration may involve mucosal administration (e.g., buccal, sublingual, supralingual administration) such that the compound enters the bloodstream through the oral mucosa.

Formulations suitable for oral administration include solid, semi-solid and liquid systems such as tablets; soft or hard capsules containing multi- or nano-particulates, liquids, or powders; lozenges which may be liquid-filled; chews; gels; fast dispersing dosage forms; films; ovules; sprays; and buccal or mucoadhesive patches. Liquid formulations include suspensions, solutions, syrups and elixirs. Such formulations may be employed as fillers in soft or hard capsules (made, e.g., from gelatin or hydroxypropylmethylcellulose) and typically comprise a carrier (e.g., water, ethanol, polyethylene glycol, propylene glycol, methylcellulose, or a suitable oil) and one or more emulsifying agents, suspending agents or both. Liquid formulations may also be prepared by the reconstitution of a solid (e.g., from a sachet).

Compound (I) may also be used in fast-dissolving, fast-disintegrating dosage forms such as those described in Liang and Chen, Expert Opinion in Therapeutic Patents (2001) 11(6):981-986.

For tablet dosage forms, depending on dose, the active pharmaceutical ingredient (API) may comprise from about 1 wt % to about 80 wt % of the dosage form or more typically from about 5 wt % to about 60 wt % of the dosage form. In addition to the API, tablets may include one or more disintegrants, binders, diluents, surfactants, glidants, lubricants, anti-oxidants, colorants, flavoring agents, preservatives, and taste-masking agents. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, C₁₋₆ alkyl-substituted hydroxypropylcellulose, starch, pregelatinized starch, and sodium alginate. Generally, the disintegrant will comprise from about 1 wt % to about 25 wt % or from about 5 wt % to about 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropylcellulose and hydroxypropylmethylcellulose. Tablets may also contain diluents, such as lactose (monohydrate, spray-dried monohydrate, anhydrous), mannitol, xylitol, dextrose, sucrose, sorbitol, microcrystalline cellulose, starch and dibasic calcium phosphate dihydrate.

Tablets may also include surface active agents, such as sodium lauryl sulfate and polysorbate 80, and glidants such as silicon dioxide and talc. When present, surface active agents may comprise from about 0.2 wt % to about 5 wt % of the tablet, and glidants may comprise from about 0.2 wt % to about 1 wt % of the tablet.

Tablets may also contain lubricants such as magnesium stearate, calcium stearate, zinc stearate, sodium stearyl fumarate, and mixtures of magnesium stearate with sodium lauryl sulfate. Lubricants may comprise from about 0.25 wt % to about 10 wt % or from about 0.5 wt % to about 3 wt % of the tablet.

Tablet blends may be compressed directly or by roller compaction to form tablets. Tablet blends or portions of blends may alternatively be wet-, dry-, or melt-granulated, melt congealed, or extruded before tableting. If desired, prior to blending one or more of the components may be sized by screening or milling or both. The final dosage form may comprise one or more layers and may be coated, uncoated, or encapsulated. Exemplary tablets may contain up to about 80 wt % of API, from about 10 wt % to about 90 wt % of binder, from about 0 wt % to about 85 wt % of diluent, from about 2 wt % to about 10 wt % of disintegrant, and from about 0.25 wt % to about 10 wt % of lubricant. For a discussion of blending, granulation, milling, screening, tableting, coating, as well as a description of alternative techniques for preparing drug products, see A. R. Gennaro (ed.), Remington: The Science and Practice of Pharmacy (20th ed., 2000); H. A. Lieberman et al. (ed.), Pharmaceutical Dosage Forms: Tablets, Vol. 1-3 (2d ed., 1990); and D. K. Parikh & C. K. Parikh, Handbook of Pharmaceutical Granulation Technology, Vol. 81 (1997).

Consumable oral films for human or veterinary use are pliable water-soluble or water-swellable thin film dosage forms which may be rapidly dissolving or mucoadhesive. In addition to the API, a typical film includes one or more film-forming polymers, binders, solvents, humectants, plasticizers, stabilizers or emulsifiers, viscosity-modifying agents, and solvents. Other film ingredients may include anti-oxidants, colorants, flavorants and flavor enhancers, preservatives, salivary stimulating agents, cooling agents, co-solvents (including oils), emollients, bulking agents, anti-foaming agents, surfactants, and taste-masking agents. Some components of the formulation may perform more than one function.

In addition to dosing requirements, the amount of API in the film may depend on its solubility. If water soluble, the API would typically comprise from about 1 wt % to about 80 wt % of the non-solvent components (solutes) in the film or from about 20 wt % to about 50 wt % of the solutes in the film. A less soluble API may comprise a greater proportion of the composition, typically up to about 88 wt % of the non-solvent components in the film.

The film-forming polymer may be selected from natural polysaccharides, proteins, or synthetic hydrocolloids and typically comprises from about 0.01 wt % to about 99 wt % or from about 30 wt % to about 80 wt % of the film.

Film dosage forms are typically prepared by evaporative drying of thin aqueous films coated onto a peelable backing support or paper, which may be carried out in a drying oven or tunnel (e.g., in a combined coating-drying apparatus), in lyophilization equipment, or in a vacuum oven.

Useful solid formulations for oral administration may include immediate release formulations and modified release formulations. Modified release formulations include delayed-, sustained-, pulsed-, controlled-, targeted-, and programmed-release. For a general description of suitable modified release formulations, see U.S. Pat. No. 6,106,864. For details of other useful release technologies, such as high energy dispersions and osmotic and coated particles, see Verma et al, Pharmaceutical Technology On-line (2001) 25(2):1-14.

Compound (I) may also be administered directly into the blood stream, muscle, or an internal organ of the subject. Suitable techniques for parenteral administration include intravenous, intraarterial, intraperitoneal, intrathecal, intraventricular, intraurethral, intrasternal, intracranial, intramuscular, intrasynovial, and subcutaneous administration. Suitable devices for parenteral administration include needle injectors, including microneedle injectors, needle-free injectors, and infusion devices.

Parenteral formulations are typically aqueous solutions which may contain excipients such as salts, carbohydrates and buffering agents (e.g., pH of from about 3 to about 9). For some applications, however, Compound (I) may be more suitably formulated as a sterile non-aqueous solution or as a dried form to be used in conjunction with a suitable vehicle such as sterile, pyrogen-free water. The preparation of parenteral formulations under sterile conditions (e.g., by lyophilization) may be readily accomplished using standard pharmaceutical techniques.

The solubility of compounds which are used in the preparation of parenteral solutions may be increased through appropriate formulation techniques, such as the incorporation of solubility-enhancing agents. Formulations for parenteral administration may be formulated to be immediate or modified release. Modified release formulations include delayed, sustained, pulsed, controlled, targeted, and programmed release. Thus, Compounds (I) may be formulated as a suspension, a solid, a semi-solid, or a thixotropic liquid for administration as an implanted depot providing modified release of the active compound. Examples of such formulations include drug-coated stents and semi-solids and suspensions comprising drug-loaded poly(DL-lactic-coglycolic)acid (PGLA) microspheres.

Compound (I) may also be administered topically, intradermally, or transdermally to the skin or mucosa. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical carriers may include alcohol, water, mineral oil, liquid petrolatum, white petrolatum, glycerin, polyethylene glycol and propylene glycol. Topical formulations may also include penetration enhancers. See, e.g., Finnin and Morgan, J. Pharm. Sci. 88(10):955-958 (1999).

Other means of topical administration include delivery by electroporation, iontophoresis, phonophoresis, sonophoresis and microneedle or needle-free (e.g., Powderject™ and Bioject™) injection. Formulations for topical administration may be formulated to be immediate or modified release as described above.

Compound (I) may also be administered intranasally or by inhalation, typically in the form of a dry powder, an aerosol spray, or nasal drops. An inhaler may be used to administer the dry powder, which comprises the API alone, a powder blend of the API and a diluent, such as lactose, or a mixed component particle that includes the API and a phospholipid, such as phosphatidylcholine. For intranasal use, the powder may include a bioadhesive agent, e.g., chitosan or cyclodextrin. A pressurized container, pump, sprayer, atomizer, or nebulizer, may be used to generate the aerosol spray from a solution or suspension comprising the API, one or more agents for dispersing, solubilizing, or extending the release of the API (e.g., EtOH with or without water), one or more solvents (e.g., 1,1,1,2-tetrafluoroethane or 1,1,1,2,3,3,3-heptafluoropropane) which serve as a propellant, and an optional surfactant, such as sorbitan trioleate, oleic acid, or an oligolactic acid. An atomizer using electrohydrodynamics may be used to produce a fine mist.

Prior to use in a dry powder or suspension formulation, the drug product is usually comminuted to a particle size suitable for delivery by inhalation (typically 90% of the particles, based on volume, having a largest dimension less than 5 microns). This may be achieved by any appropriate size reduction method, such as spiral jet milling, fluid bed jet milling, supercritical fluid processing, high pressure homogenization, or spray drying.

Capsules, blisters and cartridges (made, for example, from gelatin or hydroxypropylmethyl cellulose) for use in an inhaler or insufflator may be formulated to contain a powder mixture of the active compound, a suitable powder base such as lactose or starch, and a performance modifier such as L-leucine, mannitol, or magnesium stearate. The lactose may be anhydrous or monohydrated. Other suitable excipients include dextran, glucose, maltose, sorbitol, xylitol, fructose, sucrose, and trehalose.

A suitable solution formulation for use in an atomizer using electrohydrodynamics to produce a fine mist may contain from about 1 μg to about 20 mg of the API per actuation and the actuation volume may vary from about 1 μL to about 100 μL. A typical formulation may comprise one or more Compound (I), propylene glycol, sterile water, EtOH, and NaCl. Alternative solvents, which may be used instead of propylene glycol, include glycerol and polyethylene glycol.

Formulations for inhaled administration, intranasal administration, or both, may be formulated to be immediate or modified release using, for example, PGLA. Suitable flavors, such as menthol and levomenthol, or sweeteners, such as saccharin or sodium saccharin, may be added to formulations intended for inhaled/intranasal administration.

In the case of dry powder inhalers and aerosols, the dosage unit is determined by means of a valve that delivers a metered amount. Units are typically arranged to administer a metered dose or “puff” containing from about 10 μg to about 1000 μg of the API. The overall daily dose will typically range from about 100 μg to about 10 mg which may be administered in a single dose or, more usually, as divided doses throughout the day.

The active compounds may be administered rectally or vaginally, e.g., in the form of a suppository, pessary, or enema. Cocoa butter is a traditional suppository base, but various alternatives may be used as appropriate. Formulations for rectal or vaginal administration may be formulated to be immediate or modified release as described above.

Compound (I) may also be administered directly to the eye or ear, typically in the form of drops of a micronized suspension or solution in isotonic, pH-adjusted, sterile saline. Other formulations suitable for ocular and aural administration include ointments, gels, biodegradable implants (e.g., absorbable gel sponges, collagen), non-biodegradable implants (e.g., silicone), wafers, lenses, and particulate or vesicular systems, such as niosomes or liposomes. The formulation may include one or more polymers and a preservative, such as benzalkonium chloride. Typical polymers include crossed-linked polyacrylic acid, polyvinylalcohol, hyaluronic acid, cellulosic polymers (e.g., hydroxypropylmethylcellulose, hydroxyethylcellulose, methyl cellulose), and heteropolysaccharide polymers (e.g., gelan gum). Such formulations may also be delivered by iontophoresis. Formulations for ocular or aural administration may be formulated to be immediate or modified release as described above.

To improve their solubility, dissolution rate, taste-masking, bioavailability, or stability, Compound (I) may be combined with soluble macromolecular entities, including cyclodextrin and its derivatives and polyethylene glycol-containing polymers. For example, API-cyclodextrin complexes are generally useful for most dosage forms and routes of administration. Both inclusion and non-inclusion complexes may be used. As an alternative to direct complexation with the API, the cyclodextrin may be used as an auxiliary additive, i.e., as a carrier, diluent, or solubilizer. Alpha-, beta- and gamma-cyclodextrins are commonly used for these purposes. See, e.g., WO 91/11172, WO 94/02518, and WO 98/55148.

As noted above, one or more compounds of Formula (I), including compounds specifically named above, and their pharmaceutically active complexes, salts, solvates and hydrates, may be combined with each other or with one or more other pharmaceutically active compounds to treat various diseases, conditions and disorders. In such cases, the compounds may be combined in a single dosage form as described above or may be provided in the form of a kit which is suitable for coadministration of the compositions. The kit comprises (1) two or more different pharmaceutical compositions, at least one of which contains Compound (I); and (2) a device for separately retaining the two pharmaceutical compositions, such as a divided bottle or a divided foil packet. An example of such a kit is the familiar blister pack used for the packaging of tablets or capsules. The kit is suitable for administering different types of dosage forms (e.g., oral and parenteral) or for administering different pharmaceutical compositions at separate dosing intervals, or for titrating the different pharmaceutical compositions against one another. To assist with patient compliance, the kit typically comprises directions for administration and may be provided with a memory aid.

For administration to human patients, the total daily dose of the claimed and disclosed compounds is typically in the range of about 0.1 mg to about 3000 mg depending on the route of administration. For example, oral administration may require a total daily dose of from about 1 mg to about 3000 mg, while an intravenous dose may only require a total daily dose of from about 0.1 mg to about 300 mg. The total daily dose may be administered in single or divided doses and, at the physician's discretion, may fall outside of the typical ranges given above. Although these dosages are based on an average human subject having a mass of about 60 kg to about 70 kg, the physician will be able to determine the appropriate dose for a patient (e.g., an infant) whose mass falls outside of this weight range.

As noted above, Compound (I) may be used to treat diseases, disorders and/or conditions associated with NLRP3, i.e., diseases, disorders and/or conditions for which inhibition of the NLRP3 inflammasome pathway is indicated, including diseases, disorders and/or conditions associated with a heterozygous gain of function mutation in the NLRP3 gene, such as a cryopyrin-associated periodic syndrome (CAPS). These may include neonatal-onset multisystem inflammatory disease (NOMID/CINCA), Muckle-Wells syndrome (MWS), and familial cold autoinflammatory syndrome (FCAS).

Compound (I) may be used to treat neurodegenerative diseases and/or conditions associated with NLRP3. These may include Parkinson's disease, Alzheimer's disease, Huntington's disease, amyotrophic lateral sclerosis, prion disease and other forms of dementia (i.e., major or mild neurocognitive disorders) associated with one or more medical conditions, including frontotemporal lobar degeneration, Lewy body disease, vascular disease, traumatic brain injury, substance or medication use, HIV infection, prion disease, Parkinson's disease, and Huntington's disease. Compound (I) may also be used to treat major or mild neurocognitive disorders associated with depression, schizophrenia, bipolar disorder, and autism. In addition, Compound (I) may also be used to treat obesity with certain additional risk factors for cardiovascular disease.

The claimed and disclosed compounds may be combined with one or more other pharmacologically active compounds or therapies to treat one or more disorders, diseases and/or conditions for which inhibition of the NLRP3 inflammasome pathway is indicated. Such combinations may offer significant therapeutic advantages, including fewer side effects, improved ability to treat underserved patient populations, or synergistic activity. For example, compounds of Formula (I), which include compounds specifically named above, and their pharmaceutically acceptable complexes, salts, solvates and hydrates, may be administered simultaneously, sequentially or separately in combination with one or more pharmacologically active compound(s) or therapies for treating Alzheimer's disease, including beta-secretase inhibitors, gamma-secretase inhibitors, HMG-CoA reductase inhibitors, nonsteroidal anti-inflammatory drugs (NSAIDs, such as apazone, aspirin, celecoxib, diclofenac (with and without misoprostol), diflunisal, etodolac, fenoprofen, flurbiprofen, ibuprofen, indomethacin, ketoprofen, meclofenamate sodium, mefenamic acid, meloxicam, nabumetone, naproxen, oxaprozin, phenylbutazone, piroxicam, choline and magnesium salicylates, salsalate, and sulindac), vitamin E, and anti-amyloid antibodies. Specific examples of compounds used to treat Alzheimer's disease include donepezil, rivastigmine, memantine, and galantamine.

In addition to drugs used to improve cognition, Compound (I) may be combined with sedatives, hypnotics, anxiolytics, antipsychotics, tranquilizers, and other medications that are used in the treatment of Alzheimer's disease. For example, Compound (I) may be combined with one or more pharmacologically active agent(s) for treating depression (antidepressants) and/or schizophrenia (atypical or typical antipsychotics) including amitriptyline, amoxapine, aripiprazole, asenapine, bupropion, chlordiazepoxide, citalopram, chlorpromazine, clozapine, desipramine, desvenlafaxine, doxepin, duloxetine, escitalopram, fluoxetine, fluoxetine, fluphenazine, haloperidol, iloperidone, imipramine, isocarboxazid, lamotrigine, levomilnacipran, lurasidone, mirtazapine, nefazodone, nortriptyline, olanzapine, paliperidone, paroxetine, perphenazine, phenelzine, protriptyline, quetiapine, risperidone, selegiline, sertraline, tranylcypromine, trazodone, trimipramine, venlafaxine, vilazodone, and vortioxetine, and ziprasidone.

Likewise, Compound (I) may be combined with one or more pharmaceutically active agent(s) for treating anxiety (anxiolytics) including benzodiazepines (alprazolam, chlordiazepoxide, clobazepam, clonazepam, clorazepate, diazepam, estazolam, flurazepam, lorazepam, midazolam, oxazepam, prazepam, quazepam, temazepam, and triazolam), antihistamines (hydroxyzine), non-benzodiazepines (eszopiclone, zaleplon, zolpidem, and zopiclone) and buspirone.

Compound (I) may also be combined with one or more pharmaceutically active agents for treating epilepsy (antiepileptics or anticonvulsants) including acetazolamide, carbamazepine, clobazam, clonazepam, eslicarbazepine acetate, ethosuximide, gabapentin, lacosamide, lamotrigine, levetiracetam, nitrazepam, oxcarbazepine, perampanel, piracetam, phenobarbital, phenytoin, pregabalin, primidone, retigabine, rufinamide, sodium valproate, stiripentol, tiagabine, topiramate, vigabatrin, and zonisamide.

### EXAMPLES

The present invention is explained in detail in the following by referring to Examples, Experimental Examples and Formulation Examples, which are not to be construed as limitative, and the invention may be changed within the scope of the present invention.

In the following Examples, the “room temperature” generally means about 10° C. to about 35° C. The ratios indicated for mixed solvents are volume mixing ratios, unless otherwise specified. % means wt %, unless otherwise specified.

The elution by column chromatography in the Examples was performed under the observation by TLC (Thin Layer Chromatography) unless otherwise specified. In the observation by TLC, 60 F₂₅₄ manufactured by Merck was used as a TLC plate, the solvent used as an elution solvent in column chromatography was used as a developing solvent, and UV detector was used for the detection.

In silica gel column chromatography, the indication of NH means use of aminopropylsilane-bonded silica gel and the indication of Diol means use of 3-(2,3-dihydroxypropoxy)propylsilane-bonded silica gel.

¹H Nuclear magnetic resonance (NMR) spectra were obtained for many of the compounds in the following examples. Characteristic chemical shifts (6) are given in parts-per-million downfield from tetramethylsilane using conventional abbreviations for designation of major peaks, including s (singlet), d (doublet), t (triplet), q (quartet), m (multiplet), and br (broad). The following abbreviations are used for common solvents: CDCl₃ (deuterochloroform), DMSO-d₆ (deuterodimethylsulfoxide). For the analysis of ¹H NMR, ACD/SpecManager (trade name) software and the like were used. Peaks of a hydroxyl group, an amino group and the like, having very mild proton peak, are not sometimes described.

MS was measured by LC/MS. As the ionization method, ESI method, or APCI method was used. The data indicates actual measured value (found). While molecular ion peak is generally observed, a fragment ion is sometimes observed. For example, in the case of a compound having a tert-butoxycarbonyl group, a peak after elimination of a tert-butoxycarbonyl group or a tert-butyl group may be observed as a fragment ion. In the case of a compound having a hydroxy group, a peak after elimination of H₂O may be observed as a fragment ion. In the case of a salt, a molecular ion peak or fragment ion peak of free form is generally observed.

Where indicated, intermediate preparations and example compounds are purified by HPLC. Tables 1 to 3 list the column, mobile phases and gradients used for some of the HPLC separations.

TABLE 1
HPLC Method A

| | |
|---|---|
| Column | CHIRALPAK IA(VF003) 20 mmID*250 mmL, 5 μm |
| Mobile Phase | Hexane/Ethanol = 650/350 (v/v) |

TABLE 2
HPLC Method B

| | |
|---|---|
| Column | CHIRALPAK_IA (AU001) 20 mmID*250 mmL, 5 μm |
| Mobile Phase | Hexane/Ethanol = 900/100 (v/v) |

TABLE 3
HPLC Method C

| | |
|---|---|
| Column | CHIRALPAK IG(WL003) 20 mmID*250 mmL, 5 μm |
| Mobile Phase | Ethyl acetate |

The preparations and examples may employ supercritical fluid chromatography (SFC) to separate enantiomers. Table 4 lists equipment, materials, and conditions for some of the SFC separations.

TABLE 4
SFC Method A

| | |
|---|---|
| Column | Phenomenex Cellulose-2, 21.2 mmID*150 mmL, 5 μm |
| Mobile Phase | 15-35% MeOH/CO₂. Methanol contained 0.1% NH₄OH |

In Examples, the following abbreviations are used.
MS: mass spectrum
M: mol concentration
CDCl₃: deuterochloroform
DMSO-d₆: deuterodimethyl sulfoxide
¹H NMR: proton nuclear magnetic resonance
LC/MS: liquid chromatograph mass spectrometer
ESI: electrospray ionization
APCI: atmospheric pressure chemical ionization
Ar: argon
CaCl₂: calcium chloride
DAST: (diethylamino)sulfur trifluoride
DMAP: 4-dimethylaminopyridine
DMF: N,N-dimethylformamide
DMSO: dimethyl sulfoxide
EtOAc: ethyl acetate
EtOH: ethanol
HCl: hydrogen chloride
IPE: diisopropyl ether
K₃PO₄: potassium triphosphate
mCPBA: m-chloroperbenzoic acid
MeOH: methanol
MgSO₄: magnesium sulfate
NaOEt: sodium ethoxide
NaHCO₃: sodium hydrogen carbonate
NaOH: sodium hydroxide
NaOMe: sodium methoxide
Na₂SO₄: sodium sulfate
Na₂S₂O₃: sodium thiosulfate
NBS: N-bromosuccinimide
NH₄Cl: ammonium chloride
PTSA: p-toluenesulfonic acid
TEA: triethylamine
t-BuOH: tert-butanol
TFA: trifluoroacetic acid
THF: tetrahydrofuran

### Example 24

### 2-(4-bromo-2-fluoro-6-methylphenyl)-6-ethoxy-2,5-dihydro-4H-pyrazolo[3,4-d]pyrimidin-4-one

### A) 4,6-dichloro-5-(1,3-dioxolan-2-yl)-2-(methylsulfanyl)pyrimidine

PTSA acid monohydrate (0.043 g) was added to a solution of 4,6-dichloro-2-(methylthio)pyrimidine-5-carbaldehyde (1.02 g) and ethane-1,2-diol (0.767 mL) in toluene (30 mL) at room temperature. The mixture was heated at reflux using Dean-Stark apparatus for 10 hours. The mixture was neutralized with saturated aqueous sodium hydrogen carbonate solution at room temperature and extracted with EtOAc. The organic layer was separated, washed with water and brine, dried over magnesium sulfate, and concentrated under reduced pressure. The residue was purified by silica gel column chromatography (EtOAc/hexane) to give the title compound (1.200 g). ¹H NMR (300 MHz, DMSO-d₆) δ 2.56 (3H, s), 3.94-4.09 (2H, m), 4.13-4.26 (2H, m), 6.18 (1H, s).

### B) 4,6-dichloro-5-(1,3-dioxolan-2-yl)-2-(methanesulfonyl)pyrimidine

A mixture of 4,6-dichloro-5-(1,3-dioxolan-2-yl)-2-(methylsulfanyl)pyrimidine (200 mg) and mCPBA (442 mg) in DMF (3 mL) was stirred at room temperature for 12 hours. Additional mCPBA (220 mg) was added to the mixture. The mixture was stirred at room temperature for 1.5 hours. To the mixture were added water and aqueous sodium thiosulfate solution. The mixture was extracted with EtOAc. The organic layer was dried over magnesium sulfate, and concentrated under reduced pressure. The residue was purified by silica gel column chromatography (EtOAc/hexane) to give the title compound (123 mg). ¹H NMR (400 MHz, DMSO-d₆) δ 3.43 (3H, s), 4.04-4.13 (2H, m), 4.21-4.29 (2H, m), 6.31 (1H, s).

### C) 4,6-dichloro-5-(1,3-dioxolan-2-yl)-2-ethoxypyrimidine

To a solution of 4,6-dichloro-5-(1,3-dioxolan-2-yl)-2-(methanesulfonyl)pyrimidine (50 mg) in THE (2 mL) was added 20% sodium ethoxide in EtOH (59.7 mg) at 0° C. The mixture was stirred at 0° C. for 30 minutes. To the mixture was added water. The mixture was extracted with EtOAc. The organic layer was separated, washed with water and brine, dried over anhydrous sodium sulfate, and concentrated under reduced pressure. The residue was purified by silica gel column chromatography (EtOAc/hexane) to give the title compound (32.6 mg). MS: [M+H]⁺ 264.9.

### D) 2-(4-bromo-2-fluoro-6-methylphenyl)-4-chloro-6-ethoxy-2H-pyrazolo[3,4-d]pyrimidine

A solution of sodium nitrite (0.558 g) in water (15 mL) was slowly added to a solution of 4-bromo-2-fluoro-6-methylaniline (1.5 g) in 6 M HCl (20 mL) at 0° C. After being stirred at 0° C. for 20 minutes, tin(II) chloride (4.18 g) in 6 M HCl (30 mL) was added to the mixture. The mixture was stirred at 0° C. for 30 minutes. To the mixture was added 8 M NaOH aqueous solution at 0° C. The mixture was extracted with EtOAc. The organic layer was separated, washed with brine, dried over anhydrous sodium sulfate, and concentrated under reduced pressure to give a pale brown solid (1.450 g). To a solution of the 4,6-dichloro-5-(1,3-dioxolan-2-yl)-2-ethoxypyrimidine (100 mg) in THE (1.0 mL) were added the solid (83 mg) and TEA (0.158 mL) at room temperature. The mixture was stirred at 60° C. overnight. The mixture was poured into water and extracted with EtOAc. The organic layer was separated, washed with brine, dried over anhydrous sodium sulfate, and concentrated under reduced pressure. To the residue were added toluene (2 mL) and PTSA monohydrate (3.59 mg) at room temperature. The mixture was stirred at room temperature for 1 hour. The mixture was poured into saturated aqueous sodium hydrogen carbonate solution, and extracted with EtOAc. The organic layer was separated, washed with brine, dried over anhydrous sodium sulfate, and concentrated under reduced pressure. The residue was purified by silica gel column chromatography (EtOAc/hexane) to give the title compound (130 mg). MS: [M+H]⁺ 384.9.

### E) 2-(4-bromo-2-fluoro-6-methylphenyl)-6-ethoxy-2,5-dihydro-4H-pyrazolo[3,4-d]pyrimidin-4-one

To a solution of 2-(4-bromo-2-fluoro-6-methylphenyl)-4-chloro-6-ethoxy-2H-pyrazolo[3,4-d]pyrimidine (337 mg) in THF (10 mL) was added 8 M NaOH aqueous solution (2 mL). The mixture was stirred at room temperature overnight. To the mixture were added EtOAc and 6 M HCl aqueous solution (3 mL). Then, saturated aqueous NAHCO₃ was added to the mixture. The organic layer was separated, dried over anhydrous sodium sulfate, and concentrated under reduced pressure. The residue was purified by silica gel column chromatography (EtOAc/hexane) to give the title compound (300 mg). ¹H NMR (400 MHz, DMSO-d₆) δ 1.34 (3H, t, J=7.1 Hz), 2.12 (3H, s), 4.40 (2H, q, J=7.1 Hz), 7.60 (1H, s), 7.73 (1H, d, J=9.2 Hz), 8.75 (1H, s), 11.85 (1H, s).

### Example 39

### 2-(4-cyclopropyl-2-fluoro-6-methylphenyl)-6-ethoxy-2,5-dihydro-4H-pyrazolo[3,4-d]pyrimidin-4-one

A mixture of 2-(4-bromo-2-fluoro-6-methylphenyl)-6-ethoxy-2,5-dihydro-4H-pyrazolo[3,4-d]pyrimidin-4-one (240 mg), cyclopropylboronic acid (168 mg), K₃PO₄ (416 mg), palladium(II) acetate (29.3 mg), and tricyclohexylphosphine (73.3 mg) in toluene (5 mL) and water (1 mL) was stirred at 100° C. under nitrogen atmosphere overnight. After cooling to room temperature, EtOAc and water were added to the mixture. The organic layer was separated, washed with brine and dried over anhydrous sodium sulfate, and concentrated under reduced pressure. The residue was purified by silica gel column chromatography (EtOAc/hexane) to give the title compound (100 mg). ¹H NMR (400 MHz, DMSO-d₆) δ 0.75-0.83 (2H, m), 0.99-1.06 (2H, m), 1.34 (3H, t, J=7.1 Hz), 1.95-2.05 (1H, m), 2.06 (3H, s), 4.40 (2H, q, J=7.1 Hz), 6.99-7.03 (2H, m), 8.67 (1H, s), 11.80 (1H, s).

### Example 142

### 2-(2-bromo-4-(difluoromethoxy)-6-methylphenyl)-6-ethoxy-2,5-dihydro-4H-pyrazolo[3,4-d]pyrimidin-4-one

### A) 2-bromo-4-(difluoromethoxy)-6-methylaniline

NBS (1.752 g) was added portionwise to a solution of 4-(difluoromethoxy)-2-methylaniline (1.55 g) in DMF (30 mL) at 0° C. The mixture was stirred at 0° C. under a dry atmosphere (CaC₂ tube) for 1 hour. The mixture was quenched with saturated aqueous NaHCO₃ and 1 M Na₂S₂O₃ aqueous solution at 0° C. and extracted with EtOAc. The organic layer was separated, washed with water and brine, dried over MgSO₄ and concentrated in vacuo. The residue was purified by basic silica gel column chromatography (EtOAc/hexane) to give the title compound (1.450 g). MS: [M+H]⁺ 252.0.

### B) (2-bromo-4-(difluoromethoxy)-6-methylphenyl)hydrazine

The solution of sodium nitrite (0.437 g) in water (15 mL) was added dropwise to a solution of 2-bromo-4-(difluoromethoxy)-6-methylaniline (1.45 g) in 6 M HCl aqueous solution (35 mL) at 0° C. After being stirred at 0° C. for 20 minutes, tin(II) chloride (3.27 g) in 6 M HCl aqueous solution (10 mL) was added to the reaction mixture. The mixture was stirred at 0° C. for 30 minutes. 8 M NaOH aqueous solution was added to bring the pH of the solution to the basic range. EtOAc and celite was added to the mixture. The precipitate was removed by filtration through celite pad, and washed with EtOAc. The organic layer was separated, washed with water and brine, dried over MgSO₄ and concentrated in vacuo. The residue was purified by silica gel column chromatography (EtOAc/hexane) to give the title compound (1.060 g). ¹H NMR (300 MHz, DMSO-d₆) δ 2.38 (3H, s), 4.17 (2H, br s), 5.55 (1H, s), 6.83-7.41 (3H, m).

### C) 4-(2-(2-bromo-4-(difluoromethoxy)-6-methylphenyl)hydrazineyl)-6-chloro-5-(1,3-dioxolan-2-yl)-2-ethoxypyrimidine

(2-Bromo-4-(difluoromethoxy)-6-methylphenyl)hydrazine hydrochloride (4 g) was added to a solution of 4,6-dichloro-5-(1,3-dioxolan-2-yl)-2-ethoxypyrimidine (3.84 g) and TEA (9.18 mL) in THE (130 mL) at room temperature. The mixture was stirred at 70° C. under Ar for 5 hours. The mixture was poured into water at room temperature and extracted with EtOAc. The organic layer was separated, washed with brine, dried over MgSO₄ and concentrated in vacuo. The residue was purified by silica gel column chromatography (EtOAc/hexane) to give the title compound (3.89 g). MS: [M+H]⁺ 495.0.

### D) 2-(2-bromo-4-(difluoromethoxy)-6-methylphenyl)-6-ethoxy-2,5-dihydro-4H-pyrazolo[3,4-d]pyrimidin-4-one

TFA (24.18 mL) was added dropwise to a solution of 4-(2-(2-bromo-4-(difluoromethoxy)-6-methylphenyl)hydrazineyl)-6-chloro-5-(1,3-dioxolan-2-yl)-2-ethoxypyrimidine (3.89 g) in THE (75 mL) at 0° C. After being stirred at 0° C. for 5 minutes, the mixture was stirred at room temperature under Ar for 1.5 hours. The mixture was poured into 2 M NaOH aqueous solution (157 mL) and saturated aqueous NaHCO₃ was added to the mixture at 0° C. The mixture was extracted with EtOAc/THF (2:1). The organic layer was separated, washed with brine, dried over MgSO₄ and concentrated in vacuo. The residue was purified by silica gel column chromatography (EtOAc/hexane) to give the title compound (2.71 g). MS: [M+H]⁺ 414.9.

### E) 2-(2-bromo-4-(difluoromethoxy)-6-methylphenyl)-6-ethoxy-2,5-dihydro-4H-pyrazolo[3,4-d]pyrimidin-4-one

2-(2-bromo-4-(difluoromethoxy)-6-methylphenyl)-6-ethoxy-2,5-dihydro-4H-pyrazolo[3,4-d]pyrimidin-4-one (3.0 g) was dissolved in hot EtOAc (74 mL). Hexane (41 mL) was added to the mixture at 70° C. The mixture was stirred overnight at room temperature. The precipitate was collected by filtration, washed with hexane, and dried in vacuo to give the title compound (2.21 g). ¹H NMR (300 MHz, DMSO-d₆) δ 1.34 (3H, t, J=7.2 Hz), 2.05 (3H, s), 4.41 (2H, q, J=7.0 Hz), 7.34 (1H, d, J=2.3 Hz), 7.40 (1H, t, J=73.9 Hz), 7.56 (1H, d, J=2.6 Hz), 8.67 (1H, s), 11.83 (1H, s).

### Example 146-1

### 2-(4-cyclopropyl-2,6-dimethylphenyl)-6-methoxy-2,5-dihydro-4H-pyrazolo[3,4-d]pyrimidin-4-one

### A) (4-bromo-2,6-dimethylphenyl)hydrazine hydrochloride

A mixture of sodium nitrite (18.97 g) and water (400 mL) was added dropwise to a mixture of 4-bromo-2,6-dimethylaniline (50 g) and 6 M HCl aqueous solution (1000 mL) at 0° C. After being stirred at 0° C. for 30 minutes, a mixture of tin(II) chloride (142 g) and 6 M HCl aqueous solution (200 mL) was added to the reaction mixture. The mixture was stirred at 0° C. for 30 minutes. The precipitate was collected by filtration, washed with ice-cooled 2 M HCl aqueous solution (300 mL×2) and ice-cooled IPE (300 mL×3), and then dried in vacuo to give the title compound (55.0 g). ¹H NMR (400 MHz, DMSO-d₆) δ 2.38 (6H, s), 6.42-6.94 (1H, m), 7.32 (2H, s), 9.73 (3H, br s).

### B) 2,4,6-trichloro-5-(1,3-dioxolan-2-yl)pyrimidine

PTSA monohydrate (2.371 g) was added to a mixture of 2,4,6-trichloropyrimidine-5-carbaldehyde (52.7 g) and ethane-1,2-diol (41.8 mL) in toluene (500 mL) at room temperature. The mixture was stirred at 120° C. for 1 hour while the generated water was removed by Dean-Stark trap. The mixture was poured into saturated aqueous NaHCO₃ at room temperature and extracted with EtOAc. The organic layer was separated, washed with brine, dried over MgSO₄ and concentrated in vacuo. The residue was suspended with IPE and the precipitate was collected by filtration, washed with IPE and dried in vacuo to give the title compound (39.4 g). ¹H NMR (400 MHz, DMSO-d₆) δ 3.98-4.11 (2H, m), 4.17-4.28 (2H, m), 6.22 (1H, s).

### C) 4-(2-(4-bromo-2,6-dimethylphenyl)hydrazineyl)-2,6-dichloro-5-(1,3-dioxolan-2-yl)pyrimidine

To a mixture of (4-bromo-2,6-dimethylphenyl)hydrazine hydrochloride (40.7 g) and MeOH (250 mL) was added 2,4,6-trichloro-5-(1,3-dioxolan-2-yl)pyrimidine (39.4 g) at 0° C. TEA (64.5 mL) was added dropwise to the mixture at 0° C. The mixture was stirred at 0° C. for 2 hours. The resulting solid was collected by filtration, washed with MeOH (100 mL) and hexane (100 mL), and dried in vacuo to give the title compound (50.5 g). This product was subjected to the next reaction without further purification. MS: [M+H]⁺ 432.9.

### D) 2-(4-bromo-2,6-dimethylphenyl)-6-chloro-2,5-dihydro-4H-pyrazolo[3,4-d]pyrimidin-4-one

TFA (22.18 mL) was added dropwise to a mixture of 4-(2-(4-bromo-2,6-dimethylphenyl)hydrazineyl)-2,6-dichloro-5-(1,3-dioxolan-2-yl)pyrimidine (50 g) and toluene (500 mL) at room temperature. The mixture was stirred at room temperature for 3 hours. The mixture was added dropwise to a mixture of K₃PO₄ (68 g) and water (230 mL) at 0° C. and extracted with EtOAc/THF. The organic layer was separated, washed with brine, dried over Na₂SO₄ and concentrated in vacuo. To a mixture of the residue and THE (dry) (400 mL) was added 4 M NaOH aqueous solution (101 mL) at room temperature. The mixture was stirred at 60° C. for 20 minutes. The mixture was acidified with 2 M HCl aqueous solution (180 mL) at 0° C. and extracted with EtOAc. The organic layer was separated, washed with saturated aqueous NaHCO₃ and brine, and dried over Na₂SO₄. The mixture was filtered through silica gel pad using EtOAc, and the filtrate was concentrated in vacuo. The resulting solid was collected by filtration, washed with EtOAc and IPE, and dried in vacuo to give the title compound (23.10 g). MS: [M+H]⁺ 353.0.

### E) 2-(4-bromo-2,6-dimethylphenyl)-6-methoxy-2,5-dihydro-4H-pyrazolo[3,4-d]pyrimidin-4-one

To a mixture of 2-(4-bromo-2,6-dimethylphenyl)-6-chloro-2,5-dihydro-4H-pyrazolo[3,4-d]pyrimidin-4-one (23.1 g) and THE (dry) (230 mL) was added 28% NaOMe solution in MeOH (37.8 g) at room temperature. The mixture was stirred at 70° C. for 8 hours. The mixture was poured into saturated aqueous NH₄Cl at 0° C. and extracted with EtOAc. The organic layer was separated, washed with saturated aqueous NaHCO₃ and brine, and dried over Na₂SO₄. The mixture was filtered through a pad with two layers of basic silica gel and silica gel using EtOAc. The filtrate was concentrated in vacuo, and the resulting solid was collected by filtration using IPE, washed with XXX, and dried in vacuo to give the title compound (21.40 g). MS: [M+H]⁺ 349.0

### F) 2-(4-cyclopropyl-2,6-dimethylphenyl)-6-methoxy-2,5-dihydro-4H-pyrazolo[3,4-d]pyrimidin-4-one

To a mixture of 2-(4-bromo-2,6-dimethylphenyl)-6-methoxy-2,5-dihydro-4H-pyrazolo[3,4-d]pyrimidin-4-one (5.0 g), toluene (100 mL) and water (20 mL) were added cyclopropylboronic acid (3.69 g), dichloro[1,1′-bis(di-t-butylphosphino)ferrocene]palladium(II) (0.467 g) and K₃PO₄ (4.56 g) at room temperature. The mixture was stirred at 100° C. under Ar for 3 hours. To a mixture of 2-(4-bromo-2,6-dimethylphenyl)-6-methoxy-2,5-dihydro-4H-pyrazolo[3,4-d]pyrimidin-4-one (15.0 g), toluene (300 mL) and water (60 mL) were added cyclopropylboronic acid (11.1 g), dichloro[1,1′-bis(di-t-butylphosphino)ferrocene]palladium(II) (1.40 g) and K₃PO₄ (13.7 g) at room temperature. The mixture was stirred at 100° C. under Ar for 3 hours. The two reaction mixtures were poured into water and extracted with EtOAc. The organic layer was separated, washed with brine, and dried over Na₂SO₄. The mixture was filtered through basic silica gel pad using EtOAc. The filtrate was concentrated in vacuo. The residue was purified by silica gel column chromatography (EtOAc/hexane) to give the title compound (15.5 g). The solid was dissolved in EtOAc (800 mL) and filtered to remove the dust. The filtrate was concentrated in vacuo. The solid was dissolved in EtOAc (400 mL) at 70° C. The mixture was stirred overnight at room temperature. The precipitate was collected by filtration, washed with hexane, and dried in vacuo to give the title compound (12.00 g). ¹H NMR (400 MHz, DMSO-d₆) δ 0.69-0.78 (2H, m), 0.91-1.02 (2H, m), 1.88-1.99 (7H, m), 3.93 (3H, s), 6.94 (2H, s), 8.55 (1H, s), 11.83 (1H, s).

### Example 146-2

### 2-(4-cyclopropyl-2,6-dimethylphenyl)-6-methoxy-2,5-dihydro-4H-pyrazolo[3,4-d]pyrimidin-4-one

### A) 2,4-dichloro-6-[2-(4-cyclopropyl-2,6-dimethylphenyl)hydrazinyl]-5-(1,3-dioxolan-2-yl)pyrimidine

To a solution of (4-cyclopropyl-2,6-dimethylphenyl)hydrazine hydrochloride (11.0 g) in MeOH (60 mL) was added 2,4,6-trichloro-5-(1,3-dioxolan-2-yl)pyrimidine (11.0 g) at 0° C. TEA (18.0 mL) was added dropwised to the mixture at 0° C. The mixture was stirred at 0° C. for 3 hours. The reaction mixture was filtered and the filter cake was washed with MeOH (15 mL) and hexane (15 mL) to afford the title compound (12.3 g). ¹H NMR (400 MHz, DMSO-d₆) δ 0.47-0.62 (2H, m), 0.74-0.88 (2H, m), 1.67-1.78 (1H, m), 2.31 (6H, s), 3.92-3.99 (2H, m), 4.19-4.27 (2H, m), 5.94 (1H, s), 6.60 (2H, s), 6.89 (1H, d, J=3.2 Hz), 8.86 (1H, d, J=3.2 Hz).

### B) 4,6-dichloro-2-(4-cyclopropyl-2,6-dimethylphenyl)-2H-pyrazolo[3,4-d]pyrimidine

4 M HCl in MTBE (123 mL) was added dropwise to a solution of 2,4-dichloro-6-[2-(4-cyclopropyl-2,6-dimethylphenyl)hydrazinyl]-5-(1,3-dioxolan-2-yl)pyrimidine (12.3 g) in THF (120 mL) at 0° C. under N₂ atmosphere. The mixture was stirred at 20° C. for 1 hour. The reaction mixture was added dropwise to saturated aqueous NaHCO₃ (200 mL) at 0° C., and then extracted with EtOAc (100 mL×2). The combined organic layers were washed with brine (100 mL×2), dried over anhydrous Na₂SO₄, filtered and concentrated under reduced pressure to give a residue. The residue was purified by flash silica gel column chromatography (EtOAc/petroleum ether) to afford the title compound (7.4 g). MS: [M+H]⁺ 333.1.

### C) 6-chloro-2-(4-cyclopropyl-2,6-dimethylphenyl)-2,5-dihydro-4H-pyrazolo[3,4-d]pyrimidin-4-one

To a solution of compound 4,6-dichloro-2-(4-cyclopropyl-2,6-dimethylphenyl)-2H-pyrazolo[3,4-d]pyrimidine (7.4 g) in THE (74 mL) was added 4 M NaOH aqueous solution (19.4 mL) at 20° C. The mixture was stirred at 60° C. for 1 hour. The mixture was acidified with 2 M HCl aqueous solution (40 mL) at 0° C. and extracted with EtOAc (100 mL×3). The organic layer was separated, washed with saturated aqueous NaHCO₃ (100 mL) and brine (100 mL), and dried over Na₂SO₄. The mixture was filtered through silica gel pad using EtOAc, and the filtrate was concentrated in vacuum to afford the title compound (7 g). ¹H NMR (400 MHz, DMSO-d₆) δ 0.70-0.77 (2H, m), 0.95-1.02 (2H, m), 1.89-1.97 (7H, m), 6.96 (2H, s), 8.76 (1H, s), 12.85 (1H, s).

### D) 2-(4-cyclopropyl-2,6-dimethylphenyl)-6-methoxy-2,5-dihydro-4H-pyrazolo[3,4-d]pyrimidin-4-one

To a solution of 6-chloro-2-(4-cyclopropyl-2,6-dimethylphenyl)-2,5-dihydro-4H-pyrazolo[3,4-d]pyrimidin-4-one (7 g) in THE (70 mL) was added 30% NaOMe solution in methanol (12.0 g). The mixture was stirred at 70° C. for 8 hours. The reaction mixture was quenched by addition of saturated aqueous NH₄Cl (100 mL) at 0° C., and then extracted with EtOAc (100 mL×3). The combined organic layers were washed with brine (50 mL×2), dried over anhydrous Na₂SO₄, filtered and concentrated under reduced pressure to afford the title compound (6.5 g). ¹H NMR (400 MHz, DMSO-d₆) δ 0.69-0.76 (2H, m), 0.94-1.01 (2H, m), 1.87-1.96 (7H, m), 3.93 (3H, s), 6.94 (2H, s), 8.55 (1H, s), 11.82 (1H, br s).

### E) 2-(4-cyclopropyl-2,6-dimethylphenyl)-6-methoxy-2,5-dihydro-4H-pyrazolo[3,4-d]pyrimidin-4-one

2-(4-Cyclopropyl-2,6-dimethylphenyl)-6-methoxy-2,5-dihydro-4H-pyrazolo[3,4-d]pyrimidin-4-one (45.8 g) was dissolved in EtOAc (1470 mL) at 70° C. The solution was filtered. The filtrate (suspension) was heated at 70° C. To the clear solution was added dropwise heptane (400 mL) at 65-70° C. To the mixture was added a seed crystal and stirred at 66° C. for 40 minutes. To the suspension was added dropwise heptane (600 mL) at 65° C. The suspension was cooled to room temperature gradually. The precipitate was collected by filtration, washed with a mixture of EtOAc/heptane (v/v=1/1, 400 mL) and dried to give the title compound (34.1 g). ¹H NMR (400 MHz, DMSO-d₆) δ 0.65-0.81 (2H, m), 0.90-1.08 (2H, m), 1.89-1.97 (7H, m), 3.94 (3H, s), 6.94 (2H, s), 8.56 (1H, s), 11.84 (1H, s).

### Example 186

### 2-(4-cyclopropyl-2-(difluoromethyl)-6-fluorophenyl)-6-(2-methoxyethoxy)-2,5-dihydro-4H-pyrazolo[3,4-d]pyrimidin-4-one

### A) 1-(difluoromethyl)-3-fluoro-2-nitrobenzene

DAST (2.81 mL) was added to a solution of 3-fluoro-2-nitrobenzaldehyde (1.2 g) in toluene (20 mL) at 0° C. The mixture was stirred at room temperature under N₂ for 2 hours. The mixture was quenched with water at 0° C. and extracted with EtOAc. The organic layer was separated, washed with water and brine, dried over Na₂SO₄ and concentrated in vacuo. The residue was purified by silica gel column chromatography (EtOAc/hexane) to give the title compound (1.120 g). ¹H NMR (400 MHz, CDCl₃) δ 6.81-7.18 (1H, m), 7.43 (1H, t, J=8.9 Hz), 7.57 (1H, d, J=7.8 Hz), 7.66 (1H, td, J=8.2, 4.9 Hz).

### B) 4-bromo-2-(difluoromethyl)-6-fluoroaniline

A mixture of 1-(difluoromethyl)-3-fluoro-2-nitrobenzene (9.5 g) and Pd—C (10%, wetted with 55% water, 2.351 g) in EtOH (50 mL) was hydrogenated under balloon pressure at room temperature for 16 hours. The catalyst was removed by filtration and the filtrate was concentrated in vacuo The residue was dissolved in DMF (dry) (100 mL) at 0° C. and NBS (9.28 g) was added. The mixture was stirred at 0° C. under N₂ for 1 hour. Then water (100 mL) was added, and the resulting mixture was extracted with EtOAc (100 mL×2). The combined organic solvent was washed with water, brine and dried with anhydrous Na₂SO₄. The solvent was removed under vacuum. The residue was purified by silica gel column chromatography (EtOAc/hexane) to give the title compound (10 g). MS: [M+H]⁺ 240.0.

### C) 4-cyclopropyl-2-(difluoromethyl)-6-fluoroaniline

A mixture of 4-bromo-2-(difluoromethyl)-6-fluoroaniline (10 g), cyclopropylboronic acid (5.37 g), dichloro[1,1′-bis(di-t-butylphosphino)ferrocene]palladium(II) (0.815 g), and K₃PO₄ (17.69 g) in toluene (100 mL) and water (20 mL) was stirred at 100° C. under N₂ for 3 hours. The insoluble material was removed by filtration and washed with EtOAc. The organic layer was separated, washed with water and brine, dried over Na₂SO₄ and concentrated in vacuo. The residue was purified by silica gel column chromatography (EtOAc/hexane) to give the title compound (7.60 g). MS: [M+H]⁺ 202.0.

### D) (4-cyclopropyl-2-(difluoromethyl)-6-fluorophenyl)hydrazine

To a stirred suspension of 4-cyclopropyl-2-(difluoromethyl)-6-fluoroaniline (1 g) in 6 M HCl aqueous solution (36 mL) was added dropwise a solution of sodium nitrite (0.377 g) in water (18 mL) at 0° C. After being stirred at 0° C. for 30 minutes, CH₃CN (24 mL) was added. To the stirred mixture (solution) was added dropwise a solution of tin(II) chloride (2.83 g) in 6 M HCl aqueous solution (18 mL) at 0° C. The mixture was stirred at 0° C. for 1 hour. The resulting mixture was quenched with 8 M NaOH aqueous solution (ca. 42 mL) at 0° C., and diluted with brine and THE (90 mL). The insoluble material was removed by filtration and the filtrate was extracted with THF. The organic layer was separated, dried over anhydrous MgSO₄, and concentrated in vacuo. The crude material (1 g) was used in the next reaction without further purification. ¹H NMR (300 MHz, DMSO-d₆) 0.51-0.68 (2H, m), 0.82-0.95 (2H, m), 1.81-1.96 (1H, m), 4.40 (2H, s), 6.39 (1H, br d, J=3.0 Hz), 6.85-6.93 (1H, m), 7.03 (1H, s), 7.53-8.02 (1H, m).

### E) 4,6-dichloro-5-(1,3-dioxolan-2-yl)-2-(methylthio)pyrimidine

PTSA monohydrate (0.128 g) was added to a solution of 4,6-dichloro-2-(methylthio)pyrimidine-5-carbaldehyde (3 g) and ethylene glycol (2.256 mL) in toluene (100 mL) at room temperature. The mixture was refluxed for 3 hours while the generated water was removed by Dean-Stark trap. The mixture was neutralized with saturated aqueous NaHCO₃ at room temperature and extracted with EtOAc. The organic layer was separated, washed with water and brine, dried over MgSO₄ and concentrated in vacuo. The residue was purified by silica gel column chromatography (EtOAc/hexane) to give the title compound (3.37 g). MS: [M+H]⁺ 266.9.

### F) 4,6-dichloro-5-(1,3-dioxolan-2-yl)-2-(methylsulfonyl)pyrimidine

m-CPBA (25.00 g) was added to a solution of 4,6-dichloro-5-(1,3-dioxolan-2-yl)-2-(methylthio)pyrimidine (12.9 g) in DME (150 mL) at 0° C. The mixture was stirred at room temperature overnight. The resulting solid was collected by filtration using DME, washed with DME and dried in vacuo to give the title compound (9.00 g). MS: [M+H]⁺ 298.9.

### G) 4,6-dichloro-5-(1,3-dioxolan-2-yl)-2-(2-methoxyethoxy)pyrimidine

To a stirred solution of 2-methoxyethanol (22.82 mL) in DME (70 mL) was added portionwise 60% NaH (3.70 g) at 0° C. The mixture was stirred at room temperature for 30 minutes, and then added dropwise using a dropping funnel to the mixture of 4,6-dichloro-5-(1,3-dioxolan-2-yl)-2-(methylsulfonyl)pyrimidine (8.66 g) in DME (70 mL) at 0° C. The mixture was stirred at room temperature for 1 hour, diluted with water, and extracted with EtOAc. The organic layer was washed with saturated aqueous NaHCO₃ (two times) and saturated aqueous NaCl, dried over MgSO₄ and concentrated in vacuo. The residue was purified by silica gel column chromatography (EtOAc/hexane) to give the title compound (3.94 g). MS: [M+H]⁺ 295.1.

### H) 4-chloro-6-(2-(4-cyclopropyl-2-(difluoromethyl)-6-fluorophenyl)hydrazineyl)-5-(1,3-dioxolan-2-yl)-2-(2-methoxyethoxy)pyrimidine

To a solution of 4,6-dichloro-5-(1,3-dioxolan-2-yl)-2-(2-methoxyethoxy)pyrimidine (1.467 g) in THE (dry) (40 mL) were added (4-cyclopropyl-2-(difluoromethyl)-6-fluorophenyl)hydrazine (1.075 g) and Et₃N (2.425 mL) at room temperature. The mixture was stirred at 65° C. for 16 hours, treated with water and extracted with EtOAc. The organic layer was dried over MgSO₄ and concentrated in vacuo. The residue was purified by silica gel column chromatography (EtOAc/hexane) to give the title compound (1.020 g). MS: [M+H]⁺ 475.2.

### I) 2-(4-cyclopropyl-2-(difluoromethyl)-6-fluorophenyl)-6-(2-methoxyethoxy)-2,5-dihydro-4H-pyrazolo[3,4-d]pyrimidin-4-one

To a solution of 4-chloro-6-(2-(4-cyclopropyl-2-(difluoromethyl)-6-fluorophenyl)hydrazineyl)-5-(1,3-dioxolan-2-yl)-2-(2-methoxyethoxy)pyrimidine (976.6 mg) in THF (dry) (30 mL) was added TFA (7.92 mL) at room temperature. The mixture was stirred at room temperature for 2 hours. The mixture was basified with saturated aqueous NaHCO₃ and extracted with EtOAc. The organic layer was separated, washed with brine, dried over MgSO₄ and concentrated in vacuo. The residue was purified by silica gel column chromatography (EtOAc/hexane) to give the title compound (763 mg). MS: [M+H]⁺ 395.2.

### J) 2-(4-cyclopropyl-2-(difluoromethyl)-6-fluorophenyl)-6-(2-methoxyethoxy)-2,5-dihydro-4H-pyrazolo[3,4-d]pyrimidin-4-one

2-(4-cyclopropyl-2-(difluoromethyl)-6-fluorophenyl)-6-(2-methoxyethoxy)-2,5-dihydro-4H-pyrazolo[3,4-d]pyrimidin-4-one (793.3 mg) was dissolved in hot EtOAc (12 mL) at 75° C. To the solution was added dropwise heptane (3 mL) at 50° C. (oil bath) and stirred at 50° C. for 5 minutes. To the suspension was added heptane (3 mL), and the suspension was stirred at 50° C. for 1 minute. Additional heptane (6 mL) was added and the oil bath was removed. After addition of heptane (6 mL), the suspension was stirred at room temperature for 12 hours. After addition of heptane (14 mL), the suspension was cooled to 0° C. The suspension was stirred for 10 minutes and the white crystals were collected by filtration, rinsed with heptane/EtOAc (5:1) and dried to give the title compound (679 mg). ¹H NMR (400 MHz, DMSO-d₆) δ 0.84-0.90 (2H, m), 1.04-1.15 (2H, m), 2.13-2.21 (1H, m), 3.31 (3H, s), 3.64-3.71 (2H, m), 4.46-4.53 (2H, m), 6.88 (1H, t, J=56.0 Hz), 7.39-7.44 (2H, m), 8.80 (1H, s), 11.96 (1H, s).

### Example 224

### 2-(4-cyclopropyl-2,6-dimethylphenyl)-6-((difluoromethoxy)methyl)-2,5-dihydro-4H-pyrazolo[3,4-d]pyrimidin-4-one

### A) 3-amino-i-(4-bromo-2,6-dimethylphenyl)-1H-pyrazole-4-carbonitrile

To a solution of 5-amino-1H-pyrazole-4-carbonitrile (5.10 g) in pyridine (100 mL) were added (4-bromo-2,6-dimethylphenyl)boronic acid (9.0 g) and copper(II) acetate (7.14 g) at room temperature. The mixture was vigroulsly stirred at 90° C. under open air for 3 hours. The mixture was poured into saturated aqueous NH₄Cl at room temperature and extracted with EtOAc. The organic layer was separated, washed with brine, and dried over Na₂SO₄. The mixture was filtered through silica gel and celite pad using EtOAc. The filtrate was concentrated in vacuo. The residue was purified by silica gel column chromatography (EtOAc/hexane) to give the title compound (2.500 g). MS: [M+H]⁺ 291.0.

### B) 3-amino-i-(4-cyclopropyl-2,6-dimethylphenyl)-1H-pyrazole-4-carbonitrile

Cyclopropylboronic acid (2.213 g), K₃PO₄ (9.11 g) and dichloro[1,1′-bis(di-t-butylphosphino)ferrocene]palladium(II) (0.392 g) were added to a mixture of 3-amino-1-(4-bromo-2,6-dimethylphenyl)-1H-pyrazole-4-carbonitrile (2.50 g) in toluene (50 mL) and water (10.00 mL). The mixture was stirred at 100° C. under Ar for 1 hour. The mixture was poured into water and extracted with EtOAc. The organic layer was separated, washed with brine, dried over Na₂SO₄ and concentrated in vacuo. The residue was purified by silica gel column chromatography (EtOAc/hexane) to give the title compound (2.160 g). MS: [M+H]⁺ 253.1.

### C) 3-amino-i-(4-cyclopropyl-2,6-dimethylphenyl)-1H-pyrazole-4-carboxamide

30% hydrogen peroxide in water (31.5 mL) was added dropwise to a mixture of 3-amino-i-(4-cyclopropyl-2,6-dimethylphenyl)-1H-pyrazole-4-carbonitrile (5.18 g) and K₂CO₃ (8.51 g, 61.59 mmol) in DMSO (150 mL) while keeping the reaction temperature in a water bath. The mixture was stirred at room temperature for 1.5 hours. The mixture was quenched with water and saturated aqueous NH₄Cl at room temperature and extracted with EtOAc and THF. The organic layer was separated, washed with water and brine, dried over Na₂SO₄ and concentrated in vacuo. The resulting solid was triturated with EtOAc/IPE/hexane to give the title compound (4.91 g). MS: [M+H]⁺ 271.2.

### D) 1-(4-cyclopropyl-2,6-dimethylphenyl)-3-(2-(difluoromethoxy)acetamido)-1H-pyrazole-4-carboxamide

Oxalyl chloride (1.907 mL) and 4 drops of DMF were added to a mixture of 2-(difluoromethoxy)acetic acid (2.497 g) in THE (40 mL) at 0° C. The mixture was stirred at room temperature for 1 hour. Then the resulting solution was added dropwise using a droping funnel over 1 hour to the mixture of 3-amino-1-(4-cyclopropyl-2,6-dimethylphenyl)-1H-pyrazole-4-carboxamide (3.8243 g) and DIEA (9.88 mL) in THE (130 mL) at −5° C. The mixture was stirred at −5° C. for 10 minutes and stirred at room temperature for 16 hours. The mixture was poured into water and the resulting precipitates were collected by filtration, washed with water and dried to give the title compound (4.83 g). MS: [M+H]⁺ 379.2.

### E) 2-(4-cyclopropyl-2,6-dimethylphenyl)-6-((difluoromethoxy)methyl)-2,5-dihydro-4H-pyrazolo[3,4-d]pyrimidin-4-one

2 M Na₂CO₃ aqueous solution (128 mL) was added to a mixture of 1-(4-cyclopropyl-2,6-dimethylphenyl)-3-(2-(difluoromethoxy)acetamido)-1H-pyrazole-4-carboxamide (4.83 g) in EtOH (300 mL). The mixture was stirred at 80° C. for 16 hours and then EtOH was removed under reduced pressure. To the residue was added NH₄Cl aqueous solution and the mixture was extracted with EtOAc. The organic phase was separated, washed with saturated aqueous NH₄Cl and brine, dried over Na₂SO₄ and concentrated in vacuo. The residue was purified by silica gel column chromatography (EtOAc/hexane) to afford the title compound (3.77 g). MS: [M+H]⁺ 361.2.

### F) 2-(4-cyclopropyl-2,6-dimethylphenyl)-6-((difluoromethoxy)methyl)-2,5-dihydro-4H-pyrazolo[3,4-d]pyrimidin-4-one

2-(4-Cyclopropyl-2,6-dimethylphenyl)-6-((difluoromethoxy)methyl)-2,5-dihydro-4H-pyrazolo[3,4-d]pyrimidin-4-one (4.3872 g) was dissolved in EtOAc (55 mL) at 75-77° C. Then heptane (38 mL) was added dropwise to the mixture at 75-77° C. Then a seed crystal was added to the mixture. The mixture was stirred at the same temperature under N₂ for 1 hour. Then the mixture was gradually cooled to room temperature and stirred under N₂ overnight. Additional heptane (17 mL) was added to the mixture at room temperature. The mixture was stirred at room temperature for 1 hour, then cooled to 0° C., and stirred for further 1 hour. The resulting solid was collected by filtration, rinsed with cold EtOAc/heptane (1:2, 20 mL×2), and dried in vacuo under heating (65° C.) for 1 hour to afford the title compound (3.96 g). ¹H NMR (400 MHz, DMSO-d₆) δ 0.69-0.77 (2H, m), 0.95-1.03 (2H, m), 1.88-1.99 (7H, m), 4.80 (2H, s), 6.64-7.07 (3H, m), 8.70 (1H, s), 12.01 (1H, s).

### Example 235

### 2-[4-(1-fluorocyclopropyl)-2,6-dimethylphenyl]-6-methoxy-2,5-dihydro-4H-pyrazolo[3,4-d]pyrimidin-4-one

### A) 2-bromo-1,3-dimethyl-5-vinylbenzene

Potassium tert-butoxide (6.3 g) was added to a mixture of methyltriphenylphosphonium bromide (20.0 g) in THE (150 mL) at 0° C. After being stirred at 0° C. for 30 minutes, 4-bromo-3,5-dimethylbenzaldehyde (10 g) in THE (100 mL) was added to the reaction mixture. The mixture was stirred at room temperature under N₂ for 1 hour. The mixture was poured into saturated aqueous NH₄Cl at room temperature and extracted with EtOAc. The organic layer was separated, washed with water and brine, dried over MgSO₄ and concentrated in vacuo. The residue was purified by basic silica gel column chromatography (EtOAc/hexane) to give the title compound (9.50 g). ¹H NMR (300 MHz, DMSO-d₆) δ 2.35 (6H, s), 5.28 (1H, dd, J=11.1, 1.1 Hz), 5.85 (1H, dd, J=17.6, 1.1 Hz), 6.64 (1H, dd, J=17.6, 11.1 Hz), 7.27 (2H, s).

### B) 2-bromo-5-(2-bromo-1-fluoroethyl)-1,3-dimethylbenzene

NBS (16.02 g) was added to a solution of 2-bromo-1,3-dimethyl-5-vinylbenzene (9.50 g) in CH₂Cl₂ (100 mL) at 0° C. After being stirred at 0° C. for 5 minutes, triethylamine trihydrofluoride (22.01 mL) was added to the reaction mixture. The mixture was stirred at room temperature under N₂ overnight. The mixture was poured into water at room temperature and extracted with EtOAc. The organic layer was separated, washed with saturated aqueous NaHCO₃ and water, dried over MgSO₄ and concentrated in vacuo. The residue was purified by silica gel column chromatography (EtOAc/hexane) to give the title compound (9.90 g). ¹H NMR (300 MHz, DMSO-d₆) δ 2.38 (6H, s), 3.82-4.01 (2H, m), 5.60-5.87 (1H, m), 7.27 (2H, s).

### C) 2-bromo-5-(1-fluorovinyl)-1,3-dimethylbenzene

A solution of 2-bromo-5-(2-bromo-1-fluoroethyl)-1,3-dimethylbenzene (9.90 g) and potassium tert-butoxide (5.38 g) in THE (200 mL) was refluxed for 1 hour. The mixture was poured into saturated aqueous NH₄Cl at room temperature and extracted with EtOAc. The organic layer was separated, washed with water and brine, dried over MgSO₄ and concentrated in vacuo. The residue was purified by silica gel column chromatography (EtOAc/hexane) to give the title compound (7.10 g). ¹H NMR (300 MHz, DMSO-d₆) δ 2.38 (6H, s), 4.88-5.05 (1H, m), 5.21-5.57 (1H, m), 7.42 (2H, s).

### D) 2-bromo-5-(1-fluorocyclopropyl)-1,3-dimethylbenzene

Zinc (5.99 g) was added to a solution of chloromethyl benzoate (15.64 g), 5,10,15,20-tetraphenyl-21H,23H-porphine iron monochloride (2.151 g) and sodium iodide (13.74 g) in THE (500 mL) at room temperature. After being stirred at room temperature for 5 minutes, 2-bromo-5-(1-fluorovinyl)-1,3-dimethylbenzene (7.0 g) was added to the reaction mixture. The mixture was stirred at 60° C. under Ar overnight. The solid was removed by filtration, and the filtrate was concentrated in vacuo. The residue was purified by basic silica gel column chromatography (EtOAc/hexane) to give the title compound (5.50 g). ¹H NMR (400 MHz, DMSO-d₆) δ 1.06-1.22 (2H, m), 1.39-1.51 (2H, m), 2.37 (6H, s), 7.09 (2H, s).

### E) di-tert-butyl 1-(4-(1-fluorocyclopropyl)-2,6-dimethylphenyl)hydrazine-1,2-dicarboxylate

n-BuLi (2.3 M in hexane) (1.770 mL) was added to a solution of 2-bromo-5-(1-fluorocyclopropyl)-1,3-dimethylbenzene (450 mg) in THE (5 mL) at −78° C. After being stirred at −78° C. for 1 hour, di-tert-butyl (Z)-diazene-1,2-dicarboxylate (469 mg) was added to the reaction mixture. The mixture was stirred at −78° C. under N₂ for 2 hours. The mixture was poured into saturated aqueous NH₄Cl at room temperature and extracted with EtOAc. The residue was purified by silica gel column chromatography (EtOAc/hexane) to give the title compound (210 mg). ¹H NMR (400 MHz, DMSO-d₆) δ 0.80-0.93 (m, 2H) 1.09-1.13 (m, 2H) 1.35-1.42 (m, 18H) 2.24-2.32 (m, 6H) 6.88-7.03 (m, 2H) 9.34-9.43 (m, 1H).

### F) (4-(1-fluorocyclopropyl)-2,6-dimethylphenyl)hydrazine hydrochloride

4 M HCl solution in EtOAc (3 mL) was added to a solution of di-tert-butyl 1-(4-(1-fluorocyclopropyl)-2,6-dimethylphenyl)hydrazine-1,2-dicarboxylate (200 mg) in EtOAc (5 mL) at room temperature. The mixture was stirred at 50° C. under N₂ overnight. The precipitate was collected by filtration to give the title compound (93 mg). ¹H NMR (400 MHz, DMSO-d₆) δ 1.06-1.15 (2H, m), 1.38-1.49 (2H, m), 2.39 (6H, s), 6.59-6.89 (1H, m), 7.01 (2H, s), 9.6G2 (3H, br s).

### G) 4,6-dichloro-5-(1,3-dioxolan-2-yl)-2-methoxypyrimidine

NaOMe (ca. 5 M in MeOH) (0.962 mL) was added to a solution of 4,6-dichloro-5-(1,3-dioxolan-2-yl)-2-(methylsulfonyl)pyrimidine (1.37 g) in THE (60.0 mL) at −78° C. The mixture was stirred at −78° C. under N₂ for 30 minutes. The mixture was quenched with water at −78° C. and extracted with EtOAc. The organic layer was separated, washed with water and brine, dried over Na₂SO₄ and concentrated in vacuo. The residue was purified by silica gel column chromatography (EtOAc/hexane) to give the title compound (0.941 g). MS: [M+H]⁺ 251.0.

### H) 4-chloro-5-(1,3-dioxolan-2-yl)-6-(2-(4-(1-fluorocyclopropyl)-2,6-dimethylphenyl)hydrazineyl)-2-methoxypyrimidine

DIPEA (0.2 mL) was added to a solution of 4,6-dichloro-5-(1,3-dioxolan-2-yl)-2-methoxypyrimidine (80 mg) and (4-(1-fluorocyclopropyl)-2,6-dimethylphenyl)hydrazine hydrochloride (90 mg) in THE (5 mL) at room temperature. The mixture was stirred at 50° C. under N₂ for 3 hours. The mixture was poured into saturated aqueous NH₄Cl at room temperature and extracted with EtOAc. The organic layer was separated, washed with water and brine, dried over MgSO₄ and concentrated in vacuo. The residue was purified by silica gel column chromatography (EtOAc/hexane) to give the title compound (45.0 mg). MS: [M+H]⁺ 409.0.

### I) 2-[4-(1-fluorocyclopropyl)-2,6-dimethylphenyl]-6-methoxy-2,5-dihydro-4H-pyrazolo[3,4-d]pyrimidin-4-one

TFA (0.5 mL) was added to a solution of 4-chloro-5-(1,3-dioxolan-2-yl)-6-(2-(4-(1-fluorocyclopropyl)-2,6-dimethylphenyl)hydrazineyl)-2-methoxypyrimidine (45 mg) in THE (1.5 mL) at room temperature. The mixture was stirred at room temperature under N₂ for 3 hours. The mixture was poured into saturated aqueous NaHCO₃ at room temperature and extracted with EtOAc. The organic layer was separated, washed with water and brine, dried over MgSO₄ and concentrated in vacuo. The residue was purified by silica gel column chromatography (EtOAc/hexane) to give the title compound (28.0 mg). ¹H NMR (400 MHz, DMSO-d₆) δ 1.16-1.28 (2H, m), 1.44-1.59 (2H, m), 1.99 (6H, s), 3.93 (3H, s), 7.16 (2H, s), 8.61 (1H, s), 11.86 (1H, br s).

### Example 321

### 2-(4-cyclopropyl-2-(difluoromethyl)-6-methylphenyl)-6-(methoxymethyl)-2,5-dihydro-4H-pyrazolo[3,4-d]pyrimidin-4-one

### A) 1-(difluoromethyl)-2-fluoro-3-methyl-5-nitrobenzene

DAST (5.77 mL) was added dropwise to a solution of 2-fluoro-3-methyl-5-nitrobenzaldehyde (5.33 g) in anhydrous toluene (60 mL) at 0° C. The mixture was stirred at room temperature under N₂ for 2 hours. The mixture was quenched with water and saturated aqueous NaHCO₃ at 0° C. and extracted with EtOAc. The organic layer was separated, washed with saturated aqueous NaHCO₃, water and brine, dried over MgSO₄ and concentrated in vacuo. The residue was purified by silica gel column chromatography (EtOAc/hexane) to give the title compound (5.52 g). ¹H NMR (400 MHz, CDCl₃) δ 2.43 (3H, d, J=1.8 Hz), 6.76-7.08 (1H, m), 8.21-8.28 (1H, m), 8.32-8.39 (1H, m).

### B) 3-amino-i-(2-(difluoromethyl)-6-methyl-4-nitrophenyl)-1H-pyrazole-4-carbonitrile

A mixture of 1-(difluoromethyl)-2-fluoro-3-methyl-5-nitrobenzene (5.52 g), 3-amino-1H-pyrazole-4-carbonitrile (2.91 g), and K₂CO₃ (4.46 g) in DMSO (25 mL) was stirred at room temperature under a dry atmosphere overnight. The mixture was quenched with water at room temperature and extracted with EtOAc. The organic layer was separated, washed with water and brine, dried over MgSO₄, and filtered through a short-pad of silica-gel using EtOAc. The filtrate was concentrated in vacuo, and the residue was washed with IPE, collected and dried in vacuo to give the title compound (6.83 g). MS: [M−H]⁻ 291.9.

### C) tert-butyl (tert-butoxycarbonyl)(4-cyano-1-(2-(difluoromethyl)-6-methyl-4-nitrophenyl)-1H-pyrazol-3-yl)carbamate

Di-tert-butyl dicarbonate (11.90 mL) was added to a mixture of 3-amino-1-(2-(difluoromethyl)-6-methyl-4-nitrophenyl)-1H-pyrazole-4-carbonitrile (6.83 g), TEA (6.49 mL) and DMAP (0.285 g) in anhydrous THE (50 mL) at room temperature. The mixture was stirred at 70° C. for 1.5 hours. The mixture was quenched with water at room temperature and extracted with EtOAc/THF. The organic layer was separated, washed with saturated aqueous NH₄Cl and brine, dried over MgSO₄, filtered through a short pad of silica-gel and concentrated in vacuo. The residue was washed with IPE, collected and dried to give the title compound (8.46 g). MS: [M+Na]⁺ 516.2.

### D) tert-butyl (1-(4-amino-2-(difluoromethyl)-6-methylphenyl)-4-cyano-1H-pyrazol-3-yl)(tert-butoxycarbonyl)carbamate

tert-Butyl (tert-butoxycarbonyl)(4-cyano-1-(2-(difluoromethyl)-6-methyl-4-nitrophenyl)-1H-pyrazol-3-yl)carbamate (8.46 g) was dissolved in THE (61 mL) and MeOH (56 mL) in a warmed-water bath (ca. 70° C.). Then 1% Pt/C, STAF-1M (wetted with water) (5.64 g) was added portionwise to the mixture and the whole was hydrogenated under balloon pressure at room temperature for 3.5 hours. The catalyst was removed by filtration and the filtrate was concentrated in vacuo to give the title compound (7.96 g). MS: [M−H]⁻ 462.2.

### E) tert-butyl (tert-butoxycarbonyl)(4-cyano-1-(2-(difluoromethyl)-4-iodo-6-methylphenyl)-1H-pyrazol-3-yl)carbamate

n-Amyl nitrite (3.51 mL) was added to a mixture of tert-butyl (1-(4-amino-2-(difluoromethyl)-6-methylphenyl)-4-cyano-1H-pyrazol-3-yl)(tert-butoxycarbonyl)carbamate (7.96 g) in anhydrous CH₃CN (70 mL) at 0° C., followed by addition of copper(I) iodide (4.91 g) and diiodomethane (6.93 mL). After the ice-water bath was removed, the system was filled with N₂ and the mixture was stirred for 10 minutes. Then the mixture was stirred at 58-63° C. under N₂ for 2 hours. The mixture was quenched with saturated aqueous NH₄Cl at 0° C. and diluted with EtOAc. The insoluble material was removed by filtration, and the organic layer was separated, washed with water and brine, dried over MgSO₄. Then silica-gel was added to the mixture and the whole was concentrated in vacuo. The crude mixture supported on silica-gel was purified by silica gel column chromatography (EtOAc/hexane) and concentrated. The residue was washed with IPE, collected and dried in vacuo to give the title compound (7.23 g). MS: [M+Na]⁺ 597.1.

### F) tert-butyl (tert-butoxycarbonyl)(4-cyano-1-(4-cyclopropyl-2-(difluoromethyl)-6-methylphenyl)-1H-pyrazol-3-yl)carbamate

3 M K₃PO₄ aqueous solution (0.564 mL) was added to a mixture of tert-butyl (tert-butoxycarbonyl)(4-cyano-1-(2-(difluoromethyl)-4-iodo-6-methylphenyl)-1H-pyrazol-3-yl)carbamate (243 mg), cyclopropylboronic acid (109 mg) and dichloro[1,1′-bis(di-tert-butylphosphino)ferrocene]palladium(II) (19.30 mg) in toluene (2.0 mL), DME (2.0 mL) and water (0.2 mL) at room temperature. The mixture was stirred at 100° C. under N₂ for 1 hour. The mixture was partitioned between EtOAc and water. The organic layer was washed with brine, dried over Na₂SO₄ and concentrated in vacuo. The residue was purified by silica gel column chromatography (EtOAc/hexane) to give the title compound (200 mg). MS: [M+Na]⁺ 511.2.

### G) tert-butyl (4-carbamoyl-1-(4-cyclopropyl-2-(difluoromethyl)-6-methylphenyl)-1H-pyrazol-3-yl)carbamate

35% H₂O₂ in water (18.47 mL) was added dropwise to a mixture of tert-butyl (tert-butoxycarbonyl)(4-cyano-1-(4-cyclopropyl-2-(difluoromethyl)-6-methylphenyl)-1H-pyrazol-3-yl)carbamate (6.44 g) and K₂CO₃ (7.29 g) in DMSO (55 mL) while keeping the reaction temperature in a water bath. Then additional DMSO (35 mL) was added. The mixture was stirred at room temperature for 90 minutes. Water was added to the mixture and the resulting precipitates were collected, dissolved in THF and concentrated in vacuo to give the title compound. This product was subjected to the next reaction without further purification. MS: [M−H]⁻ 405.1.

### H) 3-amino-1-(4-cyclopropyl-2-(difluoromethyl)-6-methylphenyl)-1H-pyrazole-4-carboxamide

6 M HCl aqueous solution (30 mL) was added to a suspension of tert-butyl (4-carbamoyl-1-(4-cyclopropyl-2-(difluoromethyl)-6-methylphenyl)-1H-pyrazol-3-yl)carbamate (5.36 g) in CH₃CN (60 mL) at room temperature. The mixture was stirred at 40° C. for 1.5 hours and then neutralized with 8 M NaOH aqueous solution (23 mL) at 0° C. The mixture was partitioned between EtOAc and water. The organic layer was separated, washed with water and brine, dried over MgSO₄ and concentrated in vacuo. The residue was crystallized from THF/IPE to give the title compound (3.49 g). MS: [M+H]⁺ 307.0.

### I) 2-(4-cyclopropyl-2-(difluoromethyl)-6-methylphenyl)-6-(methoxymethyl)-2,5-dihydro-4H-pyrazolo[3,4-d]pyrimidin-4-one

To a solution of 3-amino-i-(4-cyclopropyl-2-(difluoromethyl)-6-methylphenyl)-1H-pyrazole-4-carboxamide (600 mg) in THE (11 mL) at room temperature was added 2-methoxyacetyl chloride (0.546 mL). Then the mixture was stirred at room temperature for 30 minutes. The reaction mixture was concentrated in vacuo. Potassium tert-butoxide (769 mg) was added to a solution of the residue obtained above in t-BuOH (23 mL) at room temperature. The mixture was stirred at 90° C. under N₂ for 2 hours. The mixture was quenched with water at room temperature and extracted with EtOAc. The organic layer was separated, washed with saturated aqueous NH₄Cl and brine, dried over Na₂SO₄. The first aqueous phase was acidified with 1 M HCl aqueous solution and extracted with EtOAc. The organic layer was separated, washed with brine, dried over Na₂SO₄. The combined organic layers were concentrated in vacuo. The residue was purified by silica gel column chromatography (EtOAc/hexane) and concentrated to give the title compound (595 mg). The product (595 mg) was dissolved in EtOAc (45 mL) at 70° C. (oil bath temp.). Heptane (55 mL) was added dropwise to the mixture at 70° C. (oil bath temp.) to form white precipitates. After being stirred at the same temperature under N₂ for 1 hour, the mixture was cooled to room temperature and stirred under N₂ overnight, then stirred at 0° C. for 20 minutes. The resulting precipitates were collected and washed with cold EtOAc/heptane (v/v=1/1) and dried in vacuo under heating to give the title compound (489 mg). The product (489 mg) was dissolved to EtOAc (40 mL) at 70° C. (oil bath temp.). Heptane (40 mL) was added dropwise to the mixture at 70° C. (oil bath temp.) to form white precipitates. After being stirred at the same temperature under N₂ for 1 hour, the mixture was cooled to room temperature and stirred under N₂ overnight, then stirred at 0° C. for 20 minutes. The resulting precipitates were collected, washed with cold EtOAc/heptane (v/v=1/1), and dried in vacuo under heating to give the title compound (433 mg). ¹H NMR (400 MHz, DMSO-d₆) δ 0.78-0.85 (2H, m), 1.03-1.10 (2H, m), 1.97 (3H, s), 2.06-2.15 (1H, m), 3.38 (3H, s), 4.32 (2H, s), 6.40-6.72 (1H, m), 7.32 (1H, s), 7.35 (1H, s), 8.76 (1H, s), 11.79 (1H, s).

The compounds of Examples are shown in Table 5-1 to Table 5-30. MS in the tables means actual measured value. The compounds of Examples 1-326 in the following tables were produced according to the methods described in the above-mentioned Examples, or methods analogous thereto.

TABLE 5-1

| Example | | | MS |
|---|---|---|---|
| number | Compound name | Structure | [M + H]⁺ |
| 1 | 6-ethoxy-2-(2-hydroxy-4,6- dimethylphenyl)-2,5-dihydro-4H- pyrazolo[3,4- d]pyrimidin-4-one | 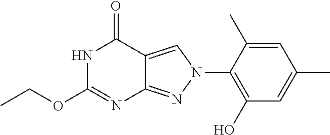 [SMILES: CCOc1nc2nn(-c3c(C)cc(C)cc3O)cc2c(=O)[nH]1] | 301.1 |
| 2 | 2-(2-hydroxy-4,6-dimethylphenyl)- 6-(pyrimidin-4-yl)-2,5-dihydro-4H- pyrazolo[3,4- d]pyrimidin-4-one | 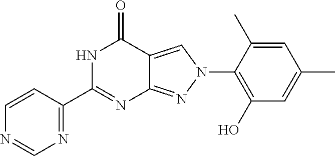 [SMILES: Cc1cc(C)c(-n2cc3c(=O)[nH]c(-c4ccncn4)nc3n2)c(O)c1] | 335.0 |
| 3 | 2-(2-hydroxy-4,6-dimethylphenyl)- 6-methoxy-2,5-dihydro-4H- pyrazolo[3,4-d]pyrimidin-4-one | 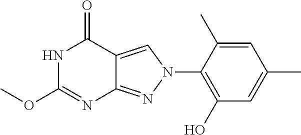 | 287.2 |
| 4 | 2-(2-hydroxy-4,6-dimethylphenyl)- 6-propoxy-2,5-dihydro-4H- pyrazolo[3,4-d]pyrimidin-4-one | 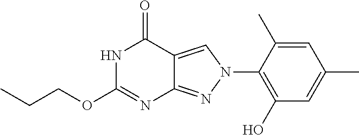 [SMILES: CCCOc1nc2nn(-c3c(C)cc(C)cc3O)cc2c(=O)[nH]1] | 315.2 |
| 5 | 2-(2-hydroxy-4,6-dimethylphenyl)- 6-phenyl-2,5-dihydro-4H- pyrazolo[3,4-d]pyrimidin-4-one | 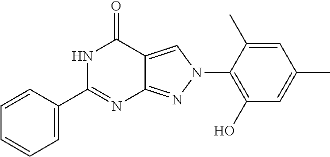 [SMILES: Cc1cc(C)c(-n2cc3c(=O)[nH]c(-c4ccccc4)nc3n2)c(O)c1] | 333.1 |
| 6 | 6-cyclopropyl-2-(2-hydroxy-4,6- dimethylphenyl)-2,5-dihydro-4H- pyrazolo[3,4-d]pyrimidin-4-one | 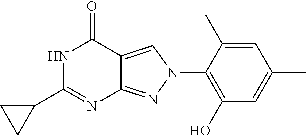 [SMILES: Cc1cc(C)c(-n2cc3c(=O)[nH]c(C4CC4)nc3n2)c(O)c1] | 297.0 |
| 7 | 2-(2-hydroxy-4,6-dimethylphenyl)-6- [(propan-2-yl)oxy]-2,5-dihydro-4H- pyrazolo[3,4-d]pyrimidin-4-one | 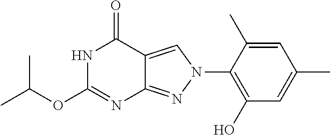 [SMILES: Cc1cc(C)c(-n2cc3c(=O)[nH]c(OC(C)C)nc3n2)c(O)c1] | 315.1 |
| 8 | 2-(2-hydroxy-4,6-dimethylphenyl)- 6-(methoxymethyl)-5-methyl-2,5- dihydro-4H-pyrazolo [3,4-d]pyrimidin-4-one | 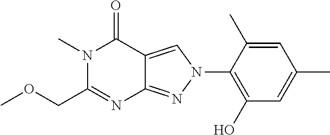 | 315.2 |
| 9 | 2-(2-hydroxy-4,6-dimethylphenyl)-6- (pyridin-2-yl)-2,5-dihydro-4H- pyrazolo[3,4-d]pyrimidin-4-one | 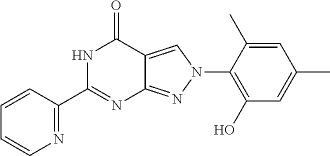 [SMILES: Cc1cc(C)c(-n2cc3c(=O)[nH]c(-c4ccccn4)nc3n2)c(O)c1] | 334.1 |
| 10 | 2-(2-hydroxy-4,6-dimethylphenyl)-6- (propan-2-yl)-2,5-dihydro-4H- pyrazolo[3,4-d]pyrimidin-4-one | 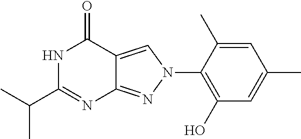 [SMILES: Cc1cc(C)c(-n2cc3c(=O)[nH]c(C(C)C)nc3n2)c(O)c1] | 299.2 |

TABLE 5-2

| | | | |
|---|---|---|---|
| 11 | 2-(2-cyclopropyl-6-hydroxy-4- methylphenyl)-6-ethoxy-2,5-dihydro-4H- pyrazolo[3,4-d]pyrimidin-4-one | 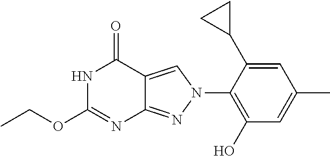 [SMILES: CCOc1nc2nn(-c3c(O)cc(C)cc3C3CC3)cc2c(=O)[nH]1] | 327.1 |
| 12 | racemic mixture of 2-(2-hydroxy-4,6- dimethylphenyl)-6-[(1R,2R)-2- methoxycyclohexyl]-2,5-dihydro-4H- pyrazolo[3,4-d]pyrimidin-4-one and 2-(2-hydroxy-4,6-dimethylphenyl)-6- [(1S,2S)-2-methoxycyclohexyl]-2,5- dihydro-4H-pyrazolo[3,4-d]pyrimidin-4-one | 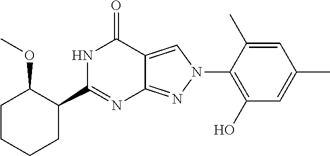 [SMILES: CO[C@@H]1CCCC[C@@H]1c1nc2nn(-c3c(C)cc(C)cc3O)cc2c(=O)[nH]1] | 369.2 |
| 13 | 6-(5-fluoropyridin-2-yl)-2-(2-hydroxy- 4,6-dimethylphenyl)-2,5-dihydro- 4H-pyrazolo[3,4-d]pyrimidin-4-one | 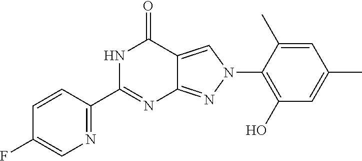 [SMILES: Cc1cc(C)c(-n2cc3c(=O)[nH]c(-c4ccc(F)cn4)nc3n2)c(O)c1] | 352.0 |
| 14 | 2-(2-hydroxy-4,6-dimethylphenyl)-6- {[(3S)-oxolan-3-yl]oxy}-2,5-dihydro- 4H-pyrazolo[3,4-d]pyrimidin-4-one | 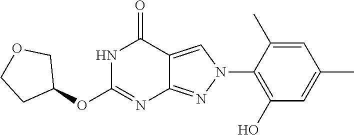 [SMILES: Cc1cc(C)c(-n2cc3c(=O)[nH]c(O[C@H]4CCOC4)nc3n2)c(O)c1] | 343.1 |
| 15 | 2-(2-hydroxy-4,6-dimethylphenyl)- 6-(2-methylpyrimidin-4-yl)-2,5- dihydro-4H-pyrazolo [3,4-d]pyrimidin-4-one | 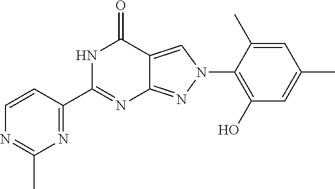 [SMILES: Cc1cc(C)c(-n2cc3c(=O)[nH]c(-c4ccnc(C)n4)nc3n2)c(O)c1] | 349.0 |
| 16 | 6-ethoxy-2-(2-hydroxy-3,6- dimethylphenyl)-2,5-dihydro-4H- pyrazolo[3,4-d]pyrimidin-4-one | 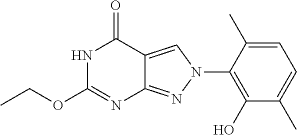 [SMILES: CCOc1nc2nn(-c3c(C)ccc(C)c3O)cc2c(=O)[nH]1] | 301.1 |
| 17 | 6-butyl-2-(2-hydroxy-4,6-dimethylphenyl)- 2,5-dihydro-4H-pyrazolo[3,4- d]pyrimidin-4-one | 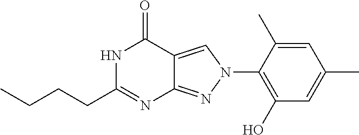 [SMILES: CCCCc1nc2nn(-c3c(C)cc(C)cc3O)cc2c(=O)[nH]1] | 313.2 |
| 18 | 2-(2-hydroxy-4,6-dimethylphenyl)- 6-(oxan-2-yl)-2,5-dihydro-4H- pyrazolo[3,4-d]pyrimidin-4-one | 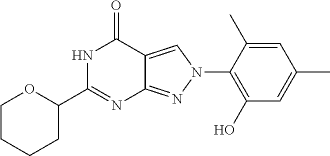 [SMILES: Cc1cc(C)c(-n2cc3c(=O)[nH]c(C4CCCCO4)nc3n2)c(O)c1] | 341.1 |
| 19 | 2-[4-bromo-2-fluoro-6-(trifluoromethyl) phenyl]-6-ethoxy-2,5-dihydro-4H- pyrazolo[3,4-d]pyrimidin-4-one | 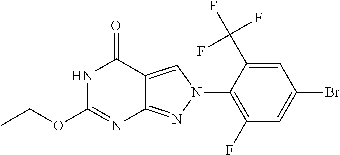 [SMILES: CCOc1nc2nn(-c3c(F)cc(Br)cc3C(F)(F)F)cc2c(=O)[nH]1] | 421.0 |
| 20 | 6-(2-fluorophenyl)-2-(2-hydroxy-4,6- dimethylphenyl)-2,5-dihydro-4H- pyrazolo[3,4-d]pyrimidin-4-one | 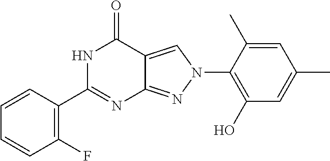 [SMILES: Cc1cc(C)c(-n2cc3c(=O)[nH]c(-c4ccccc4F)nc3n2)c(O)c1] | 351.0 |
| 21 | 2-(2-hydroxy-4,6-dimethylphenyl)-6- (5-methoxypyridin-2-yl)-2,5-dihydro- 4H-pyrazolo[3,4-d]pyrimidin-4-one | 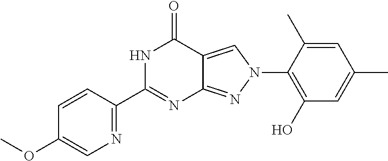 | 364.1 |

TABLE 5-3

| | | | |
|---|---|---|---|
| 22 | 2-(2-hydroxy-4,6-dimethylphenyl)-6- (2-methoxyethoxy)-2,5-dihydro-4H- pyrazolo [3,4-d]pyrimidin-4-one | 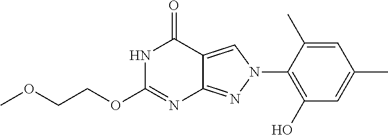 | 331.2 |
| 23 | 2-(2-hydroxy-4,6-dimethylphenyl)-6- (1-methyl-1H-pyrazol-3-yl)-2,5- dihydro-4H-pyrazolo[3,4-d]pyrimidin-4-one | 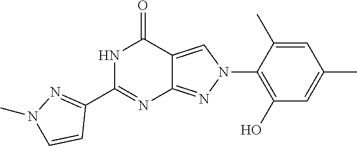 | 337.1 |
| 24 | 2-(4-bromo-2-fluoro-6-methylphenyl)- 6-ethoxy-2,5-dihydro-4H- pyrazolo[3,4-d]pyrimidin-4-one | 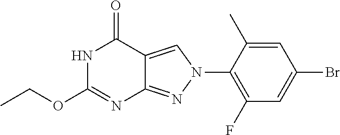 [SMILES: CCOc1nc2nn(-c3c(C)cc(Br)cc3F)cc2c(=O)[nH]1] | 366.8 |
| 25 | 2-(2-hydroxy-4,6-dimethylphenyl)-6- (1-methylcyclopropyl)-2,5-dihydro- 4H-pyrazolo[3,4-d]pyrimidin-4-one | 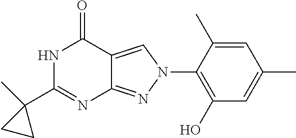 | 311.2 |
| 26 | 3-chloro-6-ethoxy-2-(2-hydroxy-4,6- dimethylphenyl)-2,5-dihydro-4H- pyrazolo[3,4-d]pyrimidin-4-one | 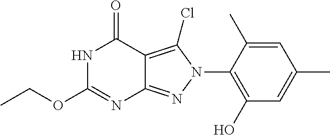 [SMILES: CCOc1nc2nn(-c3c(C)cc(C)cc3O)c(Cl)c2c(=O)[nH]1] | 334.9 |
| 27 | 2-(2-hydroxy-4,6-dimethylphenyl)-6- (2-methoxypropyl)-2,5-dihydro-4H- pyrazolo[3,4-d]pyrimidin-4-one | 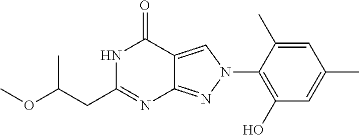 | 329.1 |
| 28 | 2-(2-hydroxy-4,6-dimethylphenyl)-6- (methoxymethyl)-2,5-dihydro-4H- pyrazolo[3,4-d]pyrimidin-4-one | 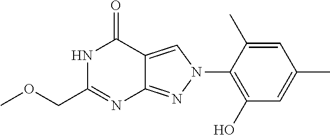 | 301.1 |
| 29 | 2-(2-hydroxy-4,6-dimethylphenyl)-6- (oxolan-2-yl)-2,5-dihydro-4H- pyrazolo[3,4-d]pyrimidin-4-one | 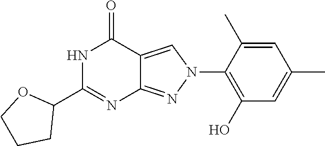 [SMILES: Cc1cc(C)c(-n2cc3c(=O)[nH]c(C4CCCO4)nc3n2)c(O)c1] | 327.2 |
| 30 | 2-(2-hydroxy-4,6-dimethylphenyl)-6- (pyridazin-3-yl)-2,5-dihydro-4H- pyrazolo[3,4-d]pyrimidin-4-one | 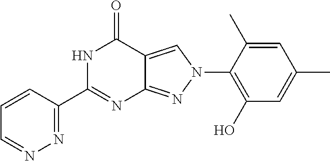 [SMILES: Cc1cc(C)c(-n2cc3c(=O)[nH]c(-c4cccnn4)nc3n2)c(O)c1] | 335.0 |
| 31 | 3-chloro-6-ethoxy-2-(2-hydroxy-4- methylphenyl)-2,5-dihydro-4H- pyrazolo[3,4-d]pyrimidin-4-one | 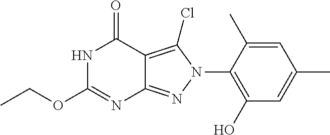 [SMILES: CCOc1nc2nn(-c3c(C)cc(C)cc3O)c(Cl)c2c(=O)[nH]1] | 321.0 |
| 32 | 6-(2,2-difluoroethoxy)-2-(2-hydroxy- 4,6-dimethylphenyl)-2,5-dihydro- 4H-pyrazolo[3,4-d]pyrimidin-4-one | 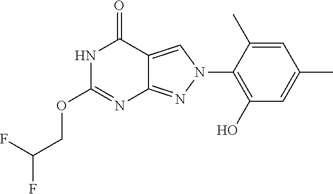 [SMILES: Cc1cc(C)c(-n2cc3c(=O)[nH]c(OCC(F)F)nc3n2)c(O)c1] | 337.1 |

TABLE 5-4

| | | | |
|---|---|---|---|
| 33 | 2-(2-hydroxy-4,6-dimethylphenyl)-6- [(oxolan-2-yl)methyl]-2,5-dihydro- 4H-pyrazolo[3,4-d]pyrimidin-4-one | 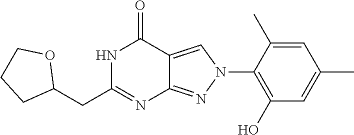 [SMILES: Cc1cc(C)c(-n2cc3c(=O)[nH]c(CC4CCCO4)nc3n2)c(O)c1] | 341.2 |
| 34 | 2-(2-hydroxy-4,6-dimethylphenyl)-6- (pyrimidin-2-yl)-2,5-dihydro-4H- pyrazolo[3,4-d]pyrimidin-4-one | 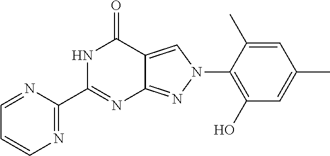 [SMILES: Cc1cc(C)c(-n2cc3c(=O)[nH]c(-c4ncccn4)nc3n2)c(O)c1] | 335.1 |
| 35 | 2-(2-hydroxy-4,6-dimethylphenyl)-6- (1-methoxycyclobutyl)-2,5-dihydro- 4H-pyrazolo[3,4-d]pyrimidin-4-one | 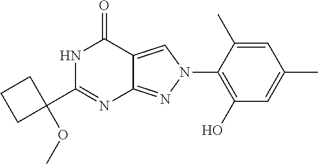 [SMILES: COC1(c2nc3nn(-c4c(C)cc(C)cc4O)cc3c(=O)[nH]2)CCC1] | 341.2 |
| 36 | racemic mixture of 2-(2-hydroxy-4,6- dimethylphenyl)-6-[(1R,2S)-2- methoxycyclohexyl]-2,5-dihydro-4H- pyrazolo[3,4-d]pyrimidin-4-one and 2-(2-hydroxy-4,6-dimethylphenyl)-6- [(1S,2R)-2-methoxycyclohexyl]-2,5- dihydro-4H-pyrazolo [3,4-d]pyrimidin-4-one | 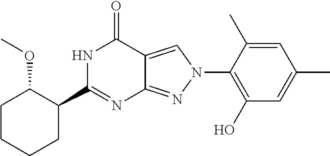 [SMILES: CO[C@H]1CCCC[C@@H]1c1nc2nn(-c3c(C)cc(C)cc3O)cc2c(=O)[nH]1] | 369.2 |
| 37 | 6-ethoxy-2-(2-hydroxy-4,6-dimethylphenyl)- 3-methyl-2,5-dihydro-4H- pyrazolo[3,4-d]pyrimidin-4-one | 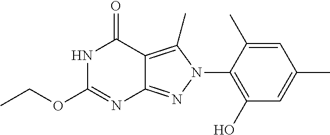 [SMILES: CCOc1nc2nn(-c3c(C)cc(C)cc3O)c(C)c2c(=O)[nH]1] | 315.1 |
| 38 | 2-(2-hydroxy-4,6-dimethylphenyl)-6- (2-methoxyethyl)-2,5-dihydro-4H- pyrazolo[3,4-d]pyrimidin-4-one | 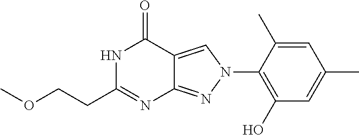 | 315.1 |
| 39 | 2-(4-cyclopropyl-2-fluoro-6-methylphenyl)- 6-ethoxy-2,5-dihydro-4H- pyrazolo[3,4-d]pyrimidin-4-one | 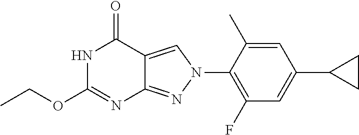 [SMILES: CCOc1nc2nn(-c3c(C)cc(C4CC4)cc3F)cc2c(=O)[nH]1] | 329.0 |
| 40 | 6-(3,3-difluorobutyl)-2-(2-hydroxy- 4,6-dimethylphenyl)-2,5-dihydro-4H- pyrazolo[3,4-d]pyrimidin-4-one | 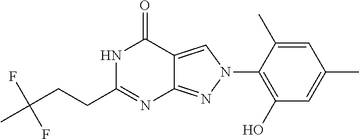 [SMILES: Cc1cc(C)c(-n2cc3c(=O)[nH]c(CCC(C)(F)F)nc3n2)c(O)c1] | 349.1 |
| 41 | 2-(2-hydroxy-3,6-dimethylphenyl)-6- (pyrimidin-4-yl)-2,5-dihydro-4H- pyrazolo[3,4-d]pyrimidin-4-one | 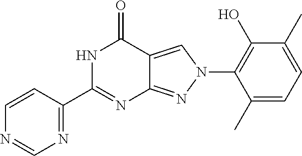 [SMILES: Cc1ccc(C)c(-n2cc3c(=O)[nH]c(-c4ccncn4)nc3n2)c1O] | 335.1 |
| 42 | 6-(2,2-difluoropropoxy)-2-(2-hydroxy- 4,6-dimethylphenyl)-2,5-dihydro- 4H-pyrazolo[3,4-d]pyrimidin-4-one | 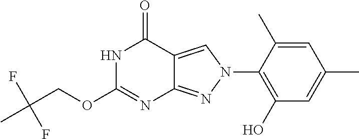 [SMILES: Cc1cc(C)c(-n2cc3c(=O)[nH]c(OCC(C)(F)F)nc3n2)c(O)c1] | 351.1 |
| 43 | 2-(2-hydroxy-4,6-dimethylphenyl)-6- [(1,1,1-trifluoropropan-2-yl)oxy]-2,5- dihydro-4H-pyrazolo[3,4-d]pyrimidin-4-one | 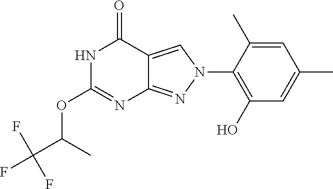 [SMILES: Cc1cc(C)c(-n2cc3c(=O)[nH]c(OC(C)C(F)(F)F)nc3n2)c(O)c1] | 369.1 |

TABLE 5-5

| | | | |
|---|---|---|---|
| 44 | 6-ethoxy-2-[2-hydroxy-4-(propan-2-yl) phenyl]-3-methyl-2,5-dihydro-4H- pyrazolo[3,4-d]pyrimidin-4-one | 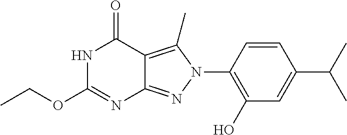 [SMILES: CCOc1nc2nn(-c3ccc(C(C)C)cc3O)c(C)c2c(=O)[nH]1] | 329.0 |
| 45 | 6-(2-chlorophenyl)-2-(2-hydroxy-4,6- dimethylphenyl)-2,5-dihydro-4H- pyrazolo[3,4-d]pyrimidin-4-one | 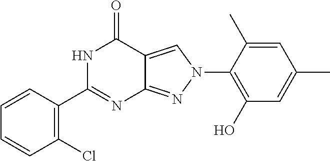 [SMILES: Cc1cc(C)c(-n2cc3c(=O)[nH]c(-c4ccccc4Cl)nc3n2)c(O)c1] | 367.1 |
| 46 | 6-(ethoxymethyl)-2-(2-hydroxy-4,6- dimethylphenyl)-2,5-dihydro-4H- pyrazolo[3,4-d]pyrimidin-4-one | 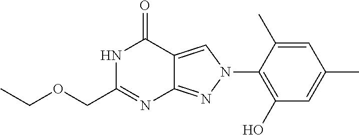 [SMILES: CCOCc1nc2nn(-c3c(C)cc(C)cc3O)cc2c(=O)[nH]1] | 315.1 |
| 47 | 2-(2-hydroxy-4,6-dimethylphenyl)-6- (2-methoxyphenyl)-2,5-dihydro-4H- pyrazolo[3,4-d]pyrimidin-4-one | 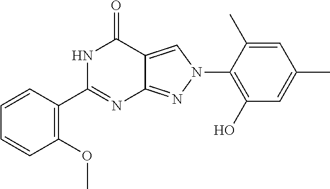 [SMILES: COc1ccccc1-c1nc2nn(-c3c(C)cc(C)cc3O)cc2c(=O)[nH]1] | 363.2 |
| 48 | 2-(4-cyclopropyl-2-hydroxyphenyl)-6- ethoxy-3-methyl-2,5-dihydro-4H- pyrazolo[3,4-d]pyrimidin-4-one | 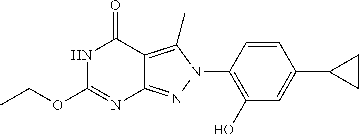 [SMILES: CCOc1nc2nn(-c3ccc(C4CC4)cc3O)c(C)c2c(=O)[nH]1] | 327.0 |
| 49 | 6-ethoxy-2-(4-ethyl-2-hydroxyphenyl)- 3-methyl-2,5-dihydro-4H- pyrazolo[3,4-d]pyrimidin-4-one | 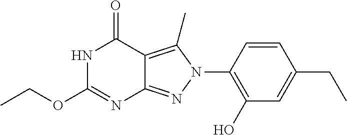 [SMILES: CCOc1nc2nn(-c3ccc(CC)cc3O)c(C)c2c(=O)[nH]1] | 315.1 |
| 50 | 6-[2-(dimethylamino)ethoxy]-2-(2- hydroxy-4,6-dimethylphenyl)-2,5- dihydro-4H-pyrazolo[3,4-d]pyrimidin-4-one | 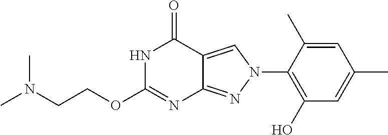 | 344.1 |
| 51 | 6-ethoxy-2-(2-fluoro-6-hydroxy-4- methylphenyl)-3-methyl-2,5-dihydro- 4H-pyrazolo[3,4-d]pyrimidin-4-one | 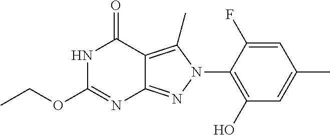 [SMILES: CCOc1nc2nn(-c3c(O)cc(C)cc3F)c(C)c2c(=O)[nH]1] | 318.9 |
| 52 | 2-(2-hydroxy-4,6-dimethylphenyl)-6- (1-methoxyethyl)-2,5-dihydro-4H- pyrazolo[3,4-d]pyrimidin-4-one | 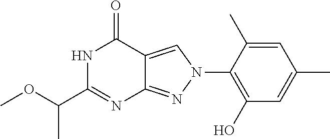 | 315.2 |
| 53 | 2-(2-hydroxy-3,6-dimethylphenyl)-6- (methoxymethyl)-2,5-dihydro-4H- pyrazolo[3,4-d]pyrimidin-4-one | 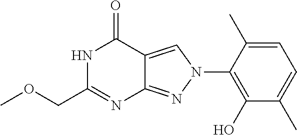 | 301.2 |
| 54 | 2-[2-bromo-6-fluoro-4-(trifluoro methyl) phenyl]-6-ethoxy-2,5-dihydro-4H- pyrazolo[3,4-d]pyrimidin-4-one | 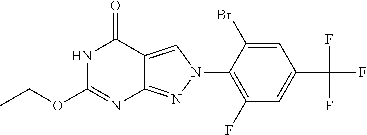 [SMILES: CCOc1nc2nn(-c3c(F)cc(C(F)(F)F)cc3Br)cc2c(=O)[nH]1] | 420.7 |

TABLE 5-6

| | | | |
|---|---|---|---|
| 55 | 2-(2-hydroxy-4,6-dimethylphenyl)-6- (2,2,2-trifluoroethoxy)-2,5-dihydro- 4H-pyrazolo[3,4-d]pyrimidin-4-one | 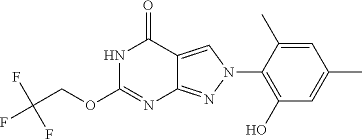 [SMILES: Cc1cc(C)c(-n2cc3c(=O)[nH]c(OCC(F)(F)F)nc3n2)c(O)c1] | 355.1 |
| 56 | 6-ethoxy-2-(2-hydroxy-4-methylphenyl)- 2,5-dihydro-4H-pyrazolo[3,4- d]pyrimidin-4-one | 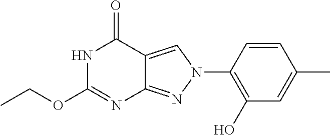 [SMILES: CCOc1nc2nn(-c3ccc(C)cc3O)cc2c(=O)[nH]1] | 287.0 |
| 57 | 2-(2-bromo-6-fluoro-4-methylphenyl)- 6-ethoxy-2,5-dihydro-4H- pyrazolo[3,4-d]pyrimidin-4-one | 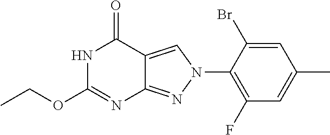 [SMILES: CCOc1nc2nn(-c3c(F)cc(C)cc3Br)cc2c(=O)[nH]1] | 366.9 |
| 58 | 2-(2-hydroxy-4,6-dimethylphenyl)-6- (6-methylpyrimidin-4-yl)-2,5- dihydro-4H-pyrazolo[3,4-d]pyrimidin-4-one | 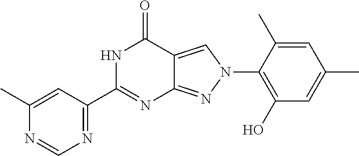 | 349.0 |
| 59 | 6-ethoxy-2-[2-hydroxy-4-(trifluoromethyl) phenyl]-3-methyl-2,5-dihydro- 4H-pyrazolo[3,4-d]pyrimidin-4-one | 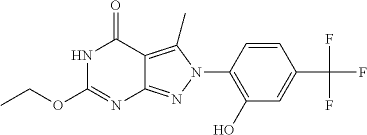 [SMILES: CCOc1nc2nn(-c3ccc(C(F)(F)F)cc3O)c(C)c2c(=O)[nH]1] | 354.9 |
| 60 | 2-(4-chloro-2-hydroxyphenyl)-6- ethoxy-3-methyl-2,5-dihydro-4H- pyrazolo[3,4-d]pyrimidin-4-one | 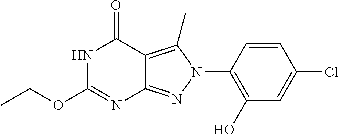 [SMILES: CCOc1nc2nn(-c3ccc(Cl)cc3O)c(C)c2c(=O)[nH]1] | 321.0 |
| 61 | 2-(2-hydroxy-4,6-dimethylphenyl)- 6-(pyrazin-2-yl)-2,5-dihydro-4H- pyrazolo[3,4-d]pyrimidin-4-one | 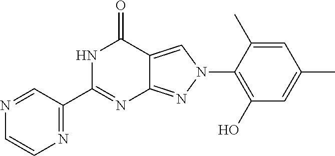 [SMILES: Cc1cc(C)c(-n2cc3c(=O)[nH]c(-c4cnccn4)nc3n2)c(O)c1] | 335.1 |
| 62 | 2-(2-hydroxy-4,6-dimethylphenyl)- 6-[(morpholin-4-yl)methyl]-2,5- dihydro-4H-pyrazolo[3,4-d]pyrimidin-4-one | 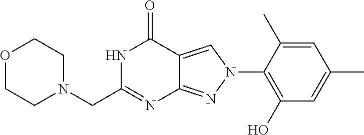 [SMILES: Cc1cc(C)c(-n2cc3c(=O)[nH]c(CN4CCOCC4)nc3n2)c(O)c1] | 356.2 |
| 63 | 2-[2-(difluoro methyl)-6-fluoro-4- methylphenyl]-6-ethoxy-2,5-dihydro-4H- pyrazolo[3,4-d]pyrimidin-4-one | 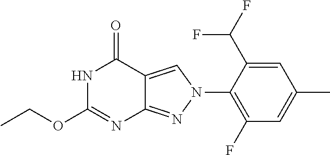 [SMILES: CCOc1nc2nn(-c3c(F)cc(C)cc3C(F)F)cc2c(=O)[nH]1] | 339.0 |
| 64 | 6-ethoxy-2-[2-fluoro-6-methyl-4- (trifluoromethyl)phenyl]-2,5-dihydro-4H- pyrazolo[3,4-d]pyrimidin-4-one | 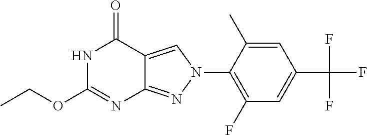 [SMILES: CCOc1nc2nn(-c3c(C)cc(C(F)(F)F)cc3F)cc2c(=O)[nH]1] | 356.9 |
| 65 | 2-(4-chloro-2-fluoro-6-methylphenyl)- 6-ethoxy-2,5-dihydro-4H- pyrazolo[3,4-d]pyrimidin-4-one | 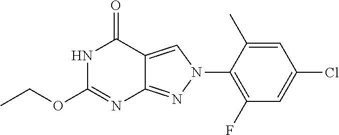 [SMILES: CCOc1nc2nn(-c3c(C)cc(Cl)cc3F)cc2c(=O)[nH]1] | 323.0 |

TABLE 5-7

| | | | |
|---|---|---|---|
| 66 | 2-(2-hydroxy-4,6-dimethylphenyl)-6- (1-methyl-2-oxo-1,2-dihydropyridin- 3-yl)-2,5-dihydro-4H-pyrazolo[3,4-d]pyrimidin-4-one | 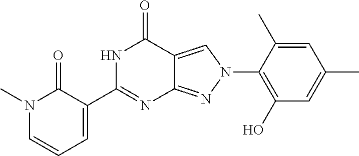 | 364.1 |
| 67 | 2-(2-hydroxy-4,6-dimethylphenyl)-6- [(oxan-2-yl)methyl]-2,5-dihydro-4H- pyrazolo[3,4-d]pyrimidin-4-one | 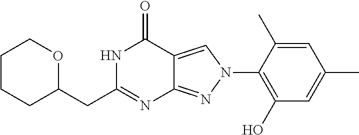 [SMILES: Cc1cc(C)c(-n2cc3c(=O)[nH]c(CC4CCCCO4)nc3n2)c(O)c1] | 355.1 |
| 68 | 6-(2,2-dimethylpropyl)-2-(2-hydroxy- 4,6-dimethylphenyl)-2,5-dihydro- 4H-pyrazolo[3,4-d]pyrimidin-4-one | 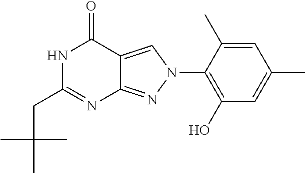 [SMILES: Cc1cc(C)c(-n2cc3c(=O)[nH]c(CC(C)(C)C)nc3n2)c(O)c1] | 327.2 |
| 69 | 6-ethoxy-2-[2-methyl-4-(trifluoromethyl) phenyl]-2,5-dihydro-4H- pyrazolo[3,4-d]pyrimidin-4-one | 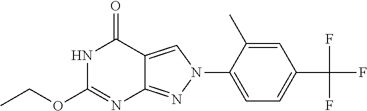 [SMILES: CCOc1nc2nn(-c3ccc(C(F)(F)F)cc3C)cc2c(=O)[nH]1] | 339.0 |
| 70 | 2-(2-chloro-4,6-dimethylphenyl)-6- ethoxy-2,5-dihydro-4H-pyrazolo[3,4- d]pyrimidin-4-one | 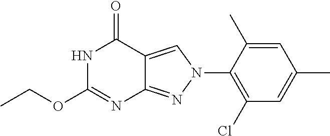 [SMILES: CCOc1nc2nn(-c3c(C)cc(C)cc3Cl)cc2c(=O)[nH]1] | 319.0 |
| 71 | 6-tert-butyl-2-(2-hydroxy-4,6- dimethylphenyl)-2,5-dihydro-4H- pyrazolo[3,4-d]pyrimidin-4-one | 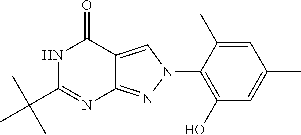 [SMILES: Cc1cc(C)c(-n2cc3c(=O)[nH]c(C(C)(C)C)nc3n2)c(O)c1] | 313.1 |
| 72 | 6-ethoxy-2-(3-ethyl-2-hydroxy-6- methylphenyl)-2,5-dihydro-4H- pyrazolo[3,4-d]pyrimidin-4-one | 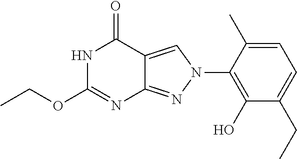 [SMILES: CCOc1nc2nn(-c3c(C)ccc(CC)c3O)cc2c(=O)[nH]1] | 315.1 |
| 73 | 6-ethoxy-2-[2-fluoro-4-methyl-6- (trifluoromethyl)phenyl]-2,5-dihydro-4H- pyrazolo[3,4-d]pyrimidin-4-one | 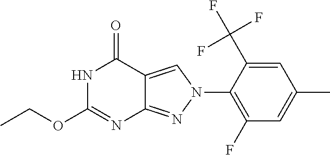 [SMILES: CCOc1nc2nn(-c3c(F)cc(C)cc3C(F)(F)F)cc2c(=O)[nH]1] | 357.0 |
| 74 | 2-(2-hydroxy-4,6-dimethylphenyl)- 6-methyl-2,5-dihydro-4H- pyrazolo[3,4-d]pyrimidin-4-one | 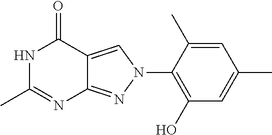 | 271.1 |
| 75 | 2-(2-hydroxy-4,6-dimethylphenyl)-6- [(1H-pyrazol-1-yl)methyl]-2,5- dihydro-4H-pyrazolo[3,4-d]pyrimidin-4-one | 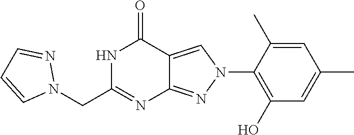 [SMILES: Cc1cc(C)c(-n2cc3c(=O)[nH]c(Cn4cccn4)nc3n2)c(O)c1] | 337.1 |
| 76 | 6-ethoxy-2-(2-ethyl-6-fluoro-4- methylphenyl)-2,5-dihydro-4H- pyrazolo[3,4-d]pyrimidin-4-one | 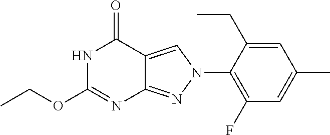 [SMILES: CCOc1nc2nn(-c3c(F)cc(C)cc3CC)cc2c(=O)[nH]1] | 317.1 |

TABLE 5-8

| | | | |
|---|---|---|---|
| 77 | 2-(6-hydroxy-2,3,4-trimethylphenyl)-6- (pyrimidin-4-yl)-2,5-dihydro-4H- pyrazolo[3,4-d]pyrimidin-4-one | 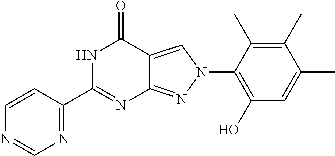 [SMILES: Cc1cc(O)c(-n2cc3c(=O)[nH]c(-c4ccncn4)nc3n2)c(C)c1C] | 349.0 |
| 78 | 6-ethoxy-2-(2-fluoro-4,6-dimethylphenyl)- 2,5-dihydro-4H-pyrazolo[3,4- d]pyrimidin-4-one | 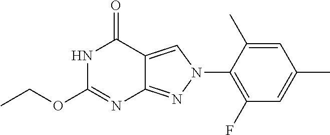 [SMILES: CCOc1nc2nn(-c3c(C)cc(C)cc3F)cc2c(=O)[nH]1] | 303.1 |
| 79 | 2-[2-(2,2-difluoro cyclopropyl)-6- fluoro-4-methylphenyl]-6-ethoxy-2,5- dihydro-4H-pyrazolo[3,4-d]pyrimidin-4-one | 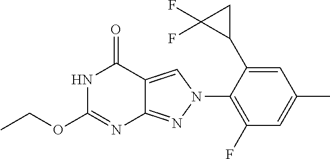 [SMILES: CCOc1nc2nn(-c3c(F)cc(C)cc3C3CC3(F)F)cc2c(=O)[nH]1] | 365.0 |
| 80 | 6-ethoxy-2-(2-hydroxy-4-propylphenyl)- 3-methyl-2,5-dihydro-4H- pyrazolo[3,4-d]pyrimidin-4-one | 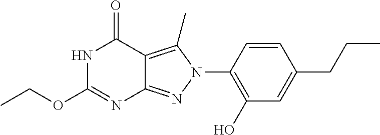 [SMILES: CCCc1ccc(-n2nc3nc(OCC)[nH]c(=O)c3c2C)c(O)c1] | 328.9 |
| 81 | 2-(2-hydroxy-3,4,6-trimethylphenyl)- 6-(oxan-2-yl)-2,5-dihydro-4H- pyrazolo[3,4-d]pyrimidin-4-one | 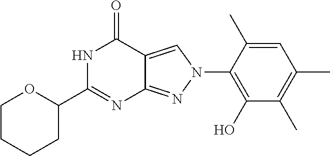 [SMILES: Cc1cc(C)c(-n2cc3c(=O)[nH]c(C4CCCCO4)nc3n2)c(O)c1C] | 355.2 |
| 82 | 2-(2,4-dimethylphenyl)-6-ethoxy- 2,5-dihydro-4H-pyrazolo[3,4- d]pyrimidin-4-one | 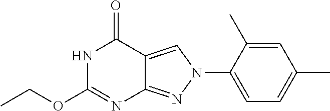 [SMILES: CCOc1nc2nn(-c3ccc(C)cc3C)cc2c(=O)[nH]1] | 285.0 |
| 83 | 2-[2-(difluoromethoxy)-4-methylphenyl]- 6-ethoxy-2,5-dihydro-4H- pyrazolo[3,4-d]pyrimidin-4-one | 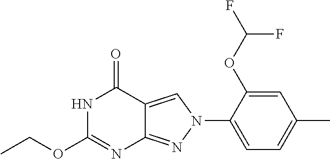 [SMILES: CCOc1nc2nn(-c3ccc(C)cc3OC(F)F)cc2c(=O)[nH]1] | 336.9 |
| 84 | 2-(2,4-dimethylphenyl)-6-(pyrimidin-4- yl)-2,5-dihydro-4H-pyrazolo[3,4- d]pyrimidin-4-one | 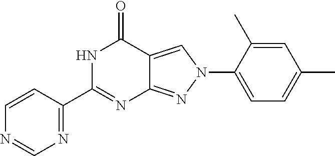 [SMILES: Cc1ccc(-n2cc3c(=O)[nH]c(-c4ccncn4)nc3n2)c(C)c1] | 319.3 |
| 85 | 2-(2-hydroxy-4,6-dimethylphenyl)- 6-(2-methoxypropan-2-yl)-2,5- dihydro-4H-pyrazolo[3,4-d]pyrimidin-4-one | 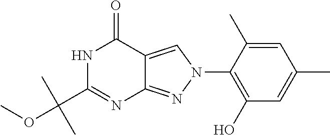 | 329.2 |
| 86 | 6-(2,2-difluoropropoxy)-2-(2-hydroxy- 4-methylphenyl)-2,5-dihydro-4H- pyrazolo[3,4-d]pyrimidin-4-one | 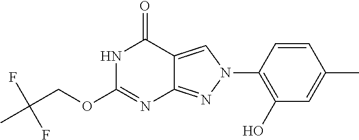 [SMILES: Cc1ccc(-n2cc3c(=O)[nH]c(OCC(C)(F)F)nc3n2)c(O)c1] | 337.0 |
| 87 | 6-(cyclobutyloxy)-2-(2,4-dimethylphenyl)- 2,5-dihydro-4H-pyrazolo[3,4- d]pyrimidin-4-one | 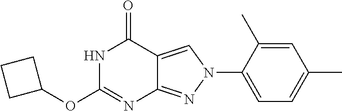 [SMILES: Cc1ccc(-n2cc3c(=O)[nH]c(OC4CCC4)nc3n2)c(C)c1] | 310.9 |

TABLE 5-9

| | | | |
|---|---|---|---|
| 88 | 2-(2-hydroxy-4,6-dimethylphenyl)-6- (4-methylmorpholin-2-yl)-2,5- dihydro-4H-pyrazolo[3,4-d]pyrimidin-4-one | 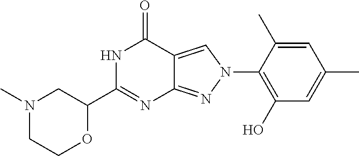 | 356.1 |
| 89 | 2-(2,4-dimethylphenyl)-6-(2- hydroxypropoxy)-2,5-dihydro-4H- pyrazolo[3,4-d]pyrimidin-4-one | 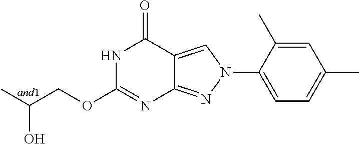 [SMILES: Cc1ccc(-n2cc3c(=O)[nH]c(OCC(C)O)nc3n2)c(C)c1] | 315.0 |
| 90 | 2-(2-hydroxy-4,6-dimethylphenyl)-6- (2-methyloxan-2-yl)-2,5-dihydro-4H- pyrazolo[3,4-d]pyrimidin-4-one | 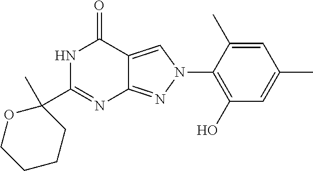 [SMILES: Cc1cc(C)c(-n2cc3c(=O)[nH]c(C4(C)CCCCO4)nc3n2)c(O)c1] | 355.2 |
| 91 | 6-(2,2-difluoro propoxy)-2-(2-hydroxy- 4-methylphenyl)-3-methyl-2,5- dihydro-4H-pyrazolo[3,4-d]pyrimidin-4-one | 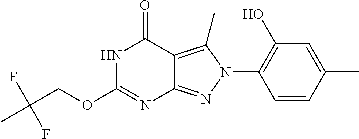 [SMILES: Cc1ccc(-n2nc3nc(OCC(C)(F)F)[nH]c(=O)c3c2C)c(O)c1] | 351.1 |
| 92 | 2-(3,6-difluoro-2,4-dimethylphenyl)- 6-ethoxy-2,5-dihydro-4H- pyrazolo[3,4-d]pyrimidin-4-one | 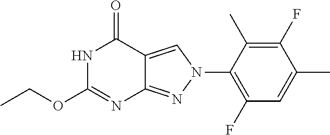 [SMILES: CCOc1nc2nn(-c3c(F)cc(C)c(F)c3C)cc2c(=O)[nH]1] | 320.9 |
| 93 | 6-ethoxy-2-(2-fluoro-6-methoxy-4- methylphenyl)-2,5-dihydro-4H- pyrazolo[3,4-d]pyrimidin-4-one | 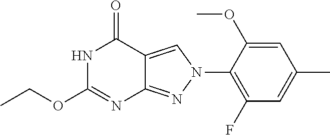 [SMILES: CCOc1nc2nn(-c3c(F)cc(C)cc3OC)cc2c(=O)[nH]1] | 318.9 |
| 94 | 6-(difluoromethyl)-2-(2-hydroxy-4,6- dimethylphenyl)-2,5-dihydro-4H- pyrazolo[3,4-d]pyrimidin-4-one | 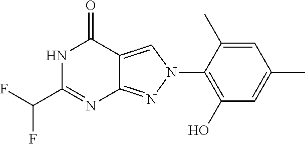 [SMILES: Cc1cc(C)c(-n2cc3c(=O)[nH]c(C(F)F)nc3n2)c(O)c1] | 307.1 |
| 95 | 2-[2-(difluoromethyl)-6-ethoxy-4- (trifluoromethyl)phenyl]-6-ethoxy-2,5- dihydro-4H-pyrazolo[3,4-d]pyrimidin-4-one | 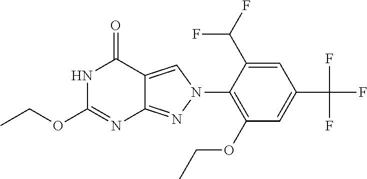 [SMILES: CCOc1nc2nn(-c3c(OCC)cc(C(F)(F)F)cc3C(F)F)cc2c(=O)[nH]1] | 418.9 |
| 96 | 2-(2,3-difluoro-4,6-dimethylphenyl)- 6-ethoxy-2,5-dihydro-4H- pyrazolo[3,4-d]pyrimidin-4-one | 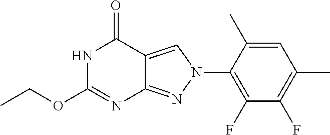 [SMILES: CCOc1nc2nn(-c3c(C)cc(C)c(F)c3F)cc2c(=O)[nH]1] | 320.9 |
| 97 | 2-(6-bromo-2,3-difluoro-4-methylphenyl)- 6-ethoxy-2,5-dihydro-4H- pyrazolo[3,4-d]pyrimidin-4-one | 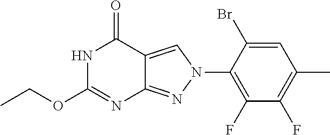 [SMILES: CCOc1nc2nn(-c3c(Br)cc(C)c(F)c3F)cc2c(=O)[nH]1] | 384.7 |
| 98 | 2-[2-(2,2-difluoroethoxy)-4-methylphenyl]- 6-ethoxy-2,5-dihydro-4H- pyrazolo[3,4-d]pyrimidin-4-one | 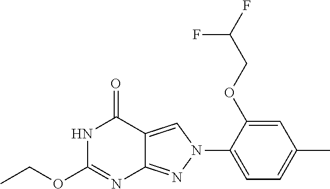 [SMILES: CCOc1nc2nn(-c3ccc(C)cc3OCC(F)F)cc2c(=O)[nH]1] | 350.9 |

TABLE 5-10

| | | | |
|---|---|---|---|
| 99 | 6-[2-(benzyloxy)propoxy]-2-(2,4- dimethylphenyl)-2,5-dihydro-4H- pyrazolo[3,4-d]pyrimidin-4-one | 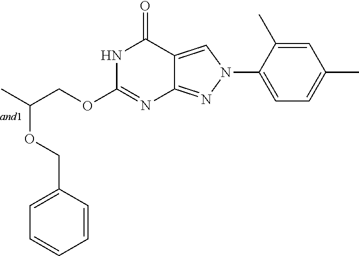 [SMILES: Cc1ccc(-n2cc3c(=O)[nH]c(OCC(C)OCc4ccccc4)nc3n2)c(C)c1] | 405.2 |
| 100 | 2-(2,4-dimethylphenyl)-6-phenoxy- 2,5-dihydro-4H-pyrazolo[3,4- d]pyrimidin-4-one | 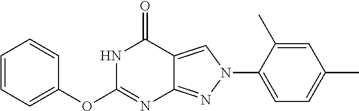 [SMILES: Cc1ccc(-n2cc3c(=O)[nH]c(Oc4ccccc4)nc3n2)c(C)c1] | 333.0 |
| 101 | 6-ethoxy-2-(2-fluoro-3,6-dimethylphenyl)- 2,5-dihydro-4H-pyrazolo[3,4- d]pyrimidin-4-one | 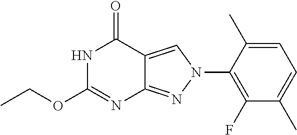 [SMILES: CCOc1nc2nn(-c3c(C)ccc(C)c3F)cc2c(=O)[nH]1] | 302.9 |
| 102 | 2-(4-chloro-2-methylphenyl)-6-(1- methylpiperidin-3-yl)-2,5-dihydro-4H- pyrazolo[3,4-d]pyrimidin-4-one | 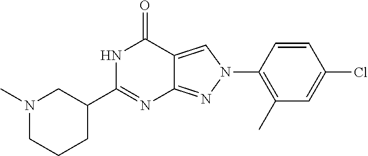 [SMILES: Cc1cc(Cl)ccc1-n1cc2c(=O)[nH]c(C3CCCN(C)C3)nc2n1] | 358.1 |
| 103 | 2-(4-cyclopropyl-2,6-difluorophenyl)- 6-ethoxy-2,5-dihydro-4H- pyrazolo[3,4-d]pyrimidin-4-one | 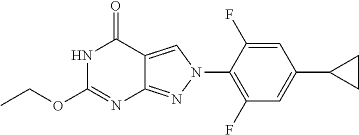 [SMILES: CCOc1nc2nn(-c3c(F)cc(C4CC4)cc3F)cc2c(=O)[nH]1] | 332.9 |
| 104 | 2-[2-bromo-6-fluoro-4-(trifluoromethoxy) phenyl]-6-ethoxy-2,5-dihydro- 4H-pyrazolo[3,4-d]pyrimidin-4-one | 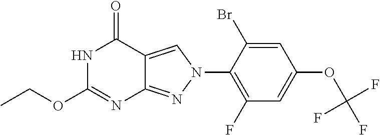 [SMILES: CCOc1nc2nn(-c3c(F)cc(OC(F)(F)F)cc3Br)cc2c(=O)[nH]1] | 437.1 |
| 105 | 2-(2-cyclo propyl-6-fluoro-4-methylphenyl)- 6-ethoxy-2,5-dihydro-4H- pyrazolo[3,4-d]pyrimidin-4-one | 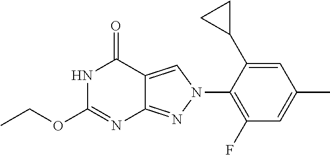 [SMILES: CCOc1nc2nn(-c3c(F)cc(C)cc3C3CC3)cc2c(=O)[nH]1] | 329.0 |
| 106 | 6-ethoxy-2-(2,4,6-trimethylphenyl)- 2,5-dihydro-4H-pyrazolo[3,4- d]pyrimidin-4-one | 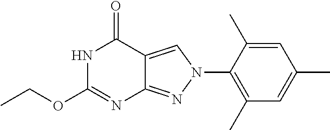 [SMILES: CCOc1nc2nn(-c3c(C)cc(C)cc3C)cc2c(=O)[nH]1] | 299.0 |
| 107 | 2-[4-cyclopropyl-2-(difluoromethoxy)- 6-fluorophenyl]-6-ethoxy-2,5- dihydro-4H-pyrazolo[3,4-d]pyrimidin-4-one | 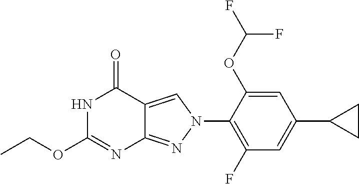 [SMILES: CCOc1nc2nn(-c3c(F)cc(C4CC4)cc3OC(F)F)cc2c(=O)[nH]1] | 380.9 |
| 108 | 2-[2-(difluoromethoxy)-6-fluoro-4- methylphenyl-6-ethoxy-2,5-dihydro- 4H-pyrazolo[3,4-d]pyrimidin-4-one | 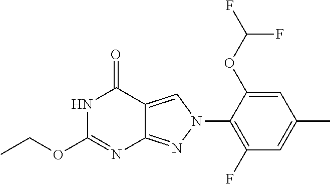 [SMILES: CCOc1nc2nn(-c3c(F)cc(C)cc3OC(F)F)cc2c(=O)[nH]1] | 355.0 |
| 109 | 2-[4-(difluoromethoxy)-2-fluoro-6- methylphenyl-6-ethoxy-2,5-dihydro- 4H-pyrazolo[3,4-d]pyrimidin-4-one | 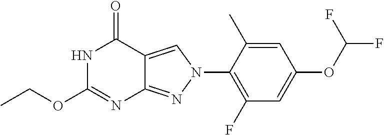 [SMILES: CCOc1nc2nn(-c3c(C)cc(OC(F)F)cc3F)cc2c(=O)[nH]1] | 354.9 |

TABLE 5-11

| | | | |
|---|---|---|---|
| 110 | 2-[4-cyclopropyl-2-(difluoromethyl)- 6-fluorophenyl]-6-ethoxy-2,5- dihydro-4H-pyrazolo[3,4-d]pyrimidin-4-one | 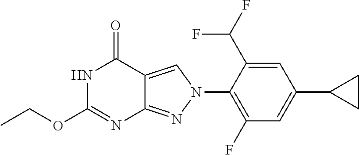 [SMILES: CCOc1nc2nn(-c3c(F)cc(C4CC4)cc3C(F)F)cc2c(=O)[nH]1] | 364.8 |
| 111 | 2-(4-cyclopropyl-2-fluorophenyl)-6- ethoxy-3-methyl-2,5-dihydro-4H- pyrazolo[3,4-d]pyrimidin-4-one | 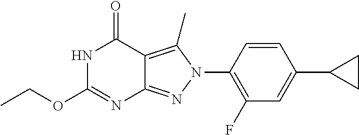 [SMILES: CCOc1nc2nn(-c3ccc(C4CC4)cc3F)c(C)c2c(=O)[nH]1] | 328.9 |
| 112 | 2-[4-(2,2-difluorocyclopropyl)-2-fluoro- 6-methylphenyl]-6-ethoxy-2,5- dihydro-4H-pyrazolo[3,4-d]pyrimidin-4-one | 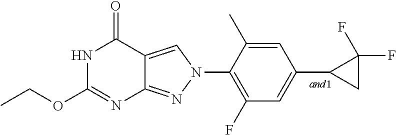 [SMILES: CCOc1nc2nn(-c3c(C)cc(C4CC4(F)F)cc3F)cc2c(=O)[nH]1] | 364.9 |
| 113 | 6-ethoxy-2-(2-fluoro-4-methylphenyl)- 2,5-dihydro-4H-pyrazolo[3,4- d]pyrimidin-4-one | 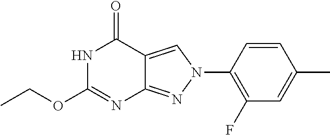 [SMILES: CCOc1nc2nn(-c3ccc(C)cc3F)cc2c(=O)[nH]1] | 288.9 |
| 114 | 2-(2-bromo-4-cyclopropyl-6-fluorophenyl)- 6-ethoxy-2,5-dihydro-4H- pyrazolo[3,4-d]pyrimidin-4-one | 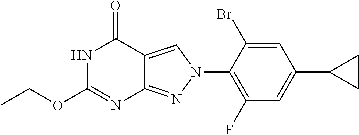 [SMILES: CCOc1nc2nn(-c3c(F)cc(C4CC4)cc3Br)cc2c(=O)[nH]1] | 392.8 |
| 115 | 6-[3-(dimethylamino)propoxy]-2-(2- hydroxy-4,6-dimethylphenyl)-2,5- dihydro-4H-pyrazolo[3,4-d]pyrimidin-4-one | 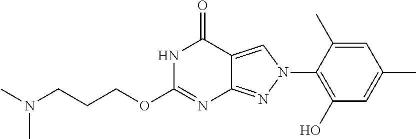 | 358.1 |
| 116 | 2-[2-(difluoromethoxy)-6-hydroxy- 4-methylphenyl]-6-ethoxy-2,5- dihydro-4H-pyrazolo[3,4-d]pyrimidin-4-one | 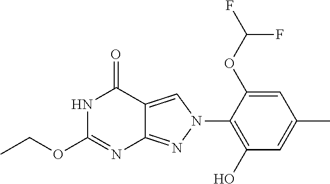 [SMILES: CCOc1nc2nn(-c3c(O)cc(C)cc3OC(F)F)cc2c(=O)[nH]1] | 352.9 |
| 117 | 2-[4-cyclopropyl-2-fluoro-6- (trifluoromethyl)phenyl]-6-ethoxy-2,5- dihydro-4H-pyrazolo[3,4-d]pyrimidin-4-one | 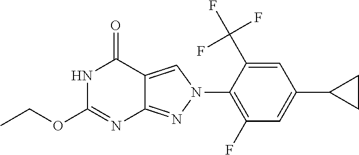 [SMILES: CCOc1nc2nn(-c3c(F)cc(C4CC4)cc3C(F)(F)F)cc2c(=O)[nH]1] | 382.9 |
| 118 | 2-(2-bromo-4,6-dimethylphenyl)-6- ethoxy-2,5-dihydro-4H-pyrazolo[3,4- d]pyrimidin-4-one | 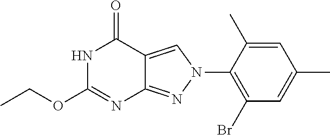 [SMILES: CCOc1nc2nn(-c3c(C)cc(C)cc3Br)cc2c(=O)[nH]1] | 362.8 |
| 119 | 2-[2-bromo-4-(difluoromethoxy)-6- fluorophenyl]-6-ethoxy-2,5-dihydro- 4H-pyrazolo[3,4-d]pyrimidin-4-one | 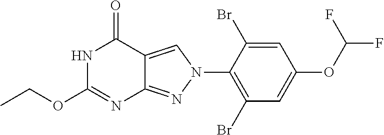 [SMILES: CCOc1nc2nn(-c3c(Br)cc(OC(F)F)cc3Br)cc2c(=O)[nH]1] | 418.7 |
| 120 | 2-(4-ethenyl-2-fluoro-6-methylphenyl)- 6-ethoxy-2,5-dihydro-4H- pyrazolo[3,4-d]pyrimidin-4-one | 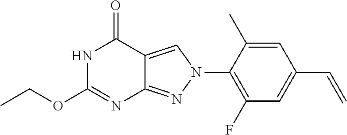 [SMILES: C=Cc1cc(C)c(-n2cc3c(=O)[nH]c(OCC)nc3n2)c(F)c1] | 315.0 |

TABLE 5-12

| | | | |
|---|---|---|---|
| 121 | 6-ethoxy-2-(2-fluoro-4-methoxy- 6-methylphenyl)-2,5-dihydro-4H- pyrazolo[3,4-d]pyrimidin-4-one | 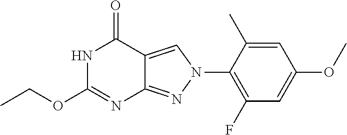 [SMILES: CCOc1nc2nn(-c3c(C)cc(OC)cc3F)cc2c(=O)[nH]1] | 319.0 |
| 122 | 6-ethoxy-2-(4-ethyl-2-fluoro-6- methylphenyl)-2,5-dihydro-4H- pyrazolo[3,4-d]pyrimidin-4-one | 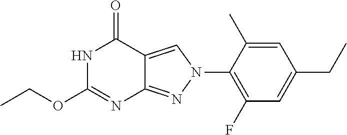 [SMILES: CCOc1nc2nn(-c3c(C)cc(CC)cc3F)cc2c(=O)[nH]1] | 316.9 |
| 123 | 2-(4-cyclopropyl-2,6-difluorophenyl)- 6-ethoxy-3-methyl-2,5-dihydro-4H- pyrazolo[3,4-d]pyrimidin-4-one | 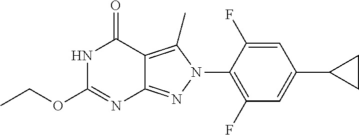 [SMILES: CCOc1nc2nn(-c3c(F)cc(C4CC4)cc3F)c(C)c2c(=O)[nH]1] | 347.0 |
| 124 | 2-(2-hydroxy-4-methylphenyl)-3-methyl- 6-(pyridin-2-yl)-2,5-dihydro-4H- pyrazolo[3,4-d]pyrimidin-4-one | 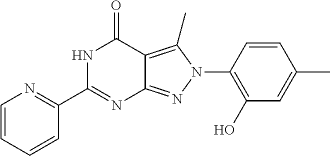 [SMILES: Cc1ccc(-n2nc3nc(-c4ccccn4)[nH]c(=O)c3c2C)c(O)c1] | 333.8 |
| 125 | 6-ethoxy-2-[2-fluoro-6-methyl-4- (trifluoromethoxy)phenyl]-2,5-dihydro- 4H-pyrazolo[3,4-d]pyrimidin-4-one | 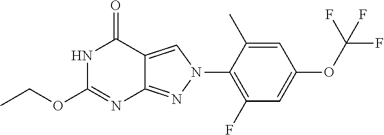 [SMILES: CCOc1nc2nn(-c3c(C)cc(OC(F)(F)F)cc3F)cc2c(=O)[nH]1] | 372.9 |
| 126 | 2-(2,4-difluoro-6-methylphenyl)-6- ethoxy-2,5-dihydro-4H-pyrazolo[3,4- d]pyrimidin-4-one | 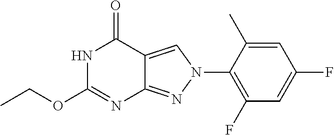 [SMILES: CCOc1nc2nn(-c3c(C)cc(F)cc3F)cc2c(=O)[nH]1] | 306.9 |
| 127 | 6-ethoxy-2-(2-hydroxy-4-methylphenyl)- 3-methyl-2,5-dihydro-4H- pyrazolo[3,4-d]pyrimidin-4-one | 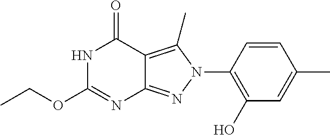 [SMILES: CCOc1nc2nn(-c3ccc(C)cc3O)c(C)c2c(=O)[nH]1] | 301.3 |
| 128 | 2-(2,4-dibromo-6-fluorophenyl)-6- ethoxy-2,5-dihydro-4H-pyrazolo[3,4- d]pyrimidin-4-one | 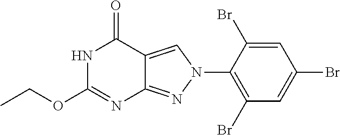 [SMILES: CCOc1nc2nn(-c3c(Br)cc(Br)cc3Br)cc2c(=O)[nH]1] | 431.2 |
| 129 | 6-[4-(dimethylamino)butoxy]-2- (2-hydroxy-4,6-dimethylphenyl)-2,5- dihydro-4H-pyrazolo[3,4-d]pyrimidin-4-one | 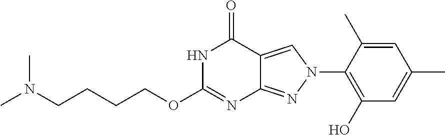 | 372.0 |
| 130 | 2-(5-cyclopropylpyridin-2-yl)-6- ethoxy-2,5-dihydro-4H-pyrazolo[3,4- d]pyrimidin-4-one | 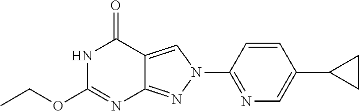 [SMILES: CCOc1nc2nn(-c3ccc(C4CC4)cn3)cc2c(=O)[nH]1] | 298.0 |
| 131 | 2-(4-bromo-2,6-dimethylphenyl)- 6-ethoxy-2,5-dihydro-4H-pyrazolo[3,4- d]pyrimidin-4-one | 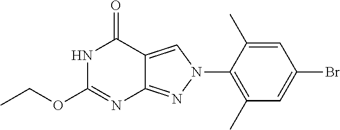 [SMILES: CCOc1nc2nn(-c3c(C)cc(Br)cc3C)cc2c(=O)[nH]1] | 362.9 |

TABLE 5-13

| | | | |
|---|---|---|---|
| 132 | 2-(4-cyclopropyl-2,6-dimethylphenyl)- 6-ethoxy-2,5-dihydro-4H- pyrazolo[3,4-d]pyrimidin-4-one | 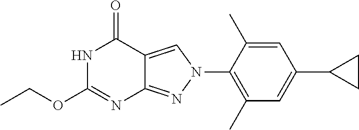 [SMILES: CCOc1nc2nn(-c3c(C)cc(C4CC4)cc3C)cc2c(=O)[nH]1] | 325.0 |
| 133 | 2-(4-bromo-2-chloro-6-fluorophenyl)- 6-ethoxy-2,5-dihydro-4H- pyrazolo[3,4-d]pyrimidin-4-one | 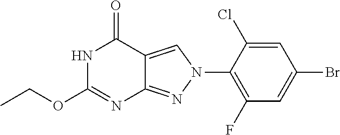 [SMILES: CCOc1nc2nn(-c3c(F)cc(Br)cc3Cl)cc2c(=O)[nH]1] | 386.5 |
| 134 | 6-ethoxy-2-[2-fluoro-4-methyl-6-(2,2,2- trifluoroethyl)phenyl]-2,5-dihydro- 4H-pyrazolo[3,4-d]pyrimidin-4-one | 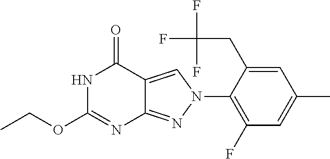 [SMILES: CCOc1nc2nn(-c3c(F)cc(C)cc3CC(F)(F)F)cc2c(=O)[nH]1] | 371.0 |
| 135 | 2-(2-chloro-6-fluoro-4-methylphenyl)- 6-ethoxy-2,5-dihydro-4H- pyrazolo[3,4-d]pyrimidin-4-one | 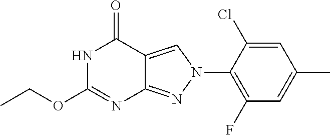 [SMILES: CCOc1nc2nn(-c3c(F)cc(C)cc3Cl)cc2c(=O)[nH]1] | 322.8 |
| 136 | 2-[4-(difluoromethyl)-2-fluoro-6- methylphenyl]-6-ethoxy-2,5-dihydro-4H- pyrazolo[3,4-d]pyrimidin-4-one | 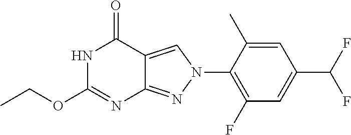 [SMILES: CCOc1nc2nn(-c3c(C)cc(C(F)F)cc3F)cc2c(=O)[nH]1] | 338.9 |
| 137 | 2-(2-chloro-4-cyclopropyl-6-fluorophenyl)- 6-ethoxy-2,5-dihydro-4H- pyrazolo[3,4-d]pyrimidin-4-one | 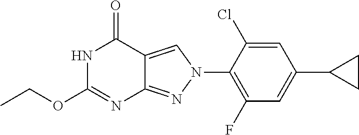 [SMILES: CCOc1nc2nn(-c3c(F)cc(C4CC4)cc3Cl)cc2c(=O)[nH]1] | 349.0 |
| 138 | 2-(4-bromo-2,6-dimethylphenyl)- 6-methoxy-2,5-dihydro-4H- pyrazolo[3,4-d]pyrimidin-4-one | 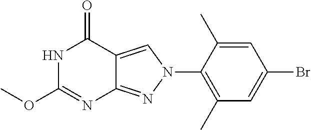 [SMILES: COc1nc2nn(-c3c(C)cc(Br)cc3C)cc2c(=O)[nH]1] | 349.1 |
| 139 | 2-[2,6-dimethyl-4-(1H-pyrazol-1-yl) phenyl]-6-ethoxy-2,5-dihydro-4H- pyrazolo[3,4-d]pyrimidin-4-one | 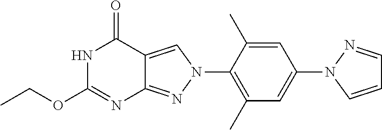 [SMILES: CCOc1nc2nn(-c3c(C)cc(-n4cccn4)cc3C)cc2c(=O)[nH]1] | 351.0 |
| 140 | 6-ethoxy-3-ethyl-2-(2-hydroxy-4- methylphenyl)-2,5-dihydro-4H- pyrazolo[3,4-d]pyrimidin-4-one | 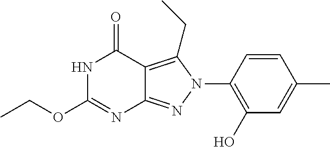 [SMILES: CCOc1nc2nn(-c3ccc(C)cc3O)c(CC)c2c(=O)[nH]1] | 314.8 |
| 141 | 2-(4-cyclopropyl-2,6-dimethylphenyl)- 6-ethoxy-5-methyl-2,5-dihydro- 4H-pyrazolo[3,4-d]pyrimidin-4-one | 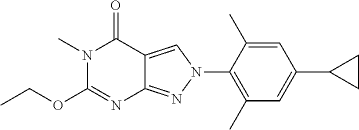 [SMILES: CCOc1nc2nn(-c3c(C)cc(C4CC4)cc3C)cc2c(=O)n1C] | 338.9 |
| 142 | 2-[2-bromo-4-(difluoromethoxy)- 6-methylphenyl]-6-ethoxy-2,5-dihydro- 4H-pyrazolo[3,4-d]pyrimidin-4-one | 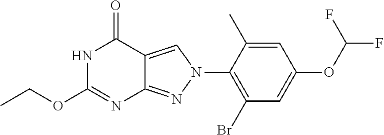 [SMILES: CCOc1nc2nn(-c3c(C)cc(OC(F)F)cc3Br)cc2c(=O)[nH]1] | 414.8 |

TABLE 5-14

| | | | |
|---|---|---|---|
| 143 | 2-(2-bromo-4-methoxy-6-methylphenyl)- 6-ethoxy-2,5-dihydro-4H- pyrazolo[3,4-d]pyrimidin-4-one | 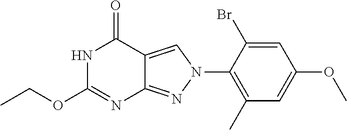 [SMILES: CCOc1nc2nn(-c3c(C)cc(OC)cc3Br)cc2c(=O)[nH]1] | 378.9 |
| 144 | 2-[4-bromo-2-methyl-6-(1H-pyrazol- 1-yl)phenyl]-6-ethoxy-2,5-dihydro- 4H-pyrazolo[3,4-d]pyrimidin-4-one | 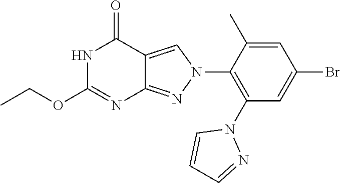 [SMILES: CCOc1nc2nn(-c3c(C)cc(Br)cc3-n3cccn3)cc2c(=O)[nH]1] | 414.9 |
| 145 | 6-ethoxy-2-(4-methoxy-2,6- dimethylphenyl)-2,5-dihydro-4H- pyrazolo[3,4-d]pyrimidin-4-one | 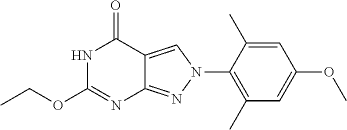 [SMILES: CCOc1nc2nn(-c3c(C)cc(OC)cc3C)cc2c(=O)[nH]1] | 315.0 |
| 146 | 2-(4-cyclopropyl-2,6-dimethylphenyl)- 6-methoxy-2,5-dihydro-4H- pyrazolo[3,4-d]pyrimidin-4-one | 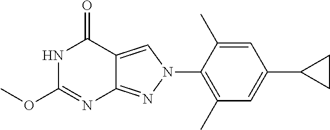 [SMILES: COc1nc2nn(-c3c(C)cc(C4CC4)cc3C)cc2c(=O)[nH]1] | 311.0 |
| 147 | 2-[4-(difluoromethoxy)-2,6-dimethylphenyl]- 6-ethoxy-2,5-dihydro-4H- pyrazolo[3,4-d]pyrimidin-4-one | 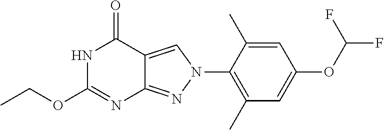 [SMILES: CCOc1nc2nn(-c3c(C)cc(OC(F)F)cc3C)cc2c(=O)[nH]1] | 350.9 |
| 148 | 2-[5-bromo-3-(trifluoromethyl)pyridin- 2-yl]-6-ethoxy-2,5-dihydro-4H- pyrazolo[3,4-d]pyrimidin-4-one | 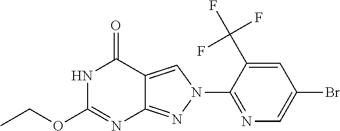 [SMILES: CCOc1nc2nn(-c3ncc(Br)cc3C(F)(F)F)cc2c(=O)[nH]1] | 403.7 |
| 149 | 2-[2-(difluoromethyl)-6-fluoro-4- methoxyphenyl]-6-ethoxy-2,5-dihydro- 4H-pyrazolo[3,4-d]pyrimidin-4-one | 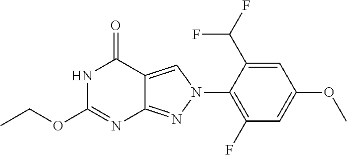 [SMILES: CCOc1nc2nn(-c3c(F)cc(OC)cc3C(F)F)cc2c(=O)[nH]1] | 354.9 |
| 150 | 2-(5-bromo-3-methylpyridin-2- yl)-6-ethoxy-2,5-dihydro-4H- pyrazolo[3,4-d]pyrimidin-4-one | 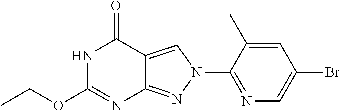 [SMILES: CCOc1nc2nn(-c3ncc(Br)cc3C)cc2c(=O)[nH]1] | 349.8 |
| 151 | 2-(2-bromo-4-cyclopropyl-6-methylphenyl)- 6-ethoxy-2,5-dihydro-4H- pyrazolo[3,4-d]pyrimidin-4-one | 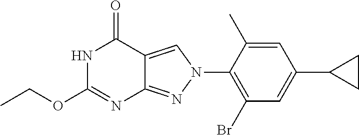 [SMILES: CCOc1nc2nn(-c3c(C)cc(C4CC4)cc3Br)cc2c(=O)[nH]1] | 389.0 |
| 152 | 2-(6-ethoxy-4-oxo-4,5-dihydro-2H- pyrazolo[3,4-d]pyrimidin-2-yl)-3,5- dimethylbenzonitrile | 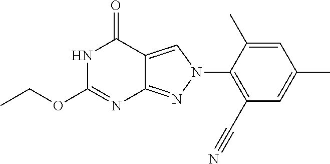 [SMILES: CCOc1nc2nn(-c3c(C)cc(C)cc3C#N)cc2c(=O)[nH]1] | 310.1 |
| 153 | 2-[5-cyclopropyl-3-(trifluoromethyl) pyridin-2-y]-6-ethoxy-2,5-dihydro- 4H-pyrazolo[3,4-d]pyrimidin-4-one | 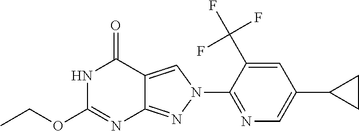 [SMILES: CCOc1nc2nn(-c3ncc(C4CC4)cc3C(F)(F)F)cc2c(=O)[nH]1] | 366.0 |

TABLE 5-15

| | | | |
|---|---|---|---|
| 154 | 2-(5-cyclopropyl-3-methylpyridin- 2-yl)-6-ethoxy-2,5-dihydro-4H- pyrazolo[3,4-d]pyrimidin-4-one | 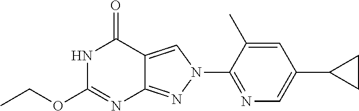 [SMILES: CCOc1nc2nn(-c3ncc(C4CC4)cc3C)cc2c(=O)[nH]1] | 312.1 |
| 155 | 2-[2-bromo-4-cyclopropyl-6- (methoxymethyl)phenyl]-6-ethoxy-2,5- dihydro-4H-pyrazolo[3,4-d]pyrimidin-4-one | 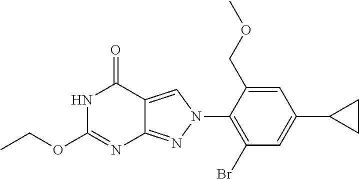 [SMILES: CCOc1nc2nn(-c3c(Br)cc(C4CC4)cc3COC)cc2c(=O)[nH]1] | 419.0 |
| 156 | 6-ethoxy-2-[2-fluoro-6-methyl-4-(2,2,2- trifluoroethyl)phenyl]-2,5-dihydro- 4H-pyrazolo[3,4-d]pyrimidin-4-one | 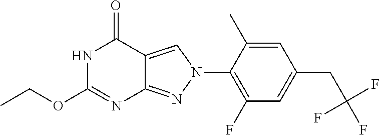 [SMILES: CCOc1nc2nn(-c3c(C)cc(CC(F)(F)F)cc3F)cc2c(=O)[nH]1] | 371.0 |
| 157 | 2-(2-bromo-6-fluoro-4-methoxyphenyl)- 6-ethoxy-2,5-dihydro-4H- pyrazolo[3,4-d]pyrimidin-4-one | 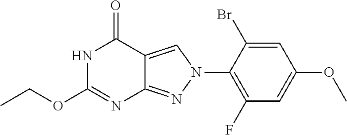 [SMILES: CCOc1nc2nn(-c3c(F)cc(OC)cc3Br)cc2c(=O)[nH]1] | 382.8 |
| 158 | 2-(2-bromo-4-ethoxy-6-fluorophenyl)- 6-ethoxy-2,5-dihydro-4H- pyrazolo [3,4-d]pyrimidin-4-one | 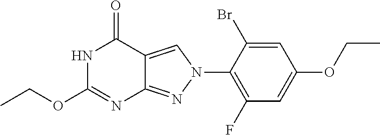 [SMILES: CCOc1cc(F)c(-n2cc3c(=O)[nH]c(OCC)nc3n2)c(Br)c1] | 396.9 |
| 159 | 2-[2-(difluoromethyl)-4,6-dimethylphenyl]- 6-ethoxy-2,5-dihydro-4H- pyrazolo[3,4-d]pyrimidin-4-one | 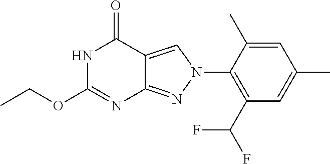 [SMILES: CCOc1nc2nn(-c3c(C)cc(C)cc3C(F)F)cc2c(=O)[nH]1] | 334.9 |
| 160 | 6-ethoxy-2-(4-ethoxy-2-fluoro-6- methylphenyl)-2,5-dihydro-4H- pyrazolo[3,4-d]pyrimidin-4-one | 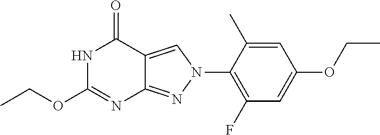 [SMILES: CCOc1cc(C)c(-n2cc3c(=O)[nH]c(OCC)nc3n2)c(F)c1] | 333.0 |
| 161 | 2-(3,5-dibromopyridin-2-yl)-6- ethoxy-2,5-dihydro-4H-pyrazolo[3,4- d]pyrimidin-4-one | 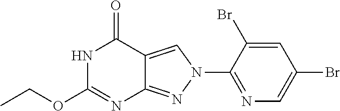 [SMILES: CCOc1nc2nn(-c3ncc(Br)cc3Br)cc2c(=O)[nH]1] | 413.6 |
| 162 | 6-ethoxy-2-[2-fluoro-4-methoxy- 6-(2,2,2-trifluoroethyl)phenyl]-2,5- dihydro-4H-pyrazolo[3,4-d]pyrimidin-4-one | 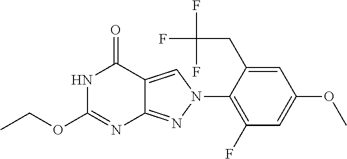 [SMILES: CCOc1nc2nn(-c3c(F)cc(OC)cc3CC(F)(F)F)cc2c(=O)[nH]1] | 386.9 |
| 163 | 2-[4-(difluoromethoxy)-2-(difluoromethyl)- 6-fluorophenyl]-6-ethoxy-2,5- dihydro-4H-pyrazolo[3,4-d]pyrimidin-4-one | 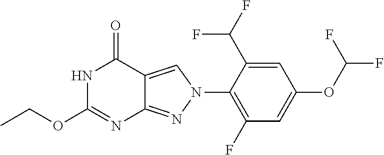 [SMILES: CCOc1nc2nn(-c3c(F)cc(OC(F)F)cc3C(F)F)cc2c(=O)[nH]1] | 390.9 |
| 164 | 2-[4-cyclopropyl-2-(methoxymethyl)- 6-methylphenyl]-6-ethoxy-2,5- dihydro-4H-pyrazolo[3,4-d]pyrimidin-4-one | 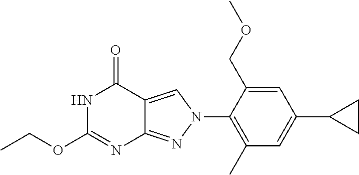 [SMILES: CCOc1nc2nn(-c3c(C)cc(C4CC4)cc3COC)cc2c(=O)[nH]1] | 355.1 |

TABLE 5-16

| | | | |
|---|---|---|---|
| 165 | 2-(4-bromo-2,6-dimethylphenyl)-6-(methoxymethyl)-2,5-dihydro- 4H-pyrazolo[3,4-d]pyrimidin-4-one | 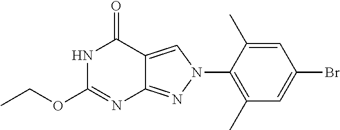 [SMILES: CCOc1nc2nn(-c3c(C)cc(Br)cc3C)cc2c(=O)[nH]1] | 363.1 |
| 166 | 2-(4-cyclopropyl-2,6-dimethylphenyl)-6-(methoxymethyl)-2,5- dihydro-4H-pyrazolo[3,4-d]pyrimidin-4-one | 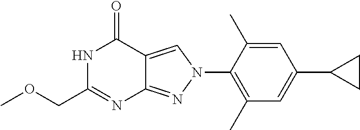 [SMILES: COCc1nc2nn(-c3c(C)cc(C4CC4)cc3C)cc2c(=O)[nH]1] | 325.1 |
| 167 | 6-cyclopropyl-2-(4-cyclopropyl-2,6-dimethylphenyl)-2,5-dihydro- 4H-pyrazolo[3,4-d]pyrimidin-4-one | 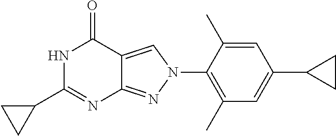 [SMILES: Cc1cc(C2CC2)cc(C)c1-n1cc2c(=O)[nH]c(C3CC3)nc2n1] | 321.1 |
| 168 | 2-[4-(difluoromethoxy)-2-methyl-6-(2,2,2-trifluoroethyl)phenyl]-6- ethoxy-2,5-dihydro-4H-pyrazolo[3,4-d]pyrimidin-4-one | 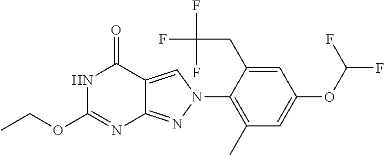 [SMILES: CCOc1nc2nn(-c3c(C)cc(OC(F)F)cc3CC(F)(F)F)cc2c(=O)[nH]1] | 419.1 |
| 169 | 2-[4-cyclopropyl-2-(difluoro methyl)-6-methoxyphenyl]-6- methoxy-2,5-dihydro-4H-pyrazolo[3,4-d]pyrimidin-4-one | 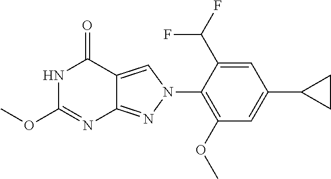 [SMILES: COc1nc2nn(-c3c(OC)cc(C4CC4)cc3C(F)F)cc2c(=O)[nH]1] | 363.1 |
| 170 | 2-[2-(4-bromo-2,6-dimethylphenyl)-4-oxo-4,5-dihydro-2H-pyrazolo [3,4-d]pyrimidin-6-yl]propanoic acid | 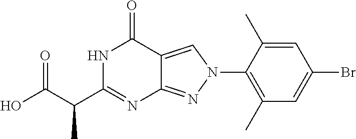 [SMILES: Cc1cc(Br)cc(C)c1-n1cc2c(=O)[nH]c([C@@H](C)C(=O)O)nc2n1] | 391.2 |
| 171 | 2-(4-cyclopropyl-2,6-dimethylphenyl)-6-(pyrimidin-4-yl)-2,5- dihydro-4H-pyrazolo[3,4-d]pyrimidin-4-one | 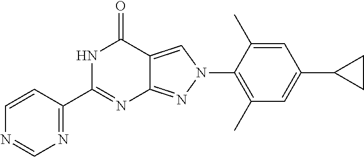 [SMILES: Cc1cc(C2CC2)cc(C)c1-n1cc2c(=O)[nH]c(-c3ccncn3)nc2n1] | 359.0 |
| 172 | 2-(4-cyclopropyl-2,6-dimethylphenyl)-6-(2-hydroxypropan-2-yl)- 2,5-dihydro-4H-pyrazolo[3,4-d]pyrimidin-4-one | 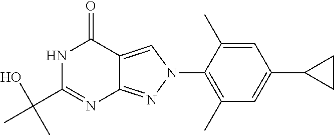 [SMILES: Cc1cc(C2CC2)cc(C)c1-n1cc2c(=O)[nH]c(C(C)(C)O)nc2n1] | 339.2 |
| 173 | 6-cyclopropyl-2-[4-cyclopropyl-2-(difluoromethyl)-6- fluorophenyl]-2,5-dihydro-4H-pyrazolo[3,4-d]pyrimidin-4-one | 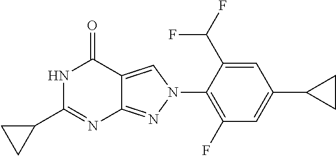 [SMILES: O=c1[nH]c(C2CC2)nc2nn(-c3c(F)cc(C4CC4)cc3C(F)F)cc12] | 360.9 |
| 174 | 2-(6-cyclopropyl-2,4-dimethylpyridin-3-yl)-6-ethoxy-2,5-dihydro- 4H-pyrazolo[3,4-d]pyrimidin-4-one | 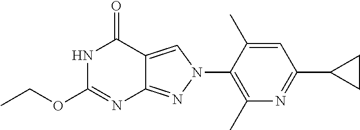 [SMILES: CCOc1nc2nn(-c3c(C)cc(C4CC4)nc3C)cc2c(=O)[nH]1] | 326.0 |
| 175 | 2-(4-bromo-2,6-dimethylphenyl)-6-(2-methoxyethoxy)-2,5- dihydro-4H-pyrazolo[3,4-d]pyrimidin-4-one | 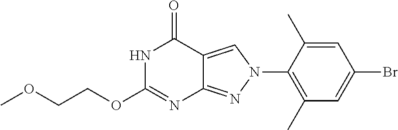 [SMILES: COCCOc1nc2nn(-c3c(C)cc(Br)cc3C)cc2c(=O)[nH]1] | 392.9 |

TABLE 5-17

| | | | |
|---|---|---|---|
| 176 | 2-(2,4-dimethylphenyl)-6-[(oxan-4-yl)oxy]-2,5-dihydro- 4H-pyrazolo[3,4-d]pyrimidin-4-one | 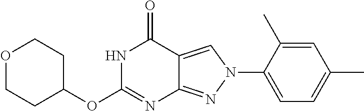 [SMILES: Cc1ccc(-n2cc3c(=O)[nH]c(OC4CCOCC4)nc3n2)c(C)c1] | 340.9 |
| 177 | 2-[4-cyclopropyl-2-(2,2-difluoro-1-hydroxyethyl)- 6-fluorophenyl]-6-ethoxy-2,5-dihydro-4H- pyrazolo[3,4-d]pyrimidin-4-one | 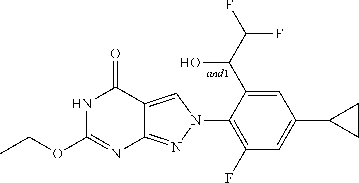 [SMILES: CCOc1nc2nn(-c3c(F)cc(C4CC4)cc3C(O)C(F)F)cc2c(=O)[nH]1] | 395.0 |
| 178 | 2-[2-bromo-6-fluoro-4-(1-fluorocyclopropyl)phenyl]- 6-ethoxy-2,5-dihydro-4H-pyrazolo[3,4-d]pyrimidin-4-one | 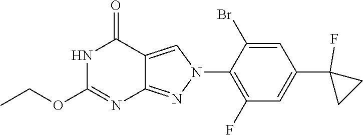 [SMILES: CCOc1nc2nn(-c3c(F)cc(C4(F)CC4)cc3Br)cc2c(=O)[nH]1] | 411.1 |
| 179 | 5-cyclo propyl-2-(6-ethoxy-4-oxo-4,5-dihydro-2H- pyrazolo[3,4-d]pyrimidin-2-yl)-3-methylbenzonitrile | 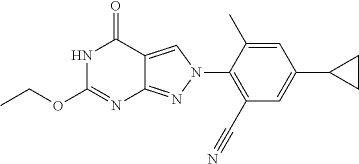 [SMILES: CCOc1nc2nn(-c3c(C)cc(C4CC4)cc3C#N)cc2c(=O)[nH]1] | 336.1 |
| 180 | 2-(4-cyclopropyl-2,6-dimethylphenyl)-6-methyl-2,5- dihydro-4H-pyrazolo[3,4-d]pyrimidin-4-one | 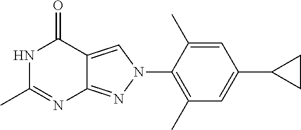 [SMILES: Cc1nc2nn(-c3c(C)cc(C4CC4)cc3C)cc2c(=O)[nH]1] | 295.1 |
| 181 | 2-(4-cyclopropyl-2,6-dimethylphenyl)-6-(2-hydroxy-2- methylpropyl)-2,5-dihydro-4H-pyrazolo[3,4-d]pyrimidin- 4-one | 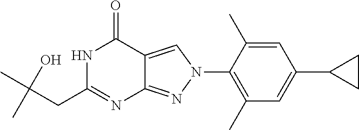 [SMILES: Cc1cc(C2CC2)cc(C)c1-n1cc2c(=O)[nH]c(CC(C)(C)O)nc2n1] | 353.2 |
| 182 | 6-ethoxy-2-[2-fluoro-4-(1-fluorocyclo propyl)-6- methylphenyl]-2,5-dihydro-4H-pyrazolo[3,4-d]pyrimidin- 4-one | 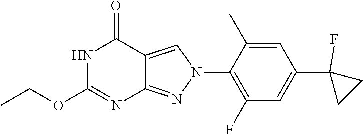 [SMILES: CCOc1nc2nn(-c3c(C)cc(C4(F)CC4)cc3F)cc2c(=O)[nH]1] | 347.1 |
| 183 | 6-ethoxy-2-[2-fluoro-4-(1-fluorocyclopropyl)phenyl]-2,5- dihydro-4H-pyrazolo[3,4-d]pyrimidin-4-one | 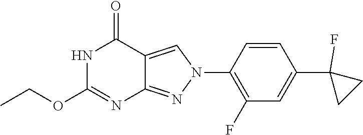 [SMILES: CCOc1nc2nn(-c3ccc(C4(F)CC4)cc3F)cc2c(=O)[nH]1] | 333.0 |
| 184 | 2-[2-bromo-4-(difluoromethoxy)-6-(difluoromethyl)phenyl]- 6-ethoxy-2,5-dihydro-4H-pyrazolo[3,4-d]pyrimidin-4-one | 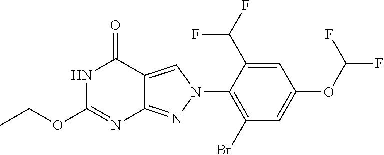 [SMILES: CCOc1nc2nn(-c3c(Br)cc(OC(F)F)cc3C(F)F)cc2c(=O)[nH]1] | 450.8 |
| 185 | 2-(4-bromo-2,6-dimethylphenyl)-6-(pyrimidin-4-yl)- 2,5-dihydro-4H-pyrazolo[3,4-d]pyrimidin-4-one | 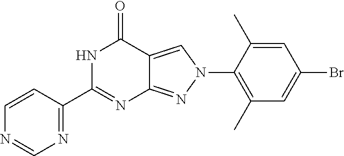 [SMILES: Cc1cc(Br)cc(C)c1-n1cc2c(=O)[nH]c(-c3ccncn3)nc2n1] | 397.0 |
| 186 | 2-[4-cyclopropyl-2-(difluoromethyl)-6-fluorophenyl]- 6-(2-methoxyethoxy)-2,5-dihydro-4H- pyrazolo[3,4-d]pyrimidin-4-one | 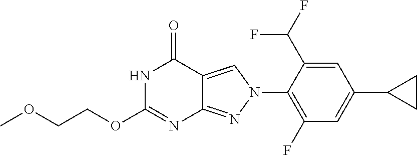 [SMILES: COCCOc1nc2nn(-c3c(F)cc(C4CC4)cc3C(F)F)cc2c(=O)[nH]1] | 394.9 |

TABLE 5-18

| | | | |
|---|---|---|---|
| 187 | 2-(4-bromo-2,6-dimethylphenyl)-6-methyl-2,5-dihydro- 4H-pyrazolo[3,4-d]pyrimidin-4-one | 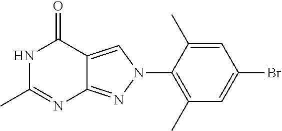 [SMILES: Cc1nc2nn(-c3c(C)cc(Br)cc3C)cc2c(=O)[nH]1] | 332.7 |
| 188 | 2-(4-bromo-2,6-dimethylphenyl)-6-(1-fluorocyclopropyl)- 2,5-dihydro-4H-pyrazolo[3,4-d]pyrimidin-4-one | 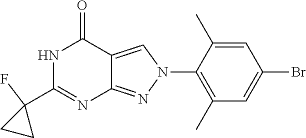 [SMILES: Cc1cc(Br)cc(C)c1-n1cc2c(=O)[nH]c(C3(F)CC3)nc2n1] | 376.8 |
| 189 | 2-(4-bromo-2,6-dimethylphenyl)-6-(2,2-difluoro cyclopropyl)- 2,5-dihydro-4H-pyrazolo[3,4-d]pyrimidin-4-one | 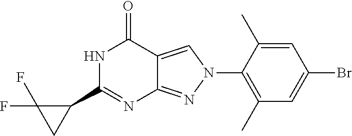 [SMILES: Cc1cc(Br)cc(C)c1-n1cc2c(=O)[nH]c([C@H]3CC3(F)F)nc2n1] | 394.8 |
| 190 | 2-(4-cyclopropyl-2,6-dimethylphenyl)-6-(1-fluorocyclopropyl)- 2,5-dihydro-4H-pyrazolo[3,4-d]pyrimidin-4-one | 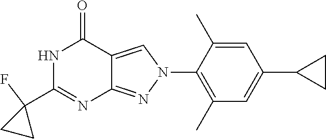 [SMILES: Cc1cc(C2CC2)cc(C)c1-n1cc2c(=O)[nH]c(C3(F)CC3)nc2n1] | 339.0 |
| 191 | 2-(4-cyclopropyl-2,6-dimethylphenyl)-6-(2,2- difluorocyclopropyl)-2,5-dihydro-4H-pyrazolo[3,4-d]pyrimidin- 4-one | 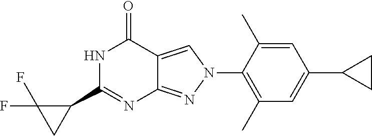 [SMILES: Cc1cc(C2CC2)cc(C)c1-n1cc2c(=O)[nH]c([C@H]3CC3(F)F)nc2n1] | 356.9 |
| 192 | 2-[4-cyclopropyl-2-fluoro-6-(2,2,2-trifluoro-1- hydroxyethyl)phenyl]-6-ethoxy-2,5-dihydro-4H- pyrazolo[3,4-d]pyrimidin-4-one | 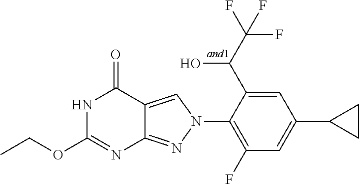 [SMILES: CCOc1nc2nn(-c3c(F)cc(C4CC4)cc3C(O)C(F)(F)F)cc2c(=O)[nH]1] | 413.3 |
| 193 | 2-[4-cyclopropyl-2-(difluoromethyl)-6-fluorophenyl]- 6-methoxy-2,5-dihydro-4H-pyrazolo[3,4-d]pyrimidin-4-one | 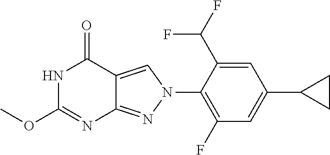 [SMILES: COc1nc2nn(-c3c(F)cc(C4CC4)cc3C(F)F)cc2c(=O)[nH]1] | 351.0 |
| 194 | 2-(4-cyclo propyl-2,6-dimethylphenyl)-6-(2-methoxyethoxy)- 2,5-dihydro-4H-pyrazolo[3,4-d]pyrimidin-4-one | 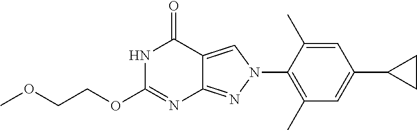 [SMILES: COCCOc1nc2nn(-c3c(C)cc(C4CC4)cc3C)cc2c(=O)[nH]1] | 355.1 |
| 195 | 2-[4-(difluoromethoxy)-2-(difluoromethyl)-6-methylphenyl]- 6-ethoxy-2,5-dihydro-4H-pyrazolo[3,4-d]pyrimidin-4-one | 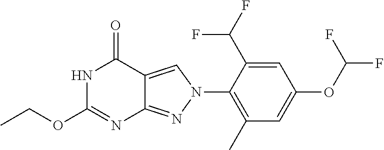 [SMILES: CCOc1nc2nn(-c3c(C)cc(OC(F)F)cc3C(F)F)cc2c(=O)[nH]1] | 387.1 |
| 196 | 2-(2,4-dimethylphenyl)-6-(4,4,4-trifluorobutoxy)- 2,5-dihydro-4H-pyrazolo[3,4-d]pyrimidin-4-one | 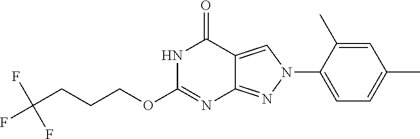 [SMILES: Cc1ccc(-n2cc3c(=O)[nH]c(OCCCC(F)(F)F)nc3n2)c(C)c1] | 367.1 |
| 197 | 2-(2,4-dimethylphenyl)-6-(3,3,3-trifluoropropoxy)- 2,5-dihydro-4H-pyrazolo [3,4-d]pyrimidin-4-one | 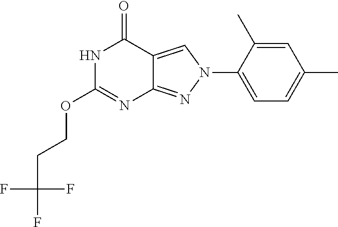 [SMILES: Cc1ccc(-n2cc3c(=O)[nH]c(OCCC(F)(F)F)nc3n2)c(C)c1] | 353.1 |

TABLE 5-19

| | | | |
|---|---|---|---|
| 198 | 2-(4-cyclo propyl-2,6-dimethylphenyl)-6-(oxan-4-yl)- 2,5-dihydro-4H-pyrazolo[3,4-d]pyrimidin-4-one | 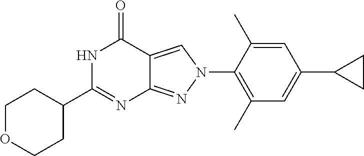 [SMILES: Cc1cc(C2CC2)cc(C)c1-n1cc2c(=O)[nH]c(C3CCOCC3)nc2n1] | 365.2 |
| 199 | 2-(2,4-dimethylphenyl)-6-[(2-oxaspiro[3.3]heptan-6- yl)oxy]-2,5-dihydro-4H-pyrazolo[3,4-d]pyrimidin-4-one | 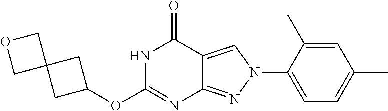 [SMILES: Cc1ccc(-n2cc3c(=O)[nH]c(OC4CC5(COC5)C4)nc3n2)c(C)c1] | 353.1 |
| 200 | 2-[4-cyclopropyl-2-(difluoro methyl)-6- fluorophenyl]-6-[(2R)-2-methoxypropoxy]-2,5- dihydro-4H-pyrazolo[3,4-d]pyrimidin-4-one | 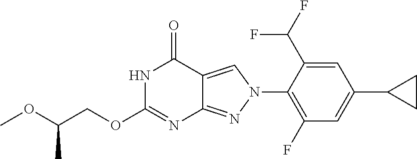 [SMILES: CO[C@H](C)COc1nc2nn(-c3c(F)cc(C4CC4)cc3C(F)F)cc2c(=O)[nH]1] | 409.1 |
| 201 | 2-(4-cyclopropyl-2,6-dimethylphenyl)-6-(oxolan-3-yl)- 2,5-dihydro-4H-pyrazolo [3,4-d]pyrimidin-4-one | 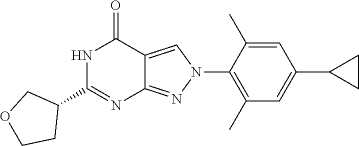 [SMILES: Cc1cc(C2CC2)cc(C)c1-n1cc2c(=O)[nH]c([C@@H]3CCOC3)nc2n1] | 351.2 |
| 202 | 2-(4-cyclopropyl-2,6-dimethylphenyl)-6-[(trifluoro- methoxy)methyl]-2,5-dihydro-4H-pyrazolo[3,4- d]pyrimidin-4-one | 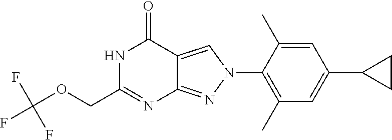 [SMILES: Cc1cc(C2CC2)cc(C)c1-n1cc2c(=O)[nH]c(COC(F)(F)F)nc2n1] | 379.1 |
| 203 | 2-[4-cyclopropyl-2-(difluoromethyl)-6-fluorophenyl]- 6-[(2S)-2-methoxypropoxy]-2,5-dihydro-4H- pyrazolo[3,4-d]pyrimidin-4-one | 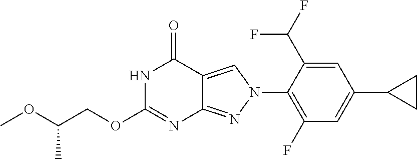 [SMILES: CO[C@@H](C)COc1nc2nn(-c3c(F)cc(C4CC4)cc3C(F)F)cc2c(=O)[nH]1] | 409.2 |
| 204 | 2-[2-bromo-6-fluoro-4-(1-fluorocyclopropyl)phenyl]- 6-methoxy-2,5-dihydro-4H-pyrazolo[3,4- d]pyrimidin-4-one | 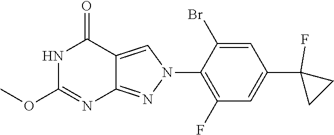 [SMILES: COc1nc2nn(-c3c(F)cc(C4(F)CC4)cc3Br)cc2c(=O)[nH]1] | 397.0 |
| 205 | 2-[4-cyclopropyl-2-(difluoromethyl)-6-fluorophenyl]- 6-{[(2S)-1-methoxypropan-2-yl]oxy}-2,5-dihydro- 4H-pyrazolo[3,4-d]pyrimidin-4-one | 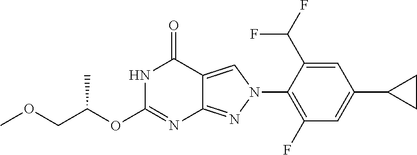 [SMILES: COC[C@H](C)Oc1nc2nn(-c3c(F)cc(C4CC4)cc3C(F)F)cc2c(=O)[nH]1] | 409.2 |
| 206 | 2-[4-cyclopropyl-2-fluoro-6-(2,2,2- trifluoroethyl)phenyl]-6-ethoxy-2,5-dihydro-4H- pyrazolo[3,4-d]pyrimidin-4-one | 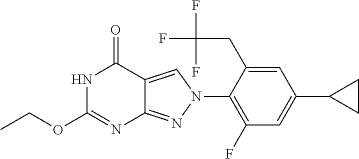 [SMILES: CCOc1nc2nn(-c3c(F)cc(C4CC4)cc3CC(F)(F)F)cc2c(=O)[nH]1] | 397.1 |
| 207 | 2-[4-cyclopropyl-2-(difluoromethyl)-6-fluoro- phenyl]-6-{[(2R)-1-methoxypropan-2-yl]oxy}- 2,5-dihydro-4H-pyrazolo[3,4-d]pyrimidin-4-one | 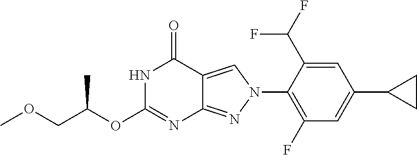 [SMILES: COC[C@@H](C)Oc1nc2nn(-c3c(F)cc(C4CC4)cc3C(F)F)cc2c(=O)[nH]1] | 409.1 |
| 208 | 2-[2-fluoro-4-(1-fluorocyclopropyl)-6- methylphenyl]-6-methoxy-2,5-dihydro-4H- pyrazolo[3,4-d]pyrimidin-4-one | 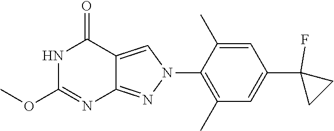 [SMILES: COc1nc2nn(-c3c(C)cc(C4(F)CC4)cc3C)cc2c(=O)[nH]1] | 333.1 |

TABLE 5-20

| | | | |
|---|---|---|---|
| 209 | 2-[4-cyclopropyl-2-(difluoromethyl)- 6-methylphenyl]-6-methoxy-2,5- dihydro-4H-pyrazolo[3,4-d]pyrimidin-4-one | 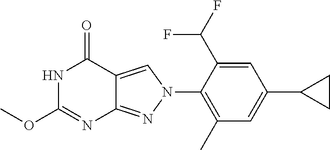 [SMILES: COc1nc2nn(-c3c(C)cc(C4CC4)cc3C(F)F)cc2c(=O)[nH]1] | 347.1 |
| 210 | 2-[4-cyclopropyl-2-(difluoromethyl)- 6-methylphenyl]-6-(2-methoxyethoxy)- 2,5-dihydro-4H-pyrazolo[3,4-d]pyrimidin-4-one | 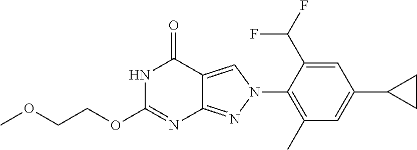 [SMILES: COCCOc1nc2nn(-c3c(C)cc(C4CC4)cc3C(F)F)cc2c(=O)[nH]1] | 391.1 |
| 211 | 2-(4-cyclopropyl-2,6-dimethylphenyl)-6- (2,2,2-trifluoroethyl)-2,5- dihydro-4H-pyrazolo[3,4-d]pyrimidin-4-one | 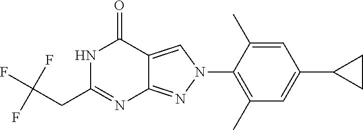 [SMILES: Cc1cc(C2CC2)cc(C)c1-n1cc2c(=O)[nH]c(CC(F)(F)F)nc2n1] | 363.1 |
| 212 | 2-(4-cyclopropyl-2,6-dimethylphenyl)- 6-[(2,2-difluoroethoxy)methyl]-2,5- dihydro-4H-pyrazolo[3,4-d]pyrimidin-4-one | 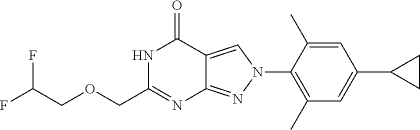 [SMILES: Cc1cc(C2CC2)cc(C)c1-n1cc2c(=O)[nH]c(COCC(F)F)nc2n1] | 375.2 |
| 213 | 2-[4-bromo-2-(difluoro methyl)-6- methylphenyl]-6-ethoxy-2,5-dihydro- 4H-pyrazolo[3,4-d]pyrimidin-4-one | 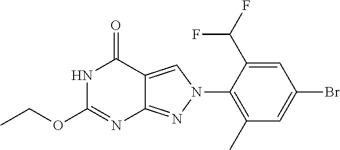 [SMILES: CCOc1nc2nn(-c3c(C)cc(Br)cc3C(F)F)cc2c(=O)[nH]1] | 399.0 |
| 214 | 2-[4-bromo-2-(difluoromethyl)-6- methylphenyl]-6-methoxy-2,5-dihydro- 4H-pyrazolo[3,4-d]pyrimidin-4-one | 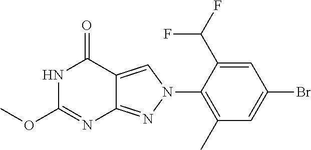 [SMILES: COc1nc2nn(-c3c(C)cc(Br)cc3C(F)F)cc2c(=O)[nH]1] | 385.0 |
| 215 | 2-[4-bromo-2-(difluoromethyl)-6- methylphenyl]-6-(2-methoxyethoxy)- 2,5-dihydro-4H-pyrazolo[3,4-d]pyrimidin-4-one | 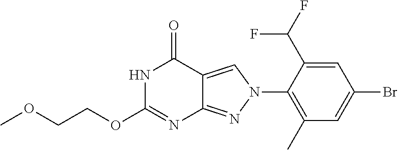 [SMILES: COCCOc1nc2nn(-c3c(C)cc(Br)cc3C(F)F)cc2c(=O)[nH]1] | 429.0 |
| 216 | 2-[4-cyclopropyl-2-(difluoromethyl)- 6-methylphenyl]-6-ethoxy-2,5- dihydro-4H-pyrazo lo[3,4-d]pyrimidin-4-one | 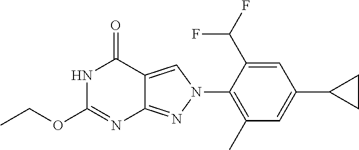 [SMILES: CCOc1nc2nn(-c3c(C)cc(C4CC4)cc3C(F)F)cc2c(=O)[nH]1] | 361.1 |
| 217 | 2-(4-cyclopropyl-2,6-dimethylphenyl)- 6-(1-methyl-1H-pyrazol-4-yl)-2,5- dihydro-4H-pyrazolo[3,4-d]pyrimidin-4-one | 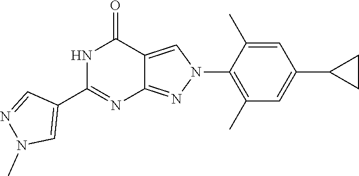 [SMILES: Cc1cc(C2CC2)cc(C)c1-n1cc2c(=O)[nH]c(-c3cnn(C)c3)nc2n1] | 361.1 |
| 218 | 2-(4-cyclo propyl-2,6-dimethylphenyl)- 6-(6,7-dihydro-4H-pyrazolo[5,1-c][1,4]oxazin- 2-yl)-2,5-dihydro-4H-pyrazolo[3,4-d]pyrimidin- 4-one | 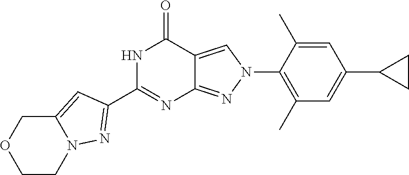 [SMILES: Cc1cc(C2CC2)cc(C)c1-n1cc2c(=O)[nH]c(-c3cc4n(n3)CCOC4)nc2n1] | 403.3 |
| 219 | 3-{[2-(2,4-dimethylphenyl)-4-oxo- 4,5-dihydro-2H-pyrazolo[3,4-d]pyrimidin- 6-yl]oxy}cyclobutane-1-carbonitrile | 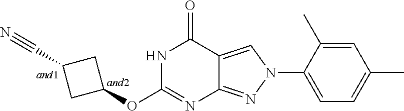 [SMILES: Cc1ccc(-n2cc3c(=O)[nH]c(O[C@H]4C[C@H](C#N)C4)nc3n2)c(C)c1] | 336.1 |

TABLE 5-21

| | | | |
|---|---|---|---|
| 220 | 6-(2,2-difluoroethoxy)-2-(2,4-dimethylphenyl)- 2,5-dihydro-4H-pyrazolo [3,4- d]pyrimidin-4-one | 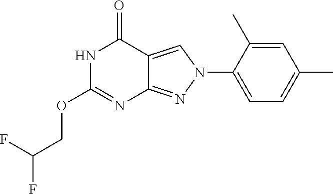 [SMILES: Cc1ccc(-n2cc3c(=O)[nH]c(OCC(F)F)nc3n2)c(C)c1] | 321.0 |
| 221 | 2-[4-bromo-2-(difluoromethoxy)- 6-methylphenyl]-6-ethoxy-2,5-dihydro- 4H-pyrazolo[3,4-d]pyrimidin-4-one | 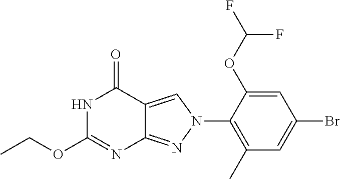 [SMILES: CCOc1nc2nn(-c3c(C)cc(Br)cc3OC(F)F)cc2c(=O)[nH]1] | 415.0 |
| 222 | 2-[4-cyclopropyl-2-(difluoromethoxy)- 6-methylphenyl]-6-ethoxy-2,5- dihydro-4H-pyrazolo[3,4-d]pyrimidin-4-one | 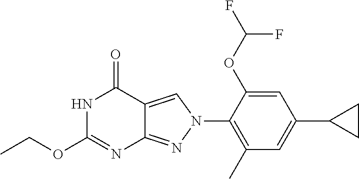 [SMILES: CCOc1nc2nn(-c3c(C)cc(C4CC4)cc3OC(F)F)cc2c(=O)[nH]1] | 377.1 |
| 223 | 2-(4-cyclopropyl-2,6-dimethylphenyl)- 6-(oxolan-2-yl)-2,5-dihydro-4H- pyrazolo[3,4-d]pyrimidin-4-one | 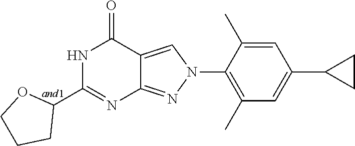 [SMILES: Cc1cc(C2CC2)cc(C)c1-n1cc2c(=O)[nH]c(C3CCCO3)nc2n1] | 351.2 |
| 224 | 2-(4-cyclo propyl-2,6-dimethylphenyl)- 6-[(difluoromethoxy)methyl]-2,5- dihydro-4H-pyrazolo[3,4-d]pyrimidin-4-one | 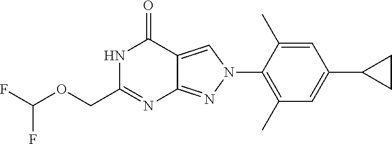 [SMILES: Cc1cc(C2CC2)cc(C)c1-n1cc2c(=O)[nH]c(COC(F)F)nc2n1] | 361.1 |
| 225 | 2-[4-cyclopropyl-2-(difluoro methoxy)- 6-methylphenyl]-6-(2-methoxyethoxy)- 2,5-dihydro-4H-pyrazolo[3,4-d]pyrimidin-4-one | 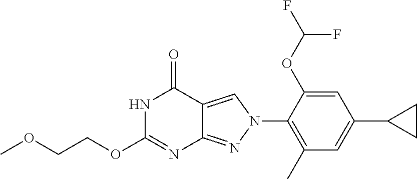 [SMILES: COCCOc1nc2nn(-c3c(C)cc(C4CC4)cc3OC(F)F)cc2c(=O)[nH]1] | 407.1 |
| 226 | 2-[6-(difluoromethoxy)-2,4-dimethylpyridin- 3-yl]-6-ethoxy-2,5-dihydro- 4H-pyrazolo[3,4-d]pyrimidin-4-one | 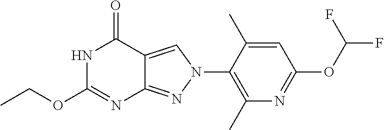 [SMILES: CCOc1nc2nn(-c3c(C)cc(OC(F)F)nc3C)cc2c(=O)[nH]1] | 352.1 |
| 227 | 2-(4-cyclopropyl-2,6-dimethylphenyl)- 6-[(2R)-oxolan-2-yl]-2,5-dihydro- 4H-pyrazolo[3,4-d]pyrimidin-4-one or 2-(4-cyclopropyl-2,6- dimethylphenyl)-6-[(2S)-oxolan-2-yl]- 2,5-dihydro-4H-pyrazolo[3,4- d]pyrimidin-4-one (HPLC Method A: tR1) | 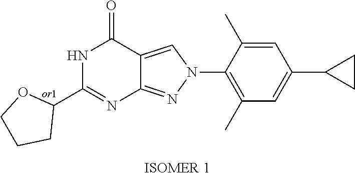 [SMILES: Cc1cc(C2CC2)cc(C)c1-n1cc2c(=O)[nH]c(C3CCCO3)nc2n1] | 351.2 |
| 228 | 2-(4-cyclopropyl-2,6-dimethylphenyl)- 6-[(2S)-oxolan-2-yl]-2,5-dihydro- 4H-pyrazolo[3,4-d]pyrimidin-4-one or 2-(4-cyclo propyl-2,6- dimethylphenyl)-6-[(2R)-oxolan-2-yl]- 2,5-dihydro-4H-pyrazolo[3,4- d]pyrimidin-4-one (HPLC Method A: tR2) | 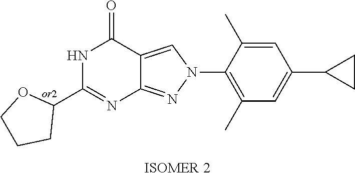 [SMILES: Cc1cc(C2CC2)cc(C)c1-n1cc2c(=O)[nH]c(C3CCCO3)nc2n1] | 351.2 |
| 229 | 2-(4-cyclopropyl-2,6-dimethylphenyl)- 6-(1-methyl-1H-1,2,3-triazol-4-yl)- 2,5-dihydro-4H-pyrazolo[3,4-d]pyrimidin- 4-one | 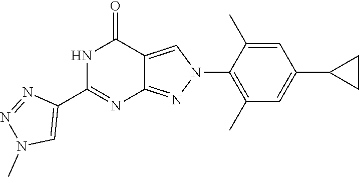 [SMILES: Cc1cc(C2CC2)cc(C)c1-n1cc2c(=O)[nH]c(-c3cn(C)nn3)nc2n1] | 362.2 |
| 230 | 2-[4-cyclopropyl-2-methyl-6-(2,2,2- trifluoroethyl)phenyl]-6-methoxy-2,5- dihydro-4H-pyrazolo[3,4-d]pyrimidin- 4-one | 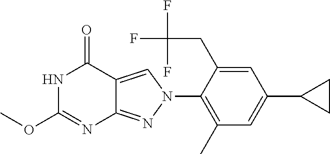 [SMILES: COc1nc2nn(-c3c(C)cc(C4CC4)cc3CC(F)(F)F)cc2c(=O)[nH]1] | 379.2 |

TABLE 5-22

| | | | |
|---|---|---|---|
| 231 | 2-[4-cyclopropyl-2-methyl-6-(2,2,2- trifluoroethyl)phenyl]-6-(2-methoxyethoxy)- 2,5-dihydro-4H-pyrazolo[3,4-d]pyrimidin- 4-one | 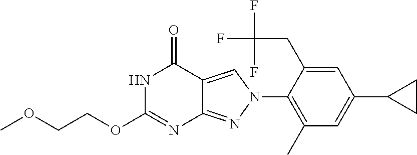 [SMILES: COCCOc1nc2nn(-c3c(C)cc(C4CC4)cc3CC(F)(F)F)cc2c(=O)[nH]1] | 423.2 |
| 232 | 2-(4-cyclopropyl-2,6-dimethylphenyl)- 6-(1,4-dioxan-2-yl)-2,5-dihydro- 4H-pyrazolo[3,4-d]pyrimidin-4-one | 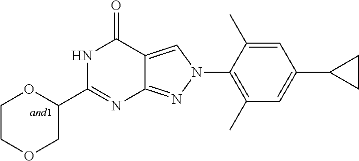 [SMILES: Cc1cc(C2CC2)cc(C)c1-n1cc2c(=O)[nH]c(C3COCCO3)nc2n1] | 367.2 |
| 233 | 2-(4-cyclopropyl-2,6-dimethylphenyl)- 6-(oxan-2-yl)-2,5-dihydro-4H- pyrazolo[3,4-d]pyrimidin-4-one | 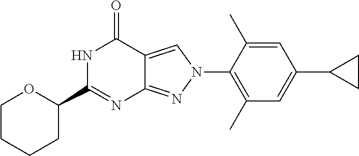 [SMILES: Cc1cc(C2CC2)cc(C)c1-n1cc2c(=O)[nH]c([C@H]3CCCCO3)nc2n1] | 365.1 |
| 234 | 2-(4-cyclopropyl-2,6-dimethylphenyl)- 6-(2,2-diethoxyethyl)-2,5-dihydro- 4H-pyrazolo[3,4-d]pyrimidin-4-one | 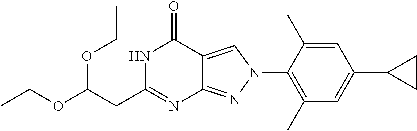 [SMILES: CCOC(Cc1nc2nn(-c3c(C)cc(C4CC4)cc3C)cc2c(=O)[nH]1)OCC] | 397.3 |
| 235 | 2-[4-(1-fluorocyclopropyl)-2,6- dimethylphenyl]-6-methoxy-2,5-dihydro- 4H-pyrazolo[3,4-d]pyrimidin-4-one | 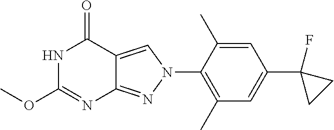 [SMILES: COc1nc2nn(-c3c(C)cc(C4(F)CC4)cc3C)cc2c(=O)[nH]1] | 329.1 |
| 236 | 2-(4-cyclopropyl-2,6-dimethylphenyl)- 6-{[(oxetan-3-yl)oxy]methyl}-2,5- dihydro-4H-pyrazolo[3,4-d]pyrimidin-4-one | 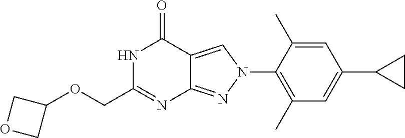 [SMILES: Cc1cc(C2CC2)cc(C)c1-n1cc2c(=O)[nH]c(COC3COC3)nc2n1] | 367.1 |
| 237 | 2-(4-cyclopropyl-2,6-dimethylphenyl)- 6-[(1,2-oxazolidin-2-yl)methyl]-2,5- dihydro-4H-pyrazolo[3,4-d]pyrimidin-4-one | 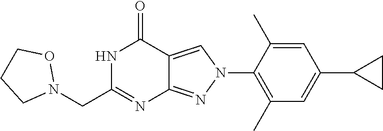 [SMILES: Cc1cc(C2CC2)cc(C)c1-n1cc2c(=O)[nH]c(CN3CCCO3)nc2n1] | 366.5 |
| 238 | 2-(4-cyclopropyl-2,6-dimethylphenyl)- 6-(4-fluorooxan-4-yl)-2,5-dihydro- 4H-pyrazolo[3,4-d]pyrimidin-4-one | 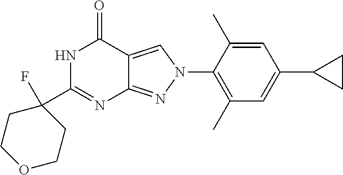 [SMILES: Cc1cc(C2CC2)cc(C)c1-n1cc2c(=O)[nH]c(C3(F)CCOCC3)nc2n1] | 383.1 |
| 239 | 2-(4-cyclopropyl-2,6-dimethylphenyl)- 6-(6,7-dihydro-5H-pyrazolo[5,1- b][1,3]oxazin-2-yl)-2,5-dihydro-4H- pyrazolo[3,4-d]pyrimidin-4-one | 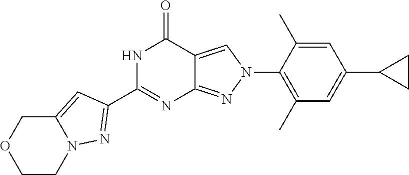 [SMILES: Cc1cc(C2CC2)cc(C)c1-n1cc2c(=O)[nH]c(-c3cc4n(n3)CCOC4)nc2n1] | 403.2 |
| 240 | 2-(4-cyclopropyl-2,6-dimethylphenyl)- 6-propyl-2,5-dihydro-4H- pyrazolo[3,4-d]pyrimidin-4-one | 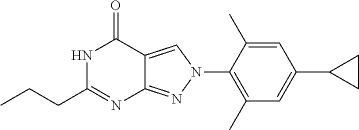 [SMILES: CCCc1nc2nn(-c3c(C)cc(C4CC4)cc3C)cc2c(=O)[nH]1] | 323.1 |
| 241 | 2-(4-cyclopropyl-2,6-dimethylphenyl)- 6-ethyl-2,5-dihydro-4H- pyrazolo[3,4-d]pyrimidin-4-one | 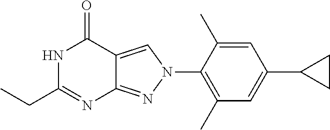 [SMILES: CCc1nc2nn(-c3c(C)cc(C4CC4)cc3C)cc2c(=O)[nH]1] | 309.1 |

TABLE 5-23

| | | | |
|---|---|---|---|
| 242 | 2-(4-cyclopropyl-2,6-dimethylphenyl)-6- [5-(trifluoromethyl)-1H-imidazol- 2-yl]-2,5-dihydro-4H-pyrazolo[3,4-d]pyrimidin- 4-one | 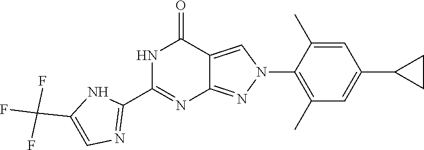 [SMILES: Cc1cc(C2CC2)cc(C)c1-n1cc2c(=O)[nH]c(-c3ncc(C(F)(F)F)[nH]3)nc2n1] | 415.2 |
| 243 | 2-(2,4-dimethylphenyl)-6-{[(1s,3s)-3- methoxycyclobutyl]oxy}-2,5- dihydro-4H-pyrazolo[3,4-d]pyrimidin- 4-one | 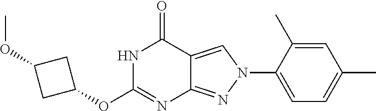 [SMILES: CO[C@H]1C[C@@H](Oc2nc3nn(-c4ccc(C)cc4C)cc3c(=O)[nH]2)C1] | 341.1 |
| 244 | 2-(2,4-dimethylphenyl)-6-{[(1r,3r)-3- methoxycyclobutyl]oxy}-2,5-dihydro- 4H-pyrazolo[3,4-d]pyrimidin-4-one | 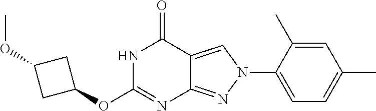 [SMILES: CO[C@H]1C[C@H](Oc2nc3nn(-c4ccc(C)cc4C)cc3c(=O)[nH]2)C1] | 341.1 |
| 245 | 6-ethoxy-2-[4-(1-fluorocyclopropyl)-2,6- dimethylphenyl]-2,5-dihydro-4H- pyrazolo[3,4-d]pyrimidin-4-one | 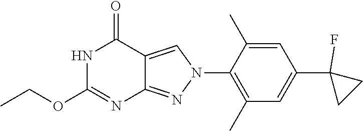 [SMILES: CCOc1nc2nn(-c3c(C)cc(C4(F)CC4)cc3C)cc2c(=O)[nH]1] | 343.1 |
| 246 | 2-[4-(1-fluorocyclopropyl)-2,6- dimethylphenyl]-6-(2-methoxyethoxy)- 2,5-dihydro-4H-pyrazolo[3,4-d]pyrimidin- 4-one | 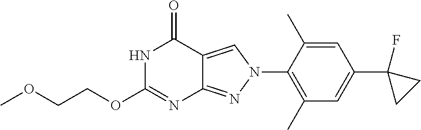 [SMILES: COCCOc1nc2nn(-c3c(C)cc(C4(F)CC4)cc3C)cc2c(=O)[nH]1] | 373.1 |
| 247 | 2-(4-cyclopropyl-2,6-dimethylphenyl)-6- (3-fluorooxolan-3-yl)-2,5-dihydro- 4H-pyrazolo[3,4-d]pyrimidin-4-one | 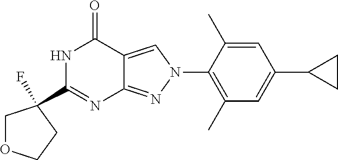 [SMILES: Cc1cc(C2CC2)cc(C)c1-n1cc2c(=O)[nH]c([C@@]3(F)CCOC3)nc2n1] | 369.2 |
| 248 | 2-(4-cyclopropyl-2,6-dimethylphenyl)- 6-[(1s,3s)-3-methoxycyclobutyl]- 2,5-dihydro-4H-pyrazolo[3,4-d]pyrimidin- 4-one | 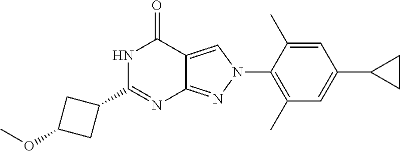 [SMILES: CO[C@H]1C[C@@H](c2nc3nn(-c4c(C)cc(C5CC5)cc4C)cc3c(=O)[nH]2)C1] | 365.2 |
| 249 | 2-(4-cyclopropyl-2,6-dimethylphenyl)- 6-[(1s,3s)-3-fluorocyclobutyl]-2,5- dihydro-4H-pyrazolo[3,4-d]pyrimidin- 4-one | 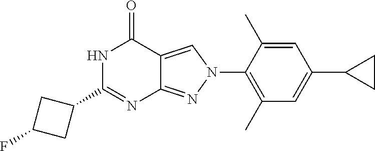 [SMILES: Cc1cc(C2CC2)cc(C)c1-n1cc2c(=O)[nH]c([C@H]3C[C@@H](F)C3)nc2n1] | 353.2 |
| 250 | 2-[2-bromo-4-cyclopropyl-6- (difluoromethyl)phenyl]-6-ethoxy-2,5- dihydro-4H-pyrazolo[3,4-d]pyrimidin-4-one | 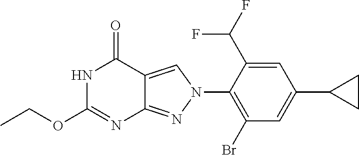 [SMILES: CCOc1nc2nn(-c3c(Br)cc(C4CC4)cc3C(F)F)cc2c(=O)[nH]1] | 425.1 |
| 251 | 2-[2,4-dibromo-6-(difluoromethyl)phenyl]- 6-ethoxy-2,5-dihydro-4H- pyrazolo[3,4-d]pyrimidin-4-one | 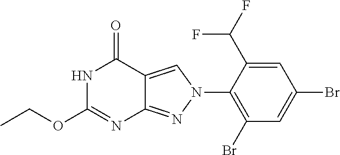 [SMILES: CCOc1nc2nn(-c3c(Br)cc(Br)cc3C(F)F)cc2c(=O)[nH]1] | 462.9 |
| 252 | 2-[4-cyclopropyl-2-(difluoromethoxy)- 6-methylphenyl]-6-methoxy-2,5- dihydro-4H-pyrazolo [3,4-d]pyrimidin-4-one | 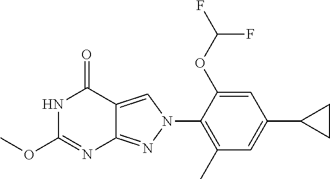 [SMILES: COc1nc2nn(-c3c(C)cc(C4CC4)cc3OC(F)F)cc2c(=O)[nH]1] | 363.1 |

TABLE 5-24

| | | | |
|---|---|---|---|
| 253 | racemic mixture of 2-(4-cyclopropyl-2,6- dimethylphenyl)-6-[(1R,2S)-2- methoxycyclobutyl]-2,5-dihydro-4H- pyrazolo[3,4-d]pyrimidin-4-one and 2-(4-cyclopropyl-2,6-dimethylphenyl)- 6-[(1S,2R)-2-methoxycyclobutyl]- 2,5-dihydro-4H-pyrazolo[3,4-d]pyrimidin- 4-one | 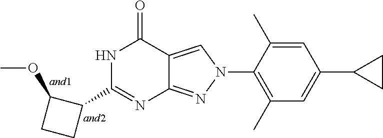 [SMILES: CO[C@@H]1CC[C@H]1c1nc2nn(-c3c(C)cc(C4CC4)cc3C)cc2c(=O)[nH]1] | 365.2 |
| 254 | 5-cyclopropyl-3-(difluoromethyl)-2- (6-ethoxy-4-oxo-4,5-dihydro-2H- pyrazolo[3,4-d]pyrimidin-2- yl)benzonitrile | 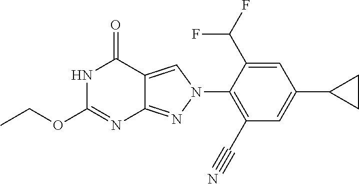 [SMILES: CCOc1nc2nn(-c3c(CN)cc(C4CC4)cc3C(F)F)cc2c(=O)[nH]1] | 372.1 |
| 255 | 2-(4-cyclopropyl-2,6-dimethylphenyl)- 6-[(1r,3r)-3-fluorocyclobutyl]-2,5- dihydro-4H-pyrazolo [3,4-d]pyrimidin-4-one | 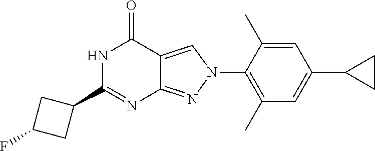 [SMILES: Cc1cc(C2CC2)cc(C)c1-n1cc2c(=O)[nH]c([C@H]3C[C@H](F)C3)nc2n1] | 353.3 |
| 256 | 6-[(difluoromethoxy)methyl]-2-[2- (difluoromethyl)-4-nitrophenyl]-2,5- dihydro-4H-pyrazolo [3,4-d]pyrimidin-4-one | 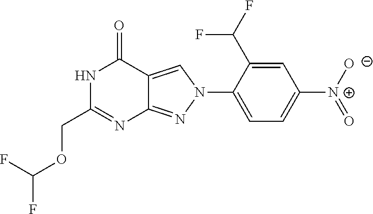 [SMILES: O=c1[nH]c(COC(F)F)nc2nn(-c3ccc([N+](=O)[O-])cc3C(F)F)cc12] | 388.0 |
| 257 | 2-(4-cyclopropyl-2,6-dimethylphenyl)- 6-(2-ethyl-2H-1,2,3-triazol-4-yl)- 2,5-dihydro-4H-pyrazolo[3,4-d]pyrimidin- 4-one | 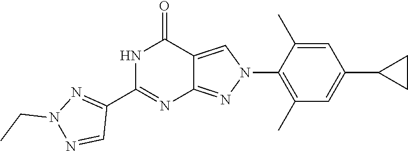 [SMILES: CCn1ncc(-c2nc3nn(-c4c(C)cc(C5CC5)cc4C)cc3c(=O)[nH]2)n1] | 376.2 |
| 258 | 2-(4-cyclopropyl-2,6-dimethylphenyl)- 6-(2-methyl-2H-1,2,3-triazol-4-yl)- 2,5-dihydro-4H-pyrazolo[3,4-d]pyrimidin- 4-one | 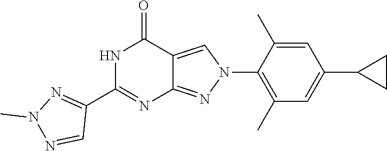 [SMILES: Cc1cc(C2CC2)cc(C)c1-n1cc2c(=O)[nH]c(-c3cnn(C)n3)nc2n1] | 362.1 |
| 259 | 2-(4-cyclopropyl-2,6-dimethylphenyl)- 6-(1,1-difluoroethyl)-2,5-dihydro- 4H-pyrazolo[3,4-d]pyrimidin-4-one | 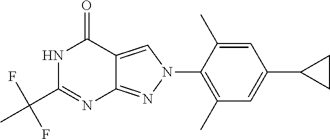 [SMILES: Cc1cc(C2CC2)cc(C)c1-n1cc2c(=O)[nH]c(C(C)(F)F)nc2n1] | 345.1 |
| 260 | 2-(4-cyclopropyl-2,6-dimethylphenyl)- 6-(3-fluorooxan-3-yl)-2,5-dihydro- 4H-pyrazolo[3,4-d]pyrimidin-4-one | 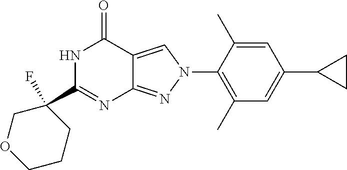 [SMILES: Cc1cc(C2CC2)cc(C)c1-n1cc2c(=O)[nH]c([C@@]3(F)CCCOC3)nc2n1] | 383.2 |
| 261 | 2-[4-amino-2-(difluoromethyl)phenyl]- 6-[(difluoromethoxy)methyl]-2,5- dihydro-4H-pyrazolo[3,4-d]pyrimidin-4-one | 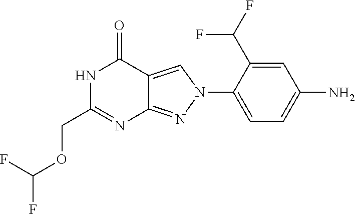 [SMILES: Nc1ccc(-n2cc3c(=O)[nH]c(COC(F)F)nc3n2)c(C(F)F)c1] | 358 |
| 262 | 2-(4-cyclopropyl-2,6-dimethylphenyl)- 6-(3,3-difluorocyclobutyl)-2,5- dihydro-4H-pyrazolo[3,4-d]pyrimidin- 4-one | 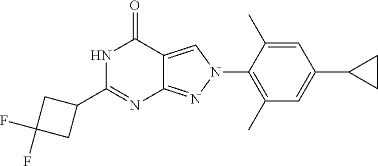 [SMILES: Cc1cc(C2CC2)cc(C)c1-n1cc2c(=O)[nH]c(C3CC(F)(F)C3)nc2n1] | 371.2 |
| 263 | 6-[(difluoromethoxy)methyl]-2-[4- (1-fluorocyclopropyl)-2,6- dimethylphenyl]-2,5-dihydro-4H- pyrazolo[3,4-d]pyrimidin-4-one | 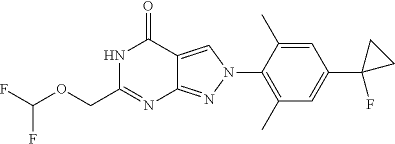 [SMILES: Cc1cc(C2(F)CC2)cc(C)c1-n1cc2c(=O)[nH]c(COC(F)F)nc2n1] | 379.1 |

TABLE 5-25

| | | | |
|---|---|---|---|
| 264 | 2-(4-cyclopropyl-2,6-dimethylphenyl)- 6-(5,6-dihydro-8H-imidazo[2,1- c][1,4]oxazin-2-yl)-2,5-dihydro-4H- pyrazolo[3,4-d]pyrimidin-4-one | 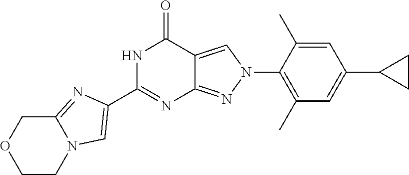 [SMILES: Cc1cc(C2CC2)cc(C)c1-n1cc2c(=O)[nH]c(-c3cn4c(n3)COCC4)nc2n1] | 403.2 |
| 265 | racemic mixture of 2-(4-cyclopropyl- 2,6-dimethylphenyl)-6-[(1R,2S)-2- methoxycyclopropyl]-2,5-dihydro- 4H-pyrazolo[3,4-d]pyrimidin-4-one and 2-(4-cyclopropyl-2,6-dimethylphenyl)- 6-[(1S,2R)-2-methoxycyclopropyl]-2,5- dihydro-4H-pyrazolo[3,4-d]pyrimidin-4-one | 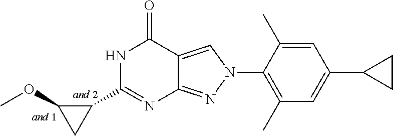 [SMILES: CO[C@@H]1C[C@H]1c1nc2nn(-c3c(C)cc(C4CC4)cc3C)cc2c(=O)[nH]1] | 351.2 |
| 266 | 2-(4-cyclopropyl-2,6-dimethylphenyl)- 6-(1-methoxycyclopropyl)-2,5- dihydro-4H-pyrazolo[3,4-d]pyrimidin- 4-one | 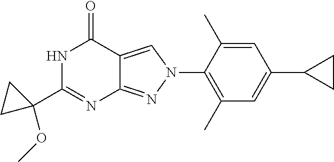 [SMILES: COC1(c2nc3nn(-c4c(C)cc(C5CC5)cc4C)cc3c(=O)[nH]2)CC1] | 351.2 |
| 267 | 2-[4-bromo-2-(difluoromethyl)phenyl]- 6-[(difluoromethoxy)methyl]-2,5- dihydro-4H-pyrazolo[3,4-d]pyrimidin-4-one | 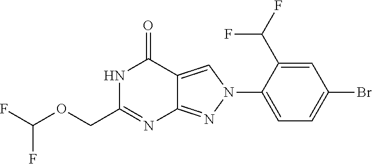 [SMILES: O=c1[nH]c(COC(F)F)nc2nn(-c3ccc(Br)cc3C(F)F)cc12] | 421 |
| 268 | 6-[2-(benzyloxy)ethyl]-2-(4-cyclopropyl- 2,6-dimethylphenyl)-2,5-dihydro- 4H-pyrazolo[3,4-d]pyrimidin-4-one | 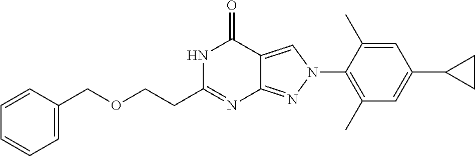 [SMILES: Cc1cc(C2CC2)cc(C)c1-n1cc2c(=O)[nH]c(CCOCc3ccccc3)nc2n1] | 415.2 |
| 269 | 2-[6-cyclopropyl-2-methyl-4- (trifluoromethyl)pyridin-3-yl]-6-ethoxy- 2,5-dihydro-4H-pyrazolo[3,4-d]pyrimidin- 4-one | 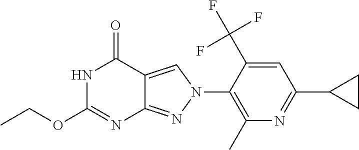 [SMILES: CCOc1nc2nn(-c3c(C(F)(F)F)cc(C4CC4)nc3C)cc2c(=O)[nH]1] | 380.1 |
| 270 | 2-(4-cyclopropyl-2,6-dimethylphenyl)- 6-(2,3-dihydropyrazolo[5,1- b][1,3]oxazol-6-yl)-2,5-dihydro-4H- pyrazolo[3,4-d]pyrimidin-4-one | 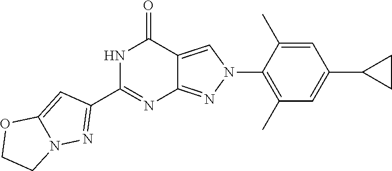 [SMILES: Cc1cc(C2CC2)cc(C)c1-n1cc2c(=O)[nH]c(-c3cc4n(n3)CCO4)nc2n1] | 389.2 |
| 271 | 2-[4-cyclopropyl-2-(difluoromethyl)phenyl]- 6-[(difluoromethoxy)methyl]-2,5- dihydro-4H-pyrazolo[3,4-d]pyrimidin-4-one | 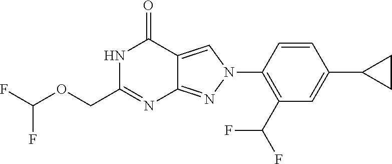 [SMILES: O=c1[nH]c(COC(F)F)nc2nn(-c3ccc(C4CC4)cc3C(F)F)cc12] | 383.1 |
| 272 | 2-(4-cyclopropyl-2,6-dimethylphenyl)- 6-(2-methyl-1,3-oxazol-4-yl)-2,5- dihydro-4H-pyrazolo[3,4-d]pyrimidin- 4-one | 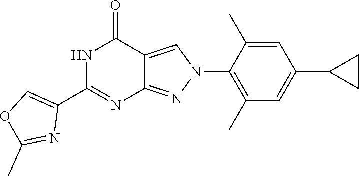 [SMILES: Cc1nc(-c2nc3nn(-c4c(C)cc(C5CC5)cc4C)cc3c(=O)[nH]2)co1] | 362.1 |
| 273 | 2-(4-cyclopropyl-2,6-dimethylphenyl)- 6-(2-methyl-1,3-oxazol-5-yl)-2,5- dihydro-4H-pyrazolo[3,4-d]pyrimidin- 4-one | 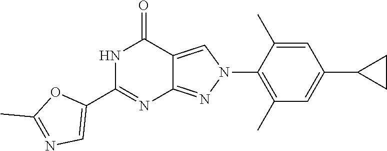 [SMILES: Cc1ncc(-c2nc3nn(-c4c(C)cc(C5CC5)cc4C)cc3c(=O)[nH]2)o1] | 362.2 |
| 274 | 2-(4-cyclopropyl-2,6-dimethylphenyl)- 6-(5-methyl-1,2-oxazol-3-yl)-2,5- dihydro-4H-pyrazolo[3,4-d]pyrimidin- 4-one | 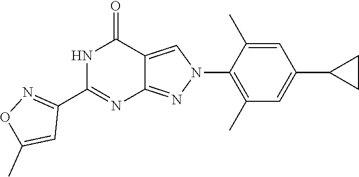 [SMILES: Cc1cc(-c2nc3nn(-c4c(C)cc(C5CC5)cc4C)cc3c(=O)[nH]2)no1] | 362.2 |

TABLE 5-26

| | | | |
|---|---|---|---|
| 275 | 2-(4-cyclopropyl-2,6-dimethylphenyl)- 6-(3-methyl-1,2-oxazol-5-yl)-2,5-dihydro- 4H-pyrazolo[3,4-d]pyrimidin-4-one | 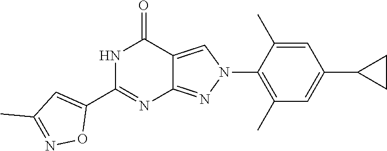 [SMILES: Cc1cc(-c2nc3nn(-c4c(C)cc(C5CC5)cc4C)cc3c(=O)[nH]2)on1] | 362.1 |
| 276 | 2-(4-cyclopropyl-2,6-dimethylphenyl)- 6-(2-hydroxyethyl)-2,5-dihydro- 4H-pyrazolo[3,4-d]pyrimidin-4-one | 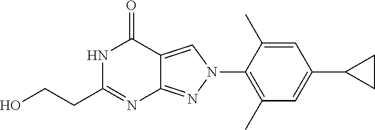 [SMILES: Cc1cc(C2CC2)cc(C)c1-n1cc2c(=O)[nH]c(CCO)nc2n1] | 325.1 |
| 277 | 2-(4-cyclopropyl-2,6-dimethylphenyl)- 6-(1-methoxycyclobutyl)-2,5- dihydro-4H-pyrazolo[3,4-d]pyrimidin- 4-one | 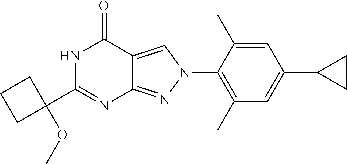 [SMILES: COC1(c2nc3nn(-c4c(C)cc(C5CC5)cc4C)cc3c(=O)[nH]2)CCC1] | 365.2 |
| 278 | 2-(4-cyclopropyl-2,6-dimethylphenyl)- 6-[1-(2-methoxyethyl)-1H-pyrazol- 3-yl]-2,5-dihydro-4H-pyrazolo[3,4- d]pyrimidin-4-one | 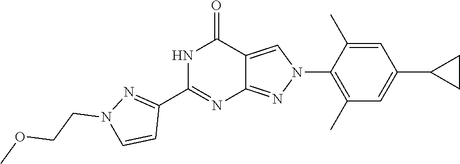 [SMILES: COCCn1ccc(-c2nc3nn(-c4c(C)cc(C5CC5)cc4C)cc3c(=O)[nH]2)n1] | 405.2 |
| 279 | 2-[4-bromo-2-(difluoromethyl)-6- methylphenyl]-6-[(difluoromethoxy)methyl]- 2,5-dihydro-4H-pyrazolo[3,4-d]pyrimidin-4- one | 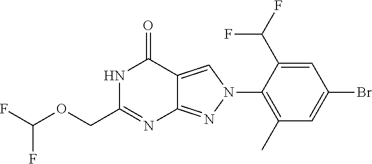 [SMILES: Cc1cc(Br)cc(C(F)F)c1-n1cc2c(=O)[nH]c(COC(F)F)nc2n1] | 435.1 |
| 280 | 2-[3,4-dibromo-6-(difluoromethyl)- 2-methylphenyl]-6-[(difluoromethoxy)methyl]- 2,5-dihydro-4H-pyrazolo[3,4-d]pyrimidin-4- one | 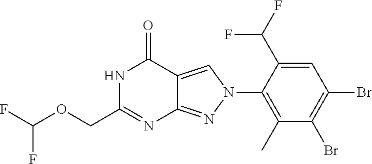 [SMILES: Cc1c(Br)c(Br)cc(C(F)F)c1-n1cc2c(=O)[nH]c(COC(F)F)nc2n1] | 513 |
| 281 | 2-[4-cyclopropyl-3-(difluoromethyl)- 2-fluorophenyl]-6-ethyl-2,5-dihydro- 4H-pyrazolo[3,4-d]pyrimidin-4-one | 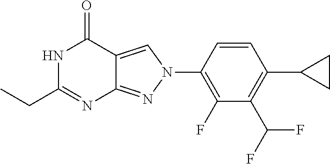 [SMILES: CCc1nc2nn(-c3ccc(C4CC4)c(C(F)F)c3F)cc2c(=O)[nH]1] | 349.1 |
| 282 | 2-[4-cyclopropyl-3-(difluoromethyl)- 2-ethoxyphenyl]-6-ethyl-2,5-dihydro- 4H-pyrazolo[3,4-d]pyrimidin-4-one | 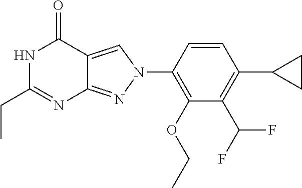 [SMILES: CCOc1c(-n2cc3c(=O)[nH]c(CC)nc3n2)ccc(C2CC2)c1C(F)F] | 375.1 |
| 283 | 2-[4-cyclopropyl-2-(difluoromethyl)- 6-methylphenyl]-6-[(difluoromethoxy)methyl]- 2,5-dihydro-4H-pyrazolo[3,4-d]pyrimidin-4- one | 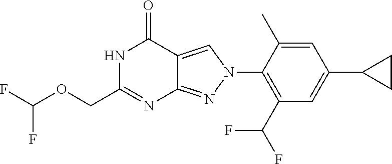 [SMILES: Cc1cc(C2CC2)cc(C(F)F)c1-n1cc2c(=O)[nH]c(COC(F)F)nc2n1] | 397.1 |
| 284 | 2-[4-(difluoromethoxy)-2-(difluoromethyl)- 6-methylphenyl]-6-methoxy- 2,5-dihydro-4H-pyrazolo[3,4-d]pyrimidin- 4-one | 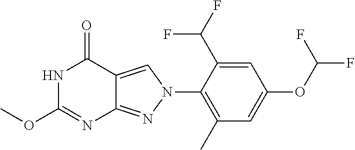 [SMILES: COc1nc2nn(-c3c(C)cc(OC(F)F)cc3C(F)F)cc2c(=O)[nH]1] | 373 |
| 285 | 2-(4-cyclopropyl-2,6-dimethylphenyl)- 6-(2,2-difluoroethoxy)-2,5- dihydro-4H-pyrazolo[3,4-d]pyrimidin-4-one | 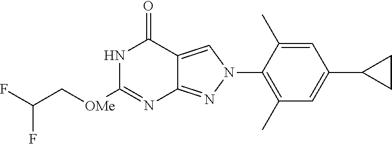 | 361.1 |

TABLE 5-27

| | | | |
|---|---|---|---|
| 286 | 2-(4-cyclopropyl-2,6-dimethylphenyl)- 6-{[(3S)-oxolan-3-yl]oxy}-2,5- dihydro-4H-pyrazolo [3,4-d]pyrimidin-4- one | 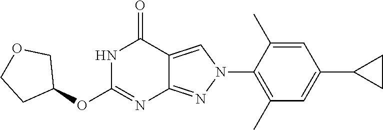 [SMILES: Cc1cc(C2CC2)cc(C)c1-n1cc2c(=O)[nH]c(O[C@H]3CCOC3)nc2n1] | 367.1 |
| 287 | 2-(4-cyclopropyl-2,6-dimethylphenyl)- 6-(2-methylpyrimidin-4-yl)-2,5- dihydro-4H-pyrazolo[3,4-d]pyrimidin- 4-one | 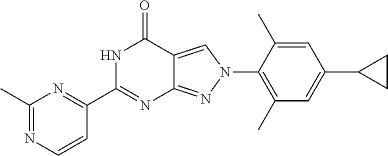 [SMILES: Cc1nccc(-c2nc3nn(-c4c(C)cc(C5CC5)cc4C)cc3c(=O)[nH]2)n1] | 373.2 |
| 288 | 2-(4-cyclopropyl-2,6-dimethylphenyl)- 6-[2-(difluoromethoxy)ethoxy]-2,5- dihydro-4H-pyrazolo[3,4-d]pyrimidin- 4-one | 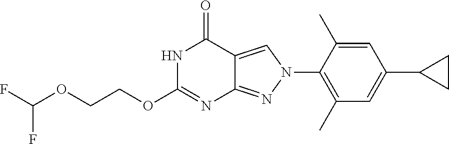 [SMILES: Cc1cc(C2CC2)cc(C)c1-n1cc2c(=O)[nH]c(OCCOC(F)F)nc2n1] | 391.1 |
| 289 | 2-(4-cyclopropyl-2,6-dimethylphenyl)-6- [(3R)-3-fluorooxolan-3-yl]-2,5- dihydro-4H-pyrazolo[3,4-d]pyrimidin-4-one or 2-(4-cyclopropyl-2,6-dimethylphenyl)- 6-[(3S)-3-fluorooxolan-3-yl]-2,5-dihydro-4H- pyrazolo[3,4-d]pyrimidin-4-one (HPLC Method B: tR1) | 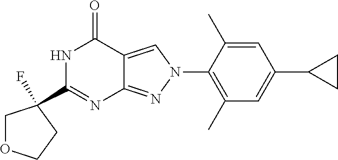 [SMILES: Cc1cc(C2CC2)cc(C)c1-n1cc2c(=O)[nH]c([C@@]3(F)CCOC3)nc2n1] | 369.1 |
| 290 | 2-(4-cyclopropyl-2,6-dimethylphenyl)- 6-[(3S)-3-fluorooxolan-3-yl]-2,5-dihydro- 4H-pyrazolo[3,4-d]pyrimidin-4-one or 2-(4-cyclopropyl-2,6-dimethylphenyl)- 6-[(3R)-3-fluorooxolan-3-yl]-2,5-dihydro- 4H-pyrazolo[3,4-d]pyrimidin-4-one (HPLC Method B: tR2) | 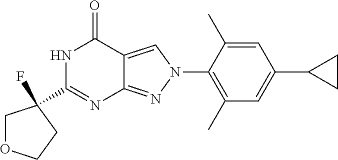 [SMILES: Cc1cc(C2CC2)cc(C)c1-n1cc2c(=O)[nH]c([C@]3(F)CCOC3)nc2n1] | 369.2 |
| 291 | 2-(4-cyclopropyl-2-ethyl-6-methylphenyl)- 6-[(difluoromethoxy)methyl]-2,5- dihydro-4H-pyrazolo[3,4-d]pyrimidin-4-one | 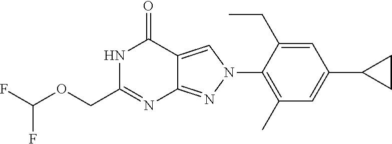 [SMILES: CCc1cc(C2CC2)cc(C)c1-n1cc2c(=O)[nH]c(COC(F)F)nc2n1] | 375.1 |
| 292 | 2-[4-(1-fluorocyclopropyl)-2,6- dimethylphenyl]-6-(1-methyl-1H-pyrazol- 3-yl)-2,5-dihydro-4H-pyrazolo[3,4-d]pyrimidin- 4-one | 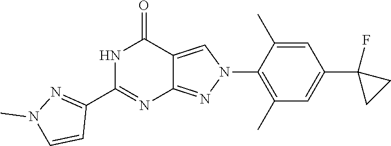 [SMILES: Cc1cc(C2(F)CC2)cc(C)c1-n1cc2c(=O)[nH]c(-c3ccn(C)n3)nc2n1] | 379.1 |
| 293 | 2-(4-cyclopropyl-2,6-dimethylphenyl)- 6-{[(3R)-oxolan-3-yl]oxy}-2,5-dihydro- 4H-pyrazolo[3,4-d]pyrimidin-4-one | 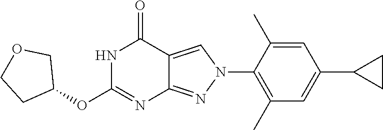 [SMILES: Cc1cc(C2CC2)cc(C)c1-n1cc2c(=O)[nH]c(O[C@@H]3CCOC3)nc2n1] | 367.2 |
| 294 | 2-(2-chloro-4-cyclopropyl-6-methylphenyl)- 6-[(difluoromethoxy)methyl]-2,5- dihydro-4H-pyrazolo[3,4-d]pyrimidin-4-one | 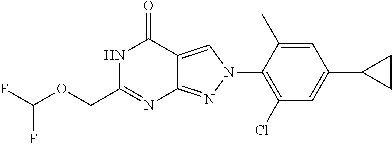 [SMILES: Cc1cc(C2CC2)cc(Cl)c1-n1cc2c(=O)[nH]c(COC(F)F)nc2n1] | 381.1 |
| 295 | 2-[4-cyclopropyl-2-(difluoromethyl)- 6-fluorophenyl]-6-ethyl-2,5-dihydro- 4H-pyrazolo[3,4-d]pyrimidin-4-one | 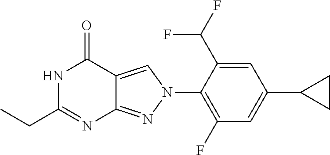 [SMILES: CCc1nc2nn(-c3c(F)cc(C4CC4)cc3C(F)F)cc2c(=O)[nH]1] | 349.1 |
| 296 | 2-[4-cyclopropyl-2-(difluoromethyl)- 6-fluorophenyl]-6-(oxolan-3-yl)-2,5- dihydro-4H-pyrazolo [3,4-d]pyrimidin-4-one | 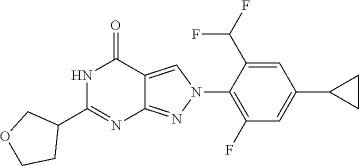 [SMILES: O=c1[nH]c(C2CCOC2)nc2nn(-c3c(F)cc(C4CC4)cc3C(F)F)cc12] | 391.1 |

TABLE 5-28

| | | | |
|---|---|---|---|
| 297 | 2-(4-cyclopropyl-2,6-dimethylphenyl)- 6-(1-methyl-1H-pyrazol-3-yl)-2,5- dihydro-4H-pyrazolo[3,4-d]pyrimidin- 4-one | 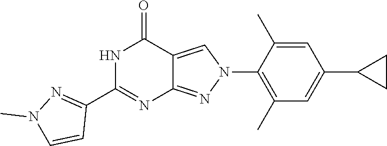 [SMILES: Cc1cc(C2CC2)cc(C)c1-n1cc2c(=O)[nH]c(-c3ccn(C)n3)nc2n1] | 361.1 |
| 298 | 2-[4-cyclopropyl-2-(difluoromethyl)-6- fluorophenyl]-6-(2-methyl-1,3-oxazol- 4-yl)-2,5-dihydro-4H-pyrazolo[3,4-d]pyrimidin- 4-one | 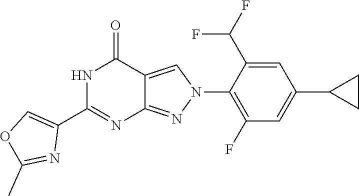 [SMILES: Cc1nc(-c2nc3nn(-c4c(F)cc(C5CC5)cc4C(F)F)cc3c(=O)[nH]2)co1] | 402.1 |
| 299 | 2-[4-cyclopropyl-2-(difluoromethyl)- 6-fluorophenyl]-6-(1-methyl-1H- pyrazol-3-yl)-2,5-dihydro-4H-pyrazolo[3,4- d]pyrimidin-4-one | 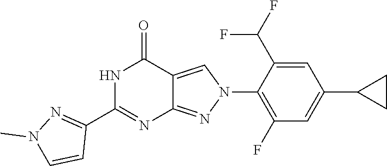 [SMILES: Cn1ccc(-c2nc3nn(-c4c(F)cc(C5CC5)cc4C(F)F)cc3c(=O)[nH]2)n1] | 401.2 |
| 300 | 2-[4-cyclopropyl-2-(difluoromethyl)-6- fluorophenyl]-6-(2-methoxyethyl)- 2,5-dihydro-4H-pyrazolo[3,4-d]pyrimidin- 4-one | 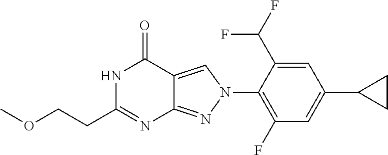 [SMILES: COCCc1nc2nn(-c3c(F)cc(C4CC4)cc3C(F)F)cc2c(=O)[nH]1] | 379.1 |
| 301 | 2-[2-ethyl-4-(1-fluorocyclopropyl)-6- methylphenyl]-6-methoxy-2,5- dihydro-4H-pyrazolo[3,4-d]pyrimidin- 4-one | 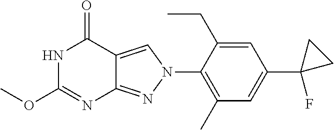 [SMILES: CCc1cc(C2(F)CC2)cc(C)c1-n1cc2c(=O)[nH]c(OC)nc2n1] | 343.1 |
| 302 | 2-[4-cyclopropyl-2-(difluoromethyl)-6- fluorophenyl]-6-[(difluoromethoxy)methyl]- 2,5-dihydro-4H-pyrazolo[3,4-d]pyrimidin-4- one | 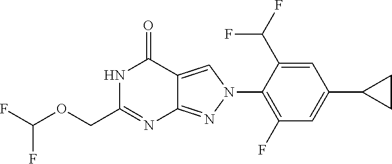 [SMILES: O=c1[nH]c(COC(F)F)nc2nn(-c3c(F)cc(C4CC4)cc3C(F)F)cc12] | 401.1 |
| 303 | 2-[6-cyclopropyl-4-methyl-2- (trifluoromethyl)pyridin-3-yl]-6-ethoxy-2,5- dihydro-4H-pyrazolo[3,4-d]pyrimidin-4-one | 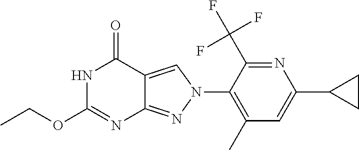 [SMILES: CCOc1nc2nn(-c3c(C)cc(C4CC4)nc3C(F)(F)F)cc2c(=O)[nH]1] | 380.1 |
| 304 | 2-(4-cyclopropyl-2,6-dimethylphenyl)- 6-(hydroxymethyl)-2,5-dihydro- 4H-pyrazolo[3,4-d]pyrimidin-4-one | 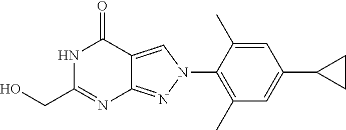 [SMILES: Cc1cc(C2CC2)cc(C)c1-n1cc2c(=O)[nH]c(CO)nc2n1] | 311.1 |
| 305 | 2-[4-cyclopropyl-2-(difluoromethyl)- 6-fluorophenyl]-6-(1,4-dioxan-2-yl)- 2,5-dihydro-4H-pyrazolo[3,4-d]pyrimidin-4-one | 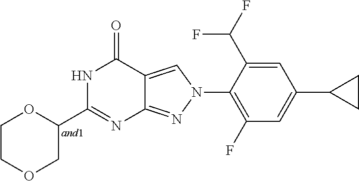 [SMILES: O=c1[nH]c(C2COCCO2)nc2nn(-c3c(F)cc(C4CC4)cc3C(F)F)cc12] | 407.2 |
| 306 | 2-(4-cyclopropyl-2,6-dimethylphenyl)- 6-(3,4-dihydro-2H-pyran-4-yl)-2,5- dihydro-4H-pyrazolo[3,4-d]pyrimidin-4-one | 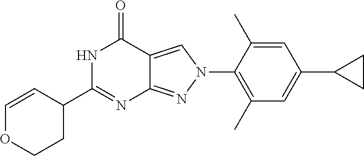 [SMILES: Cc1cc(C2CC2)cc(C)c1-n1cc2c(=O)[nH]c(C3C=COCC3)nc2n1] | 363.1 |
| 307 | 2-(4-cyclopropyl-2,6-dimethylphenyl)- 6-(3,6-dihydro-2H-pyran-4-yl)-2,5- dihydro-4H-pyrazolo[3,4-d]pyrimidin-4-one | 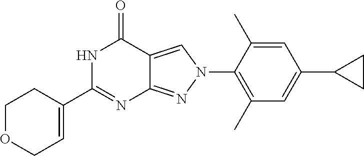 [SMILES: Cc1cc(C2CC2)cc(C)c1-n1cc2c(=O)[nH]c(C3=CCOCC3)nc2n1] | 363.1 |

TABLE 5-29

| | | | |
|---|---|---|---|
| 308 | 2-[4-cyclopropyl-2-(difluoromethyl)-6- fluorophenyl]-6-(methoxymethyl)- 2,5-dihydro-4H-pyrazolo[3,4-d]pyrimidin- 4-one | 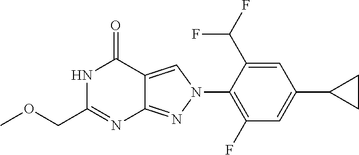 [SMILES: COCc1nc2nn(-c3c(F)cc(C4CC4)cc3C(F)F)cc2c(=O)[nH]1] | 365.1 |
| 309 | 2-[4-cyclopropyl-2-(2,2-difluoroethyl)-6- fluorophenyl]-6-methoxy-2,5-dihydro- 4H-pyrazolo [3,4-d]pyrimidin-4-one | 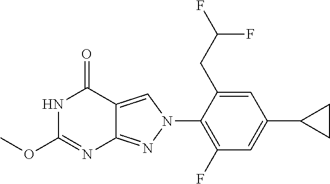 [SMILES: COc1nc2nn(-c3c(F)cc(C4CC4)cc3CC(F)F)cc2c(=O)[nH]1] | 365.1 |
| 310 | racemic mixture of 1,5-anhydro-3-[2- (4-cyclopropyl-2,6-dimethylphenyl)- 4-oxo-4,5-dihydro-2H-pyrazolo[3,4- d]pyrimidin-6-yl]-2,3-dideoxy- 4-O-methyl-D-erythro-pentitol and 1,5-anhydro-3-[2-(4-cyclopropyl-2,6- dimethylphenyl)-4-oxo-4,5-dihydro-2H- pyrazolo[3,4-d]pyrimidin-6-yl]-3,4- dideoxy-2-O-methyl-D-erythro-pentitol | 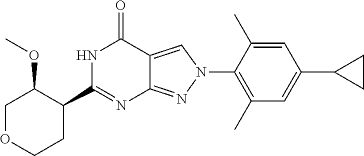 [SMILES: CO[C@@H]1COCC[C@@H]1c1nc2nn(-c3c(C)cc(C4CC4)cc3C)cc2c(=O)[nH]1] | 395.2 |
| 311 | racemic mixture of 1,5-anhydro-3-[2- (4-cyclopropyl-2,6-dimethylphenyl)-4-oxo- 4,5-dihydro-2H-pyrazolo[3,4-d]pyrimidin- 6-yl]-2,3-dideoxy-4-O-methyl-L-threo- pentitol and 1,5-anhydro-3-[2-(4- cyclopropyl-2,6-dimethylphenyl)-4- oxo-4,5-dihydro-2H-pyrazolo[3,4-d]pyrimidin- 6-yl]-2,3-dideoxy-4-O-methyl-D-threo-pentitol | 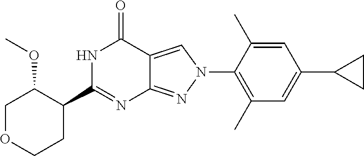 [SMILES: CO[C@H]1COCC[C@@H]1c1nc2nn(-c3c(C)cc(C4CC4)cc3C)cc2c(=O)[nH]1] | 395.2 |
| 312 | 2-[4-cyclopropyl-2-(difluoromethyl)- 6-fluorophenyl]-6-[(2R)-1,4-dioxan- 2-yl]-2,5-dihydro-4H-pyrazolo[3,4- d]pyrimidin-4-one or 2-[4-cyclopropyl- 2-(difluoro methyl)-6-fluorophenyl]-6- [(2S)-1,4-dioxan-2-yl]-2,5-dihydro-4H- pyrazolo [3,4-d]pyrimidin-4-one (HPLC Method C: tR1) | 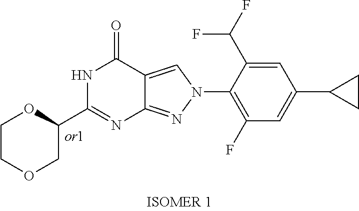 [SMILES: O=c1[nH]c([C@H]2COCCO2)nc2nn(-c3c(F)cc(C4CC4)cc3C(F)F)cc12] | 407.1 |
| 313 | 2-[4-cyclopropyl-2-(difluoromethyl)-6- fluorophenyl]-6-[(2S)-1,4-dioxan-2-yl]- 2,5-dihydro-4H-pyrazolo[3,4-d]pyrimidin- 4-one or 2-[4-cyclopropyl-2-(difluoromethyl)- 6-fluorophenyl]-6-[(2R)-1,4-dioxan-2-yl]- 2,5-dihydro-4H-pyrazolo[3,4-d]pyrimidin-4-one (HPLC Method C: tR2) | 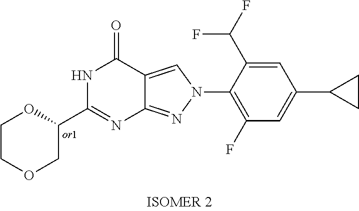 [SMILES: O=c1[nH]c([C@@H]2COCCO2)nc2nn(-c3c(F)cc(C4CC4)cc3C(F)F)cc12] | 407.1 |
| 314 | 2-[4-cyclopropyl-2-(difluoromethyl)-6- fluorophenyl]-6-(ethoxymethyl)- 2,5-dihydro-4H-pyrazolo[3,4-d]pyrimidin- 4-one | 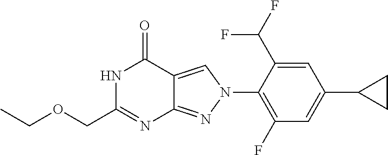 [SMILES: CCOCc1nc2nn(-c3c(F)cc(C4CC4)cc3C(F)F)cc2c(=O)[nH]1] | 379.1 |
| 315 | 2-(4-cyclopropyl-2,6-dimethylphenyl)- 6-(3-methyloxetan-3-yl)-2,5-dihydro- 4H-pyrazolo [3,4-d]pyrimidin-4-one | 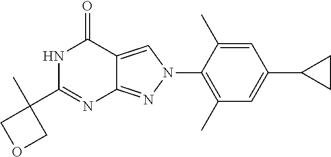 [SMILES: Cc1cc(C2CC2)cc(C)c1-n1cc2c(=O)[nH]c(C3(C)COC3)nc2n1] | 351.2 |
| 316 | 2-[4-cyclopropyl-2-(difluoromethyl)- 6-methylphenyl]-6-(1,4-dioxan-2-yl)- 2,5-dihydro-4H-pyrazolo[3,4-d]pyrimidin- 4-one | 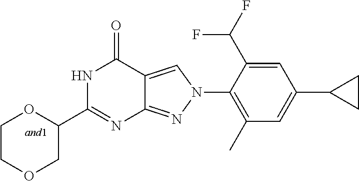 [SMILES: Cc1cc(C2CC2)cc(C(F)F)c1-n1cc2c(=O)[nH]c(C3COCCO3)nc2n1] | 403.1 |
| 317 | [2-(4-cyclopropyl-2,6-dimethylphenyl)- 4-oxo-4,5-dihydro-2H-pyrazolo[3,4- d]pyrimidin-6-yl]acetonitrile | 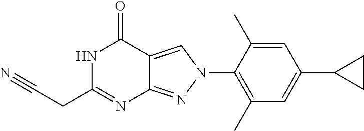 [SMILES: Cc1cc(C2CC2)cc(C)c1-n1cc2c(=O)[nH]c(CC#N)nc2n1] | 320.1 |
| 318 | 1-[2-(4-cyclopropyl-2,6-dimethylphenyl)- 4-oxo-4,5-dihydro-2H-pyrazolo[3,4- d]pyrimidin-6-yl]cyclopropane-1-carbonitrile | 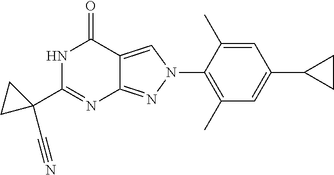 [SMILES: Cc1cc(C2CC2)cc(C)c1-n1cc2c(=O)[nH]c(C3(C#N)CC3)nc2n1] | 346.2 |

TABLE 5-30

| | | | |
|---|---|---|---|
| 319 | 2-[4-cyclopropyl-2-(difluoromethyl)- 6-methylphenyl]-6-(1-fluorocyclopropyl)- 2,5-dihydro-4H-pyrazolo[3,4-d]pyrimidin-4- one | 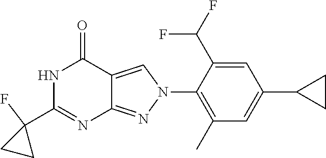 [SMILES: Cc1cc(C2CC2)cc(C(F)F)c1-n1cc2c(=O)[nH]c(C3(F)CC3)nc2n1] | 375.1 |
| 320 | 2-[3,4-dicyclopropyl-6-(difluoromethyl)- 2-methylphenyl)-6-(methoxymethyl)-2,5- dihydro-4H-pyrazolo[3,4-d]pyrimidin-4-one | 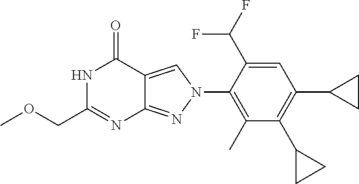 [SMILES: COCc1nc2nn(-c3c(C(F)F)cc(C4CC4)c(C4CC4)c3C)cc2c(=O)[nH]1] | 401.1 |
| 321 | 2-[4-cyclopropyl-2-(difluoromethyl)-6- methylphenyl]-6-(methoxymethyl)- 2,5-dihydro-4H-pyrazolo[3,4-d]pyrimidin- 4-one | 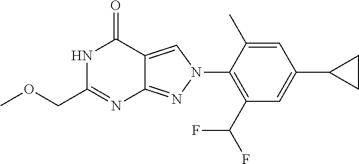 [SMILES: COCc1nc2nn(-c3c(C)cc(C4CC4)cc3C(F)F)cc2c(=O)[nH]1] | 361.1 |
| 322 | 2-[4-cyclopropyl-2-(2,2-difluoroethyl)-6- fluorophenyl]-6-(methoxymethyl)- 2,5-dihydro-4H-pyrazolo[3,4-d]pyrimidin- 4-one | 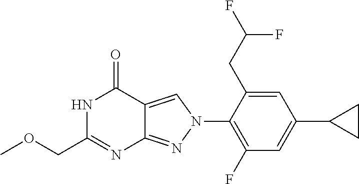 [SMILES: COCc1nc2nn(-c3c(F)cc(C4CC4)cc3CC(F)F)cc2c(=O)[nH]1] | 379.1 |
| 323 | 2-[4-cyclopropyl-2-(2,2-difluoroethyl)-6- methylphenyl]-6-(methoxymethyl)-2,5- dihydro-4H-pyrazolo[3,4-d]pyrimidin-4-one | 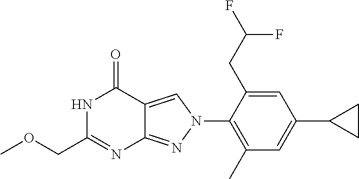 [SMILES: COCc1nc2nn(-c3c(C)cc(C4CC4)cc3CC(F)F)cc2c(=O)[nH]1] | 375.1 |
| 324 | 2-[4-cyclopropyl-2-(2,2-difluoroethyl)- 6-fluorophenyl]-6-(2-methoxymethyl)-2,5- dihydro-4H-pyrazolo[3,4-d]pyrimidin-4-one | 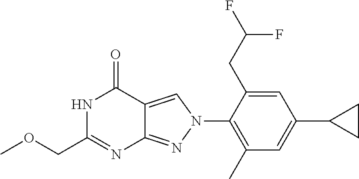 [SMILES: COCc1nc2nn(-c3c(C)cc(C4CC4)cc3CC(F)F)cc2c(=O)[nH]1] | 375.1 |
| 325 | 2-[4-cyclopropyl-2-(difluoromethyl)-6- methylphenyl]-6-[(2R)-1,4-dioxan-2-yl]-2,5- dihydro-4H-pyrazolo[3,4-d]pyrimidin-4-one or 2-[4-cyclopropyl-2-(difluoromethyl)-6- methylphenyl]-6-[(2S)-1,4-dioxan-2-yl]- 2,5-dihydro-4H-pyrazolo[3,4-d]pyrimidin-4-one (SFC Method A: tR1) | 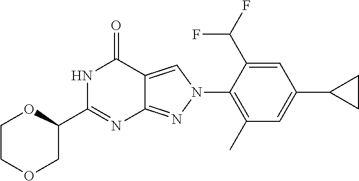 [SMILES: Cc1cc(C2CC2)cc(C(F)F)c1-n1cc2c(=O)[nH]c([C@H]3COCCO3)nc2n1] | 403.1 |
| 326 | 2-[4-cyclopropyl-2-(difluoromethyl)-6- methylphenyl]-6-[(2S)-1,4-dioxan-2-yl]- 2,5-dihydro-4H-pyrazolo[3,4-d]pyrimidin- 4-one or 2-[4-cyclopropyl-2-(difluoromethyl)- 6-methylphenyl]-6-[(2R)-1,4-dioxan-2-yl]- 2,5-dihydro-4H-pyrazolo[3,4-d]pyrimidin-4-one (SFC Method A: tR2) | 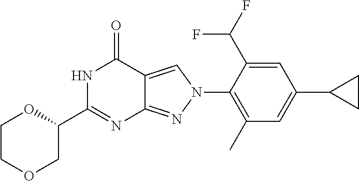 [SMILES: Cc1cc(C2CC2)cc(C(F)F)c1-n1cc2c(=O)[nH]c([C@@H]3COCCO3)nc2n1] | 403.1 |

### Biological Activity

The biological activity of the compound of Formula 1 with respect to NLRP3 was determined using the following in vitro method.

IL-1β TR-FRET Assay (reported as percentage of inhibition at 300 nM, 6 μM or 10 μM)

Monocytic THP-1 cells (ATCC: TIB-202) were maintained in accordance with the provider's instructions in RPMI media (Life Technologies, Cat #A10491-01); RPMI was supplemented with 10% heat inactivated fetal bovine serum (Corning, Cat #35-010-CV). The cells were differentiated into macrophages by the addition of 25 ng/mL IFN-γ (PeproTech, Cat #AF-300-02-100UG) for 24 hours at 37° C./5% CO₂. Media was exchanged with fresh media with no FBS, and the cells were treated with 50 ng/mL LPS (priming step) (LPS-EK: Invivogen, Cat #tlrl-peklps). The cells were plated at 20,000 cells per well in 384-well flat-bottom cell culture plates (FALCON, Cat #353962) and were incubated for 24 hours at 37° C./5% CO₂. Compounds were serially diluted (half log or 5-fold dilutions) with DMSO and were finally diluted with Media with no FBS. The compounds were added to the cells in 384-well plates (added in 1:3) and then the plates were incubated for 30 minutes at 37° C./5% CO₂. The NLRP3 inflammasome was activated with the addition of 20 mM ATP (Sigma Cat #A3377-25G) and the cells were incubated for 2 hours at 37° C./5% CO₂. At the end of the incubation period, 30 μL supernatant was transferred to another 384-well plate and mixed on a plate shaker for 1 minute. The supernatant was mixed with HTRF Antibody (Human IL1 beta kit, Cisbio, 62HIL1BPEH) in assay plates (Greiner Bio-One, Cat #784075) and the assay plates were incubated in shading box at room temperature for 16-24 hours. HTRF signal was measured by EnVision (Perkinelmer) in accordance with the manufacturer's instructions.

### TNF-α Assay (Reported as IC₅₀)

Monocytic THP-1 cells (ATCC: TIB-202) were maintained in accordance with the provider's instructions in RPMI media (Life Technologies, Cat #A10491-01); RPMI was supplemented with 10% heat inactivated fetal bovine serum (Hyclone Cat #SH30396.03). The cells were differentiated into macrophages by the addition of 25 ng/mL IFN-7 for 24 hours at 37° C./5% CO₂. Media was exchanged with fresh media with no FBS. The cells were plated at 40,000 cells per well in 384-well flat-bottom cell culture plates (Costar 3764) containing compounds (added in 1:1000) in a 1:3.16 serial dilution series in DMSO and were incubated for 30 minutes at 37° C./5% CO₂. The NF-κB pathway was activated with the addition of 600 ng/mL LPS and the cells were incubated for 3 hours at 37° C./5% CO₂. At the end of the incubation period, supernatant (40 L) was removed, and TNF-alpha were monitored using an ELISA (Human TNF-α ELISA, R&D systems, Cat #DY210) according to the manufacturer's instructions. 100% inhibition is determined with positive control, TPCA-1.

### Data Interpretation

The percentage of inhibition at 300 nM, 6 μM or 10 μM in the IL-1βTR-FRET assay was calculated by the following formula: Percentage of inhibition=[1−(HTRF signalₜₑₛₜ−HTRF signal_{Low})/(HTRF signal_{High}−HTRF signal_{Low})]*100, where HTRF signalₜₑₛₜ is the HTRF signal in the well to which test compound is added, HTRF signal_{Low} is the HTRF signal in the well to which 3 μM MCC-950 is added, and the HTRF signal_{High} is the HTRF signal in the well to which DMSO is added.

The IC₅₀ values in the TNF-α assay were calculated from a plot of percentage of inhibition versus the inhibitor concentration by a logistics curve fit according to: Y=[Bottom+(Top−Bottom)]/(1+10\hat{ }[(Log IC₅₀−X)·Hill Slope], where Y was the 0% inhibition at the inhibitor concentration, X, “Bottom” was the lowest inhibition value, i.e., 0%, “Top” was the maximum inhibition value, i.e., 100%, and the “Hill Slope” described the slope of the sigmoidal curve between the “Bottom” and “Top” values. The curve fitting was conducted with internally developed software.

Table 6 lists in vitro biological assay data (IL-1β and TNF-αL assay) for the compounds shown in the examples. These assays are described in the section entitled Biological Activity, above.

TABLE 6
Biological Assay Data

| | IL-1β | IL-1β | IL-1β | |
|---|---|---|---|---|
| | inhibitory | inhibitory | inhibitory | |
| Example | rate at | rate at | rate at | TNF-α IC₅₀ |
| No. | 300 nM (%) | 6 μM (%) | 10 μM (%) | (μM) |
| 1 | 100 | | | >30 |
| 2 | 100 | | | >30 |
| 3 | 94 | | | >30 |
| 4 | 92 | | | >30 |
| 5 | 99 | | | >30 |
| 6 | 98 | | | >30 |
| 7 | 99 | | | >30 |
| 8 | 92 | | | >30 |
| 9 | 100 | | | >30 |
| 10 | 98 | | | >30 |
| 11 | 99 | | | >30 |
| 12 | 99 | | | 3.4 |
| 13 | 99 | | | >30 |
| 14 | 96 | | | >30 |
| 15 | 99 | | | >30 |
| 16 | 101 | | | >30 |
| 17 | 99 | | | >30 |
| 18 | 99 | | | >30 |
| 19 | 95 | | | >30 |
| 20 | 100 | | | >30 |
| 21 | 98 | | | >30 |
| 22 | 101 | | | >30 |
| 23 | 100 | | | >30 |
| 24 | 99 | | | >30 |
| 25 | 100 | | | >30 |
| 26 | 94 | | | >30 |
| 27 | 94 | | | >30 |
| 28 | 101 | | | >30 |
| 29 | 97 | | | >30 |
| 30 | 100 | | | >30 |
| 31 | 99 | | | >30 |
| 32 | 105 | | | >30 |
| 33 | 97 | | | >30 |
| 34 | 98 | | | >30 |
| 35 | 96 | | | >30 |
| 36 | 97 | | | >30 |
| 37 | 101 | | | >30 |
| 38 | 100 | | | >30 |
| 39 | 100 | | | >30 |
| 40 | 100 | | | >30 |
| 41 | 99 | | | >30 |
| 42 | 97 | | | >30 |
| 43 | 98 | | | >30 |
| 44 | 101 | | | >30 |
| 45 | 100 | | | >30 |
| 46 | 95 | | | >30 |
| 47 | 98 | | | >30 |
| 48 | 100 | | | >30 |
| 49 | 98 | | | >30 |
| 50 | 101 | | | >30 |
| 51 | 100 | | | >30 |
| 52 | 95 | | | >30 |
| 53 | 95 | | | >30 |
| 54 | 100 | | | >30 |
| 55 | 98 | | | >30 |
| 56 | 100 | | | >30 |
| 57 | 99 | | | >30 |
| 58 | 100 | | | >30 |
| 59 | 98 | | | >30 |
| 60 | 100 | | | >30 |
| 61 | 100 | | | >30 |
| 62 | 95 | | | >30 |
| 63 | 100 | | | >30 |
| 64 | 100 | | | >30 |
| 65 | 96 | | | >30 |
| 66 | 97 | | | >30 |
| 67 | 98 | | | >30 |
| 68 | 97 | | | 0.279 |
| 69 | 100 | | | >30 |
| 70 | 100 | | | >30 |
| 71 | 96 | | | 1.69 |
| 72 | 94 | | | >30 |
| 73 | 97 | | | >30 |
| 74 | 99 | | | >30 |
| 75 | 98 | | | >30 |
| 76 | 94 | | | >30 |
| 77 | 99 | | | >30 |
| 78 | 98 | | | |
| 79 | 95 | | | >30 |
| 80 | 99 | | | |
| 81 | 97 | | | >30 |
| 82 | 98 | | | >30 |
| 83 | 99 | | | >30 |
| 84 | 97 | | | >30 |
| 85 | 92 | | | >30 |
| 86 | 85 | | | >30 |
| 87 | 97 | | | >30 |
| 88 | 86 | | | >30 |
| 89 | 79 | | | >30 |
| 90 | 85 | | | >30 |
| 91 | 76 | | | >30 |
| 92 | 82 | | | >30 |
| 93 | 80 | | | >30 |
| 94 | 83 | | | >30 |
| 95 | 72 | | | >30 |
| 96 | 55 | | | >30 |
| 97 | 66 | | | >30 |
| 98 | 44 | | | >30 |
| 99 | 41 | | | >30 |
| 100 | | | 100 | >30 |
| 101 | | | 95 | >30 |
| 102 | | | 97 | >30 |
| 103 | 97 | | | >30 |
| 104 | 99 | | | >30 |
| 105 | 99 | | | >30 |
| 106 | 97 | | | >30 |
| 107 | 99 | | | >30 |
| 108 | 98 | | | >30 |
| 109 | 99 | | | >30 |
| 110 | 99 | | | >30 |
| 111 | 95 | | | >30 |
| 112 | 98 | | | >30 |
| 113 | 67 | | | >30 |
| 114 | 98 | | | >30 |
| 115 | 99 | | | >30 |
| 116 | 96 | | | >30 |
| 117 | 100 | | | >30 |
| 118 | 101 | | | >30 |
| 119 | 101 | | | >30 |
| 120 | 101 | | | >30 |
| 121 | 97 | | | >30 |
| 122 | 99 | | | >30 |
| 123 | 95 | | | >30 |
| 124 | 97 | | | >30 |
| 125 | 100 | | | >30 |
| 126 | 51 | | | >30 |
| 127 | 98 | | | >30 |
| 128 | 97 | | | >30 |
| 129 | 98 | | | >30 |
| 130 | | | 37 | >30 |
| 131 | 100 | | | >30 |
| 132 | 99 | | | >30 |
| 133 | 96 | | | >30 |
| 134 | 96 | | | >30 |
| 135 | 98 | | | >30 |
| 136 | 92 | | | >30 |
| 137 | 98 | | | >30 |
| 138 | 93 | | | >30 |
| 139 | — | | 95 | >30 |
| 140 | 93 | | | >30 |
| 141 | | | 95 | >30 |
| 142 | 93 | | | >30 |
| 143 | 93 | | | >30 |
| 144 | 96 | | | >30 |
| 145 | 106 | | | >30 |
| 146 | 94 | | | >30 |
| 147 | 103 | | | >30 |
| 148 | 96 | | | >30 |
| 149 | 99 | | | |
| 150 | 70 | | | >30 |
| 151 | 98 | | | >30 |
| 152 | 92 | | | >30 |
| 153 | 101 | | | >30 |
| 154 | 93 | | | >30 |
| 155 | 99 | | | >30 |
| 156 | | | 97 | >30 |
| 157 | 102 | | | >30 |
| 158 | 99 | | | >30 |
| 159 | 103 | | | >30 |
| 160 | 84 | | | >30 |
| 161 | 97 | | | >30 |
| 162 | 97 | | | >30 |
| 163 | 97 | | | >30 |
| 164 | 95 | | | >30 |
| 165 | 103 | | | >30 |
| 166 | 103 | | | >30 |
| 167 | 98 | | | >30 |
| 168 | 98 | | | >30 |
| 169 | 103 | | | |
| 170 | | | 136 | |
| 171 | 100 | | | >30 |
| 172 | 61 | | | |
| 173 | 108 | | | >30 |
| 174 | 108 | | | |
| 175 | 112 | | | >30 |
| 176 | 101 | | | >30 |
| 177 | 108 | | | |
| 178 | 101 | | | |
| 179 | 102 | | | >30 |
| 180 | 99 | | | |
| 181 | 103 | | | |
| 182 | 102 | | | >30 |
| 183 | 96 | | | |
| 184 | 94 | | | >30 |
| 185 | 114 | | | |
| 186 | 100 | | | >30 |
| 187 | 97 | | | |
| 188 | 99 | | | |
| 189 | 99 | | | |
| 190 | 97 | | | >30 |
| 191 | 109 | | | >30 |
| 192 | 95 | | | >30 |
| 193 | 101 | | | >30 |
| 194 | 100 | | | >30 |
| 195 | 101 | | | >30 |
| 196 | 55 | | | |
| 197 | 60 | | | |
| 198 | 95 | | | >30 |
| 199 | 52 | | | >30 |
| 200 | 98 | | | >30 |
| 201 | 99 | | | |
| 202 | 97 | | | >30 |
| 203 | 99 | | | >30 |
| 204 | 98 | | | >30 |
| 205 | 95 | | | >30 |
| 206 | 97 | | | |
| 207 | 98 | | | >30 |
| 208 | 102 | | | >30 |
| 209 | 103 | | | |
| 210 | 101 | | | |
| 211 | 107 | | | >30 |
| 212 | 103 | | | |
| 213 | 104 | | | >30 |
| 214 | 107 | | | >30 |
| 215 | 105 | | | >30 |
| 216 | 104 | | | >30 |
| 217 | 108 | | | |
| 218 | 106 | | | >30 |
| 219 | 96 | | | |
| 220 | 71 | | | |
| 221 | 99 | | | |
| 222 | 98 | | | >30 |
| 223 | 100 | | | |
| 224 | 104 | | | >30 |
| 225 | 100 | | | |
| 226 | | 106 | | >30 |
| 227 | 100 | | | >30 |
| 228 | 79 | | | >30 |
| 229 | 99 | | | >30 |
| 230 | | 93 | | >30 |
| 231 | | 90 | | >30 |
| 232 | 97 | | | |
| 233 | | 93 | | |
| 234 | | 90 | | |
| 235 | | 105 | | >30 |
| 236 | | 93 | | |
| 237 | | 98 | | |
| 238 | | 97 | | |
| 239 | | 100 | | >30 |
| 240 | | 101 | | |
| 241 | | 101 | | >30 |
| 242 | | 104 | | |
| 243 | | 100 | | |
| 244 | | 100 | | |
| 245 | | 101 | | >30 |
| 246 | | 101 | | >30 |
| 247 | | 100 | | >30 |
| 248 | | 100 | | |
| 249 | | 98 | | >30 |
| 250 | | 103 | | |
| 251 | | 105 | | |
| 252 | | 103 | | >30 |
| 253 | | 95 | | >30 |
| 254 | | 100 | | |
| 255 | | 98 | | |
| 256 | | 89 | | |
| 257 | | 99 | | >30 |
| 258 | | 100 | | >30 |
| 259 | | 104 | | |
| 260 | | 96 | | >30 |
| 261 | | 80 | | |
| 262 | | 100 | | >30 |
| 263 | | 104 | | >30 |
| 264 | | 100 | | |
| 265 | | 95 | | >30 |
| 266 | | 98 | | >30 |
| 267 | | 101 | | |
| 268 | | 98 | | |
| 269 | | 100 | | >30 |
| 270 | | 98 | | |
| 271 | | 102 | | |
| 272 | | 95 | | >30 |
| 273 | | 99 | | >30 |
| 274 | | 102 | | >30 |
| 275 | | 101 | | 2.26 |
| 276 | | 99 | | |
| 277 | | 96 | | >30 |
| 278 | | 95 | | >30 |
| 279 | | 100 | | |
| 280 | | 99 | | |
| 281 | | 98 | | |
| 282 | | 55 | | |
| 283 | | 99 | | >30 |
| 284 | | 103 | | >30 |
| 285 | | 100 | | >30 |
| 286 | | 98 | | >30 |
| 287 | | 99 | | >30 |
| 288 | | 99 | | >30 |
| 289 | | 101 | | >30 |
| 290 | | 99 | | |
| 291 | | 98 | | >30 |
| 292 | | 101 | | |
| 293 | | 99 | | >30 |
| 294 | | 106 | | |
| 295 | | 105 | | >30 |
| 296 | | 104 | | >30 |
| 297 | | 90 | | >30 |
| 298 | | 99 | | >30 |
| 299 | | 99 | | >30 |
| 300 | | 99 | | >30 |
| 301 | | 98 | | >30 |
| 302 | | 96 | | >30 |
| 303 | | 100 | | >30 |
| 304 | | 103 | | |
| 305 | | 103 | | |
| 306 | | 98 | | |
| 307 | | 108 | | |
| 308 | | 103 | | >30 |
| 309 | | 99 | | |
| 310 | | 99 | | >30 |
| 311 | | 102 | | >30 |
| 312 | | 104 | | >30 |
| 313 | | 104 | | >30 |
| 314 | | 105 | | |
| 315 | | 101 | | |
| 316 | | 104 | | >30 |
| 317 | | 100 | | >30 |
| 318 | | 98 | | |
| 319 | | 100 | | >30 |
| 320 | | 94 | | |
| 321 | | 103 | | >30 |
| 322 | | 104 | | >30 |
| 323 | | 103 | | |
| 324 | | 100 | | >30 |
| 325 | | | 100 | |
| 326 | | | 111 | |

The results showed that compounds of the present invention suppress the production of IL-1β. It was also confirmed that the compounds of the present invention show more selective to IL-1β than TNF-α. These indicate that the compounds inhibit the targeted NLRP3 inflammasome activation pathway with little or no interference of the NF-κB-dependent priming pathway. Considering the diversity of pro-inflammatory factors, often with opposing functions, specific inhibition of the NLRP3 inflammasome pathway is required to achieve the most desired outcome without impeding the tissue repair process.

The following in vitro assays may be used to assess the ability of a compound of Formula 1 to enter the CNS through the blood-brain barrier.

### Multidrug Resistance Protein 1 (MDR1) Substrate Screening Assay

### Method 1

Human MDR1-expressing Madine-Darby Canine Kidney (MDCK) cells were cultured, and the transcellular transport study was performed. The cells were cultured in Transwell 96-well permeable support (pore size 0.4 m, 0.143 cm² surface area) with polycarbonate membrane (Corning Life Sciences, Lowell, MA). The cells were preincubated with Hanks' Balanced Salt Solution (HBSS) for 10 minutes at 37° C. Subsequently, transcellular transport was initiated by the addition of HBSS either to apical compartments (75 μL) or to basolateral compartments (250 μL) containing 1 or 10 mol/L of each test compound. The assay was terminated by separating each assay plate after 1 hour. Aliquots (25 μL) from the opposite compartments were mixed with acetonitrile. After centrifugation, the compound concentrations in the supernatant were measured by LC-MS/MS and an Unison UK-C18 HT column (3.0 μm, 2.0×20 mm). The apparent permeability (Papp) in the receiver wells was determined and the Papp and efflux ratio (ER) for the membrane permeability test was calculated from two-point standard curve (the concentration corresponding to 10 and 100 nm/sec) using the following equations:

$$Papp = ( R - y - axis intercept of R , STD 100 and R , STD 10 ) / Slope of R , STD 100 and R , STD 10$$

where R is ratio of peak area of test compound to that of internal standard, and R,STD10 and R,STD100 are ratio of peak area of standard sample corresponding to 10 and 100 nm/see to that of internal standard.

$$ER = Papp , BtoA / Papp , AtoB$$

where Papp,AtoB and Papp,BtoA represent the apparent permeability in the apical-to-basal direction and the basal-to-apical direction, respectively.

### Method 2

### 1. Cell Culture

MDR1-MDCK I cells were seeded onto polyethylene membranes (PET) in 96-well Corning insert systems at 2.5×105 cells/mL until to 4-7 days for confluent cell monolayer formation.

### 2. Experimental Procedures

For control compounds, the transport buffer in the study was HBSS with 10.0 mM HEPES at pH 7.40±0.05. For test compounds, the transport buffer in the study was HBSS with 10.0 mM HEPES and 1% BSA at pH 7.40±0.05. Test compounds were tested at 1.00 M bi-directionally in duplicate. Digoxin was tested at 10.0 M bi-directionally in duplicate, while nadolol and metoprolol were tested at 2.00 μM in A to B direction in duplicate. Final DMSO concentration was adjusted to less than 1%. The plate was incubated for 1 hour in CO₂ incubator at 37±1° C., with 5% CO₂ at saturated humidity without shaking. And all samples after mixed with acetonitrile containing internal standard were centrifuged at 3220×g for 10 minutes. For all samples, 150 μL supernatant solution was diluted with 150 μL ultra-pure water for LC-MS/MS analysis. In addition, the efflux ratio of each compound was also determined. Test and reference compounds were quantified by LC-MS/MS analysis based on the peak area ratio of analyte/IS.

After transport assay, Lucifer yellow rejection assay was applied to determine the cell monolayer integrity. Buffers were removed from both apical and basolateral chambers, followed by the addition of 75 μL of 100 μM lucifer yellow in transport buffer and 250 μL transport buffer in apical and basolateral chambers, respectively. The plate was incubated for 30 minutes at 37° C. with 5% CO₂ and 95% relative humidity without shaking. After 30 minutes incubation, 20 μL of lucifer yellow samples were taken from the apical sides, followed by the addition of 60 μL of Transport Buffer. And then 80 μL of lucifer yellow samples were taken from the basolateral sides. The relative fluorescence unit (RFU) of lucifer yellow was measured at 425/528 nm (excitation/emission) with a microplate reader.

### 3. Data Analysis

The apparent permeability coefficient Papp (cm/s) was calculated using the equation:

$$Papp = ( dCr / dt ) \times Vr / ( A \times C 0 )$$

Where dCr/dt is the cumulative concentration of compound in the receiver chamber as a function of time (μM/s); Vr is the solution volume in the receiver chamber (0.075 mL on the apical side, 0.25 mL on the basolateral side); A is the surface area for the transport, i.e. 0.0804 cm² for the area of the monolayer; CO is the initial concentration in the donor chamber (μM).
The efflux ratio was calculated using the equation:

$$Efflux Ratio = Papp ( BA ) / Papp ( AB )$$

Percent recovery was calculated using the equation:

$$\% Solution Recovery = 100 \times [ ( Vr \times Cr ) + ( Vd \times Cd ) ] / ( Vd \times C 0 )$$

Where Vd is the volume in the donor chambers (0.075 mL on the apical side, 0.25 mL on the basolateral side); Cd and Cr are the final concentrations of transport compound in donor and receiver chambers, respectively.
Percent of lucifer yellow in basolateral well was calculated using the equation:

$$\% Lucifer Yellow = \frac{{V}_{Basolateral} \times {RFU}_{Basolateral}}{{V}_{Apical} \times {RFU}_{Apical} + {V}_{Basolateral} \times {RFU}_{Basolateral}} \times 100$$

Where RFUApical and RFUBasolateral are the relative fluorescence unit values of lucifer yellow in the apical and basolateral wells, respectively; VApical and VBasolateral are the volume of apical and basolateral wells (0.075 mL and 0.25 mL), respectively. The % Lucifer Yellow should be less than 1.0.

### Breast Cancer Resistance Protein (BCRP) Substrate Screening Assay

Human BCRP-expressing MDCKII cells were cultured, and the transcellular transport study was performed. The cells were cultured in Transwell 96-well permeable support (pore size 0.4 m, 0.143 cm² surface area) with polycarbonate membrane (Corning Life Sciences, Lowell, MA). The cells were preincubated with M199 for 10 minutes at 37° C. Subsequently, transcellular transport was initiated by the addition of M199 either to apical compartments (75 μL) or to basolateral compartments (250 μL) containing 1 mol/L of each test compound. The assay was terminated by separating each assay plate after 1 hour. Aliquots (25 μL) from the opposite compartments were mixed with acetonitrile. After centrifugation, the compound concentrations in the supernatant were measured by LC-MS/MS and an Unison UK-C18 HT column (3.0 μm, 2.0×20 mm). The apparent permeability (Papp) in the receiver wells was determined and the Papp and efflux ratio (ER) for the membrane permeability test was calculated from two-point standard curve (the concentration corresponding to 10 and 100 nm/sec) using the following equations:

$$Papp = ( R - y - axis intercept of R , STD 100 and R , STD 10 ) / Slope of R , STD 100 and R , STD 10$$

where R is ratio of peak area of test compound to that of internal standard, and R,STD10 and R,STD100 are ratio of peak area of standard sample corresponding to 10 and 100 nm/sec to that of internal standard.

$$ER = Papp , BtoA / Papp , AtoB$$

where Papp,AtoB and Papp,BtoA represent the apparent permeability in the apical-to-basal direction and the basal-to-apical direction, respectively.

### Formulation Example 1 (Production of Capsule)

| | | |
|---|---|---|
| 1) compound of Example 1 | 30 | mg |
| 2) crystalline cellulose | 10 | mg |
| 3) lactose | 19 | mg |
| 4) magnesium stearate | 1 | mg |
| total | 60 | mg |

1), 2), 3) and 4) are mixed and filled in a gelatin capsule.

### Formulation Example 2 (Production of Tablet)

| | | |
|---|---|---|
| 1) compound of Example 1 | 30 | g |
| 2) lactose | 50 | g |
| 3) cornstarch | 15 | g |
| 4) calcium carboxymethylcellulose | 44 | g |
| 5) magnesium stearate | 1 | g |

| | |
|---|---|
| 1000 tablets | 140 g in total |

The total amount of 1), 2), 3) and 30 g of 4) are kneaded with water, vacuum dried and sieved. The sieved powder is mixed with 14 g of 4) and 1 g of 5), and the mixture is punched by a tableting machine. In this way, 1000 tablets containing 30 mg of the compound of Example 1 per tablet are obtained.

As used in this specification and the appended claims, singular articles such as “a,” “an,” and “the,” may refer to a single object or to a plurality of objects unless the context clearly indicates otherwise. Thus, for example, reference to a composition containing “a compound” may include a single compound or two or more compounds. The above description is intended to be illustrative and not restrictive. Many embodiments will be apparent to those of skill in the art upon reading the above description. Therefore, the scope of the invention should be determined with reference to the appended claims and includes the full scope of equivalents to which such claims are entitled. The disclosures of all articles and references cited in the disclosure, including patents, patent applications and publications, are herein incorporated by reference in their entirety and for all purposes.

What is claimed is:

1. A compound of Formula (I), or a pharmaceutically acceptable salt thereof: 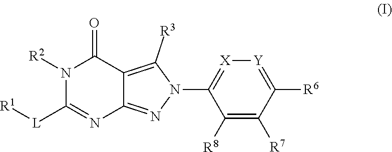
wherein
L is O or a bond;
X is N or CR⁴;
Y is N or CR⁵;
R¹ is an optionally substituted C₁₋₆ alkyl group, an optionally substituted C₃₋₈ cycloalkyl group, an optionally substituted C₆₋₁₄ aryl group, or an optionally substituted 4- to 6-membered heterocyclic group with the proviso that when L is a bond, then the 4- to 6-membered heterocyclic group is a 4- to 6-membered non-aromatic heterocyclic group which is linked to the pyrazolopyrimidone ring by a carbon-carbon bond;
R² is a hydrogen atom, an optionally substituted C₁₋₆ alkyl group, or an optionally substituted C₃₋₈ cycloalkyl group;
R³ is a hydrogen atom, an optionally substituted C₁₋₆ alkyl group, an optionally substituted C₃₋₈ cycloalkyl group, or a halogen atom; and
R⁴, R⁵, R⁶, R⁷ and R⁸ are each independently a hydrogen atom, an optionally substituted C₁₋₆ alkyl group, an optionally substituted C₃₋₈ cycloalkyl group, an optionally substituted C₂₋₆ alkenyl group, a halogen atom, a hydroxy group, an optionally substituted C₁₋₆ alkoxy group, or an optionally substituted 5- or 6-membered heterocyclic group a cyano group, an amino group or a nitro group;
with the proviso that (1) 2,5-dihydro-6-methyl-5-(1-methylethyl)-2-(3-pyridinyl)-4H-pyrazolo[3,4-d]pyrimidin-4-one, (2) 5-ethyl-2,5-dihydro-6-methyl-2-(3-pyridinyl)-4H-pyrazolo[3,4-d]pyrimidin-4-one and (3) 5-ethyl-2,5-dihydro-2-(3-pyridinyl)-6-(trifluoromethyl)-4H-pyrazolo[3,4-d]pyrimidin-4-one are excluded.

2. The compound or pharmaceutically acceptable salt thereof according to claim 1, wherein
L is O or a bond;
X is N or CR⁴;
Y is N or CR⁵;
R¹ is
(1) a C₁₋₆ alkyl group optionally substituted by 1 to 3 substituents selected from
(a) a halogen atom,
(b) a hydroxy group,
(c) an optionally halogenated C₁₋₆ alkoxy group,
(d) a di-C₁₋₆ alkylamino group,
(e) a C₇₋₁₆ aralkyloxy group,
(f) a 5- or 6-membered aromatic heterocyclic group,
(g) a 5- or 6-membered non-aromatic heterocyclic group,
(h) a carboxy group,
(i) a 4- to 6-membered non-aromatic heterocyclyloxy group, and
(j) a cyano group,
(2) a C₃₋₈ cycloalkyl group optionally substituted by 1 to 3 substituents selected from
(a) a C₁₋₆ alkyl group,
(b) a C₁₋₆ alkoxy group,
(c) a halogen atom,
(d) a cyano group, and
(e) a group represented by the formula: —(CH₂)ₐ—O—(CH₂)_{b}—, wherein each of a and b is the integer of 0 to 3 and the sum of a and b is 2 to 4,
(3) a C₆₋₁₄ aryl group optionally substituted by 1 to 3 substituents selected from
(a) a halogen atom, and
(b) a C₁₋₆ alkoxy group,
(4) a 5- or 6-membered aromatic heterocyclic group optionally substituted by 1 to 3 substituents selected from
(a) a halogen atom,
(b) a C₁₋₆ alkyl group optionally substituted by 1 to 3 substituents selected (i) halogen atom and (ii) C₁₋₆ alkoxy group,
(c) a C₁₋₆ alkoxy group, and
(d) a group represented by the formula: —(CH₂)ₐ—O—(CH₂)_{b}—, together with the 5- or 6-membered aromatic heterocyclic group to which it is attached, forming a fused 8- to 10-membered heterocyclic group, wherein each of a and b is the integer of 0 to 3 and the sum of a and b is 2 to 4, or
(5) a 4- to 6-membered non-aromatic heterocyclic group optionally substituted by 1 to 3 substituents selected from
(a) an oxo group,
(b) a C₁₋₆ alkyl group,
(c) a halogen atom, and
(d) a C₁₋₆ alkoxy group;
R² is
(1) a hydrogen atom, or
(2) a C₁₋₆ alkyl group;
R³ is
(1) a hydrogen atom,
(2) a C₁₋₆ alkyl group, or
(3) a halogen atom;
R⁴ and R⁸ are each independently
(1) a hydrogen atom,
(2) a C₁₋₆ alkyl group optionally substituted by 1 to 4 substituents selected from
(i) halogen atoms,
(ii) hydroxy group, and
(iii) C₁₋₆ alkoxy group,
(3) a C₃₋₈ cycloalkyl group optionally substituted by 1 to 3 halogen atoms,
(4) a halogen atom,
(5) a hydroxy group,
(6) a C₁₋₆ alkoxy group optionally substituted by 1 to 3 halogen atoms,
(7) a 5- or 6-membered aromatic heterocyclic group, or
(8) a cyano group;
R⁵ and R⁷ are each independently
(1) a hydrogen atom,
(2) a C₁₋₆ alkyl group optionally substituted by 1 to 3 halogen atoms,
(3) a halogen atom, or
(4) a C₃₋₈ cycloalkyl group; and
R⁶ is
(1) a hydrogen atom,
(2) a C₁₋₆ alkyl group optionally substituted by 1 to 3 halogen atoms,
(3) a C₃₋₈ cycloalkyl group optionally substituted by 1 to 3 halogen atoms,
(4) a C₂₋₆ alkenyl group,
(5) a halogen atom,
(6) a C₁₋₆ alkoxy group optionally substituted by 1 to 3 halogen atoms,
(7) a 5- or 6-membered aromatic heterocyclic group,
(8) an amino group, or
(9) a nitro group.

3. The compound or pharmaceutically acceptable salt thereof according to claim 1, wherein
L is O or bond;
X is CR⁴;
Y is CR⁵;
R¹ is a C₁₋₆ alkyl group optionally substituted by an optionally halogenated C₁₋₆ alkoxy group;
R² is a hydrogen atom;
R³ is a hydrogen atom;
R⁴ and R⁸ are each independently
(1) a C₁₋₆ alkyl group optionally substituted by 1 to 3 halogen atoms, or
(2) a halogen atom;
R⁵ and R⁷ are both hydrogen atoms; and
R⁶ is
(1) a C₃₋₈ cycloalkyl group optionally substituted by 1 to 3 halogen atoms,
(2) a halogen atom, or
(3) a C₁₋₆ alkoxy group optionally substituted by 1 to 3 halogen atoms.

4. The compound or pharmaceutically acceptable salt thereof according to claim 1, wherein
L is O;
X is N or CR⁴;
Y is N or CR⁵;
R¹ is an optionally substituted C₁₋₆ alkyl group, an optionally substituted C₃₋₈ cycloalkyl group, an optionally substituted C₆₋₁₄ aryl group, or an optionally substituted 4- to 6-membered heterocyclic group;
R² is a hydrogen atom, or an optionally substituted C₁₋₆ alkyl group;
R³ is a hydrogen atom, an optionally substituted C₁₋₆ alkyl group, or a halogen atom;
R⁴ and R⁸ are each independently a hydrogen atom, an optionally substituted C₁₋₆ alkyl group, an optionally substituted C₃₋₈ cycloalkyl group, a halogen atom, a hydroxy group, an optionally substituted C₁₋₆ alkoxy group, an optionally substituted 5- or 6-membered heterocyclic group or a cyano group;
R⁵ and R⁷ are each independently a hydrogen atom, an optionally substituted C₁₋₆ alkyl group, or a halogen atom; and
R⁶ is a hydrogen atom, an optionally substituted C₁₋₆ alkyl group, an optionally substituted C₃₋₈ cycloalkyl group, an optionally substituted C₂₋₆ alkenyl group, a halogen atom, an optionally substituted C₁₋₆ alkoxy group, or an optionally substituted 5- or 6-membered heterocyclic group.

5. The compound or pharmaceutically acceptable salt thereof according to claim 1, wherein
L is O;
X is N or CR⁴;
Y is N or CR⁵;
R¹ is
(1) a C₁₋₆ alkyl group optionally substituted by 1 to 3 substituents selected from
(a) a halogen atom,
(b) a hydroxy group,
(c) an optionally halogenated C₁₋₆ alkoxy group,
(d) a di-C₁₋₆ alkylamino group, and
(e) a C₇₋₁₆ aralkyloxy group,
(2) a C₃₋₈ cycloalkyl group optionally substituted by 1 to 3 substituents selected from
(a) a C₁₋₆ alkoxy group,
(b) a cyano group, and
(c) a group represented by the formula: —(CH₂)ₐ—O—(CH₂)_{b}—, wherein each of a and b is the integer of 0 to 3 and the sum of a and b is 2 to 4,
(3) a C₆₋₁₄ aryl group, or
(4) a 4- to 6-membered non-aromatic heterocyclic group;
R² is
(1) a hydrogen atom, or
(2) a C₁₋₆ alkyl group;
R³ is
(1) a hydrogen atom,
(2) a C₁₋₆ alkyl group, or
(3) a halogen atom;
R⁴ and R⁸ are each independently
(1) a hydrogen atom,
(2) a C₁₋₆ alkyl group optionally substituted by 1 to 4 substituents selected from
(i) halogen atoms,
(ii) hydroxy group, and
(iii) C₁₋₆ alkoxy group,
(3) a C₃₋₈ cycloalkyl group optionally substituted by 1 to 3 halogen atoms,
(4) a halogen atom,
(5) a hydroxy group,
(6) a C₁₋₆ alkoxy group,
(7) a 5- or 6-membered aromatic heterocyclic group, or
(8) a cyano group;
R⁵ and R⁷ are each independently
(1) a hydrogen atom,
(2) a C₁₋₆ alkyl group, or
(3) a halogen atom; and
R⁶ is
(1) a hydrogen atom,
(2) a C₁₋₆ alkyl group optionally substituted by 1 to 3 halogen atoms,
(3) a C₃₋₈ cycloalkyl group optionally substituted by 1 to 3 halogen atoms,
(4) a C₂₋₆ alkenyl group,
(5) a halogen atom,
(6) a C₁₋₆ alkoxy group optionally substituted by 1 to 3 halogen atoms, or
(7) a 5- or 6-membered aromatic heterocyclic group.

6. The compound or pharmaceutically acceptable salt thereof according to claim 1, wherein
L is O;
X is CR⁴;
Y is CR⁵;
R¹ is a C₁₋₆ alkyl group optionally substituted by 1 to 3 C₁₋₆ alkoxy group;
R² is a hydrogen atom;
R³ is a hydrogen atom;
R⁴ and R⁸ are each independently
(1) a C₁₋₆ alkyl group optionally substituted by 1 to 3 halogen atoms, or
(2) a halogen atom;
R⁵ and R⁷ are both hydrogen atoms; and
R⁶ is
(1) a C₃₋₈ cycloalkyl group optionally substituted by 1 to 3 halogen atoms,
(2) a halogen atom, or
(3) a C₁₋₆ alkoxy group optionally substituted by 1 to 3 halogen atoms.

7. The compound or pharmaceutically acceptable salt thereof according to claim 1, wherein
L is O;
X is CR⁴;
Y is CR⁵;
R¹ is a C₁₋₆ alkyl group;
R² is a hydrogen atom;
R³ is a hydrogen atom;
R⁴ and R⁸ are both C₁₋₆ alkyl groups;
R⁵ and R⁷ are both hydrogen atoms; and
R⁶ is a C₃₋₈ cycloalkyl group.

8. The compound or pharmaceutically acceptable salt thereof according to claim 1, wherein
L is a bond;
X is N or CR⁴;
Y is N or CR⁵;
R¹ is an optionally substituted C₁₋₆ alkyl group, an optionally substituted C₃₋₈ cycloalkyl group, an optionally substituted C₆₋₁₄ aryl group, or an optionally substituted 4- to 6-membered non-aromatic heterocyclic group;
R² is a hydrogen atom, or an optionally substituted C₁₋₆ alkyl group;
R³ is a hydrogen atom, an optionally substituted C₁₋₆ alkyl group, or a halogen atom;
R⁴ and R⁸ are each independently a hydrogen atom, an optionally substituted C₁₋₆ alkyl group, an optionally substituted C₃₋₈ cycloalkyl group, a halogen atom, a hydroxy group, an optionally substituted C₁₋₆ alkoxy group, an optionally substituted 5- or 6-membered heterocyclic group or a cyano group;
R⁵ and R⁷ are each independently a hydrogen atom, an optionally substituted C₁₋₆ alkyl group, a halogen atom, or an optionally substituted C₃₋₈ cycloalkyl group; and
R⁶ is a hydrogen atom, an optionally substituted C₁₋₆ alkyl group, an optionally substituted C₃₋₈ cycloalkyl group, an optionally substituted C₂₋₆ alkenyl group, a halogen atom, an optionally substituted C₁₋₆ alkoxy group, an optionally substituted 5- or 6-membered heterocyclic group, an amino group, or a nitro group.

9. The compound or pharmaceutically acceptable salt thereof according to claim 1, wherein
L is a bond;
X is CR⁴;
Y is CR⁵;
R¹ is
(1) a C₁₋₆ alkyl group optionally substituted by 1 to 3 substituents selected from
(a) a halogen atom,
(b) a hydroxy group,
(c) an optionally halogenated C₁₋₆ alkoxy group,
(d) a C₇₋₁₆ aralkyloxy group,
(e) a 5- or 6-membered aromatic heterocyclic group,
(f) a 5- or 6-membered non-aromatic heterocyclic group,
(g) a carboxy group,
(h) a 4- to 6-membered non-aromatic heterocyclyloxy group, and
(i) a cyano group,
(2) a C₃₋₈ cycloalkyl group optionally substituted by 1 to 3 substituents selected from
(a) a C₁₋₆ alkyl group,
(b) a C₁₋₆ alkoxy group,
(c) a halogen atom, and
(d) a cyano group,
(3) a C₆₋₁₄ aryl group optionally substituted by 1 to 3 substituents selected from
(a) a halogen atom, and
(b) a C₁₋₆ alkoxy group, or
(4) a 4- to 6-membered non-aromatic heterocyclic group optionally substituted by 1 to 3 substituents selected from
(a) an oxo group,
(b) a C₁₋₆ alkyl group,
(c) a halogen atom, and
(d) a C₁₋₆ alkoxy group;
R² is
(1) a hydrogen atom, or
(2) a C₁₋₆ alkyl group;
R³ is
(1) a hydrogen atom, or
(2) a C₁₋₆ alkyl group;
R⁴ and R⁸ are each independently
(1) a hydrogen atom,
(2) a C₁₋₆ alkyl group optionally substituted by 1 to 4 halogen atoms,
(3) a halogen atom,
(4) a hydroxy group, or
(5) a C₁₋₆ alkoxy group;
R⁵ and R⁷ are each independently
(1) a hydrogen atom,
(2) a C₁₋₆ alkyl group optionally substituted by 1 to 3 halogen atoms,
(3) a halogen atom, or
(4) a C₃₋₈ cycloalkyl group; and
R⁶ is
(1) a hydrogen atom,
(2) a C₁₋₆ alkyl group
(3) a C₃₋₈ cycloalkyl group optionally substituted by 1 to 3 halogen atoms,
(4) a halogen atom,
(5) an amino group, or
(6) a nitro group.

10. The compound or pharmaceutically acceptable salt thereof according to claim 1, wherein
L is bond;
X is CR⁴;
Y is CR⁵;
R¹ is a C₁₋₆ alkyl group optionally substituted by an optionally halogenated C₁₋₆ alkoxy group;
R² is a hydrogen atom;
R³ is a hydrogen atom;
R⁴ and R⁸ are both C₁₋₆ alkyl groups optionally substituted by 1 to 3 halogen atoms;
R⁵ and R⁷ are both hydrogen atoms; and
R⁶ is a C₃₋₈ cycloalkyl group.

11. The compound or pharmaceutically acceptable salt thereof according to claim 1, wherein the compound is selected from:
2-(4-bromo-2-fluoro-6-methylphenyl)-6-ethoxy-2,5-dihydro-4H-pyrazolo[3,4-d]pyrimidin-4-one;
2-(4-cyclopropyl-2-fluoro-6-methylphenyl)-6-ethoxy-2,5-dihydro-4H-pyrazolo[3,4-d]pyrimidin-4-one;
2-[2-bromo-4-(difluoromethoxy)-6-methylphenyl]-6-ethoxy-2,5-dihydro-4H-pyrazolo[3,4-d]pyrimidin-4-one;
2-(4-cyclopropyl-2,6-dimethylphenyl)-6-methoxy-2,5-dihydro-4H-pyrazolo[3,4-d]pyrimidin-4-one;
2-[4-cyclopropyl-2-(difluoromethyl)-6-fluorophenyl]-6-(2-methoxyethoxy)-2,5-dihydro-4H-pyrazolo[3,4-d]pyrimidin-4-one;
2-(4-cyclopropyl-2,6-dimethylphenyl)-6-[(difluoromethoxy)methyl]-2,5-dihydro-4H-pyrazolo[3,4-d]pyrimidin-4-one;
2-[4-(1-fluorocyclopropyl)-2,6-dimethylphenyl]-6-methoxy-2,5-dihydro-4H-pyrazolo[3,4-d]pyrimidin-4-one; and
2-[4-cyclopropyl-2-(difluoromethyl)-6-methylphenyl]-6-(methoxymethyl)-2,5-dihydro-4H-pyrazolo[3,4-d]pyrimidin-4-one.

12. A method of treating a disease, disorder or condition in a subject, which comprises administering to the subject an effective amount of a compound or pharmaceutically acceptable salt as defined in claim 1, wherein the disease, disorder or condition is associated with NLRP3.

13. A method of treating a disease, disorder or condition in a subject, which comprises administering to the subject an effective amount of a compound or pharmaceutically acceptable salt as defined in claim 1, wherein the disease, disorder or condition is associated with a heterozygous gain of function mutation in the NLRP3 gene.

14. A method of treating a cryopyrin-associated periodic syndrome (CAPS) in a subject, which comprises administering to the subject an effective amount of a compound or pharmaceutically acceptable salt as defined in claim 1.

15. The method according to claim 14, wherein the cryopyrin-associated periodic syndrome is selected from the group consisting of neonatal-onset multisystem inflammatory disease (NOMID/CINCA), Muckle-Wells syndrome (MWS), and familial cold autoinflammatory syndrome (FCAS).

16. A method of treating a neurodegenerative disease, disorder or condition in a subject, which comprises administering to the subject an effective amount of a compound or pharmaceutically acceptable salt as defined in claim 1.

17. A method of treating Parkinson's disease, Alzheimer's disease, Huntington's disease, amyotrophic lateral sclerosis or prion disease in a subject, which comprises administering to the subject an effective amount of a compound or pharmaceutically acceptable salt as defined in claim 1.

18. A medicament comprising a compound or pharmaceutically acceptable salt as defined in claim 1.

19. The medicament according to claim 18, which is an agent for the treatment of disease, disorder or condition associated with NLRP3.

20. The medicament according to claim 18, which is an agent for the treatment of disease, disorder or condition associated with a heterozygous gain of function mutation in the NLRP3 gene.

21. The medicament according to claim 18, which is an agent for the treatment of a cryopyrin-associated periodic syndrome (CAPS).

22. The medicament according to claim 21, wherein the cryopyrin-associated periodic syndrome is selected from neonatal-onset multisystem inflammatory disease (NOMID/CINCA), Muckle-Wells syndrome (MWS), and familial cold autoinflammatory syndrome (FCAS).

23. Use of a compound or pharmaceutically acceptable salt thereof as defined in claim 1 for the manufacture of a medicament for the treatment of disease, disorder or condition associated with NLRP3.

24. Use of a compound or pharmaceutically acceptable salt thereof as defined in claim 1 for the manufacture of a medicament for the treatment of disease, disorder or condition associated with a heterozygous gain of function mutation in the NLRP3 gene.

25. Use of a compound or pharmaceutically acceptable salt thereof as defined in claim 1 for the manufacture of a medicament for the treatment of a cryopyrin-associated periodic syndrome (CAPS).

26. The use according to claim 25 wherein the cryopyrin-associated periodic syndrome is selected from neonatal-onset multisystem inflammatory disease (NOMID/CINCA), Muckle-Wells syndrome (MWS), and familial cold autoinflammatory syndrome (FCAS).

27. A compound or pharmaceutically acceptable salt thereof as defined in claim 1 for use in treating a disease, disorder or condition associated with NLRP3.

28. A compound or pharmaceutically acceptable salt thereof as defined in claim 1 for use in treating a disease, disorder or condition associated with a heterozygous gain of function mutation in the NLRP3 gene.

29. A compound or pharmaceutically acceptable salt thereof as defined in claim 1 for use in treating a cryopyrin-associated periodic syndrome (CAPS).

30. The compound according to claim 29, wherein the cryopyrin-associated periodic syndrome is selected from neonatal-onset multisystem inflammatory disease (NOMID/CINCA), Muckle-Wells syndrome (MWS), and familial cold autoinflammatory syndrome (FCAS).
